(19) United States
(12) Patent Application Publication
(10) Pub. No.: US 2026/0250250 A1
(43) Pub. Date: Aug. 27, 2026

(54) 3-(SULPHONYL OR SULFONIMIDOYL)PROP-2-EN-1-YL]-2-OXO-1,2-DIHYDROPYRIDINE-3-CARBOXAMIDE DERIVATIVES

(71) Applicant: Flinder Therapeutics B.V., Oss (NL)

(72) Inventors: Maarten Alexander Ligtenberg, Oss (NL); Jacobus Cornelis Henricus Maria Wijkmans, Oss (NL); Ewelina Anna Urbanska, Oss (NL); Andrew Eric Shouksmith, Oss (NL); Alina Samarskaya, Oss (NL); Massimiliano Travagli, Oss (NL)

(21) Appl. No.: 18/877,500

(22) PCT Filed: Jun. 23, 2023
(86) PCT No.: PCT/NL2023/050347

§ 371 Date: Dec. 20, 2024

(30) Foreign Application Priority Data
Jun. 23, 2022 (NL) 2032265

(51) Int. Cl. C07D 215/48; A61K 31/4375; A61K 31/47; A61K 38/19; C07D 221/04; C07D 471/04

(52) U.S. Cl. CPC C07D 215/48; A61K 31/4375; A61K 31/47; A61K 38/191; C07D 221/04; C07D 471/04

(57) ABSTRACT

The present invention relates to 3-(sulphonyl or sulfonimidoyl)prop-2-en-1-yl]-2-oxo-1,2-dihydropyridine-3-carboxamide derivatives having the formula: or the pharmaceutical acceptable salt thereof. The present invention further relates to the compound according to the invention for use as a medicament and also for use in the treatment of cancer. The present invention further relates to a method for inhibiting Met1-linked ubiquitination. The present invention also relates to a pharmaceutical composition comprising the compound of the present invention.

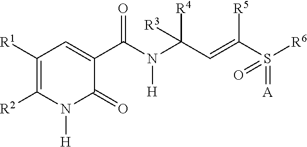

The present invention relates to 3-(sulphonyl or sulfonimidoyl)prop-2-en-1-yl]-2-oxo-1,2-dihydropyridine-3-carboxamide derivatives or the pharmaceutical acceptable salts thereof. The present invention further relates to the compound according to the invention for use as a medicament and also for use in the treatment of cancer. The present invention further relates to a method for inhibiting Met1-linked ubiquitination. The present invention also relates to a pharmaceutical composition comprising the compound of the present invention.

Cellular homeostasis depends on adaptation to the environment through numerous signalling cascades that translate extrinsic and intrinsic cues and stresses into appropriate responses. Signalling is most commonly propagated through rapid and specific post-translational modifications (PTMs) of signalling proteins, resulting in adaptive changes at the transcriptional and translational levels. Failure to adapt can cause cell damage or cell death and may lead to disease. Methionine 1 (Met1)-linked ubiquitin (also referred to as linear ubiquitin) is a PTM that over the past decade has emerged as critically important for controlling vital signalling cascades and preventing cell death. Consequently, dysregulation of Met1-linked ubiquitin modifications is associated with severe pathologies, including immune disorders, cancer, and neurodegeneration, emphasising that the balance of one single subtype of PTM can be essential for maintaining homeostasis, preventing and treating life-threatening diseases. The linear ubiquitin (Ub) chain assembly complex (LUBAC) is the only known mammalian ubiquitin ligase that makes Met1-linked ubiquitin. Consequently, by controlling the function of LUBAC, the role and regulation of the Met1-linked ubiquitin can be controlled.

LUBAC is composed of three proteins, HOIL-1-interacting protein (HOIP; also known as RNF31), Heme-Oxidised IRP2 Ub Ligase-1 (HOIL-1; also known as RBCK1), and SHANK-Associated RH Domain-Interacting Protein (SHARPIN). It was discovered that of these three proteins, HOIP (RNF31) has remarkable specificity and exclusively assembles Met1-Ub, whereas HOIL-1 and SHARPIN function as co-factors in LUBAC and are critical for HOIP activation. LUBAC was first described to regulate signalling in response to activation of tumour necrosis factor (TNF) receptor 1 (TNF-R1) and has since been found to regulate signal transduction by a wide range of NF-κB-activating immune receptors, including cytokine receptors, Toll-like receptors (TLRs), NOD-like receptors (NLRs), and antigen receptors. Therefore, LUBAC can be considered one of the core components of NF-κB-activating signalling pathways.

Further detailed information about the mechanisms and pathways involved in the Met1-linked ubiquitin PTM and subsequent control of vital signalling cascades and preventing cell death is described by, for example, Hrdinka (“The Met1-linked ubiquitin machinery: emerging themes of (De) regulation.” Molecular cell 68.2 (2017): 265-280), Ning (“Structures, functions, and inhibitors of LUBAC and its related diseases.” Journal of Leukocyte Biology (2022)), Jahan (“Met1-linked ubiquitin signalling in health and disease: inflammation, immunity, cancer, and beyond.” Cell Death & Differentiation 28.2 (2021): 473-492) and Oikawa (“Cellular and Mathematical Analyses of LUBAC Involvement in T Cell Receptor-Mediated NF-KB Activation Pathway.” Frontiers in immunology (2020): 3042).

Given the above, by controlling the function of LUBAC, the role and regulation of the Met1-linked ubiquitin can be controlled, and, consequently, the development of diseases like cancers can be controlled subsequently. It is believed that by providing Met1-linked ubiquitination inhibitors, in particular LUBAC associated HOIP (RNF31) inhibitors, promising candidates for the treatment of cancers can be identified. In a first aspect of the present invention, the present invention provides hereto a compound having the formula (I):

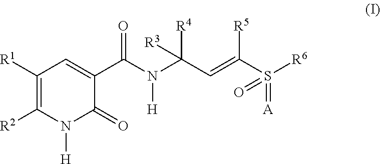

or the pharmaceutical acceptable salt thereof, wherein R¹ and R² are both hydrogen or wherein the 2-oxo-1,2-dihydropyridine moiety:

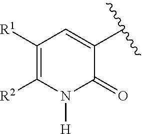

is selected from the group consisting of the following 2-oxo-1,2-dihydropyridine moieties:

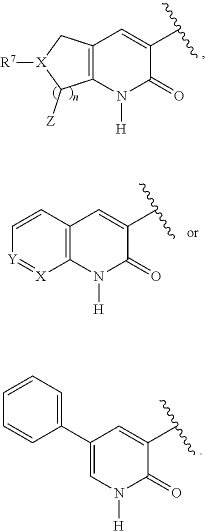

Wherein:
n is 1, 2, 3 or 4;
A is oxygen or nitrogen;
X is carbon or nitrogen or wherein R⁷—X is oxygen;
Y is carbon or nitrogen;
Z is hydrogen, (C1-C6)alkyl or di-(C1-C6)alkyl;
R³ and R⁴ are each independently selected from the group consisting of hydrogen, (C1-C4)alkyl or wherein R³ and R⁴ together with the carbon to which they are attached form (C3-C6)cycloalkyl;
R⁵ is selected from the group consisting of hydrogen, halo or (C1-C6)alkyl;
R⁶ is selected from the group consisting of (C1-C6)alkyl, (C2-C6)alkenyl, (C2-C6)alkynyl, (C3-C10)cycloalkyl, (C2-C9)heterocycloalkyl, (C6-C10)aryl, (C6-C10)aryl(C1-C6)alkyl or (C2-C9)heteroaryl, wherein the cycloalkyl, heterocycloalkyl, aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino;
when X is carbon, R⁷ is R^{7′} and R^{7″}, wherein R^{7′} is selected from the group consisting of hydrogen, halo or (C1-C6)alkyl, and wherein R^{7″} is selected from the group consisting of hydrogen, halo, (C1-C6)alkyl, (C1-C6)alkoxy, (C2-C6)alkenyl, (C2-C6)alkynyl, (C6-C10)aryl(C1-C6)alkyl, (C2-C9)heteroaryl(C1-C6)alkyl, wherein the aryl and heteroaryl groups are optionally substituted with one to three (C1-C6)alkyl;
when X is nitrogen, R⁷ is selected from the group consisting of hydrogen, (C1-C6)alkyl, (C3-C10)cycloalkyl(C1-C6)alkyl, (C6-C10)aryl(C1-C6)alkyl, (C2-C9)heteroaryl(C1-C6)alkyl, wherein the aryl and heteroaryl groups are optionally substituted with one to three (C1-C6)alkyl or wherein R⁷ is R⁸M-, wherein:
   R⁸M- is selected from the group consisting of R⁸—C(O)—, R⁸—O—C(O)—, R⁸—NH—C(O)— or R⁸—S(O)₂—; and
   R⁸ is selected from the group consisting of (C1-C6)alkyl, (C3-C10)cycloalkyl, (C3-C10)cycloalkyl(C1-C6)alkyl, (C2-C9)heterocycloalkyl, (C4-C9)spiroheterocycloalkyl, (C1-C6)alkoxy(C1-C6)alkyl, (C6-C10)aryl(C1-C6)alkyl, (C2-C9)heteroaryl, (C2-C9)heteroaryl(C1-C6)alkyl, (C4-C9)bicycloalkyl, wherein the alkyl, cycloalkyl, heterocycloalkyl, aryl, heteroaryl and bicycloalkyl groups are optionally substituted with one to four halo, (C1-C6)alkyl, (C3-C6)cycloalkyl, (C1-C6)acyl, (C6-C10)aryl, (C1-C6)alkoxy, cyano, oxo or hydroxy.

It was found that the compound of the present invention exhibits promising RNF31 inhibiting properties (in a cell-free HTRF-based HOIP inhibition assay), thus providing a promising candidate as Met1-linked ubiquitination inhibitor. It was further found that the compound of the present invention downregulates the nuclear factor kappa-light-chain-enhancer of activated B cells (NF-κB) signalling pathway and/or upregulates necrosis factor alpha (TNFα) induced cytotoxicity.

Given the compounds of the present invention, it was found that compounds wherein R³, R⁴ and R⁵ are hydrogen exhibit further improved RNF31 inhibiting properties as well as improved downregulating properties of the NF-κB signalling pathway and/or upregulating properties of TNFα induced cytotoxicity.

Similar, it was found that by selecting R⁶ from the group consisting of (C6-C10)aryl or (C2-C9)heteroaryl, wherein the aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino, even more promising candidates for Met1-linked ubiquitination inhibition are identified.

Preferably, when X is carbon, R⁷ is R^{7′} and R^{7″}, wherein R^{7′} is selected from the group consisting of hydrogen, halo or (C1-C6)alkyl, and wherein R^{7″} is selected from the group consisting of hydrogen, (C1-C6)alkyl, (C6-C10)aryl(C1-C6)alkyl, (C2-C9)heteroaryl(C1-C6)alkyl, wherein the aryl and heteroaryl groups are optionally substituted with one to three (C1-C6)alkyl;
when X is nitrogen, R⁷ is selected from the group consisting of hydrogen, (C1-C6)alkyl, (C6-C10)aryl(C1-C6)alkyl, (C2-C9)heteroaryl(C1-C6)alkyl, wherein the aryl and heteroaryl groups are optionally substituted with one to three (C1-C6)alkyl or wherein R⁷ is R⁸M-, wherein:
   R⁸M- is selected from the group consisting of R⁸—O—C(O)—, R⁸—NH—C(O)— or R⁸—S(O)₂—; and
   R⁸ is selected from the group consisting of (C1-C6)alkyl, (C3-C10)cycloalkyl, (C3-C10)cycloalkyl(C1-C6)alkyl, (C2-C9)heterocycloalkyl, (C4-C9)spiroheterocycloalkyl, (C1-C6)alkoxy(C1-C6)alkyl, (C6-C10)aryl(C1-C6)alkyl, (C2-C9)heteroaryl, (C2-C9)heteroaryl(C1-C6)alkyl, (C4-C9)bicycloalkyl, wherein the alkyl, cycloalkyl, heterocycloalkyl, aryl, heteroaryl and bicycloalkyl groups are optionally substituted with one to four halo, (C1-C6)alkyl, (C3-C6)cycloalkyl, (C1-C6)acyl, (C6-C10)aryl, (C1-C6)alkoxy, cyano, oxo or hydroxy.

In an embodiment of the present invention, n may be 1, 2 or 3 resulting in compounds having improved Met1-linked ubiquitination inhibiting properties. It is further noted that in a preferred embodiment the present invention relates to wherein when X is nitrogen, R⁷ is not hydrogen.

In a specific embodiment of the present invention, the present invention relates to a compound having the formula (II):

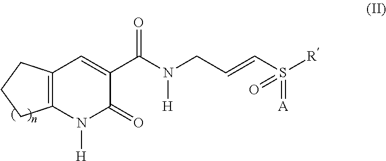

or the pharmaceutical acceptable salt thereof, wherein:
n is 1, 2 or 3;
A is oxygen or nitrogen; and
R′ is selected from the group consisting of (C6-C10)aryl or (C2-C9)heteroaryl, wherein the aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino. Preferably, in relation to formula (II) it was found that particular active compounds are obtained wherein (C6-C10)aryl is phenyl or (C2-C9)heteroaryl is pyridinyl, and wherein the phenyl and pyridinyl groups are optionally substituted with one to three fluorine, chlorine, bromine or methoxy.

In a further specific embodiment of the present invention, the present invention relates to a compound having the formula (III):

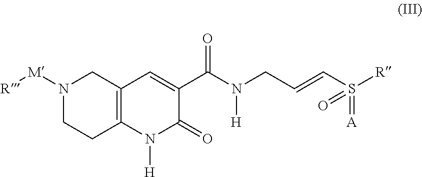

or the pharmaceutical acceptable salt thereof, wherein:
A is oxygen or nitrogen;
R″ is selected from the group consisting of (C6-C10)aryl or (C2-C9)heteroaryl, wherein the aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino;
M′ is carbon or —O—C(O)—; and
R′″ is selected from the group consisting of (C1-C6)alkyl, (C3-C10)cycloalkyl, (C3-C10)cycloalkyl(C1-C6)alkyl, (C2-C9)heterocycloalkyl, (C4-C9)spiroheterocycloalkyl, (C1-C6)alkoxy(C1-C6)alkyl, (C6-C10)aryl(C1-C6)alkyl, (C2-C9)heteroaryl, (C2-C9)heteroaryl(C1-C6)alkyl, (C4-C9)bicycloalkyl, wherein the alkyl, cycloalkyl, heterocycloalkyl, aryl, heteroaryl and bicycloalkyl groups are optionally substituted with one to four halo, (C1-C6)alkyl, (C3-C6)cycloalkyl, (C1-C6)acyl, (C6-C10)aryl, (C1-C6)alkoxy, cyano, oxo or hydroxy. Preferably, in relation to formula (III) it was found that particular active compounds are obtained wherein A is nitrogen.

Where any of the groups in the compounds of the present invention is stated to be optionally substituted, this group may be unsubstituted, or substituted by one or more substituents. Typically, such groups will be unsubstituted, or substituted by one, two or three substituents, in particular one or two substituents.

For use in medicine, the salts of the compounds of the present invention will be pharmaceutically acceptable salts. Other salts may, however, be useful in the preparation of the compounds of use in the invention or of their pharmaceutically acceptable salts.

Suitable pharmaceutically acceptable salts of the compounds of the invention include acid addition salts which may, for example, be formed by mixing a solution of the compound of use in the invention with a solution of a pharmaceutically acceptable acid such as hydrochloric acid, sulphuric acid, methane sulphonic acid, fumaric acid, maleic acid, succinic acid, acetic acid, benzoic acid, citric acid, tartaric acid or phosphoric acid. Furthermore, where the compounds of use in the invention carry an acidic moiety, e.g. carboxy, suitable pharmaceutically acceptable salts thereof may include alkali metal salts, e.g. sodium or potassium salts; alkaline earth metal salts, e.g. calcium or magnesium salts; ammonium salts; and salts formed with suitable organic ligands, e.g. quaternary ammonium salts, and meglumine salts.

The present invention includes solvates of the compounds of the present invention. Such solvates may be formed with common organic solvents, e.g. hydrocarbon solvents such as benzene or toluene; chlorinated solvents such as chloroform or dichloromethane; alcoholic solvents such as methanol, ethanol or isopropanol; ethereal solvents such as diethyl ether or tetrahydrofuran; or ester solvents such as ethyl acetate. Alternatively, the solvates of the compounds of formula (I) may be formed with water, in which case they will be hydrates.

The present invention also includes co-crystals of the compounds of the present invention. The term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity. Typical examples of co-crystal formers, which may be present in the co-crystal alongside the active pharmaceutical ingredient, include L-ascorbic acid, citric acid, glutaric acid, urea and nicotinamide.

The present invention further includes prodrugs of the compounds of the present invention. In general, such prodrugs will be functional derivatives of the compounds of the present invention which are readily convertible in vivo into the required compound of the present invention.

Suitable alkyl groups which may be present on the compounds of the present invention include straight-chained and branched (C1-C6)alkyl groups, for example (C1-C4)alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “(C1-C6)alkoxy”, and “(C1-C6)acyl” are to be construed accordingly. Further, the term “di-(C1-C6)alkyl” as used herein is intended to refer to two alkyl groups comprising 1 to 6 carbon atoms attached to the same carbon.

Suitable alkenyl groups which may be present on the compounds of the present invention include straight-chained and branched (C2-C6)alkenyl groups including vinyl and allyl. Suitable alkynyl groups which may be present in the compounds of the present invention include straight-chained and branched (C2-C6)alkynyl groups including ethynyl, propargyl and butynyl.

The term “(C3-C10)cycloalkyl” as used herein refers to monovalent groups of 3 to 10 carbon atoms derived from a saturated monocyclic hydrocarbon, and may comprise benzo-fused analogues thereof. Suitable (C3-C10)cycloalkyl groups include cyclopropyl, cyclobutyl, benzocyclobutenyl, cyclopentyl, indanyl, cyclohexyl and cycloheptyl. The term “(C3-C6)cycloalkyl” as used herein refers to monovalent groups of 3 to 6 carbon atoms derived from a saturated monocyclic hydrocarbon, such as including cyclopropyl, cyclobutyl, cyclopentyl and cyclohexyl groups. The term “(C3-C10)cycloalkyl” may also refer to cycloalkenyl groups, and may comprise benzo-fused analogues thereof.

The term “(C2-C9)heterocycloalkyl” as used herein refers to saturated monocyclic rings containing 2 to 9 carbon atoms, preferably 3 to 7 carbon atoms, and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzofuranyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, tetrahydrothiopyranyl, piperidinyl, 1,2,3,4-tetrahydroquinolinyl, 1,2,3,4-tetrahydroisoquinolinyl, piperazinyl, 1,2,3,4-tetrahydroquinoxalinyl, hexahydro-[1,2,5]thiadiazolo[2,3-a]pyrazinyl, homopiperazinyl, morpholinyl, benzoxazinyl, thiomorpholinyl, azepanyl, oxazepanyl, diazepanyl, thiadiazepanyl and azocanyl groups.

The term “(C6-C10)aryl” as used herein refers to monovalent carbocyclic aromatic groups derived from a single aromatic ring or multiple condensed aromatic rings. Suitable aryl groups include phenyl and naphthyl, preferably phenyl groups.

The term “(C2-C9)heteroaryl” as used herein refers to monovalent aromatic groups containing at least 5 atoms derived from a single ring or multiple condensed rings, wherein one or more carbon atoms have been replaced by one or more heteroatoms selected from oxygen, sulphur and nitrogen. Suitable heteroaryl groups include furyl, benzofuryl, dibenzofuryl, thienyl, benzothienyl, thieno[2,3-c]pyrazolyl, thieno[3,4-b][1,4]dioxinyl, dibenzothienyl, pyrrolyl, indolyl, pyrrolo[2,3-b]pyridinyl, pyrrolo[3,2-c]pyridinyl, pyrrolo[3,4-b]pyridinyl, diazepanyl, pyrazolyl, pyrazolo[1,5-a]pyridinyl, pyrazolo[3,4-d]pyrimidinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]thiazolyl, imidazo[1,2-a]pyridinyl, imidazo[4,5-b]pyridinyl, purinyl, imidazo[1,2-a]pyrimidinyl, imidazo[1,2-a]pyrazinyl, oxadiazolyl, thiadiazolyl, triazolyl, [1,2,4]triazolo[1,5-a]-pyrimidinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halo” as used herein is intended to include fluorine, chlorine, bromine and iodine atoms, typically fluorine, chlorine or bromine. The term “di-halo” as used herein is intended to refer to two halogens attached to the same carbon.

The term “(C4-C9)spiroheterocycloalkyl” as used herein refers to saturated bicyclic ring systems containing 4 to 9 carbon atoms and at least one heteroatom selected from oxygen, sulphur and nitrogen, in which the two rings are linked by a common atom. Suitable spiroheterocycloalkyl groups include 5-azaspiro[2.3]hexanyl, 5-azaspiro[2.4]-heptanyl, 2-azaspiro[3.3]heptanyl, 2-oxa-6-azaspiro[3.3]heptanyl, 2-oxa-6-azaspiro[3.4]-octanyl, 2-oxa-6-azaspiro[3.5]nonanyl, 7-oxa-2-azaspiro[3.5]nonanyl, 2-oxa-7-azaspiro-[3.5]nonanyl, 2,4,8-triazaspiro[4.5]decanyl, 1-oxaspiro[2.3]hexanyl, 2-oxaspiro[3.3]heptanyl, 1-oxaspiro[3.4]octanyl and 2-oxaspiro[3.5]nonanyl groups.

The term “(C5-C9)bicycloalkyl” as used herein refers to monovalent groups of 5 to 9 carbon atoms derived from a saturated bicyclic hydrocarbon. Typical bicycloalkyl groups include bicyclo[3.1.0]hexanyl, bicyclo[1.1.1]pentanyl, bicyclo[4.1.0]heptanyl, bicyclo[2.2.1]heptanyl and bicyclo[2.2.2]octanyl groups. Alternatively or in addition, instead of (C5-C9)bicycloalkyl groups, also (C4-C9)heterobicycloalkyl groups may be present on the compounds of the present invention which (C4-C9)heterobicycloalkyl groups corresponds to the (C5-C9)bicycloalkyl groups wherein one or more of the carbon atoms have been replaced by one or more heteroatoms selected from oxygen, sulphur and nitrogen. Typical heterobicycloalkyl groups include 3-azabicyclo[3.1.0]hexanyl, 2-oxa-5-azabicyclo[2.2.1]heptanyl, 6-azabicyclo[3.2.0]heptanyl, 3-azabicyclo[3.1.1]heptanyl, 3-azabicyclo[4.1.0]heptanyl, 2-oxabicyclo[2.2.2]octanyl, quinuclidinyl, 2-oxa-5-azabicyclo[2.2.2]octanyl, 3-azabicyclo[3.2.1]octanyl, 8-azabicyclo[3.2.1]octanyl, 3-oxa-8-azabicyclo[3.2.1]octanyl, 3,8-diazabicyclo[3.2.1]octanyl, 3,6-diazabicyclo[3.2.2]nonanyl, 3-oxa-7-azabicyclo[3.3.1]nonanyl and 3,9-diazabicyclo[4.2.1]nonanyl.

Further to the terms explained above, derived expressions such as “(C6-C10)aryl(C1-C6)alkyl”, “(C2-C9)heteroaryl(C1-C6)alkyl”, “(C3-C10)cycloalkyl(C1-C6)alkyl” and “(C1-C6)alkoxy(C1-C6)alkyl” are to be construed accordingly. For example, suitable (C6-C10)aryl(C1-6)alkyl groups include benzyl, phenylethyl, phenylpropyl and naphthylmethyl groups. Suitable (C2-C9)heteroaryl(C1-C6)alkyl groups include pyridylmethyl, pyridazinylmethyl, oxadiazolylmethyl, furanylethyl, oxo-pyridinylethyl and 1H-indazolylmethyl groups. Suitable (C3-C10)cycloalkyl(C1-C6)alkyl groups include cyclopropylmethyl groups. Suitable (C1-C6)alkoxy(C1-C6)alkyl groups include methoxyethyl, methoxypropanyl groups.

In addition, it is further noted that the optional substituents as defined for the present invention may comprise further substituents as such. For example, in case a specific group is optionally substituted with (C1-C6)alkoxy, the (C1-C6)alkoxy may be further substituted with, for example, halo, hydroxy, cyano, oxo, primary, secondary or tertiary amino or the like.

Where the compounds of the present invention have one or more asymmetric centres, they may accordingly exist as enantiomers. Where the compounds of use in the invention possess two or more asymmetric centres, they may additionally exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of the present invention may exist as tautomers, for example keto (CH2C═O)<->enol (CH═CHOH) tautomers, amide (NHC═O)<->hydroxyimine (N═COH) tautomers or 2-hydroxypyridine <->2-pyridone tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In a preferred embodiment of the present invention, the compound, having RNF31 activity, is selected from the group consisting of:
N-[(2E)-3-(benzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-6-benzyl-2-oxo-1,2,5,6,7,8-hexahydro-1,6-naphthyridine-3-carboxamide;
N-[(2Z)-3-(benzenesulfonyl)-3-fluoroprop-2-en-1-yl]-2-oxo-1,2,5,6,7,8-hexahydroquinoline-3-carboxamide;
N-[(2E)-3-(benzenesulfonyl)-3-fluoroprop-2-en-1-yl]-2-oxo-1,2,5,6,7,8-hexahydroquinoline-3-carboxamide;
N-[(2Z)-3-(benzenesulfonyl)prop-2-en-1-yl]-2-oxo-1,2,5,6,7,8-hexahydroquinoline-3-carboxamide;
tert-butyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-1,2,5,6,7,8-hexahydro-1,6-naphthyridine-3-carboxamide;
N-[(2E)-3-(benzenesulfonyl)prop-2-en-1-yl]-2-oxo-5-phenyl-1,2-dihydropyridine-3-carboxamide;
N-[(3E)-4-(benzenesulfonyl)-2-methylbut-3-en-2-yl]-2-oxo-1,2,5,6,7,8-hexahydroquinoline-3-carboxamide;
N-{1-[(E)-2-(benzenesulfonyl)ethenyl]cyclopropyl}-2-oxo-1,2,5,6,7,8-hexahydroquinoline-3-carboxamide;
6-acetyl-N-[(2E)-3-(benzenesulfonyl)prop-2-en-1-yl]-2-oxo-1,2,5,6,7,8-hexahydro-1,6-naphthyridine-3-carboxamide;
N-[(2E)-3-(benzenesulfonyl)prop-2-en-1-yl]-6-(3-methoxypropanoyl)-2-oxo-1,2,5,6,7,8-hexahydro-1,6-naphthyridine-3-carboxamide;
N-[(3E)-4-(benzenesulfonyl)but-3-en-2-yl]-2-oxo-1,2,5,6,7,8-hexahydroquinoline-3-carboxamide;
N-[(2E)-3-(benzenesulfonyl)prop-2-en-1-yl]-6-(2-methoxyethanesulfonyl)-2-oxo-1,2,5,6,7,8-hexahydro-1,6-naphthyridine-3-carboxamide;
N-[(2E)-3-methanesulfonylprop-2-en-1-yl]-2-oxo-1,2,5,6,7,8-hexahydroquinoline-3-carboxamide;
6-[2-(adamantan-1-yl)acetyl]-N-[(2E)-3-(benzenesulfonyl)prop-2-en-1-yl]-2-oxo-1,2,5,6,7,8-hexahydro-1,6-naphthyridine-3-carboxamide;
N-[(2E)-3-(benzenesulfonyl)prop-2-en-1-yl]-2-oxo-6-[(1r,4r)-4-methoxycyclohexanecarbonyl]-1,2,5,6,7,8-hexahydro-1,6-naphthyridine-3-carboxamide;
N-[(2E)-3-(benzenesulfonyl)prop-2-en-1-yl]-6-(4,4-difluorocyclohexanecarbonyl)-2-oxo-1,2,5,6,7,8-hexahydro-1,6-naphthyridine-3-carboxamide;
N-[(2E)-3-(benzenesulfonyl)prop-2-en-1-yl]-6-cyclopropanecarbonyl-2-oxo-1,2,5,6,7,8-hexahydro-1,6-naphthyridine-3-carboxamide;
N-[(2E)-3-(benzenesulfonyl)prop-2-en-1-yl]-6-{bicyclo[2.2.1]heptane-1-carbonyl}-2-oxo-1,2,5,6,7,8-hexahydro-1,6-naphthyridine-3-carboxamide;
N-[(2E)-3-(benzenesulfonyl)prop-2-en-1-yl]-6-(3,3-difluorocyclobutanecarbonyl)-2-oxo-1,2,5,6,7,8-hexahydro-1,6-naphthyridine-3-carboxamide;
N-[(2E)-3-(benzenesulfonyl)prop-2-en-1-yl]-6-(1-cyanocyclobutanecarbonyl)-2-oxo-1,2,5,6,7,8-hexahydro-1,6-naphthyridine-3-carboxamide;
N-[(2E)-3-(benzenesulfonyl)prop-2-en-1-yl]-2-oxo-6-(2-phenyl-2H-1,2,3-triazole-4-carbonyl)-1,2,5,6,7,8-hexahydro-1,6-naphthyridine-3-carboxamide;
N-[(2E)-3-(benzenesulfonyl)prop-2-en-1-yl]-2-oxo-6-(oxolane-3-carbonyl)-1,2,5,6,7,8-hexahydro-1,6-naphthyridine-3-carboxamide;
N-[(2E)-3-(benzenesulfonyl)prop-2-en-1-yl]-6-{3-methylbicyclo[1.1.1]pentane-1-carbonyl}-2-oxo-1,2,5,6,7,8-hexahydro-1,6-naphthyridine-3-carboxamide;
N-[(2E)-3-(4-fluorobenzenesulfonyl)prop-2-en-1-yl]-2-oxo-1,2,5,6,7,8-hexahydroquinoline-3-carboxamide;
N-[(2E)-3-(cyclopentanesulfonyl)prop-2-en-1-yl]-2-oxo-1,2,5,6,7,8-hexahydroquinoline-3-carboxamide;
2-oxo-N-[(2E)-3-(propane-2-sulfonyl)prop-2-en-1-yl]-1,2,5,6,7,8-hexahydroquinoline-3-carboxamide;
N-[(2E)-3-(3-fluoro-4-methoxybenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-6-(1-methylcyclobutanecarbonyl)-2-oxo-1,2,5,6,7,8-hexahydro-1,6-naphthyridine-3-carboxamide;
N-[(2E)-3-(benzenesulfonyl)prop-2-en-1-yl]-6-(4-cyclopropyl-1,3-thiazole-2-carbonyl)-2-oxo-1,2,5,6,7,8-hexahydro-1,6-naphthyridine-3-carboxamide;
N-[(2E)-3-(benzenesulfonyl)prop-2-en-1-yl]-6-(2-cyclopropylacetyl)-2-oxo-1,2,5,6,7,8-hexahydro-1,6-naphthyridine-3-carboxamide;
2-oxo-N-[(2Z)-3-(propane-2-sulfonyl)prop-2-en-1-yl]-1,2,5,6,7,8-hexahydroquinoline-3-carboxamide;
N-[(2E)-3-(4-chlorobenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-2-oxo-6-(1-phenylpyrrolidine-3-carbonyl)-1,2,5,6,7,8-hexahydro-1,6-naphthyridine-3-carboxamide;
2-methylpropyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(2-fluorobenzenesulfonyl)prop-2-en-1-yl]carbamoyl}-2-oxo-1,2,5,6,7,8-hexahydro-1,6-naphthyridine-6-carboxylate;
N-[(2E)-3-(2-methylpropane-2-sulfonyl)prop-2-en-1-yl]-2-oxo-1,2,5,6,7,8-hexahydroquinoline-3-carboxamide;
N-[(2E)-3-(2-fluorobenzenesulfonyl)prop-2-en-1-yl]-2-oxo-1,2,5,6,7,8-hexahydroquinoline-3-carboxamide;
tert-butyl 3-{[(2E)-3-(4-fluorobenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-6-(3,3-dimethylbutanoyl)-2-oxo-1,2,5,6,7,8-hexahydro-1,6-naphthyridine-3-carboxamide;
tert-butyl 3-{[(2E)-3-(3-fluoro-4-methoxybenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-6-[(1-methyl-1H-indazol-4-yl)methyl]-2-oxo-1,2,5,6,7,8-hexahydro-1,6-naphthyridine-3-carboxamide;
2-(4-chlorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-6-[(pyridin-3-yl)methyl]-1,2,5,6,7,8-hexahydro-1,6-naphthyridine-3-carboxamide;
N-[(2E)-3-(benzenesulfonyl)prop-2-en-1-yl]-2-oxo-6-[(pyridazin-3-yl)methyl]-1,2,5,6,7,8-hexahydro-1,6-naphthyridine-3-carboxamide;
N-[(2E)-3-(benzenesulfonyl)prop-2-en-1-yl]-6-(3-methoxy-3-methylbutanoyl)-2-oxo-1,2,5,6,7,8-hexahydro-1,6-naphthyridine-3-carboxamide;
N-[(2E)-3-(4-chlorobenzenesulfonyl)prop-2-en-1-yl]-2-oxo-1,2-dihydroquinoline-3-carboxamide;
6-benzyl-N-[(2E)-3-(4-chlorobenzenesulfonyl)prop-2-en-1-yl]-2-oxo-1,2,5,6,7,8-hexahydro-1,6-naphthyridine-3-carboxamide;
N-[(2E)-3-(4-chlorobenzenesulfonyl)prop-2-en-1-yl]-2-oxo-5-phenyl-1,2-dihydropyridine-3-carboxamide;
N-[(2Z)-3-(2-fluorobenzenesulfonyl)but-2-en-1-yl]-2-oxo-1,2,5,6,7,8-hexahydroquinoline-3-carboxamide;
N-[(2E)-3-(2-fluorobenzenesulfonyl)but-2-en-1-yl]-2-oxo-1,2,5,6,7,8-hexahydroquinoline-3-carboxamide;
cyclopropylmethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclobutyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(5-methyl-1,3,4-oxadiazol-2-yl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1,1,1-trifluoropropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-difluorocyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
oxetan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
1-methoxypropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
2-fluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
2,2-difluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclopropyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-6-(2-phenylcyclopropanecarbonyl)-1,2,5,6,7,8-hexahydro-1,6-naphthyridine-3-carboxamide;
2-oxaspiro[3.5]nonan-7-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
2-methoxyethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2,4-difluorobenzenesulfonyl)prop-2-en-1-yl]-2-oxo-1,2,5,6,7,8-hexahydroquinoline-3-carboxamide;
N-[(2E)-3-(2,4-dichlorobenzenesulfonyl)prop-2-en-1-yl]-2-oxo-1,2,5,6,7,8-hexahydroquinoline-3-carboxamide;
(3S)-oxolan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
(3R)-oxolan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2-chloro-4-fluorobenzenesulfonyl)prop-2-en-1-yl]-2-oxo-1,2,5,6,7,8-hexahydroquinoline-3-carboxamide;
N-[(2E)-3-(2,4-difluorobenzenesulfonyl)prop-2-en-1-yl]-2-oxo-5-phenyl-1,2-dihydropyridine-3-carboxamide;
N-[(2E)-3-(2,4-dichlorobenzenesulfonyl)prop-2-en-1-yl]-2-oxo-5-phenyl-1,2-dihydropyridine-3-carboxamide:
N-[(2E)-3-(2-chloro-4-fluorobenzenesulfonyl)prop-2-en-1-yl]-2-oxo-5-phenyl-1,2-dihydropyridine-3-carboxamide;
N-[(2E)-3-(2,4-difluorobenzenesulfonyl)prop-2-en-1-yl]-2-oxo-1H,2H,5H,6H,7H,8H,9H-cyclohepta[b]pyridine-3-carboxamide;
N-[(2E)-3-(4-fluorobenzenesulfonyl)prop-2-en-1-yl]-2-oxo-1H,2H,5H,6H,7H,8H,9H-cyclohepta[b]pyridine-3-carboxamide;
N-[(2E)-3-(4-chlorobenzenesulfonyl)prop-2-en-1-yl]-2-oxo-1H,2H,5H,6H,7H,8H,9H-cyclohepta[b]pyridine-3-carboxamide;
N-[(2Z)-3-(4-chlorobenzenesulfonyl)but-2-en-1-yl]-2-oxo-1,2,5,6,7,8-hexahydroquinoline-3-carboxamide;
N-[(2E)-3-(4-chlorobenzenesulfonyl)but-2-en-1-yl]-2-oxo-1,2,5,6,7,8-hexahydroquinoline-3-carboxamide;
N-[(2E)-3-(3-fluoro-4-methoxybenzenesulfonyl)prop-2-en-1-yl]-2-oxo-1H,2H,5H,6H,7H,8H,9H-cyclohepta[b]pyridine-3-carboxamide;
N-[(2E)-3-(benzenesulfonyl)prop-2-en-1-yl]-2-oxo-1H,2H,5H,6H,7H,8H,9H-cyclohepta[b]pyridine-3-carboxamide;
N-[(2Z)-3-(4-chlorobenzenesulfonyl)but-2-en-1-yl]-2-oxo-5-phenyl-1,2-dihydropyridine-3-carboxamide;
N-[(2E)-3-(4-chlorobenzenesulfonyl)but-2-en-1-yl]-2-oxo-5-phenyl-1,2-dihydropyridine-3-carboxamide;
N-[(2E)-3-(2-chlorobenzenesulfonyl)prop-2-en-1-yl]-2-oxo-5-phenyl-1,2-dihydropyridine-3-carboxamide;
N-[(2E)-3-(3-fluorobenzenesulfonyl)prop-2-en-1-yl]-2-oxo-1,2,5,6,7,8-hexahydroquinoline-3-carboxamide;
N-[(2E)-3-(4-methoxybenzenesulfonyl)prop-2-en-1-yl]-2-oxo-1,2,5,6,7,8-hexahydroquinoline-3-carboxamide;
2-(2-oxopiperidin-1-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2-chlorobenzenesulfonyl)prop-2-en-1-yl]-2-oxo-1,2,5,6,7,8-hexahydroquinoline-3-carboxamide;
cyclopentyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
(2,2-difluorocyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
oxetan-3-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(4-chlorobenzenesulfonyl)-3-fluoroprop-2-en-1-yl]-2-oxo-1,2,5,6,7,8-hexahydroquinoline-3-carboxamide;
N-[(2Z)-3-(4-chlorobenzenesulfonyl)-3-fluoroprop-2-en-1-yl]-2-oxo-1,2,5,6,7,8-hexahydroquinoline-3-carboxamide;
cyclopropylmethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(2,4-difluorobenzenesulfonyl)prop-2-en-1-yl]-2-oxo-1H,2H,5H,6H,7H-cyclopenta[b]pyridine-3-carboxamide;
N-[(2E)-3-(benzenesulfonyl)prop-2-en-1-yl]-2-oxo-1H,2H,5H,6H,7H-cyclopenta[b]pyridine-3-carboxamide;
2-(furan-2-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclohexyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1-(2,6-difluorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1-acetylpiperidin-4-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
2-fluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
1-methoxypropan-2-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-[(5-chloropyridin-2-yl)sulfonyl]prop-2-en-1-yl]-2-oxo-1,2,5,6,7,8-hexahydroquinoline-3-carboxamide;
2-oxo-N-[(2E)-3-(thiophene-2-sulfonyl)prop-2-en-1-yl]-1,2,5,6,7,8-hexahydroquinoline-3-carboxamide;
cyclopropylmethyl 3-{[(2E)-3-[(5-chloropyridin-2-yl)sulfonyl]prop-2-en-1-yl]carbamoyl}-2-oxo-1,2,5,6,7,8-hexahydro-1,6-naphthyridine-6-carboxylate;
2,2-difluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
2,2,2-trifluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(4-bromobenzenesulfonyl)prop-2-en-1-yl]-2-oxo-1,2,5,6,7,8-hexahydroquinoline-3-carboxamide;
N-[(2E)-3-(3,4-dimethoxybenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-2-oxo-6-(propan-2-yl)-1,2,5,6,7,8-hexahydroquinoline-3-carboxamide;
cyclopropylmethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-1,2-dihydro-1,8-naphthyridine-3-carboxamide;
N-[(2E)-3-(benzenesulfonyl)prop-2-en-1-yl]-6,6-dimethyl-2-oxo-1,2,5,6,7,8-hexahydroquinoline-3-carboxamide;
N-[(2E)-3-(benzenesulfonyl)prop-2-en-1-yl]-6,6-difluoro-2-oxo-1,2,5,6,7,8-hexahydroquinoline-3-carboxamide;
N3-[(2E)-3-(4-chlorobenzenesulfonyl)prop-2-en-1-yl]-N6-(cyclopropylmethyl)-2-oxo-1,2,5,6,7,8-hexahydro-1,6-naphthyridine-3,6-dicarboxamide;
N3-[(2E)-3-(4-chlorobenzenesulfonyl)prop-2-en-1-yl]-N6-cyclopropyl-2-oxo-1,2,5,6,7,8-hexahydro-1,6-naphthyridine-3,6-dicarboxamide;
N6-tert-butyl-N3-[(2E)-3-(4-chlorobenzenesulfonyl)prop-2-en-1-yl]-2-oxo-1,2,5,6,7,8-hexahydro-1,6-naphthyridine-3,6-dicarboxamide;
N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide;
N-[(2E)-3-(4-tert-butylbenzenesulfonyl)prop-2-en-1-yl]-2-oxo-1,2,5,6,7,8-hexahydroquinoline-3-carboxamide;
N-[(2E)-3-(3,4-dimethylbenzenesulfonyl)prop-2-en-1-yl]-2-oxo-1,2,5,6,7,8-hexahydroquinoline-3-carboxamide;
N-[(2E)-3-[(3,4-dimethoxyphenyl)(imino)oxo-λ⁶-sulfanyl]prop-2-en-1-yl]-2-oxo-1,2,5,6,7,8-hexahydroquinoline-3-carboxamide;
N-[(2E)-3-(benzenesulfonyl)(1,1-²H2)prop-2-en-1-yl]-2-oxo-1,2,5,6,7,8-hexahydroquinoline-3-carboxamide;
N-[(2E)-3-[4-(dimethylamino)benzenesulfonyl]prop-2-en-1-yl]-2-oxo-1,2,5,6,7,8-hexahydroquinoline-3-carboxamide;
N-[(2E)-3-(4-cyclopropylbenzenesulfonyl)prop-2-en-1-yl]-2-oxo-1,2,5,6,7,8-hexahydroquinoline-3-carboxamide;
N-[(2E)-3-(4-cyanobenzenesulfonyl)prop-2-en-1-yl]-2-oxo-1,2,5,6,7,8-hexahydroquinoline-3-carboxamide;
N-[(2E)-3-[imino(4-methoxyphenyl)oxo-λ⁶-sulfanyl]prop-2-en-1-yl]-2-oxo-1,2,5,6,7,8-hexahydroquinoline-3-carboxamide;
N-[(2E)-3-[(3,4-dimethylphenyl)(imino)oxo-λ⁶-sulfanyl]prop-2-en-1-yl]-2-oxo-1,2,5,6,7,8-hexahydroquinoline-3-carboxamide;
6,6-difluoro-N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide;
cyclopropylmethyl 3-{[(2E)-3-[(4-fluorophenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)phenyl-λ⁶-sulfanyl]prop-2-en-1-yl]-2-oxo-1,2,5,6,7,8-hexahydroquinoline-3-carboxamide;
N-[(2E)-3-[(4-tert-butylphenyl)(imino)oxo-λ⁶-sulfanyl]prop-2-en-1-yl]-2-oxo-1,2,5,6,7,8-hexahydroquinoline-3-carboxamide;
N-[(2E)-3-[(4-ethoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3,5-dimethylphenyl)oxo-λ⁶-sulfanyl]prop-2-en-1-yl]-2-oxo-1,2,5,6,7,8-hexahydroquinoline-3-carboxamide;
N-[(2E)-3-[imino(3-methoxyphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(trifluoromethoxy)phenyl]-λ⁶-sulfanyl]prop-2-en-1-yl]-2-oxo-1,2,5,6,7,8-hexahydroquinoline-3-carboxamide;
N-[(2E)-3-[(4-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[(2-fluorophenyl)(imino)oxo-λ⁶-sulfanyl]prop-2-λ⁶-1-yl]-2-oxo-1,2,5,6,7,8-hexahydroquinoline-3-carboxamide;
N-[(2E)-3-[(3-chloro-4-methoxyphenyl)(imino)oxo-λ⁶-sulfanyl]prop-2-en-1-yl]-2-oxo-1,2,5,6,7,8-hexahydroquinoline-3-carboxamide;
2,2-difluoroethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[(3-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[(2,4-difluorophenyl)(imino)oxo-λ⁶-sulfanyl]prop-2-en-1-yl]-2-oxo-1,2,5,6,7,8-hexahydroquinoline-3-carboxamide;
N-[(2E)-3-[(4-cyanophenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3-methylphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(propan-2-yloxy)phenyl]-λ⁶-sulfanyl]prop-2-en-1-yl]-2-oxo-1,2,5,6,7,8-hexahydroquinoline-3-carboxamide;
N-[(2E)-3-[imino(4-methoxy-2-methylphenyl)oxo-λ⁶-sulfanyl]prop-2-en-1-yl]-2-oxo-1,2,5,6,7,8-hexahydroquinoline-3-carboxamide;
cyclopropylmethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
2-fluoroethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
2-fluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
2,2-difluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)(4-phenoxyphenyl)-λ⁶-sulfanyl]prop-2-en-1-yl]-2-oxo-1,2,5,6,7,8-hexahydroquinoline-3-carboxamide;
N-[(2E)-3-{[4-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-6-methyl-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1,2-dihydroquinoline-3-carboxamide;
N-[(2E)-3-[(3-fluoro-4-methoxyphenyl)(imino)oxo-λ⁶-sulfanyl]prop-2-en-1-yl]-2-oxo-6-(propan-2-yl)-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-6-methoxy-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-6-(2,2,2-trifluoroethyl)-1,2,5,6,7,8-hexahydro-1,6-naphthyridine-3-carboxamide
N-[(2E)-3-[(3-fluoro-4-methoxyphenyl)(imino)oxo-λ⁶-sulfanyl]prop-2-en-1-yl]-2-oxo-1H,2H,5H,7H,8H-pyrano[4,3-b]pyridine-3-carboxamide;
N-[(2E)-3-[imino(oxo)(3-phenoxyphenyl)-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-6-(trifluoromethyl)-1,2,5,6,7,8-hexahydroquinoline-3-carboxamide;
6-tert-butyl-N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-5-phenyl-1,2-dihydropyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H,8H,9H-cyclohepta[b]pyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H-cyclopenta[b]pyridine-3-carboxamide;
tert-butyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
6-benzyl-N-[(2E)-3-[(3-fluoro-4-methoxyphenyl)(imino)oxo-λ⁶-sulfanyl]prop-2-en-1-yl]-2-oxo-1,2,5,6,7,8-hexahydro-1,6-naphthyridine-3-carboxamide;
N-[(2E)-3-[(3-fluoro-4-methoxyphenyl)(imino)oxo-λ⁶-sulfanyl]prop-2-en-1-yl]-6-[(4-methoxyphenyl)methyl]-2-oxo-1,2,5,6,7,8-hexahydro-1,6-naphthyridine-3-carboxamide;
4,4-difluorocyclohexyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
cyclohexyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)[4-(trifluoromethyl)phenyl]-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-8,8-dimethyl-2-oxo-1,2,5,6,7,8-hexahydroquinoline-3-carboxamide;
6-(cyclopropylmethyl)-N-[(2E)-3-[(3-fluoro-4-methoxyphenyl)(imino)oxo-λ⁶-sulfanyl]prop-2-en-1-yl]-2-oxo-1,2,5,6,7,8-hexahydro-1,6-naphthyridine-3-carboxamide;
N-[(2E)-3-({[1,1′-biphenyl]-4-yl}(imino)oxo-λ⁶-sulfanyl)prop-2-en-1-yl]-2-oxo-1,2,5,6,7,8-hexahydroquinoline-3-carboxamide;
N-[(2E)-3-[benzyl(imino)oxo-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1,2-dihydro-1,8-naphthyridine-3-carboxamide;
N-[(2E)-3-[(3-fluoro-4-methoxyphenyl)(imino)oxo-λ⁶-sulfanyl]prop-2-en-1-yl]-2-oxo-1,2-dihydro-1,6-naphthyridine-3-carboxamide;
6-fluoro-N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-{imino[4-(morpholin-4-yl)phenyl]oxo-λ⁶-sulfanyl}prop-2-en-1-yl]-2-oxo-1,2,5,6,7,8-hexahydroquinoline-3-carboxamide;
N-[(2E)-3-{[3-cyano-4-(piperidin-1-yl)phenyl](imino)oxo-λ⁶-sulfanyl}prop-2-en-1-yl]-2-oxo-1,2,5,6,7,8-hexahydroquinoline-3-carboxamide;
N-[(2E)-3-[imino(oxo)[4-(piperidin-1-yl)phenyl]-λ⁶-sulfanyl]prop-2-en-1-yl]-2-oxo-1,2,5,6,7,8-hexahydroquinoline-3-carboxamide;
N-[(2E)-3-[imino(oxo)[4-(pyrrolidin-1-yl)phenyl]-λ⁶-sulfanyl]prop-2-en-1-yl]-2-oxo-1,2,5,6,7,8-hexahydroquinoline-3-carboxamide;
N-[(2E)-3-{imino[4-(methylamino)phenyl]oxo-λ⁶-sulfanyl}prop-2-en-1-yl]-2-oxo-1,2,5,6,7,8-hexahydroquinoline-3-carboxamide;
N-[(2E)-3-{[3-cyano-4-(morpholin-4-yl)phenyl](imino)oxo-λ⁶-sulfanyl}prop-2-en-1-yl]-2-oxo-1,2,5,6,7,8-hexahydroquinoline-3-carboxamide;
N-[(2E)-3-[imino(oxo)[4-(2-oxopiperidin-1-yl)phenyl]-λ⁶-sulfanyl]prop-2-en-1-yl]-2-oxo-1,2,5,6,7,8-hexahydroquinoline-3-carboxamide;
N-[(2E)-3-[imino(1-methyl-1,2,3,4-tetrahydroquinolin-6-yl)oxo-λ⁶-sulfanyl]prop-2-en-1-yl]-2-oxo-1,2,5,6,7,8-hexahydroquinoline-3-carboxamide;
N-[(2E)-3-[(4-acetamidophenyl)(imino)oxo-λ⁶-sulfanyl]prop-2-en-1-yl]-2-oxo-1,2,5,6,7,8-hexahydroquinoline-3-carboxamide;
N-[(2E)-3-[(4-cyclopropoxyphenyl)(imino)oxo-λ⁶-sulfanyl]prop-2-en-1-yl]-2-oxo-1,2,5,6,7,8-hexahydroquinoline-3-carboxamide;
N-[(2E)-3-{[3-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(4-cyclohexylphenyl)(imino)oxo-λ⁶-sulfanyl]prop-2-en-1-yl]-2-oxo-1,2,5,6,7,8-hexahydroquinoline-3-carboxamide;
N-[(2E)-3-[(2,3-dihydro-1-benzofuran-5-yl)(imino)oxo-λ⁶-sulfanyl]prop-2-en-1-yl]-2-oxo-1,2,5,6,7,8-hexahydroquinoline-3-carboxamide;
N-[(2E)-3-[imino(oxo)[3-(trifluoromethoxy)phenyl]-λ⁶-sulfanyl]prop-2-en-1-yl]-2-oxo-1,2,5,6,7,8-hexahydroquinoline-3-carboxamide; and
N-[(2E)-3-[(3-fluoro-4-methoxyphenyl)(imino)oxo-λ⁶-sulfanyl]prop-2-en-1-yl]-2-oxo-1,2-dihydropyridine-3-carboxamide.

In particular promising results, in relation to improved RNF31 activity, are obtained wherein the compound is selected from the group consisting of:
N-[(2E)-3-(benzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-6-benzyl-2-oxo-1,2,5,6,7,8-hexahydro-1,6-naphthyridine-3-carboxamide;
tert-butyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-6-(3-methoxypropanoyl)-2-oxo-1,2,5,6,7,8-hexahydro-1,6-naphthyridine-3-carboxamide;
N-[(2E)-3-(benzenesulfonyl)prop-2-en-1-yl]-6-(2-methoxyethanesulfonyl)-2-oxo-1,2,5,6,7,8-hexahydro-1,6-naphthyridine-3-carboxamide;
6-[2-(adamantan-1-yl)acetyl]-N-[(2E)-3-(benzenesulfonyl)prop-2-en-1-yl]-2-oxo-1,2,5,6,7,8-hexahydro-1,6-naphthyridine-3-carboxamide;
N-[(2E)-3-(benzenesulfonyl)prop-2-en-1-yl]-2-oxo-6-[(1r,4r)-4-methoxycyclohexanecarbonyl]-1,2,5,6,7,8-hexahydro-1,6-naphthyridine-3-carboxamide;
N-[(2E)-3-(benzenesulfonyl)prop-2-en-1-yl]-6-cyclopropanecarbonyl-2-oxo-1,2,5,6,7,8-hexahydro-1,6-naphthyridine-3-carboxamide;
N-[(2E)-3-(benzenesulfonyl)prop-2-en-1-yl]-6-{bicyclo[2.2.1]heptane-1-carbonyl}-2-oxo-1,2,5,6,7,8-hexahydro-1,6-naphthyridine-3-carboxamide;
N-[(2E)-3-(benzenesulfonyl)prop-2-en-1-yl]-2-oxo-6-(2-phenyl-2H-1,2,3-triazole-4-carbonyl)-1,2,5,6,7,8-hexahydro-1,6-naphthyridine-3-carboxamide;
N-[(2E)-3-(benzenesulfonyl)prop-2-en-1-yl]-2-oxo-6-(oxolane-3-carbonyl)-1,2,5,6,7,8-hexahydro-1,6-naphthyridine-3-carboxamide;
N-[(2E)-3-(benzenesulfonyl)prop-2-en-1-yl]-6-{3-methylbicyclo[1.1.1]pentane-1-carbonyl}-2-oxo-1,2,5,6,7,8-hexahydro-1,6-naphthyridine-3-carboxamide;
N-[(2E)-3-(4-fluorobenzenesulfonyl)prop-2-en-1-yl]-2-oxo-1,2,5,6,7,8-hexahydroquinoline-3-carboxamide;
N-[(2E)-3-(3-fluoro-4-methoxybenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-6-(1-methylcyclobutanecarbonyl)-2-oxo-1,2,5,6,7,8-hexahydro-1,6-naphthyridine-3-carboxamide;
N-[(2E)-3-(benzenesulfonyl)prop-2-en-1-yl]-6-(4-cyclopropyl-1,3-thiazole-2-carbonyl)-2-oxo-1,2,5,6,7,8-hexahydro-1,6-naphthyridine-3-carboxamide;
N-[(2E)-3-(benzenesulfonyl)prop-2-en-1-yl]-6-(2-cyclopropylacetyl)-2-oxo-1,2,5,6,7,8-hexahydro-1,6-naphthyridine-3-carboxamide;
N-[(2E)-3-(4-chlorobenzenesulfonyl)prop-2-en-1-yl]-2-oxo-1,2,5,6,7,8-hexahydroquinoline-3-carboxamide;
2-methylpropyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(2-fluorobenzenesulfonyl)prop-2-en-1-yl]carbamoyl}-2-oxo-1,2,5,6,7,8-hexahydro-1,6-naphthyridine-6-carboxylate;
N-[(2E)-3-(2-fluorobenzenesulfonyl)prop-2-en-1-yl]-2-oxo-1,2,5,6,7,8-hexahydroquinoline-3-carboxamide;
tert-butyl 3-{[(2E)-3-(4-fluorobenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-6-(3,3-dimethylbutanoyl)-2-oxo-1,2,5,6,7,8-hexahydro-1,6-naphthyridine-3-carboxamide;
tert-butyl 3-{[(2E)-3-(3-fluoro-4-methoxybenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-6-[(1-methyl-1H-indazol-4-yl)methyl]-2-oxo-1,2,5,6,7,8-hexahydro-1,6-naphthyridine-3-carboxamide;
2-(4-chlorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-6-[(pyridin-3-yl)methyl]-1,2,5,6,7,8-hexahydro-1,6-naphthyridine-3-carboxamide;
N-[(2E)-3-(benzenesulfonyl)prop-2-en-1-yl]-2-oxo-6-[(pyridazin-3-yl)methyl]-1,2,5,6,7,8-hexahydro-1,6-naphthyridine-3-carboxamide;
N-[(2E)-3-(4-chlorobenzenesulfonyl)prop-2-en-1-yl]-2-oxo-1,2-dihydroquinoline-3-carboxamide;
6-benzyl-N-[(2E)-3-(4-chlorobenzenesulfonyl)prop-2-en-1-yl]-2-oxo-1,2,5,6,7,8-hexahydro-1,6-naphthyridine-3-carboxamide;
cyclopropylmethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclobutyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(5-methyl-1,3,4-oxadiazol-2-yl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1,1,1-trifluoropropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-difluorocyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
oxetan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
1-methoxypropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
2-fluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
2,2-difluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclopropyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-6-(2-phenylcyclopropanecarbonyl)-1,2,5,6,7,8-hexahydro-1,6-naphthyridine-3-carboxamide;
2-oxaspiro[3.5]nonan-7-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
2-methoxyethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2,4-difluorobenzenesulfonyl)prop-2-en-1-yl]-2-oxo-1,2,5,6,7,8-hexahydroquinoline-3-carboxamide;
N-[(2E)-3-(2,4-dichlorobenzenesulfonyl)prop-2-en-1-yl]-2-oxo-1,2,5,6,7,8-hexahydroquinoline-3-carboxamide;
(3S)-oxolan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
(3R)-oxolan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2-chloro-4-fluorobenzenesulfonyl)prop-2-en-1-yl]-2-oxo-1,2,5,6,7,8-hexahydroquinoline-3-carboxamide;
N-[(2E)-3-(2,4-difluorobenzenesulfonyl)prop-2-en-1-yl]-2-oxo-5-phenyl-1,2-dihydropyridine-3-carboxamide;
N-[(2E)-3-(2,4-dichlorobenzenesulfonyl)prop-2-en-1-yl]-2-oxo-5-phenyl-1,2-dihydropyridine-3-carboxamide:
N-[(2E)-3-(2-chloro-4-fluorobenzenesulfonyl)prop-2-en-1-yl]-2-oxo-5-phenyl-1,2-dihydropyridine-3-carboxamide;
N-[(2E)-3-(2,4-difluorobenzenesulfonyl)prop-2-en-1-yl]-2-oxo-1H,2H,5H,6H,7H,8H,9H-cyclohepta[b]pyridine-3-carboxamide;
N-[(2E)-3-(4-fluorobenzenesulfonyl)prop-2-en-1-yl]-2-oxo-1H,2H,5H,6H,7H,8H,9H-cyclohepta[b]pyridine-3-carboxamide;
N-[(2E)-3-(4-chlorobenzenesulfonyl)prop-2-en-1-yl]-2-oxo-1H,2H,5H,6H,7H,8H,9H-cyclohepta[b]pyridine-3-carboxamide;
N-[(2E)-3-(benzenesulfonyl)prop-2-en-1-yl]-2-oxo-1H,2H,5H,6H,7H,8H,9H-cyclohepta[b]pyridine-3-carboxamide;
N-[(2E)-3-(2-chlorobenzenesulfonyl)prop-2-en-1-yl]-2-oxo-5-phenyl-1,2-dihydropyridine-3-carboxamide;
N-[(2E)-3-(3-fluorobenzenesulfonyl)prop-2-en-1-yl]-2-oxo-1,2,5,6,7,8-hexahydroquinoline-3-carboxamide;
N-[(2E)-3-(4-methoxybenzenesulfonyl)prop-2-en-1-yl]-2-oxo-1,2,5,6,7,8-hexahydroquinoline-3-carboxamide;
2-(2-oxopiperidin-1-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2-chlorobenzenesulfonyl)prop-2-en-1-yl]-2-oxo-1,2,5,6,7,8-hexahydroquinoline-3-carboxamide;
cyclopentyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
(2,2-difluorocyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
oxetan-3-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
cyclopropylmethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(2,4-difluorobenzenesulfonyl)prop-2-en-1-yl]-2-oxo-1H,2H,5H,6H,7H-cyclopenta[b]pyridine-3-carboxamide;
N-[(2E)-3-(benzenesulfonyl)prop-2-en-1-yl]-2-oxo-1H,2H,5H,6H,7H-cyclopenta[b]pyridine-3-carboxamide;
2-(furan-2-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclohexyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1-(2,6-difluorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1-acetylpiperidin-4-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
2-fluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
1-methoxypropan-2-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-[(5-chloropyridin-2-yl)sulfonyl]prop-2-en-1-yl]-2-oxo-1,2,5,6,7,8-hexahydroquinoline-3-carboxamide;
2-oxo-N-[(2E)-3-(thiophene-2-sulfonyl)prop-2-en-1-yl]-1,2,5,6,7,8-hexahydroquinoline-3-carboxamide;
cyclopropylmethyl 3-{[(2E)-3-[(5-chloropyridin-2-yl)sulfonyl]prop-2-en-1-yl]carbamoyl}-2-oxo-1,2,5,6,7,8-hexahydro-1,6-naphthyridine-6-carboxylate;
2,2-difluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
2,2,2-trifluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(4-bromobenzenesulfonyl)prop-2-en-1-yl]-2-oxo-1,2,5,6,7,8-hexahydroquinoline-3-carboxamide;
N-[(2E)-3-(3,4-dimethoxybenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-2-oxo-6-(propan-2-yl)-1,2,5,6,7,8-hexahydroquinoline-3-carboxamide;
cyclopropylmethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-1,2-dihydro-1,8-naphthyridine-3-carboxamide;
N-[(2E)-3-(benzenesulfonyl)prop-2-en-1-yl]-6,6-dimethyl-2-oxo-1,2,5,6,7,8-hexahydroquinoline-3-carboxamide;
N-[(2E)-3-(benzenesulfonyl)prop-2-en-1-yl]-6,6-difluoro-2-oxo-1,2,5,6,7,8-hexahydroquinoline-3-carboxamide;
N3-[(2E)-3-(4-chlorobenzenesulfonyl)prop-2-en-1-yl]-N6-(cyclopropylmethyl)-2-oxo-1,2,5,6,7,8-hexahydro-1,6-naphthyridine-3,6-dicarboxamide;
N3-[(2E)-3-(4-chlorobenzenesulfonyl)prop-2-en-1-yl]-N6-cyclopropyl-2-oxo-1,2,5,6,7,8-hexahydro-1,6-naphthyridine-3,6-dicarboxamide;
N6-tert-butyl-N3-[(2E)-3-(4-chlorobenzenesulfonyl)prop-2-en-1-yl]-2-oxo-1,2,5,6,7,8-hexahydro-1,6-naphthyridine-3,6-dicarboxamide;
N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide;
N-[(2E)-3-(3,4-dimethylbenzenesulfonyl)prop-2-en-1-yl]-2-oxo-1,2,5,6,7,8-hexahydroquinoline-3-carboxamide;
N-[(2E)-3-[(3,4-dimethoxyphenyl)(imino)oxo-λ⁶-sulfanyl]prop-2-en-1-yl]-2-oxo-1,2,5,6,7,8-hexahydroquinoline-3-carboxamide;
N-[(2E)-3-(benzenesulfonyl)(1,1-²H2)prop-2-en-1-yl]-2-oxo-1,2,5,6,7,8-hexahydroquinoline-3-carboxamide;
N-[(2E)-3-[4-(dimethylamino)benzenesulfonyl]prop-2-en-1-yl]-2-oxo-1,2,5,6,7,8-hexahydroquinoline-3-carboxamide;
N-[(2E)-3-(4-cyclopropylbenzenesulfonyl)prop-2-en-1-yl]-2-oxo-1,2,5,6,7,8-hexahydroquinoline-3-carboxamide;
N-[(2E)-3-(4-cyanobenzenesulfonyl)prop-2-en-1-yl]-2-oxo-1,2,5,6,7,8-hexahydroquinoline-3-carboxamide;
N-[(2E)-3-[imino(4-methoxyphenyl)oxo-λ⁶-sulfanyl]prop-2-en-1-yl]-2-oxo-1,2,5,6,7,8-hexahydroquinoline-3-carboxamide;
N-[(2E)-3-[(3,4-dimethylphenyl)(imino)oxo-λ⁶-sulfanyl]prop-2-en-1-yl]-2-oxo-1,2,5,6,7,8-hexahydroquinoline-3-carboxamide;
6,6-difluoro-N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide;
cyclopropylmethyl 3-{[(2E)-3-[(4-fluorophenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)phenyl-λ⁶-sulfanyl]prop-2-en-1-yl]-2-oxo-1,2,5,6,7,8-hexahydroquinoline-3-carboxamide;
N-[(2E)-3-[(4-tert-butylphenyl)(imino)oxo-λ⁶-sulfanyl]prop-2-en-1-yl]-2-oxo-1,2,5,6,7,8-hexahydroquinoline-3-carboxamide;
N-[(2E)-3-[(4-ethoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3,5-dimethylphenyl)oxo-λ⁶-sulfanyl]prop-2-en-1-yl]-2-oxo-1,2,5,6,7,8-hexahydroquinoline-3-carboxamide;
N-[(2E)-3-[imino(3-methoxyphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(trifluoromethoxy)phenyl]-λ⁶-sulfanyl]prop-2-en-1-yl]-2-oxo-1,2,5,6,7,8-hexahydroquinoline-3-carboxamide;
N-[(2E)-3-[(4-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[(2-fluorophenyl)(imino)oxo-λ⁶-sulfanyl]prop-2-λ⁶-1-yl]-2-oxo-1,2,5,6,7,8-hexahydroquinoline-3-carboxamide;
N-[(2E)-3-[(3-chloro-4-methoxyphenyl)(imino)oxo-λ⁶-sulfanyl]prop-2-en-1-yl]-2-oxo-1,2,5,6,7,8-hexahydroquinoline-3-carboxamide;
2,2-difluoroethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[(3-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[(2,4-difluorophenyl)(imino)oxo-λ⁶-sulfanyl]prop-2-en-1-yl]-2-oxo-1,2,5,6,7,8-hexahydroquinoline-3-carboxamide;
N-[(2E)-3-[(4-cyanophenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3-methylphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(propan-2-yloxy)phenyl]-λ⁶-sulfanyl]prop-2-en-1-yl]-2-oxo-1,2,5,6,7,8-hexahydroquinoline-3-carboxamide;
N-[(2E)-3-[imino(4-methoxy-2-methylphenyl)oxo-λ⁶-sulfanyl]prop-2-en-1-yl]-2-oxo-1,2,5,6,7,8-hexahydroquinoline-3-carboxamide;
cyclopropylmethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
2-fluoroethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
2-fluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
2,2-difluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)(4-phenoxyphenyl)-λ⁶-sulfanyl]prop-2-en-1-yl]-2-oxo-1,2,5,6,7,8-hexahydroquinoline-3-carboxamide;
N-[(2E)-3-{[4-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-6-methyl-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1,2-dihydroquinoline-3-carboxamide;
N-[(2E)-3-[(3-fluoro-4-methoxyphenyl)(imino)oxo-λ⁶-sulfanyl]prop-2-en-1-yl]-2-oxo-6-(propan-2-yl)-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-6-methoxy-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-6-(2,2,2-trifluoroethyl)-1,2,5,6,7,8-hexahydro-1,6-naphthyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,7H,8H-pyrano[4,3-b]pyridine-3-carboxamide;
N-[(2E)-3-[imino(oxo)(3-phenoxyphenyl)-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-6-(trifluoromethyl)-1,2,5,6,7,8-hexahydroquinoline-3-carboxamide;
6-tert-butyl-N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-5-phenyl-1,2-dihydropyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H,8H,9H-cyclohepta[b]pyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H-cyclopenta[b]pyridine-3-carboxamide;
tert-butyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
6-benzyl-N-[(2E)-3-[(3-fluoro-4-methoxyphenyl)(imino)oxo-λ⁶-sulfanyl]prop-2-en-1-yl]-2-oxo-1,2,5,6,7,8-hexahydro-1,6-naphthyridine-3-carboxamide;
N-[(2E)-3-[(3-fluoro-4-methoxyphenyl)(imino)oxo-λ⁶-sulfanyl]prop-2-en-1-yl]-6-[(4-methoxyphenyl)methyl]-2-oxo-1,2,5,6,7,8-hexahydro-1,6-naphthyridine-3-carboxamide;
4,4-difluorocyclohexyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
cyclohexyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate
N-[(2E)-3-[imino(oxo)[4-(trifluoromethyl)phenyl]-λ⁶-sulfanyl]prop-2-en-1-yl]-2-oxo-1,2,5,6,7,8-hexahydroquinoline-3-carboxamide;
N-[(2E)-3-({[1,1′-biphenyl]-4-yl}(imino)oxo-λ⁶-sulfanyl)prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1,2-dihydro-1,8-naphthyridine-3-carboxamide;
N-[(2E)-3-[(3-fluoro-4-methoxyphenyl)(imino)oxo-λ⁶-sulfanyl]prop-2-en-1-yl]-2-oxo-1,2-dihydro-1,6-naphthyridine-3-carboxamide;
6-fluoro-N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-{imino[4-(morpholin-4-yl)phenyl]oxo-λ⁶-sulfanyl}prop-2-en-1-yl]-2-oxo-1,2,5,6,7,8-hexahydroquinoline-3-carboxamide;
N-[(2E)-3-{[3-cyano-4-(piperidin-1-yl)phenyl](imino)oxo-λ⁶-sulfanyl}prop-2-en-1-yl]-2-oxo-1,2,5,6,7,8-hexahydroquinoline-3-carboxamide;
N-[(2E)-3-[imino(oxo)[4-(piperidin-1-yl)phenyl]-λ⁶-sulfanyl]prop-2-en-1-yl]-2-oxo-1,2,5,6,7,8-hexahydroquinoline-3-carboxamide;
N-[(2E)-3-[imino(oxo)[4-(pyrrolidin-1-yl)phenyl]-λ⁶-sulfanyl]prop-2-en-1-yl]-2-oxo-1,2,5,6,7,8-hexahydroquinoline-3-carboxamide;
N-[(2E)-3-{imino[4-(methylamino)phenyl]oxo-λ⁶-sulfanyl}prop-2-en-1-yl]-2-oxo-1,2,5,6,7,8-hexahydroquinoline-3-carboxamide;
N-[(2E)-3-{[3-cyano-4-(morpholin-4-yl)phenyl](imino)oxo-λ⁶-sulfanyl}prop-2-en-1-yl]-2-oxo-1,2,5,6,7,8-hexahydroquinoline-3-carboxamide;
N-[(2E)-3-[imino(oxo)[4-(2-oxopiperidin-1-yl)phenyl]-λ⁶-sulfanyl]prop-2-en-1-yl]-2-oxo-1,2,5,6,7,8-hexahydroquinoline-3-carboxamide;
N-[(2E)-3-[imino(1-methyl-1,2,3,4-tetrahydroquinolin-6-yl)oxo-λ⁶-sulfanyl]prop-2-en-1-yl]-2-oxo-1,2,5,6,7,8-hexahydroquinoline-3-carboxamide;
N-[(2E)-3-[(4-acetamidophenyl)(imino)oxo-λ⁶-sulfanyl]prop-2-en-1-yl]-2-oxo-1,2,5,6,7,8-hexahydroquinoline-3-carboxamide;
N-[(2E)-3-[(4-cyclopropoxyphenyl)(imino)oxo-λ⁶-sulfanyl]prop-2-en-1-yl]-2-oxo-1,2,5,6,7,8-hexahydroquinoline-3-carboxamide;
N-[(2E)-3-{[3-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(4-cyclohexylphenyl)(imino)oxo-λ⁶-sulfanyl]prop-2-en-1-yl]-2-oxo-1,2,5,6,7,8-hexahydroquinoline-3-carboxamide;
N-[(2E)-3-[(2,3-dihydro-1-benzofuran-5-yl)(imino)oxo-λ⁶-sulfanyl]prop-2-en-1-yl]-2-oxo-1,2,5,6,7,8-hexahydroquinoline-3-carboxamide;
N-[(2E)-3-[imino(oxo)[3-(trifluoromethoxy)phenyl]-λ⁶-sulfanyl]prop-2-en-1-yl]-2-oxo-1,2,5,6,7,8-hexahydroquinoline-3-carboxamide; and
N-[(2E)-3-[(3-fluoro-4-methoxyphenyl)(imino)oxo-λ⁶-sulfanyl]prop-2-en-1-yl]-2-oxo-1,2-dihydropyridine-3-carboxamide.

Highly promising results in relation to improved tumor necrosis factor (TNF) and/or nuclear factor kappa-light-chain-enhancer of activated B cells (NF-κB) activity are obtained wherein the compound is selected from the group consisting of:
N-[(2E)-3-(benzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-6-benzyl-2-oxo-1,2,5,6,7,8-hexahydro-1,6-naphthyridine-3-carboxamide;
N-[(2E)-3-(4-fluorobenzenesulfonyl)prop-2-en-1-yl]-2-oxo-1,2,5,6,7,8-hexahydroquinoline-3-carboxamide;
N-[(2E)-3-(3-fluoro-4-methoxybenzenesulfonyl)prop-2-en-1-yl]-2-oxo-1,2,5,6,7,8-hexahydroquinoline-3-carboxamide;
N-[(2E)-3-(4-chlorobenzenesulfonyl)prop-2-en-1-yl]-2-oxo-1,2,5,6,7,8-hexahydroquinoline-3-carboxamide;
2-methylpropyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(2-fluorobenzenesulfonyl)prop-2-en-1-yl]carbamoyl}-2-oxo-1,2,5,6,7,8-hexahydro-1,6-naphthyridine-6-carboxylate;
N-[(2E)-3-(2-fluorobenzenesulfonyl)prop-2-en-1-yl]-2-oxo-1,2,5,6,7,8-hexahydroquinoline-3-carboxamide;
6-benzyl-N-[(2E)-3-(4-chlorobenzenesulfonyl)prop-2-en-1-yl]-2-oxo-1,2,5,6,7,8-hexahydro-1,6-naphthyridine-3-carboxamide;
2-(4-chlorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(4-chlorobenzenesulfonyl)prop-2-en-1-yl]-2-oxo-1,2-dihydroquinoline-3-carboxamide;
cyclopropylmethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclobutyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1,1,1-trifluoropropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-(2-chloro-4-fluorobenzenesulfonyl)prop-2-en-1-yl]-2-oxo-1,2,5,6,7,8-hexahydroquinoline-3-carboxamide;
N-[(2E)-3-(2,4-difluorobenzenesulfonyl)prop-2-en-1-yl]-2-oxo-1H,2H,5H,6H,7H,8H,9H-cyclohepta[b]pyridine-3-carboxamide;
N-[(2E)-3-(benzenesulfonyl)prop-2-en-1-yl]-2-oxo-1H,2H,5H,6H,7H,8H,9H-cyclohepta[b]pyridine-3-carboxamide;
N-[(2E)-3-(3-fluorobenzenesulfonyl)prop-2-en-1-yl]-2-oxo-1,2,5,6,7,8-hexahydroquinoline-3-carboxamide;
N-[(2E)-3-(4-methoxybenzenesulfonyl)prop-2-en-1-yl]-2-oxo-1,2,5,6,7,8-hexahydroquinoline-3-carboxamide;
N-[(2E)-3-(2-chlorobenzenesulfonyl)prop-2-en-1-yl]-2-oxo-1,2,5,6,7,8-hexahydroquinoline-3-carboxamide;
cyclopentyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
cyclopropylmethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
cyclohexyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1-(2,6-difluorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
2-fluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-[(5-chloropyridin-2-yl)sulfonyl]prop-2-en-1-yl]-2-oxo-1,2,5,6,7,8-hexahydroquinoline-3-carboxamide;
2-oxo-N-[(2E)-3-(thiophene-2-sulfonyl)prop-2-en-1-yl]-1,2,5,6,7,8-hexahydroquinoline-3-carboxamide;
N-[(2E)-3-(4-bromobenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-2-oxo-6-(propan-2-yl)-1,2,5,6,7,8-hexahydroquinoline-3-carboxamide;
N-[(2E)-3-(benzenesulfonyl)prop-2-en-1-yl]-6,6-dimethyl-2-oxo-1,2,5,6,7,8-hexahydroquinoline-3-carboxamide;
N-[(2E)-3-(3,4-dimethylbenzenesulfonyl)prop-2-en-1-yl]-2-oxo-1,2,5,6,7,8-hexahydroquinoline-3-carboxamide;
N-[(2E)-3-(benzenesulfonyl)(1,1-²H2)prop-2-en-1-yl]-2-oxo-1,2,5,6,7,8-hexahydroquinoline-3-carboxamide;
N-[(2E)-3-[4-(dimethylamino)benzenesulfonyl]prop-2-en-1-yl]-2-oxo-1,2,5,6,7,8-hexahydroquinoline-3-carboxamide;
N-[(2E)-3-(4-cyclopropylbenzenesulfonyl)prop-2-en-1-yl]-2-oxo-1,2,5,6,7,8-hexahydroquinoline-3-carboxamide;
N-[(2E)-3-(4-cyanobenzenesulfonyl)prop-2-en-1-yl]-2-oxo-1,2,5,6,7,8-hexahydroquinoline-3-carboxamide;
N-[(2E)-3-[imino(4-methoxyphenyl)oxo-λ⁶-sulfanyl]prop-2-en-1-yl]-2-oxo-1,2,5,6,7,8-hexahydroquinoline-3-carboxamide;
N-[(2E)-3-[(3,4-dimethylphenyl)(imino)oxo-λ⁶-sulfanyl]prop-2-en-1-yl]-2-oxo-1,2,5,6,7,8-hexahydroquinoline-3-carboxamide;
N-[(2E)-3-[(4-tert-butylphenyl)(imino)oxo-λ⁶-sulfanyl]prop-2-en-1-yl]-2-oxo-1,2,5,6,7,8-hexahydroquinoline-3-carboxamide;
N-[(2E)-3-[(4-ethoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3,5-dimethylphenyl)oxo-λ⁶-sulfanyl]prop-2-en-1-yl]-2-oxo-1,2,5,6,7,8-hexahydroquinoline-3-carboxamide;
N-[(2E)-3-[imino(3-methoxyphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(trifluoromethoxy)phenyl]-λ⁶-sulfanyl]prop-2-en-1-yl]-2-oxo-1,2,5,6,7,8-hexahydroquinoline-3-carboxamide;
N-[(2E)-3-[(4-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[(3-chloro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[(3-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3-methylphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(propan-2-yloxy)phenyl]-λ⁶-sulfanyl]prop-2-en-1-yl]-2-oxo-1,2,5,6,7,8-hexahydroquinoline-3-carboxamide;
N-[(2E)-3-[imino(4-methoxy-2-methylphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)(4-phenoxyphenyl)-λ⁶-sulfanyl]prop-2-en-1-yl]-2-oxo-1,2,5,6,7,8-hexahydroquinoline-3-carboxamide;
N-[(2E)-3-{[4-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-6-methyl-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1,2-dihydroquinoline-3-carboxamide;
N-[(2E)-3-[(3-fluoro-4-methoxyphenyl)(imino)oxo-λ⁶-sulfanyl]prop-2-en-1-yl]-2-oxo-6-(propan-2-yl)-1,2,5,6,7,8-hexahydroquinoline-3-carboxamide;
N-[(2E)-3-[imino(oxo)(3-phenoxyphenyl)-λ⁶-sulfanyl]prop-2-en-1-yl]-2-oxo-1,2,5,6,7,8-hexahydroquinoline-3-carboxamide;
6-tert-butyl-N-[(2E)-3-[(3-fluoro-4-methoxyphenyl)(imino)oxo-λ^{I}-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H,8H,9H-cyclohepta[b]pyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H-cyclopenta[b]pyridine-3-carboxamide;
6-benzyl-N-[(2E)-3-[(3-fluoro-4-methoxyphenyl)(imino)oxo-λ⁶-sulfanyl]prop-2-en-1-yl]-2-oxo-1,2,5,6,7,8-hexahydro-1,6-naphthyridine-3-carboxamide;
N-[(2E)-3-[(3-fluoro-4-methoxyphenyl)(imino)oxo-λ⁶-sulfanyl]prop-2-en-1-yl]-6-[(4-methoxyphenyl)methyl]-2-oxo-1,2,5,6,7,8-hexahydro-1,6-naphthyridine-3-carboxamide;
cyclohexyl 3-{[(2E)-3-[(3-fluoro-4-methoxyphenyl)(imino)oxo-λ^{I}-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)[4-(trifluoromethyl)phenyl]-λ⁶-sulfanyl]prop-2-en-1-yl]-2-oxo-1,2,5,6,7,8-hexahydroquinoline-3-carboxamide;
N-[(2E)-3-({[1,1′-biphenyl]-4-yl}(imino)oxo-λ⁶-sulfanyl)prop-2-en-1-yl]-2-oxo-1,2,5,6,7,8-hexahydroquinoline-3-carboxamide;
N-[(2E)-3-{imino[4-(morpholin-4-yl)phenyl]oxo-λ⁶-sulfanyl}prop-2-en-1-yl]-2-oxo-1,2,5,6,7,8-hexahydroquinoline-3-carboxamide;
N-[(2E)-3-{[3-cyano-4-(piperidin-1-yl)phenyl](imino)oxo-λ⁶-sulfanyl}prop-2-en-1-yl]-2-oxo-1,2,5,6,7,8-hexahydroquinoline-3-carboxamide;
N-[(2E)-3-[imino(oxo)[4-(piperidin-1-yl)phenyl]-λ⁶-sulfanyl]prop-2-en-1-yl]-2-oxo-1,2,5,6,7,8-hexahydroquinoline-3-carboxamide;
N-[(2E)-3-[imino(oxo)[4-(pyrrolidin-1-yl)phenyl]-λ⁶-sulfanyl]prop-2-en-1-yl]-2-oxo-1,2,5,6,7,8-hexahydroquinoline-3-carboxamide;
N-[(2E)-3-{imino[4-(methylamino)phenyl]oxo-λ⁶-sulfanyl}prop-2-en-1-yl]-2-oxo-1,2,5,6,7,8-hexahydroquinoline-3-carboxamide;
N-[(2E)-3-[imino(1-methyl-1,2,3,4-tetrahydroquinolin-6-yl)oxo-λ⁶-sulfanyl]prop-2-en-1-yl]-2-oxo-1,2,5,6,7,8-hexahydroquinoline-3-carboxamide;
N-[(2E)-3-[(4-cyclopropoxyphenyl)(imino)oxo-λ⁶-sulfanyl]prop-2-en-1-yl]-2-oxo-1,2,5,6,7,8-hexahydroquinoline-3-carboxamide;
N-[(2E)-3-{[3-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(4-cyclohexylphenyl)(imino)oxo-λ⁶-sulfanyl]prop-2-en-1-yl]-2-oxo-1,2,5,6,7,8-hexahydroquinoline-3-carboxamide;
N-[(2E)-3-[(2,3-dihydro-1-benzofuran-5-yl)(imino)oxo-λ⁶-sulfanyl]prop-2-en-1-yl]-2-oxo-1,2,5,6,7,8-hexahydroquinoline-3-carboxamide; and
N-[(2E)-3-[imino(oxo)[3-(trifluoromethoxy)phenyl]-λ⁶-sulfanyl]prop-2-en-1-yl]-2-oxo-1,2,5,6,7,8-hexahydroquinoline-3-carboxamide.

In a second aspect the present invention relates to the compound of the present invention for use as a medicament. In a preferred embodiment of the present invention, the use of the compound is combined with radiotherapy, targeted therapy, agents suitable for use in chemotherapy (chemotherapeutic agents), and/or agents suitable for use in immunotherapy (immunotherapeutic agents). In particular, the use of the compound is combined with such therapies and agents that cause increased levels of tumor necrosis factor (TNF), e.g. tumor necrosis factor alpha (TNFα) as the consequence of the treatment with such chemotherapeutic agents, immunotherapeutic agents, targeted therapy and/or radiotherapy.

Examples of radiotherapies that cause increased levels of TNF, e.g. TNFα, are, for example, described in the following publications: Rodemann, H. Peter, and Marcel A. Blaese (“Responses of normal cells to ionizing radiation.” Seminars in radiation oncology. Vol. 17. No. 2. WB Saunders, 2007), Di Maggio, Federica Maria, et al. (“Portrait of inflammatory response to ionizing radiation treatment.” Journal of inflammation 12.1 (2015): 1-11), and Meng, Guanmin, et al. (“Implications for breast cancer treatment from increased autotaxin production in adipose tissue after radiotherapy.” The FASEB Journal 31.9 (2017): 4064-4077).

Examples of targeted therapies that cause increased levels of TNF, e.g. TNFα, are, for example, described in the following publications: Tabolacci, Claudio, et al. (“Melanoma cell resistance to vemurafenib modifies inter-cellular communication signals.” Biomedicines 9.1 (2021): 79), Mercogliano, Maria Florencia, et al. (“Tumor necrosis factor α blockade: an opportunity to tackle breast cancer.” Frontiers in oncology 10 (2020): 584), and Labrie, Marilyne, et al. (“Therapy resistance: opportunities created by adaptive responses to targeted therapies in cancer.” Nature Reviews Cancer 22.6 (2022): 323-339).

Examples of chemotherapeutic and/or immunotherapeutic agents that cause increased levels of TNF, e.g. TNFα, are, for example, described in the following publications: Edwardson, Derek W., et al. (“Inflammatory cytokine production in tumor cells upon chemotherapy drug exposure or upon selection for drug resistance.” PloS one 12.9 (2017): e0183662), Mercogliano, Maria Florencia, et al. (“Tumor necrosis factor α blockade: an opportunity to tackle breast cancer.” Frontiers in oncology 10 (2020): 584), Vyas, Dinesh, Gieric Laput, and Arpitak K. Vyas (“Chemotherapy-enhanced inflammation may lead to the failure of therapy and metastasis.” OncoTargets and therapy 7 (2014): 1015), Husain, Beate, et al. (“Inflammatory markers in autoimmunity induced by checkpoint inhibitors.” Journal of Cancer Research and Clinical Oncology 147.6 (2021): 1623-1630), and Vanneman, Matthew, and Glenn Dranoff (“Combining immunotherapy and targeted therapies in cancer treatment.” Nature reviews cancer 12.4 (2012): 237-251).

In a third aspect of the present invention, the invention relates to the compound of the present invention for use in the treatment of cancer. In particular, the present invention relates to the compound of the present invention for use in the treatment of melanoma, bladder cancer, colon cancer, pancreatic cancer, ovarian cancer, breast cancer, bone cancer, lung cancer or haematological malignancies. It was found that the compound of the present invention exhibits particular promising results in the treatment of ovarian cancer, lung cancer or haematological malignancies.

In a fourth aspect of the present invention, the present invention relates to a method for inhibiting Met1-linked ubiquitination, wherein the method comprises the administration of the compound according to the present invention. Such method may relate to an in vitro method. However, in an embodiment of the present invention, the method may also relate to a method of treating a subject, such as a human. As such, the present invention relates to the compound of the present invention for inhibiting Met1-linked ubiquitination.

It is noted that the inhibition of the Met1-linked ubiquitination comprises the inhibition of linear ubiquitin (Ub) chain assembly complex (LUBAC). Inhibition of LUBAC preferably includes the inhibition of RNF31 (HOIP).

In an embodiment of the present invention, the present invention relates to a method for inhibiting Met1-linked ubiquitination, wherein the method comprises the administration of the compound according to the present invention, and wherein the inhibition of the Met1-linked ubiquitination comprises the irreversible or reversible inhibition of linear ubiquitin (Ub) chain assembly complex (LUBAC) via a covalent binding of the compound. It was found that the compounds of the present invention covalently inhibit the linear ubiquitin (Ub) chain assembly complex (LUBAC), in particular covalently inhibit RNF31.

In a fifth aspect the present invention relates to a composition comprising the compounds of the present invention wherein the composition further comprises at least one carrier.

In a sixth aspect the present invention relates to a pharmaceutical composition comprising the compounds of the present invention. Such composition may further comprise at least one pharmaceutically acceptable carrier. In an embodiment of the present invention the pharmaceutical composition may further comprise tumor necrosis factor (TNF).

In this respect, it is noted that the present invention also relates to formulations suitable for use as a pharmaceutical composition. Such formulation include, but are not limited to tablets, powders, capsules, pellets, solutions, suspensions, elixirs, emulsions, gels, creams, patch, or suppositories, including rectal and urethral suppositories.

### EXAMPLES

### Route of Synthesis

The following reaction Schemes illustrate the preparation of the compounds of the present invention. Unless otherwise indicated R⁶, R⁷, R⁸, X and A in the reaction Schemes are defined as above.

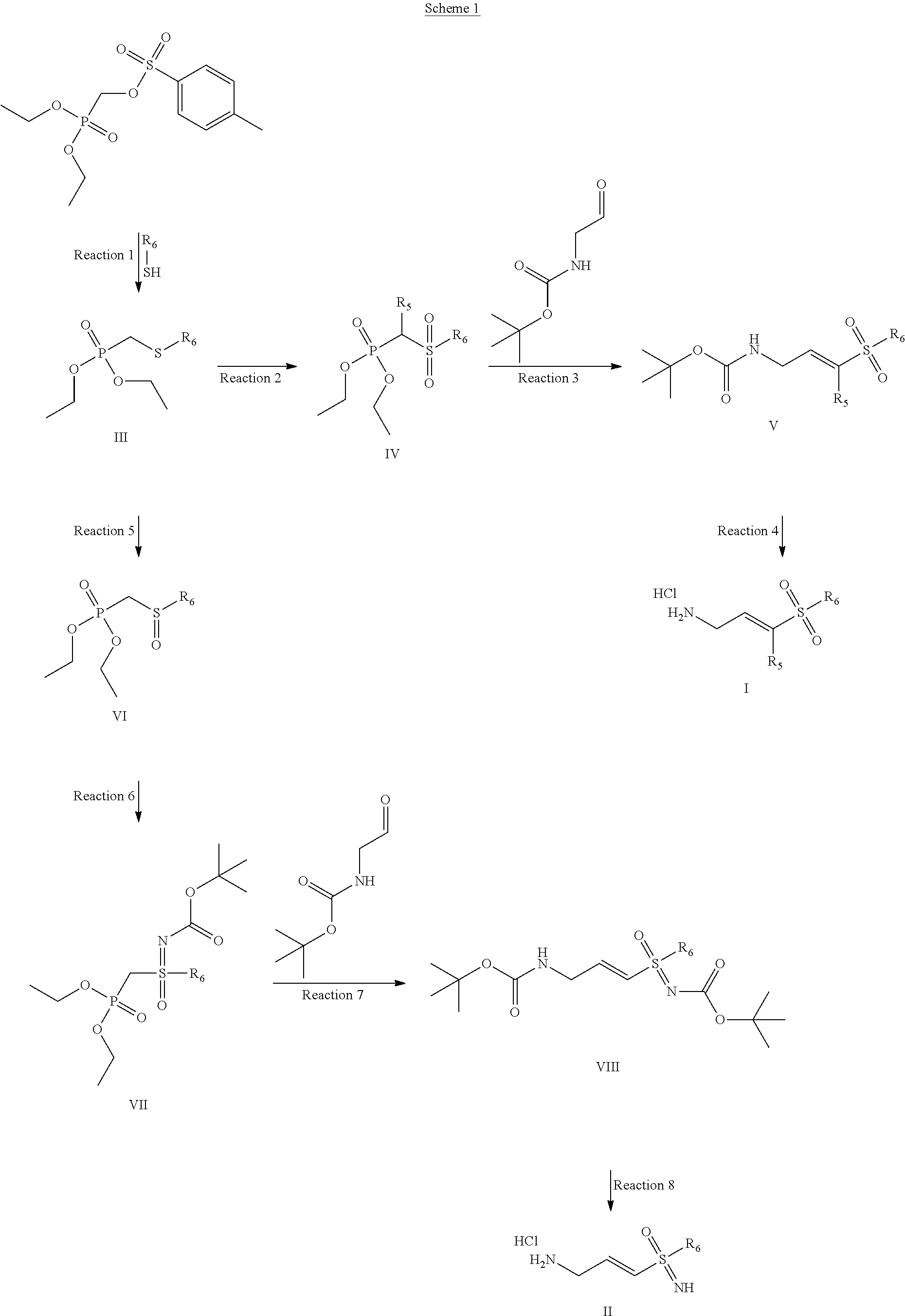

In Reaction 1 of Scheme 1, diethyl(tosyloxy)methylphosphonate is converted to the corresponding thioether of formula III by treating the tosylate with thiol in presence of inorganic base, e.g., s potassium carbonate in anhydrous in N,N-dimethylformamide. The reaction mixture is stirred at room temperature for a period of time ranging from 8 h to 72 h, preferably 12 h.

In Reaction 2 of Scheme 1, compound of formula III is oxidized to the corresponding sulfone of formula IV by treating Ill with oxidizing agent such as Oxone® monopersulfate or sodium tungstate and hydrogen peroxide in a mixture of water and a polar solvent such as ethyl acetate or alcohol solvent such as methanol. The reaction mixture is stirred at room temperature for a period of time between 8 h to 72 h, preferably 12 h. When R⁵ is not hydrogen, compound of formula IV is treated with an electrophilic agent, e.g., Selectfluor® or iodomethane, in presence of an organic base, e.g., potassium tert-butoxide in a polar aprotic solvent, e.g., tetrahydrofuran. The reaction mixture is stirred at room temperature for a period of time between 8 h min to 24 h, preferably 16 h.

In Reaction 3 of Scheme 1, compound of formula IV is converted to compound of formula V by reacting IV with (2-oxoethyl)carbamic acid tert-butyl ester in presence of an organic base such as potassium tert-butoxide in a polar aprotic solvent, e.g., tetrahydrofuran. The reaction mixture is stirred for a period of time between 1 h to 24 h, preferably 1.5 h.

In Reaction 4 of Scheme 1, removal of the protecting group from the compound of formula V is carried out by treating V with and an acid such as hydrogen chloride in an aprotic polar solvent such as 1,4-dioxane or diethyl ether. The reaction mixture is stirred at room temperature for a period of time between 10 min to 3 h, preferably 1 h.

In Reaction 5 of Scheme 1, compound of formula III is oxidized to the corresponding sulfoxide of general structure VI by treating Ill with oxidizing agent such as Oxone® monopersulfate in stochiometric amounts in a mixture of water and alcohol solvent such as methanol. The reaction is stirred for a period of time between 8 h to 72 h, preferably 12 h at room temperature.

In Reaction 6 of Scheme 1, compound of formula VI is converted in compound of formula VII by treating sulfoxide with tert-Butyl carbamate and iodobenzene diacetate in presence of rhodium (II) acetate dimer and magnesium oxide in a aprotic solvent such as dichloromethane. Reaction mixture is stirred at a temperature between 10° C. and 70° C., preferably 40° C. for a period of time between 6 h to 72 h, preferably 12 h.

In Reaction 7 of Scheme 1, compound of formula VII is converted in compound of formula VIII by reacting VII with (2-oxoethyl)carbamic acid tert-butyl ester in presence of organic base such as potassium tert-butoxide in a polar aprotic solvent such as tetrahydrofuran. The reaction mixture is stirred for a period of time between 1 h to 24 h, preferably 1.5 h.

In Reaction 8 of Scheme 1, removal of the protecting group from the compound of formula VIII is carried out by treating VIII with an acid such as hydrogen chloride in an aprotic polar solvent such as 1,4-dioxane or diethyl ether. The reaction mixture is stirred at room temperature for a period of time between 10 min to 3 h, preferably 1 h.

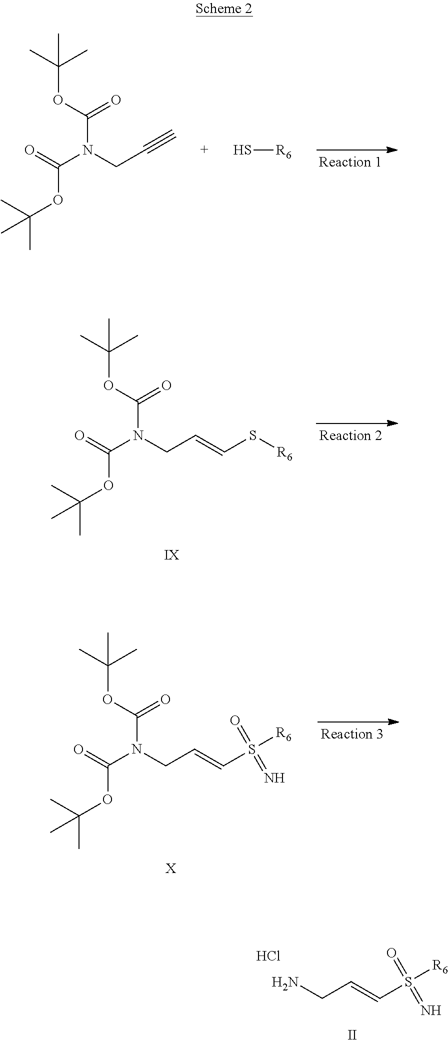

In Reaction 1 of Scheme 2,1,3-Bis(1,1-dimethylethyl) 2-(2-propyn-1-yl) imidodicarbonate is converted to the corresponding vinyl thioether of formula IX by treating alkyne with a thiol in presence of rhodium catalyst preferably tris(triphenylphosphine)rhodium(I) chloride in an aprotic solvent preferably DCE. The reaction mixture is stirred at a temperature between −10° C. to about 50° C., preferably 0° C. for a period of time between 12 h to 80 h, preferably 24 h.

In Reaction 2 of Scheme 2, compound of formula IX is oxidized to the corresponding sulfoximine of formula X by treating IX with ammonium carbonate and iodobenzene diacetate in an alcohol solvent such as methanol. The reaction mixture is stirred at a temperature between −10° C. to about 50° C., preferably 0° C. for a period of time between 10 min to 3 h, preferably 30 min.

In Reaction 3 of Scheme 2, removal of the protecting group from the compound of formula X is carried out by treating X with an acid such as hydrogen chloride in an aprotic polar solvent such as 1,4-dioxane or diethyl ether. The reaction mixture is stirred at room temperature for a period of time between 10 min to 3 h, preferably 30 min.

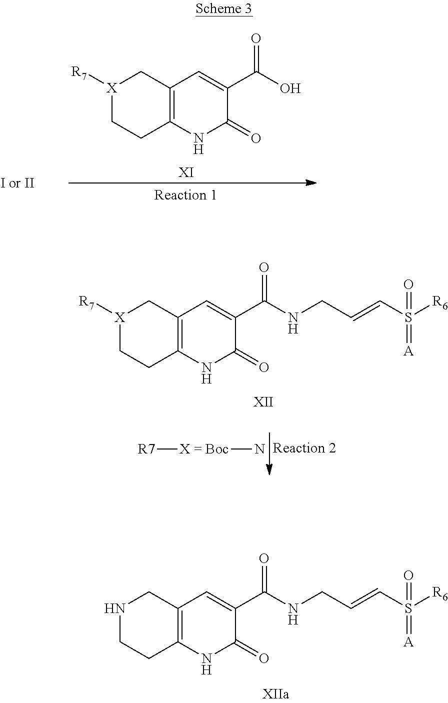

In Reaction 1 of Scheme 3, the amine of general structure I and II are converted into corresponding amide of general structure XII by treating acid with general structure XI, in presence of a amide coupling agent such as T3P (n-propanephosphonic acid anhydride) or HATU (1-[bis(dimethylamino)methylene]-1H- 1,2,3-triazolo[4,5-b]pyridinium 3-oxide hexafluorophosphate) and an organic base such as triethylamine or diisopropylethylamine in a polar solvent such as N,N-dimethylformamide or acetonitrile. The reaction mixture was stirred at room temperature for a period of time between 8 h to 72 h, preferably 16 h.

In Reaction 2 of Scheme 3, when X is tert-butyloxycarbonyl (Boc), removal of the Boc protecting group is carried out by treating XII with and acid such as hydrogen chloride in an aprotic polar solvent such 1,4-dioxane or diethyl ether. The reaction mixture is stirred at room temperature for a period of time between 10 min to 4 h, preferably 2 h.

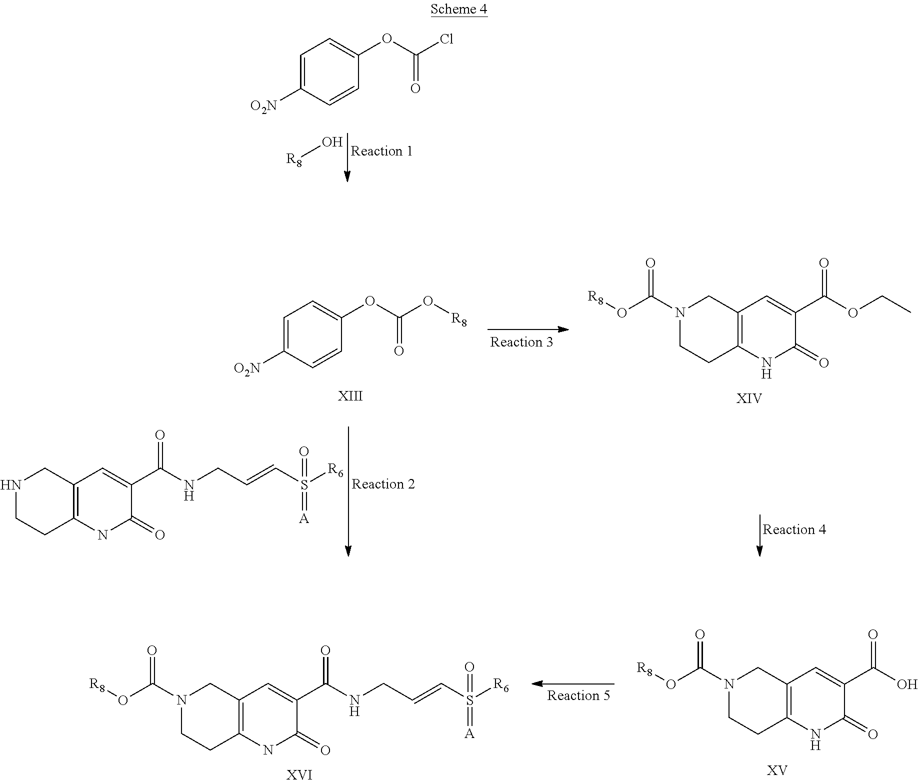

In Reaction 1 of Scheme 4, 4-nitrophenylchloroformate is converted to the corresponding carbonate of formula XIII, by treating chloroformate with alcohol in presence of an organic base such as pyridine in an aprotic chlorinated solvent such as dichloromethane. The reaction mixture is stirred at a room temperature for a period of time between 8 h to 72 h, preferably 12 h.

In Reaction 2 of Scheme 4, compound of formula XIII is converted to the correspondent carbamate of formula structure XVI by treating XIII with amine of formula XII where X is nitrogen, in presence of an inorganic base such as sodium hydrogen carbonate in a mixture of polar aprotic and protic solvents such as water and tetrahydrofuran. The reaction mixture is stirred at room temperature for a period of time between 1 h to 12 h preferably 2 h.

In Reaction 3 of Scheme 4, compound of formula XIII is converted to the correspondent carbamate of formula structure XIV by treating XIII with Ethyl 1,2,5,6,7,8-hexahydro-2-oxo-1,6-naphthyridine-3-carboxylate in presence of an inorganic base such as sodium hydrogen carbonate in a mixture of polar aprotic and protic solvents such water and tetrahydrofuran. The reaction mixture is stirred at room temperature for a period of time between 1 h to 12 h preferably 2 h.

In Reaction 4 of Scheme 4, compound of formula XII is converted to the correspondent acid XIII by treating XII with a 1.0 molar aqueous solution of inorganic base such as lithium hydroxide in polar aprotic solvent such as tetrahydrofuran. The reaction mixture is stirred at room temperature for a period of time between 8 h to 72 h preferably 12 h.

In Reaction 5 of Scheme 4, the same procedure as described in Reaction 1 of Scheme 3 is used.

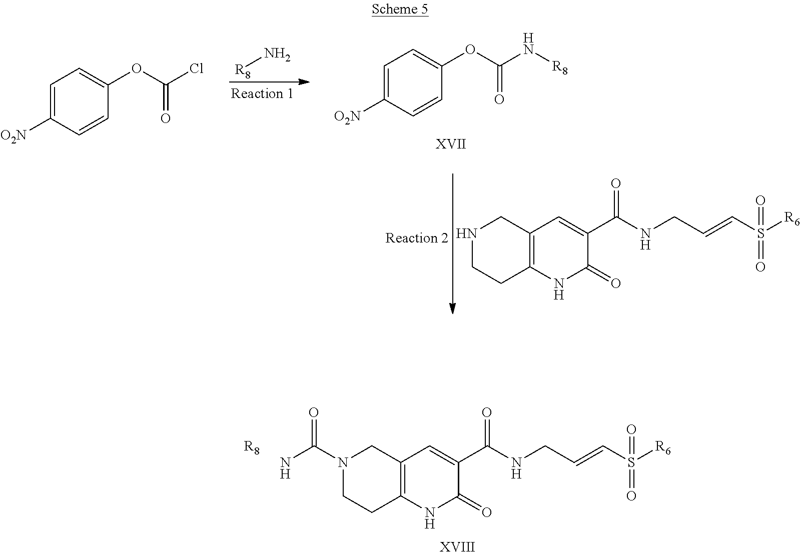

In Reaction 1 of Scheme 5, 4-nitrophenylchloroformate is converted to the corresponding carbamate of formula XVII by treating chloroformate with amine in presence of an organic base such as pyridine in an aprotic chlorinated solvent such as dichloromethane. The reaction mixture is stirred at a room temperature for a period of time between 8 h to 72 h, preferably 12 h.

In Reaction 2 of Scheme 5, compound of formula XVII is converted to the correspondent urea of formula XVIII by treating XVII with an amine of formula XII where X is nitrogen, in the presence of an inorganic base such as sodium hydrogen carbonate in a mixture of polar aprotic and protic solvents such as water and tetrahydrofuran.

The reaction mixture is stirred at room temperature for a period of time between 1 h to 12 h preferably 2 h.

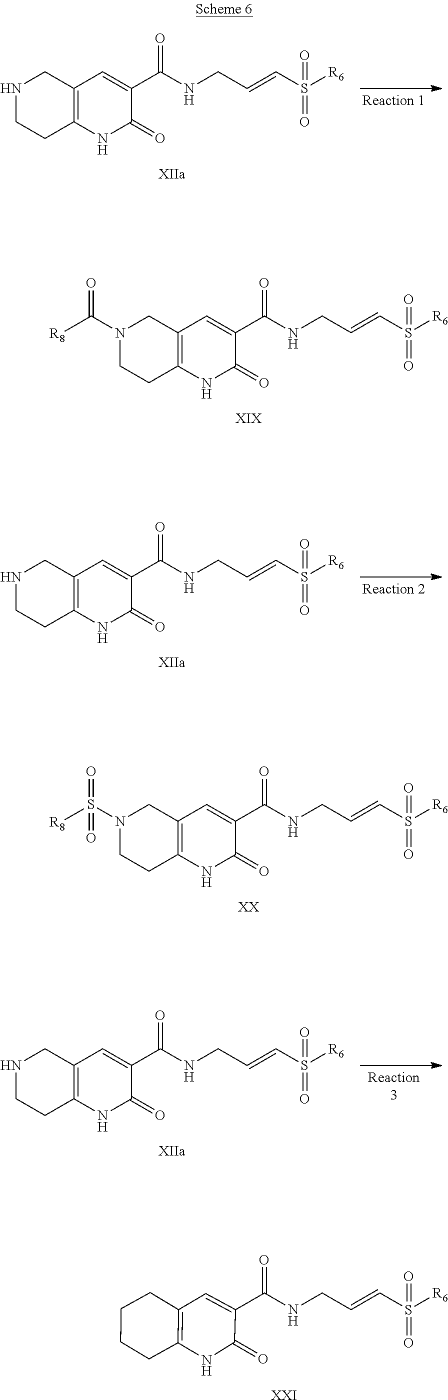

In Reaction 1 of Scheme 6, compound of formula XII, where X is nitrogen, is converted to the correspondent amide of formula structure XIX by treating XII with acid in presence of an amide coupling agent such as T3P or HATU and an organic base such as triethylamine or diisopropylethylamine in a polar solvent such as N,N-dimethylformamide or acetonitrile. The reaction mixture was stirred at room temperature for a period of time between 8 h to 72 h, preferably 16 h.

In Reaction 2 of Scheme 6, compound of formula XII, where X is nitrogen, is converted to the correspondent sulphonamide of formula structure XX by treating XII with an organic sulfonyl chloride in the presence of an organic base such as triethylamine or diisopropylethylamine in a polar solvent such as N,N-dimethylformamide or acetonitrile. The reaction mixture was stirred at room temperature for a period of time between 8 h to 72 h, preferably 16 h.

In Reaction 3 of Scheme 6, compound of formula XII, where X is nitrogen, is converted to the correspondent amino alkyl of formula XXI by treating XII with an organic bromide or chloride in presence of an inorganic base such as potassium carbonate in a polar solvent such as N,N-dimethylformamide. The reaction mixture was stirred at room temperature for a period of time between 2 h to 24 h, preferably 4 h.

Compounds The following Examples illustrate the preparation of the compounds of the present invention but is not limited to the details thereof. NMR data are reported in parts per million (δ) and are referenced to the deuterium lock signal from the sample solvent (deuteriochloroform unless otherwise specified). Commercial reagents were used without further purification. THF refers to tetrahydrofuran, DMF refers to N,N-dimethylformamide, MeOH refers to methanol, DCM refers to dichloromethane. Room temperature refers to 20-25° C.

### Example 1: Preparation of (2E)-3-(4-bromobenzenesulfonyl)prop-2-en-1-amine hydrochloride (Scheme 1)

### Scheme 1, Reaction 1: diethyl {[(4-bromophenyl)sulfanyl]methyl}phosphonate

Diethyl(tosyloxy)methylphosphonate (3.0 g, 9.1 mmol, 1.0 eq) and potassium carbonate anhydrous (3.15 g, 22.8 mmol, 2.5 eq) were suspended in anhydrous DMF (24.0 ml, 8.0 vol) before mixing with 4-bromothiophenol (1.91 g, 9.6 mmol, 1.05 eq) in one go and stirring at room temperature UPLC after stirring 12 h indicated full consumption of the starting material to give the desired product. The reaction mixture was concentrated under reduced pressure and partitioned between ethyl acetate (20 mL) and distilled water (30 mL) with saturated NaCl solution (10 mL). The layers were separated and the aqueous layer was extracted with ethyl acetate (3×10 mL). The combined organic layers were dried with MgSO₄, filtered and concentrated under reduced pressure to give a light-yellow oil (3.39 g). The oil was dissolved in minimal DCM and purified by flash chromatography, eluting with hexanes/ethyl acetate (0 to 55%). Product-containing fractions were collected and concentrated under reduced pressure to give the desired product, diethyl {[(4-bromophenyl)sulfanyl]methyl}phosphonate, as a colorless oil (3.02 g, Y: 97%). UPLC and ¹H NMR (CDCl3, 300 MHz) confirmed the structure and purity of the product.

UPLC Rt: 2.02, m/z: 341.0 [M+1]; Purity (254 nm): 99%; 1H NMR (300 MHz, Chloroform-d) δ 7.47-7.37 (m, 2H), 7.36-7.27 (m, 2H), 4.14 (dqd, J=8.2, 7.0, 1.0 Hz, 4H), 3.17 (s, 1H), 3.13 (s, 1H), 1.31 (td, J=7.1, 0.5 Hz, 6H).

### Scheme 1, Reaction 2: diethyl [(4-bromobenzenesulfonyl)methyl]phosphonate

Diethyl {[(4-bromophenyl)sulfanyl]methyl}phosphonate (0.4 g, 1.18 mmol, 1.0 eq.) was dissolved in anhydrous MeOH (4.0 ml, 10.0 vol) before addition of Oxone®, monopersulfate compound (0.91 g, 2.95 mmol, 2.5 eq.) solution in distilled water (4.0 ml, 10.0 vol), immediately turning the pale yellow solution into a bright white mixture. After stirring for 12 h, UPLC analysis indicated full conversion to the desired product. The mixture was filtered through Celite, washed through with MeOH and concentrated under reduced pressure to a low volume. The reduced filtrate was partitioned between ethyl acetate (20 mL) and water (25 mL) with saturated NaCl solution (5 mL). The layers were separated and the aqueous layer was extracted with ethyl acetate (3×10 mL). The combined organic layers were dried with MgSO4, filtered and concentrated under reduced pressure to give the desired product, diethyl [(4-bromobenzenesulfonyl)methyl]phosphonate, as a yellow oil (448 mg, Y: 99%). The product was used in the next step without any further purification. UPLC and ¹H NMR (CDCl3, 300 MHz) confirmed the structure and purity of the product.

UPLC Rt: 1.99, m/z: 373.0 [M+1]; ¹H NMR (300 MHz, Chloroform-d) δ 7.92-7.80 (m, 2H), 7.77-7.66 (m, 2H), 4.17 (dq, J=8.3, 7.1 Hz, 4H), 3.77 (s, 1H), 3.72 (s, 1H), 1.36-1.27 (m, 6H).

### Scheme 1, Reaction 3: tert-butyl N-[(2E)-3-(4-bromobenzenesulfonyl)prop-2-en-1-yl]carbamate

Potassium tert-butoxide (0.13 g, 1.18 mmol, 1.0 eq.) was suspended in anhydrous THF (9.0 ml) with cooling to 0° C. over 5 min. Diethyl [(4-bromobenzenesulfonyl)methyl]phosphonate (0.45 g, 1.18 mmol, 1.0 eq.) was added dropwise, as a solution in anhydrous THF (2.83 ml), instantly forming a yellow solution, which was stirred at 0° C. under argon for 30 min. Tert-butyl N-(2-oxoethyl)carbamate (0.20 g, 1.24 mmol, 1.05 eq.) was added, as a solution in anhydrous THF (2.37 ml, 0.5 M) over 1 min, and the reaction was stirred under argon and, subsequently, allowed to reach room temperature. After 90 min, the reaction was concentrated under reduced pressure to give a yellow oil, which was partitioned between ethyl acetate (10 mL) and saturated aqueous NaHCO₃ (20 mL). The layers were separated and the organic layer was washed with saturated NaCl solution (20 mL). The combined aqueous layers were extracted with ethyl acetate (3×10 mL) and the combined organic layers were dried with MgSO₄, filtered and concentrated under reduced pressure to give a yellow oil. The oil was dissolved in minimal DCM and purified by flash chromatography, eluting with hexanes/ethyl acetate (0 to 36%). Clean product-containing fractions were collected and concentrated under reduced pressure to give the desired product, tert-butyl N-[(2E)-3-(4-bromobenzenesulfonyl)prop-2-en-1-yl]carbamate, as a pale yellow oil (211 mg, Y: 47%). UPLC and ¹H NMR (DMSO-d₆, 300 MHz) confirmed structure and purity of the product as the (E)-isomer.

UPLC Rt: 1.87, m/z: 376.0 [M+Na]; Purity (254 nm): 99%; ¹H NMR (300 MHz, DMSO-d₆) δ 7.91-7.83 (m, 2H), 7.83-7.73 (m, 2H), 7.18 (t, J=5.2 Hz, 1H), 6.86 (dt, J=15.1, 4.4 Hz, 1H), 6.68 (d, J=15.2 Hz, 1H), 3.78 (d, J=6.0 Hz, 2H), 1.36 (s, 9H).

### Scheme 1, Reaction 4: (2E)-3-(4-bromobenzenesulfonyl)prop-2-en-1-amine hydrochloride

Refrigerated 4N HCl in dioxane, 3.7-4.3N (1.40 ml, 5.61 mmol, 10.0 eq.) was added to tert-butyl N-[(2E)-3-(4-bromobenzenesulfonyl)prop-2-en-1-yl]carbamate (0.21 g, 0.56 mmol, 1.0 eq.) and the solution was stirred at room temperature. A white solid began to form within the first 10 min and after stirring for 1 h, the reaction was concentrated under reduced pressure to give the desired product, (2E)-3-(4-bromobenzenesulfonyl)prop-2-en-1-amine hydrochloride, as a white solid (161 mg, Y: 92%), which was taken forward to the next reaction without further purification. UPLC and ¹H NMR (DMSO-d₆, 300 MHz) confirmed the structure and purity of the product.

UPLC Rt: 1.41 min, m/z: 276.0 [M+1], Br pattern; Purity (254 nm): 99%; ¹H NMR (300 MHz, DMSO-d₆) δ 8.23 (s, 3H), 7.96-7.86 (m, 2H), 7.84-7.75 (m, 2H), 7.08 (dt, J=15.3, 1.6 Hz, 1H), 6.90 (dt, J=15.3, 5.2 Hz, 1H), 3.72 (d, J=5.7 Hz, 2H).

### Example 2: Preparation of [(1E)-3-aminoprop-1-en-1-yl](4-fluorophenyl)imino-λ⁶-sulfanone dihydrochloride (Scheme 1)

### Scheme 1, Reaction 1: diethyl {[(4-fluorophenyl)sulfanyl]methyl}phosphonate

Diethyl(tosyloxy)methylphosphonate (9.86 g, 30 mmol, 1.0 eq.) and potassium carbonate anhydrous (10.4 g, 75 mmol, 2.5 eq.) were suspended in anhydrous DMF (79 ml, 8.0 vol) before mixing with 4-fluorothiophenol (4.04 g, 31.5 mmol, 1.05 eq) and stirring at room temperature. UPLC after stirring overnight indicated full consumption of the starting material with formation of the desired product. The reaction was concentrated under reduced pressure to a low volume and partitioned between ethyl acetate (20 mL) and distilled water (70 mL) with saturated NaCl solution (10 mL). The layers were separated and the aqueous layer was extracted with ethyl acetate (5×20 mL). The combined organic layers were dried with MgSO₄, filtered and concentrated under reduced pressure to give a yellow oil. The oil was dissolved in minimal DCM and purified by flash chromatography, eluting with hexanes/ethyl acetate (0 to 50%). Product-containing fractions were collected and concentrated under reduced pressure to give the desired product, diethyl {[(4-fluorophenyl)sulfanyl]methyl}phosphonate, as a light yellow oil (8.19 g, Y:98%). UPLC and ¹H NMR (CDCl₃, 300 MHz) confirmed structure and purity of the product.

UPLC Rt: 1.76, m/z: 279.10 [M+1]; Purity (254 nm): 94%; ¹H NMR (300 MHz, Chloroform-d) δ 7.55-7.41 (m, 2H), 7.07-6.95 (m, 2H), 4.13 (dqd, J=8.4, 7.0, 1.3 Hz, 4H), 3.15 (s, 1H), 3.10 (s, 1H), 1.31 (t, J=7.1 Hz, 6H).

### Scheme 1, Reaction 5: diethyl [(4-fluorobenzenesulfinyl)methyl]phosphonate

Diethyl {[(4-fluorophenyl)sulfanyl]methyl}phosphonate (8.19 g, 29.4 mmol, 1.0 eq) was dissolved in anhydrous MeOH (81.9 ml, 10.0 vol) and cooled to 0° C. for 20 min before addition of Oxone®, monopersulfate compound (9.50 g, 31 mmol, 1.05 eq.) solution in distilled water (82 ml, 10.0 vol), immediately turning the pale yellow solution into a bright white mixture, which was stirred at 0° C. for 30 min before being allowed to reach room temperature. After stirring overnight, UPLC suggested complete formation of the desired product with consumption of the starting material. The reaction was filtered through Celite, washed through with MeOH (5×20 mL) and concentrated under reduced pressure to a low volume. The concentrated filtrate was partitioned between ethyl acetate (40 mL) and distilled water (80 mL) with NaCl saturated solution (20 mL). The layers were separated and the aqueous layer was extracted with ethyl acetate (5×20 mL). The combined organic layers were dried with MgSO₄, filtered and concentrated under reduced pressure to give the desired product, diethyl [(4-fluorobenzenesulfinyl)methyl]phosphonate, as a yellow oil (8.754 g) suitable pure for the next step. UPLC and ¹H NMR (CDCl₃, 300 MHz) confirmed the structure and purity of the product.

UPLC Rt: 1.53, m/z: 295.00 [M+1]; Purity (254 nm): 99%; ¹H NMR (300 MHz, Chloroform-d) δ 7.83-7.71 (m, 2H), 7.28-7.20 (m, 2H), 4.26-4.00 (m, 4H), 3.42 (t, J=14.7 Hz, 1H), 3.28 (t, J=14.9 Hz, 1H), 1.31 (dt, J=10.5, 7.0 Hz, 6H).

### Scheme 1, Reaction 6: N-{[(diethoxyphosphoryl)methyl](4-fluorophenyl)oxo-λ⁶-sulfanylidene}carbamate

Diethyl [(4-fluorobenzenesulfinyl)methyl]phosphonate (1.0 g, 3.4 mmol, 1.0 eq) was added, as a solution in anhydrous DCM (20 ml, 0.17 M), to a vial containing rhodium(II) acetate dimer (0.045 g, 0.10 mmol, 0.03 eq), tert-butyl carbamate (0.80 g, 6.8 mmol, 2.0 eq) and magnesium oxide (0.55 g, 13.6 mmol, 4.0 eq). The mixture was degassed with argon for 20 min before addition of iodobenzene diacetate (1.642 g, 5.097 mmol, 1.5 eq) The vial was sealed before heating and after stirring 12 h at 50° C. UPLC indicated near-total consumption of the starting material. The reaction mixture was filtered through Celite, washed through with DCM (6×10 mL) and concentrated under reduced pressure to give a yellow oil (3.458 g). The oil was dissolved in minimal DCM and purified by flash chromatography, eluting with hexanes/ethyl acetate (0 to 65%). Product-containing fractions were collected and concentrated under reduced pressure to give the desired product, tert-butyl N-{[(diethoxyphosphoryl)methyl](4-fluorophenyl)oxo-λ⁶-sulfanylidene}carbamate, as a yellow oil (825 mg Y: 59%). UPLC and ¹H NMR (CDCl₃, 300 MHz) confirmed the purity and structure of the product. UPLC Rt: 1.78, m/z: 432.3 [M+Na]; Purity (254 nm): 99%; ¹H NMR (300 MHz, Chloroform-d) δ 8.14-8.04 (m, 2H), 7.31-7.20 (m, 2H), 4.39 (dd, J=16.3, 15.4 Hz, 1H), 4.21-4.07 (m, 4H), 4.07-3.96 (m, 1H), 1.43 (s, 9H), 1.27 (dtd, J=12.5, 7.1, 0.7 Hz, 6H).

### Scheme 1, Reaction 7: tert-butyl N-[(2E)-3-({[(tert-butoxy)carbonyl]imino}(4-fluorophenyl)oxo-λ⁶-sulfanyl)prop-2-en-1-yl]carbamate

Potassium tert-butoxide (0.14 g, 1.22 mmol, 1.0 eq) was suspended in anhydrous THF (9.0 ml) and the mixture was degassed with argon while cooling at 0° C. over 20 min. N-{[(diethoxyphosphoryl)methyl](4-fluorophenyl)oxo-λ⁶-sulfanylidene}carbamate (0.5 g, 1.22 mmol, 1.0 eq) was added, as a solution in anhydrous THF (3.2 ml), causing the mixture to form a yellow solution. This was stirred at 0° C. for 30 min before adding tert-butyl N-(2-oxoethyl)carbamate (0.20 g, 1.28 mmol, 1.05 eq), as a solution in anhydrous THF (2.44 ml, 0.5 M), and the reaction was allowed to reach room temperature, stirring under argon. After 90 min, the reaction was concentrated to dryness under reduced pressure before partitioning between ethyl acetate (20 mL) and saturated aqueous NaHCO₃ solution (20 mL). The layers were separated and the organic layer was washed with saturated NaCl solution (10 mL). The aqueous layers were extracted with ethyl acetate (2×10 mL) and the combined organic layers were dried with MgSO₄, filtered and concentrated under reduced pressure to give a yellow oil (589 mg). The oil was dissolved in minimal DCM and purified by flash chromatography, eluting with DCM/ethyl acetate (0 to 12%). Product-containing fractions were collected and concentrated under reduced pressure to give the desired product, tert-butyl N-{[(1E)-3-{[(tert-butoxy)carbonyl]amino}prop-1-en-1-yl](4-fluorophenyl)oxo-λ⁶-sulfanylidene}carbamate, as a yellow oil (225 mg, Y: 44%). UPLC and ¹H NMR (DMSO-d₆, 300 MHz) confirmed the structure and purity of the (E)-isomer product.

UPLC Rt: 1.87, m/z: 437.1 [M+Na]; Purity (254 nm): 99%; 1H NMR (300 MHz, DMSO-d6) δ 8.00-7.86 (m, 2H), 7.57-7.44 (m, 2H), 7.21 (t, J=5.2 Hz, 1H), 6.84 (dt, J=14.9, 4.2 Hz, 1H), 6.72 (d, J=15.1 Hz, 1H), 3.80 (s, 2H), 1.36 (s, 9H), 1.25 (s, 9H).

### Scheme 1, Reaction 8: [(1E)-3-aminoprop-1-en-1-yl](4-fluorophenyl)imino-λ⁶-sulfanone dihydrochloride

Refrigerated 4N HCl in dioxane, 3.7-4.3N (1.36 ml, 5.4 mmol, 10.0 eq.) was added to tert-butyl N-[(2E)-3-({[(tert-butoxy)carbonyl]imino}(4-fluorophenyl)oxo-λ⁶-sulfanyl)prop-2-en-1-yl]carbamate (0.23 g, 0.54 mmol, 1.0 eq) and the solution was stirred at room temperature. A white solid formed within the first 10 min. After 1 h, the reaction was concentrated under reduced pressure to give the desired product, [(1E)-3-aminoprop-1-en-1-yl](4-fluorophenyl)imino-λ⁶-sulfanone dihydrochloride, as a light brown solid (198 mg), which was taken forward to the next reaction without further purification. ¹H NMR (DMSO-d₆, 300 MHz) confirmed structure of the product.

UPLC Rt: 0.66, m/z=215.1 [M+1]; Purity (254 nM): 71% (sum of the 3 peaks); ¹H NMR (300 MHz, DMSO-d₆) δ 8.29 (s, 6H), 8.06-7.91 (m, 5H), 7.55-7.43 (m, 4H), 6.96 (d, J=15.1 Hz, 1H), 6.78 (dt, J=15.1, 5.3 Hz, 1H).

### Example 3: Preparation of (2E)-3-(4-chlorobenzenesulfonyl)-3-fluoroprop-2-en-1-amine hydrochloride (Scheme 1)

Scheme 1, Reaction 1: diethyl {[(4-chlorophenyl)sulfanyl]methyl}phosphonate Diethyl(tosyloxy)methylphosphonate (11.2 g, 34.3 mmol, 1.0 eq) and potassium carbonate anhydrous (11.8 g, 85.7 mmol, 2.5 eq) were suspended in N,N-dimethylformamide anhydrous (90.2 ml, 8.0 vol) before mixing with 4-bromothiophenol (5.6 g, 37.7 mmol, 1.1 eq) in one go and stirring at room temperature UPLC after stirring 12 h indicated full consumption of the starting material to give the desired product. The reaction mixture was concentrated under reduced pressure and partitioned between ethyl acetate (50 mL) and distilled water (750 mL) with saturated NaCl solution (50 mL). The layers were separated and the aqueous layer was extracted with ethyl acetate (4×50 mL). The combined organic layers were dried with MgSO₄, filtered and concentrated under reduced pressure to give a light-yellow oil (12.896 g). The oil was dissolved in minimal dichloromethane and purified by flash chromatography, eluting with hexanes/ethyl acetate (0 to 75%). Product-containing fractions were collected and concentrated under reduced pressure to give the desired product, diethyl {[(4-chlorophenyl)sulfanyl]methyl}phosphonate, as a light yellow oil (9.88 g, Y: 97%). UPLC and ¹H NMR (CDCl₃, 300 MHz) confirmed the structure and purity of the product.

UPLC Rt: 1.84, m/z: 295.1 [M+1] Cl pattern; Purity (254 nm): 99%; ¹H NMR (300 MHz, Chloroform-d) δ 7.44-7.37 (m, 2H), 7.33-7.26 (m, 2H), 4.25-4.07 (m, 4H), 3.20 (s, 1H), 3.15 (s, 1H), 1.33 (t, J=7.1 Hz, 6H).

### Scheme 1, Reaction 2: diethyl [(4-chlorobenzenesulfonyl)(fluoro)methyl]phosphonate

Diethyl {[(4-bromophenyl)sulfanyl]methyl}phosphonate (9.8 g, 33.5 mmol, 1.0 eq) was dissolved in anhydrous methanol (59.3 ml, 6.0 vol) before addition of Oxone® monopersulfate compound (22.6 g, 73.7 mmol, 2.2 eq) solution in distilled water (59.3 ml, 6.0 vol), immediately turning the pale yellow solution into a bright white mixture. After stirring for 12 h, UPLC analysis indicated full conversion to the desired product. The mixture was filtered through Celite, washed through with MeOH and concentrated under reduced pressure to a low volume. The reduced filtrate was partitioned between ethyl acetate (30 mL) and water (90 mL) with saturated NaCl solution (10 mL). The layers were separated and the aqueous layer was extracted with ethyl acetate (3×20 mL). The combined organic layers were dried with MgSO₄, filtered and concentrated under reduced pressure to give the desired product, diethyl [(4-chlorobenzenesulfonyl)methyl]phosphonate, as a yellow oil (10.76 g, Y: 98%). The product was used in the next step without any further purification. UPLC and ¹H NMR (CDCl₃, 300 MHz) confirmed the structure and purity of the product. UPLC Rt: 1.68, m/z: 325.1 [M−1], Cl pattern; Purity (254 nm): 98%.

Potassium tert-butoxide (0.34 g, 3.06 mmol, 1.0 eq) was added to a dried flask and suspended in tetrahydrofuran anhydrous (25.0 ml) with cooling to 0 C over 10 min. diethyl [(4-chlorobenzenesulfonyl)methyl]phosphonate (1.0 g, 3.06 mmol, 1.0 eq) was added dropwise, as a solution in tetrahydrofuran anhydrous (5.6 ml), forming a yellow solution, which was stirred at 0 C under argon for 30 min. F-TEDA-BF4 (1.14 g, 3.21 mmol, 1.05 eq) was added dropwise over 1 min, as a solution in N,N-dimethylformamide anhydrous (4.1 ml, 4.1 vol), and the reaction was allowed to reach rt and stirred for 16 h in argon atmosphere. The reaction was quenched with sat. aq. NH₄Cl solution (50 mL) and stirred for 10 min before concentrating to a low volume under reduced pressure and partitioning with EtOAc (20 mL). The layers were separated and the aqueous layer was extracted with EtOAc (3×20 mL). The combined organic layer was dried with MgSO₄, filtered and concentrated under reduced pressure to give a yellow oil (1.27 g). The oil was dissolved in minimal DCM and purified by flash chromatography, eluting with hexanes/ethyl acetate (0-40%). Product-containing fractions were collected and concentrated under reduced pressure to give diethyl [(4-chlorobenzenesulfonyl)(fluoro)methyl]phosphonate, as a light yellow oil (758 mg, Y:72%). UPLC and ¹H NMR (CDCl₃, 300 MHz) confirmed structure and purity of the product.

UPLC Rt: 1.79, m/z: 367.0 [M+Na], Cl pattern; Purity (254 nm): 98%; ¹H NMR (300 MHz, Chloroform-d) δ 8.02-7.89 (m, 2H), 7.63-7.51 (m, 2H), 5.37 (dd, J=45.4, 6.7 Hz, 1H), 4.43-4.17 (m, 4H), 1.36 (tdd, J=7.1, 3.7, 0.7 Hz, 6H).

### Scheme 1, Reaction 3: tert-butyl N-[(2E)-3-(benzenesulfonyl)-3-fluoroprop-2-en-1-yl]carbamate

Potassium tert-butoxide (0.247 g, 2.2 mmol, 1.0 eq) was suspended in anhydrous tetrahydrofuran (17.0 ml) with cooling to 0° C. over 5 min. Diethyl [(4-chlorobenzenesulfonyl)(fluoro)methyl]phosphonate (0.758 g, 2.2 mmol, 1.0 eq) was added dropwise, as a solution in anhydrous tetrahydrofuran (4.9 ml), instantly forming a yellow solution, which was stirred at 0° C. under argon for 30 min. Tert-butyl N-(2-oxoethyl)carbamate (0.368 g, 2.31 mmol, 1.05 eq) was added, as a solution in anhydrous tetrahydrofuran (4.6 ml, 0.5 M) over 1 min, and the reaction was stirred under argon and, subsequently, allowed to reach room temperature. After 90 min, the reaction was concentrated under reduced pressure to give a yellow oil, which was partitioned between ethyl acetate (10 mL) and saturated aqueous NaHCO₃ (20 mL). The layers were separated and the organic layer was washed with saturated NaCl solution (20 mL). The combined aqueous layers were extracted with ethyl acetate (4×5 mL) and the combined organic layers were dried with MgSO₄, filtered and concentrated under reduced pressure to give a yellow oil. Next, the oil was dissolved in minimal DCM and purified by flash chromatography, eluting with hexanes/ethyl acetate (0 to 15%). Clean product-containing fractions with the E-isomer and the fractions containing Z-isomer were collected separately and concentrated under reduced pressure to give tert-butyl N-[(2E)-3-(4-chlorobenzenesulfonyl)-3-fluoroprop-2-en-1-yl]carbamate, as a white solid (170 mg, Y: 22%). UPLC and ¹H NMR confirmed structure and purity, coupling constant the geometry of molecule as (E)-isomer.

UPLC Rt: 1.91, m/z: 372.0 [M+Na], Cl pattern; Purity (254 nm): 94%; 1H NMR (300 MHz, DMSO-d6) δ 7.96 (d, J=8.7 Hz, 2H), 7.87-7.74 (m, 2H), 7.22 (t, J=5.2 Hz, 1H), 6.28 (dt, J=33.7, 6.5 Hz, 1H), 3.75 (s, 2H), 1.37 (s, 9H).

### Scheme 1, Reaction 4: (2E)-3-(4-chlorobenzenesulfonyl)-3-fluoroprop-2-en-1-amine hydrochloride

Refrigerated 4N HCl in dioxane, 3.7-4.3N (1.21 ml, 4.86 mmol, 10.0 eq) was added to tert-butyl N-[(2E)-3-(4-chlorobenzenesulfonyl)-3-fluoroprop-2-en-1-yl]carbamate (0.17 g, 0.48 mmol, 1.0 eq) and the solution was stirred at room temperature. A white solid began to form within the first 10 min and after stirring for 1 h, the reaction was concentrated under reduced pressure to give the desired product, (2E)-3-(4-chlorobenzenesulfonyl)-3-fluoroprop-2-en-1-amine hydrochloride, as a white solid (138 mg, Y: 99%), which was taken forward to the next reaction without further purification. UPLC and ¹H NMR (DMSO-d₆, 300 MHz) confirmed the structure and purity of the product.

UPLC Rt: 1.41, m/z: 249.9 [M+1], Cl pattern; Purity (254 nm): 99%; ¹H NMR (300 MHz, DMSO-d6) δ 8.24 (s, 3H), 8.04-7.95 (m, 2H), 7.90-7.80 (m, 2H), 6.50 (dt, J=33.0, 6.9 Hz, 1H), 3.70 (s, 2H).

### Example 4: Preparation of [(1E)-3-aminoprop-1-en-1-yl](imino)[4-(trifluoromethoxy)phenyl]-λ⁶-sulfanone dihydrochloride (Scheme 2)

### Scheme 2, Reaction 1: tert-butyl N-[(tert-butoxy)carbonyl]-N-[(2E)-3-{[4-(trifluoromethoxy)phenyl]sulfanyl}prop-2-en-1-yl]carbamate

1,3-Bis(1,1-dimethylethyl) 2-(2-propyn-1-yl)imidodicarbonate (0.3 g, 1.12 mmol, 1.0 eq.), Tris(triphenylphosphine)rhodium(I) chloride (0.03 g, 0.033 mmol, 0.03 eq.) and 1,2-dichloroethane (4.0 ml) were degassed with argon for 30 min while cooling to 0° C. Next, 4-(trifluoromethoxy)thiophenol (0.24 g, 1.23 mmol, 1.1 eq.) was added, as a solution in 1,2-dichloroethane (1.6 ml) dropwise over 10 min, forming a dark brown solution. The reaction was stirred at room temperature under argon overnight and, subsequently, concentrated to a dark brown oil under reduced pressure. UPLC of the crude material was indicative of the desired product formation. The oil was dissolved in minimal DCM and purified by flash chromatography, eluting with hexanes/ethyl acetate (0 to 90%). Product-containing fractions were collected and concentrated under reduced pressure to give a light-yellow oil (428 mg). UPLC and ¹H NMR (CDCl₃, 300 MHz) confirmed formation of the desired product, tert-butyl N-[(tert-butoxy)carbonyl]-N-[(2E)-3-{[4-(trifluoromethoxy)phenyl]sulfanyl}prop-2-en-1-yl]carbamate, as an approximately 93:7 mixture with the vicinal alkene side-product, formed by addition of the thiophenol to the other side of the triple bond. The mixture was used without further purification in the next step.

UPLC Rt: 2.31, m/z: 472.1 [M+Na]; Purity (NMR): 93%; 1H NMR (300 MHz, Chloroform-d) δ 7.38-7.31 (m, 2H), 7.15 (dq, J=7.7, 1.0 Hz, 2H), 6.32 (dt, J=15.0, 1.2 Hz, 1H), 5.92 (dt, J=15.0, 6.3 Hz, 1H), 4.23 (dd, J=6.3, 1.3 Hz, 2H), 1.50 (s, 18H).

### Scheme 2, Reaction 2: tert-butyl N-[(tert-butoxy)carbonyl]-N-[(2E)-3-[imino(oxo)[4-(trifluoromethoxy)phenyl]-λ⁶-sulfanyl]prop-2-en-1-yl]carbamate

Tert-butyl N-[(tert-butoxy)carbonyl]-N-[(2E)-3-{[4-(trifluoromethoxy)phenyl]-sulfanyl}prop-2-en-1-yl]-carbamate (0.43 g, 0.89 mmol, 1.0 eq.) was dissolved in MeOH (17.1 ml, 40 vol), and cooled to 0° C. over 10 min before addition of ammonium carbamate (0.28 g, 3.5 mmol, 4.0 eq.). The mixture was stirred at 0° C. for 30 min before addition of iodobenzene diacetate (0.71 g, 2.2 mmol, 2.5 eq), in three portions, 10 minutes apart. Following addition of the final portion, the mixture was allowed to reach room temperature. After 30 min, UPLC indicated full consumption of the starting material. The reaction mixture was concentrated to a low volume under reduced pressure, before dissolving in minimal MeOH, adsorbing onto celite and purified by flash chromatography, eluting with hexanes/ethyl acetate (0 to 60%). Product-containing fractions were collected and concentrated under reduced pressure to give the desired product, tert-butyl N-[(tert-butoxy)carbonyl]-N-[(2E)-3-[imino(oxo)[4-(trifluoromethoxy)phenyl]-λ⁶-sulfanyl]prop-2-en-1-yl]carbamate, as a white solid (336 mg, Y:77%). UPLC and ¹H NMR (CDCl₃, 300 MHz) confirmed the structure and purity of the product.

UPLC Rt: 1.98, m/z: 481.00 [M+1]; Purity (NMR): 99%; ¹H NMR (300 MHz, Chloroform-d) δ 8.08 (d, J=8.9 Hz, 2H), 7.38 (d, J=8.4 Hz, 2H), 7.01 (dt, J=15.0, 5.1 Hz, 1H), 6.62 (d, J=15.0 Hz, 1H), 4.42 (dd, J=5.2, 1.6 Hz, 2H), 1.41 (s, 18H).

### Scheme 2, Reaction 3: [(1E)-3-aminoprop-1-en-1-yl](imino)[4-(trifluoromethoxy)phenyl]-λ⁶-sulfanone dihydrochloride

Refrigerated 4N HCl in dioxane (1.7 ml, 6.9 mmol, 10.0 eq) was added to N-[(tert-butoxy)carbonyl]-N-[(2E)-3-[imino(oxo)[4-(trifluoromethoxy)phenyl]-λ⁶-sulfanyl]prop-2-en-1-yl]carbamate (0.34 g, 0.69 mmol, 1.0 eq.) and the solution was stirred at room temperature. A white solid formed within the first 10 min. After 30 min, the reaction was concentrated under reduced pressure and the resulting solid was triturated with diethyl ether (10 mL) and dried under vacuum to give the desired product, [(1E)-3-aminoprop-1-en-1-yl](imino)[4-(trifluoromethoxy)phenyl]-λ⁶-sulfanone dihydrochloride, as a white solid (258 mg, Y: 85%), which was taken forward to the next reaction without further purification. UPLC and ¹H NMR (DMSO-d₆, 300 MHz) confirmed the structure and purity of the product.

UPLC Rt: 1.39, m/z: 281.00 [M+1]; Purity (254): 75%; ¹H NMR (300 MHz, DMSO-d₆) δ 8.43-8.22 (m, 5H), 8.11-7.96 (m, 3H), 7.69-7.59 (m, 3H), 7.04-6.93 (m, 1H), 6.82 (dt, J=15.2, 5.3 Hz, 1H), 3.76-3.63 (m, 2H).

### Example 5: Preparation of N-[(2E)-3-(4-bromobenzenesulfonyl)prop-2-en-1-yl]-2-oxo-1,2,5,6,7,8-hexahydroquinoline-3-carboxamide (Scheme 3)

2-Oxo-1,2,5,6,7,8-hexahydroquinoline-3-carboxylic acid (0.025 g, 0.13 mmol, 1.0 eq.) and T3P, 50+% w/w solution in acetonitrile (0.17 g, 0.26 mmol, 2.0 eq.) were dissolved in anhydrous DMF (1.3 ml, 0.1 M). N,N-Diisopropylethylamine, (0.09 ml, 0.52 mmol, 4.0 eq.) was added and the yellow mixture was stirred for 10 min. (2E)-3-(4-bromobenzenesulfonyl)prop-2-en-1-amine hydrochloride (0.038 g, 0.14 mmol, 1.1 eq.) was added and the mixture was stirred at room temperature for 2 h. The reaction mixture was partitioned between water and DCM, and the organic layer was collected, washed with saturated NaHCO₃ solution, saturated NaCl solution, dried over MgSO₄, filtered, and concentrated under reduced pressure. The crude product was purified by preparative TLC eluting with DCM/MeOH 5% to give N-[(2E)-3-(4-bromobenzenesulfonyl)prop-2-en-1-yl]-2-oxo-1,2,5,6,7,8-hexahydroquinoline-3-carboxamide (13 mg, Y: 18%) as yellow solid.

LCMS Rt=3.34 min, m/z=451/453 [M+1]; Purity: 98.31% (254 nm); 1H NMR (300 MHz, DMSO-d6) δ 12.26 (s, 1H), 10.01 (t, J=5.9 Hz, 1H), 8.03 (s, 1H), 7.91-7.83 (m, 2H), 7.83-7.75 (m, 2H), 6.97 (dt, J=15.1, 4.3 Hz, 1H), 6.68 (dt, J=15.1, 1.8 Hz, 1H), 4.23-4.13 (m, 2H), 2.58 (s, 2H), 1.68 (d, J=5.6 Hz, 4H).

### Example 6: Preparation of N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide (Scheme 3)

2-Oxo-1,2,5,6,7,8-hexahydroquinoline-3-carboxylic acid (0.023 g, 0.12 mmol, 1.0 eq) and HATU (0.059 g, 0.16 mmol, 1.0 eq) were dissolved in anhydrous DMF (0.5 mL). N,N-Diisopropylethylamine (0.11 ml, 0.62 mmol, 4.0 eq) was added and the yellow mixture was stirred for 10 min. [(1E)-3-aminoprop-1-en-1-yl](4-fluorophenyl)imino-λ⁶-sulfanone dihydrochloride (0.042 g, 0.14 mmol, 0.9 eq) was added and the mixture was stirred at room temperature for 2 h. The reaction mixture was partitioned between water and DCM, and the organic layer was collected, washed with saturated NaHCO₃ solution, saturated NaCl solution, dried over MgSO₄, filtered, and concentrated under reduced pressure. The residue was purified by preparative TLC eluting with ethyl acetate/MeOH 10% to obtain N-[(2E)-3-[(4-fluorophenyl)(imino)oxo-λ⁶-sulfanyl]prop-2-en-1-yl]-2-oxo-1,2,5,6,7,8-hexahydro-quinoline-3-carboxamide (0.012 g, 0.028 mmol, 18%) as an off-white solid.

LCMS Rt=1.85 m/z=390.10 [M+1]388.09 [M−1]; Purity: 92.5% (254 nm); ¹H NMR (300 MHz, DMSO-d₆) δ 12.28 (s, 1H), 10.02 (t, J=5.9 Hz, 1H), 8.03 (s, 1H), 7.98-7.85 (m, 2H), 7.48-7.36 (m, 2H), 6.79 (dt, J=14.9, 4.6 Hz, 1H), 6.57-6.43 (m, 1H), 4.67 (s, 1H), 4.21-4.07 (m, 2H), 2.59 (d, J=5.6 Hz, 2H), 1.70 (p, J=7.3, 6.8 Hz, 4H).

The title compounds of Examples 7-65, 126-177 and 186 were prepared by a method analogous to that described in Examples 5 or 6.

### Example 7: N-[(2E)-3-(benzenesulfonyl)prop-2-en-1-yl]-2-oxo-1,2,5,6,7,8-hexahydroquinoline-3-carboxamide

LCMS Rt=2.27 min, m/z=372.98 [M+1]; Purity: 98% (254 nm); ¹H NMR (400 MHz, DMSO-d6) 12.26 (s, 1H), 10.02 (t, J=6.0 Hz, 1H), 8.03 (s, 1H), 7.90-7.82 (m, 2H), 7.78-7.70 (m, 1H), 7.69-7.60 (m, 2H), 6.95 (dt, J=15.2, 4.5 Hz, 1H), 6.66 (dt, J=15.0, 1.8 Hz, 1H), 4.18 (ddd, J=6.3, 4.5, 2.0 Hz, 2H), 2.58 (t, J=5.9 Hz, 2H), 1.69 (q, J=8.2 Hz, 4H).

### Example 8: N-[(2E)-3-(benzenesulfonyl)prop-2-en-1-yl]-6-benzyl-2-oxo-1,2,5,6,7,8-hexahydro-1,6-naphthyridine-3-carboxamide

RT=2.29 min, m/z=463.90 [M+1]; Purity: 95% (254 nm); ¹H NMR (300 MHz, DMSO-d6) δ 12.42 (s, 1H), 9.98 (t, J=5.9 Hz, 1H), 8.01 (s, 1H), 7.91-7.82 (m, 2H), 7.79-7.70 (m, 1H), 7.69-7.58 (m, 2H), 7.40-7.22 (m, 5H), 6.95 (dt, J=15.1, 4.4 Hz, 1H), 6.67 (dt, J=15.1, 1.8 Hz, 1H), 4.26-4.10 (m, 2H), 3.66 (s, 2H), 3.39 (s, 2H), 2.67 (s, 4H).

### Example 9: N-[(2Z)-3-(benzenesulfonyl)prop-2-en-1-yl]-2-oxo-1,2,5,6,7,8-hexahydroquinoline-3-carboxamide

Purity: 97.2% (254 nm); RT=2.91 min, m/z=373.79 [M+1], 371.22 [M−1]; ¹H NMR (400 MHz, DMSO-d6) 12.26 (s, 1H), 10.06 (t, J=5.8 Hz, 1H), 8.05 (s, 1H), 8.02-7.95 (m, 2H), 7.79-7.73 (m, 1H), 7.72-7.64 (m, 2H), 6.59 (dt, J=11.2, 2.0 Hz, 1H), 6.38 (dt, J=11.5, 6.0 Hz, 1H), 4.52 (td, J=5.9, 2.0 Hz, 2H), 2.58 (t, J=6.0 Hz, 2H), 1.69 (q, J=8.0 Hz, 4H).

### Example 10: tert-butyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 96.8% (254 nm); RT=3.15 min, m/z=473.69 [M+1], 471.72[M−1]; ¹H NMR (400 MHz, DMSO-d6) 12.46 (s, 1H), 9.95 (t, J=5.9 Hz, 1H), 8.12 (s, 1H), 7.90-7.83 (m, 2H), 7.78-7.69 (m, 1H), 7.64 (dd, J=8.3, 6.8 Hz, 2H), 6.95 (dt, J=15.2, 4.4 Hz, 1H), 6.67 (dt, J=15.1, 1.9 Hz, 1H), 4.31 (s, 2H), 4.24-4.14 (m, 2H), 3.56 (t, J=5.8 Hz, 2H), 2.65 (t, J=5.8 Hz, 2H), 1.42 (s, 9H).

### Example 11: N-[(2E)-3-[(5-chloropyridin-2-yl)sulfonyl]prop-2-en-1-yl]-2-oxo-1,2,5,6,7,8-hexahydroquinoline-3-carboxamide

Purity: 97.9% (254 nm); RT=2.23 m/z=408.09 [M+1]406.04 [M−1]; ¹H NMR (400 MHz, DMSO-d6) δ 12.27 (s, 1H), 10.04 (t, J=5.9 Hz, 1H), 8.85 (d, J=2.3 Hz, 1H), 8.28 (dd, J=8.4, 2.4 Hz, 1H), 8.08 (d, J=8.5 Hz, 1H), 8.05 (s, 1H), 7.06 (dt, J=15.2, 4.3 Hz, 1H), 6.71 (dt, J=15.2, 1.9 Hz, 1H), 4.23 (ddd, J=6.1, 4.4, 2.0 Hz, 2H), 2.58 (t, J=5.9 Hz, 2H), 1.69 (q, J=8.0 Hz, 4H).

### Example 12: N-[(2E)-3-(benzenesulfonyl)prop-2-en-1-yl]-2-oxo-5-phenyl-1,2-dihydropyridine-3-carboxamide

Purity: 94.6%; RT=2.56 min, m/z=395.18 [M+1], 392.97 [M−1], ¹H NMR (300 MHz, DMSO-d6) 12.66 (s, 1H), 10.00 (t, J=5.9 Hz, 1H), 8.60 (d, J=2.9 Hz, 1H), 8.06 (d, J=2.9 Hz, 1H), 7.92-7.81 (m, 2H), 7.80-7.56 (m, 5H), 7.53-7.40 (m, 2H), 7.40-7.29 (m, 1H), 6.98 (dt, J=15.1, 4.3 Hz, 1H), 6.75 (dt, J=15.1, 1.8 Hz, 1H), 4.23 (tt, J=4.6, 1.9 Hz, 2H).

### Example 13: 6-acetyl-N-[(2E)-3-(benzenesulfonyl)prop-2-en-1-yl]-2-oxo-1,2,5,6,7,8-hexahydro-1,6-naphthyridine-3-carboxamide

Purity: 98.3%; Rt=2.17 min, m/z=416.17 [M+1], 414.14 [M−1]; ¹H NMR (300 MHz, DMSO-d6) 12.49 (s, 1H), 9.95 (t, J=6.0 Hz, 1H), 8.14 (d, J=5.9 Hz, 1H), 7.91-7.81 (m, 2H), 7.79-7.69 (m, 1H), 7.64 (dd, J=8.2, 6.6 Hz, 2H), 6.95 (dt, J=15.1, 4.4 Hz, 1H), 6.73-6.59 (m, 1H), 4.43 (d, J=22.9 Hz, 2H), 4.19 (d, J=5.3 Hz, 2H), 3.66 (q, J=5.5 Hz, 2H), 2.80-2.58 (m, 2H), 2.06 (d, J=8.0 Hz, 3H).

### Example 14: N-[(3E)-4-(benzenesulfonyl)but-3-en-2-yl]-2-oxo-1,2,5,6,7,8-hexahydroquinoline-3-carboxamide

Purity: 97.6%; RT=1.96 min, m/z=387.21 [M+1]385.20[M−1]; 1H NMR (400 MHz, DMSO-d6) δ 12.25 (s, 1H), 10.00 (d, J=7.7 Hz, 1H), 8.02 (s, 1H), 7.91-7.81 (m, 2H), 7.78-7.68 (m, 1H), 7.65 (dd, J=8.4, 6.9 Hz, 2H), 6.98 (dd, J=15.1, 4.7 Hz, 1H), 6.72 (dd, J=15.2, 1.7 Hz, 1H), 4.83-4.69 (m, 1H), 2.62-2.55 (m, 2H), 1.77-1.59 (m, 4H), 1.31 (d, J=7.0 Hz, 3H).

### Example 15: N-[(2E)-3-methanesulfonylprop-2-en-1-yl]-2-oxo-1,2,5,6,7,8-hexahydroquinoline-3-carboxamide

Purity: 93.1%; Rt=2.02 min, m/z=311.19 [M+1]309.22 [M−1]; ¹H NMR (400 MHz, DMSO-d6) δ 12.29 (s, 1H), 10.06 (t, J=6.0 Hz, 1H), 8.08 (s, 1H), 6.80 (dt, J=15.2, 4.3 Hz, 1H), 6.69-6.55 (m, 1H), 4.29-4.08 (m, 2H), 3.01 (s, 3H), 2.65-2.56 (m, 2H), 1.70 (q, J=7.9 Hz, 4H).

### Example 16: N-[(2E)-3-(4-fluorobenzenesulfonyl)prop-2-en-1-yl]-2-oxo-1,2,5,6,7,8-hexahydroquinoline-3-carboxamide

Purity: 97.7%; RT=1.90 min, m/z=391.14 [M+1]389.10 [M−1]; ¹H NMR (400 MHz, DMSO-d6) 12.28 (s, 1H), 10.01 (t, J=5.9 Hz, 1H), 8.03 (s, 1H), 7.98-7.89 (m, 2H), 7.54-7.45 (m, 2H), 6.96 (dt, J=15.1, 4.4 Hz, 1H), 6.68 (dt, J=15.1, 1.8 Hz, 1H), 4.18 (ddd, J=6.1, 4.4, 1.9 Hz, 2H), 2.58 (t, J=6.0 Hz, 2H), 1.68 (q, J=8.4, 7.5 Hz, 4H).

### Example 17: N-[(2E)-3-(cyclopentanesulfonyl)prop-2-en-1-yl]-2-oxo-1,2,5,6,7,8-hexahydroquinoline-3-carboxamide

Purity: 98.6%; RT=1.52 min, m/z=365.21 [M+1], 363.18 [M−1]; ¹H NMR (400 MHz, DMSO-d6) 12.29 (s, 1H), 10.05 (t, J=5.9 Hz, 1H), 8.07 (s, 1H), 6.79 (dt, J= 15.2, 4.5 Hz, 1H), 6.52 (dt, J=15.2, 1.8 Hz, 1H), 4.19 (ddd, J=6.2, 4.6, 1.9 Hz, 2H), 3.52 (tt, J=8.9, 6.7 Hz, 1H), 2.59 (t, J=6.0 Hz, 2H), 1.94-1.47 (m, 12H).

### Example 18: 2-oxo-N-[(2E)-3-(propane-2-sulfonyl)prop-2-en-1-yl]-1,2,5,6,7,8-hexahydroquinoline-3-carboxamide

Purity: 97.0%; RT=1.76 m/z=339.23 [M+1]337.16 [M−1]; ¹H NMR (400 MHz, DMSO-d6) 12.28 (s, 1H), 10.06 (t, J=5.9 Hz, 1H), 8.07 (s, 1H), 6.79 (dt, J=15.2, 4.5 Hz, 1H), 6.48 (dt, J=15.2, 1.8 Hz, 1H), 4.19 (ddd, J=6.3, 4.4, 1.9 Hz, 2H), 3.18 (p, J=6.8 Hz, 1H), 2.59 (t, J=6.0 Hz, 2H), 1.68 (d, J=12.8 Hz, 4H), 1.18 (d, J=6.8 Hz, 6H).

### Example 19: N-[(2E)-3-(3-fluoro-4-methoxybenzenesulfonyl)prop-2-en-1-yl]-2-oxo-1,2,5,6,7,8-hexahydroquinoline-3-carboxamide

Purity: 99.8%; RT=1.97 min, m/z=421.33 [M+1]419.12 [M−1]; ¹H NMR (400 MHz, DMSO-d6) 12.28 (s, 1H), 10.01 (t, J=5.9 Hz, 1H), 8.03 (s, 1H), 7.74-7.63 (m, 2H), 7.40 (t, J=8.3 Hz, 1H), 6.91 (dt, J=15.1, 4.4 Hz, 1H), 6.64 (dt, J=15.1, 1.8 Hz, 1H), 4.17 (ddd, J=6.2, 4.4, 1.9 Hz, 2H), 3.94 (s, 3H), 2.58 (t, J=5.7 Hz, 2H).

### Example 20: 2-oxo-N-[(2Z)-3-(propane-2-sulfonyl)prop-2-en-1-yl]-1,2,5,6,7,8-hexahydroquinoline-3-carboxamide

Purity: 88.9%; RT=5.45 min, m/z=339.22 [M+1]337.20 [M−1]; ¹H NMR (300 MHz, DMSO-d6) 12.27 (s, 1H), 10.04 (t, J=5.9 Hz, 1H), 8.06 (s, 1H), 6.53-6.39 (m, 2H), 4.41 (t, J=5.5 Hz, 2H), 2.57 (d, J=6.0 Hz, 2H), 1.69 (d, J=5.1 Hz, 4H), 1.26 (d, J=6.8 Hz, 6H).

### Example 21: N-[(2E)-3-(4-chlorobenzenesulfonyl)prop-2-en-1-yl]-2-oxo-1,2,5,6,7,8-hexahydroquinoline-3-carboxamide

Purity: 97.0%; RT=5.25 min, m/z=407.17 [M+1], 405.15 [M−1]; ¹H NMR (400 MHz, DMSO-d6) 12.28 (s, 1H), 10.02 (t, J=5.9 Hz, 1H), 8.03 (s, 1H), 7.91-7.84 (m, 2H), 7.75-7.68 (m, 2H), 6.97 (dt, J=15.1, 4.4 Hz, 1H), 6.68 (dt, J=15.1, 1.9 Hz, 1H), 4.18 (ddd, J=6.2, 4.5, 2.0 Hz, 2H), 2.58 (t, J=6.0 Hz, 2H), 1.69 (h, J=6.7, 6.2 Hz, 4H).

### Example 22: tert-butyl 3-{[(2E)-3-(2-fluorobenzenesulfonyl)prop-2-en-1-yl]carbamoyl}-2-oxo-1,2,5,6,7,8-hexahydro-1,6-naphthyridine-6-carboxylate

Purity: 93.6%; RT=2.19 min, m/z=492.40 [M+1]; 1H NMR (400 MHz, DMSO-d6) δ 12.48 (s, 1H), 9.99 (t, J=6.0 Hz, 1H), 8.14 (s, 1H), 7.88 (td, J=7.5, 1.8 Hz, 1H), 7.85-7.76 (m, 1H), 7.55-7.42 (m, 2H), 7.05 (dt, J=15.0, 4.4 Hz, 1H), 6.72 (d, J=15.1 Hz, 1H), 4.32 (s, 2H), 4.27-4.15 (m, 2H), 3.57 (t, J=5.8 Hz, 2H), 2.66 (s, 2H), 1.42 (s, 9H).

### Example 23: N-[(2E)-3-(2-methylpropane-2-sulfonyl)prop-2-en-1-yl]-2-oxo-1,2,5,6,7,8-hexahydroquinoline-3-carboxamide

Purity: 97.5%; RT=1.39 min, m/z=353.30 [M+1], 351.20 [M−1]; ¹H NMR (400 MHz, DMSO-d6) δ 12.29 (s, 1H), 10.07 (t, J=5.8 Hz, 1H), 8.06 (s, 1H), 6.79 (dt, J=15.2, 4.6 Hz, 1H), 6.50 (dt, J=15.1, 1.8 Hz, 1H), 4.20 (ddd, J=6.3, 4.6, 1.9 Hz, 2H), 2.62-2.55 (m, 2H), 1.77-1.62 (m, 4H), 1.24 (s, 9H).

### Example 24: N-[(2E)-3-(2-fluorobenzenesulfonyl)prop-2-en-1-yl]-2-oxo-1,2,5,6,7,8-hexahydroquinoline-3-carboxamide

Purity: 91.6%; RT=2.07 m/z=391.20 [M+1]389.10 [M−1]; ¹H NMR (400 MHz, DMSO-d6) δ 12.29 (s, 1H), 10.05 (t, J=5.9 Hz, 1H), 8.04 (s, 1H), 7.88 (td, J=7.6, 1.8 Hz, 1H), 7.84-7.76 (m, 1H), 7.55-7.40 (m, 2H), 7.05 (ddd, J=15.1, 5.1, 3.9 Hz, 1H), 6.76-6.63 (m, 1H), 4.22 (ddd, J=6.3, 4.5, 2.0 Hz, 2H), 2.58 (t, J=6.0 Hz, 2H), 1.76-1.59 (m, 4H).

### Example 25: tert-butyl 3-{[(2E)-3-(4-fluorobenzenesulfonyl)prop-2-en-1-yl]carbamoyl}-2-oxo-1,2,5,6,7,8-hexahydro-1,6-naphthyridine-6-carboxylate

Purity: 95.0%; RT=1.66 m/z=492.42 [M+1]490.18 [M−1]; ¹H NMR (400 MHz, DMSO-d6) 12.48 (s, 1H), 10.03 (s, 1H), 8.11 (s, 1H), 7.97-7.91 (m, 2H), 7.53-7.44 (m, 2H), 6.96 (dt, J=15.1, 4.3 Hz, 1H), 6.68 (dt, J=15.1, 1.9 Hz, 1H), 4.31 (s, 2H), 4.18 (ddd, J=6.4, 4.5, 1.9 Hz, 2H), 3.56 (t, J=5.8 Hz, 2H), 2.65 (t, J=6.1 Hz, 2H), 1.42 (s, 9H).

### Example 26: tert-butyl 3-{[(2E)-3-(3-fluoro-4-methoxybenzenesulfonyl)prop-2-en-1-yl]carbamoyl}-2-oxo-1,2,5,6,7,8-hexahydro-1,6-naphthyridine-6-carboxylate

Purity: 97.9%; RT=2.72 min, m/z=522.17 [M+1]520.41 [M−1]; ¹H NMR (300 MHz, DMSO-d6) 12.49 (s, 1H), 9.95 (t, J=5.9 Hz, 1H), 8.16 (d, J=19.0 Hz, 1H), 7.92-7.82 (m, 2H), 7.77-7.69 (m, 1H), 7.64 (dd, J=8.2, 6.6 Hz, 2H), 6.95 (dt, J=15.2, 4.4 Hz, 1H), 6.66 (d, J=15.1 Hz, 1H), 4.54 (s, 1H), 4.42 (s, 1H), 4.18 (s, 2H), 3.72 (d, J=6.1 Hz, 2H), 2.66 (d, J=30.6 Hz, 2H), 2.30 (d, J=4.0 Hz, 2H), 0.98 (d, J=11.6 Hz, 9H).

### Example 27: tert-butyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 94.7%; RT=3.15 min, m/z=508.12 [M+1], 505.87 [M−1]; 1H NMR (400 MHz, DMSO-d6) 12.48 (s, 1H), 10.00 (d, J=6.3 Hz, 1H), 8.12 (s, 1H), 7.92-7.83 (m, 2H), 7.77-7.68 (m, 2H), 6.98 (dt, J=15.2, 4.3 Hz, 1H), 6.69 (dt, J=15.1, 1.9 Hz, 1H), 4.32 (s, 2H), 4.24-4.14 (m, 2H), 3.56 (t, J=5.9 Hz, 2H), 2.65 (t, J=6.0 Hz, 2H), 1.42 (s, 9H).

### Example 28: N-[(2E)-3-(4-chlorobenzenesulfonyl)prop-2-en-1-yl]-2-oxo-1,2-dihydroquinoline-3-carboxamide

Purity: 96.2%; RT=3.30 min, m/z=403.12 [M+1]400.92 [M−1]; ¹H NMR (400 MHz, DMSO-d6) 12.48 (s, 1H), 9.97 (d, J=6.2 Hz, 1H), 8.82 (s, 1H), 7.94 (dd, J=8.2, 1.4 Hz, 1H), 7.91-7.85 (m, 2H), 7.75-7.70 (m, 2H), 7.66 (ddd, J=8.5, 7.1, 1.4 Hz, 1H), 7.01 (dt, J=15.2, 4.3 Hz, 1H), 6.81 (dt, J=15.0, 1.9 Hz, 1H), 4.24 (ddd, J=6.2, 4.4, 1.9 Hz, 2H).

### Example 29: 6-benzyl-N-[(2E)-3-(4-chlorobenzenesulfonyl)prop-2-en-1-yl]-2-oxo-1,2,5,6,7,8-hexahydro-1,6-naphthyridine-3-carboxamide

Purity: 82.9%; RT=2.04 min, m/z=497.84 [M+1]495.97 [M−1]; ¹H NMR (400 MHz, DMSO-d6) 12.45 (s, 1H), 10.02 (d, J=6.5 Hz, 1H), 8.00 (s, 1H), 7.90-7.84 (m, 2H), 7.76-7.68 (m, 2H), 7.39-7.24 (m, 5H), 6.97 (dt, J=15.2, 4.5 Hz, 1H), 6.69 (dt, J=14.9, 1.9 Hz, 1H), 4.17 (q, J=3.8, 1.9 Hz, 2H), 3.65 (s, 2H), 2.66 (d, J=2.7 Hz, 4H).

### Example 30: N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide

Purity: 97.7%; RT=2.18 min, m/z=420.11 [M+1]418.09 [M−1]; ¹H NMR (400 MHz, DMSO-d6) δ 12.26 (s, 1H), 9.99 (t, J=6.0 Hz, 1H), 8.04 (s, 1H), 7.68-7.59 (m, 2H), 7.35 (t, J=8.4 Hz, 1H), 6.76 (dt, J=14.9, 4.6 Hz, 1H), 6.48 (dt, J=14.9, 1.8 Hz, 1H), 4.59 (s, 1H), 4.17-4.08 (m, 2H), 3.92 (s, 3H), 2.58 (t, J=6.0 Hz, 2H), 1.69 (q, J=7.7, 6.9 Hz, 4H).

### Example 31: N-[(2E)-3-(2,4-difluorobenzenesulfonyl)prop-2-en-1-yl]-2-oxo-1,2,5,6,7,8-hexahydroquinoline-3-carboxamide

Purity: 92.6%; RT=2.46 min, m/z=407.27 [M−1]; ¹H NMR (400 MHz, DMSO-d6) δ 12.28 (s, 1H), 10.04 (t, J=5.9 Hz, 1H), 8.05 (s, 1H), 7.95 (td, J=8.7, 6.3 Hz, 1H), 7.62 (ddd, J=10.6, 9.2, 2.5 Hz, 1H), 7.42-7.31 (m, 1H), 7.11-6.99 (m, 1H), 6.75-6.64 (m, 1H), 4.22 (ddd, J=6.2, 4.4, 1.9 Hz, 2H), 2.58 (t, J=6.0 Hz, 2H), 1.79-1.59 (m, 4H).

### Example 32: N-[(2E)-3-(2,4-dichlorobenzenesulfonyl)prop-2-en-1-yl]-2-oxo-1,2,5,6,7,8-hexahydroquinoline-3-carboxamide

Purity: 97.1%; RT=2.88 min, m/z=443.00 [M+1]; ¹H NMR (400 MHz, DMSO-d6) δ 12.28 (s, 1H), 10.05 (t, J=5.9 Hz, 1H), 8.12-8.01 (m, 2H), 7.94 (d, J=2.1 Hz, 1H), 7.72 (dd, J=8.6, 2.1 Hz, 1H), 7.09 (dt, J=15.1, 4.4 Hz, 1H), 6.73 (dt, J=15.1, 1.9 Hz, 1H), 4.23 (ddd, J=6.2, 4.5, 1.9 Hz, 2H), 2.58 (t, J=6.0 Hz, 2H), 1.69 (q, J=8.4, 8.0 Hz, 4H).

### Example 33: N-[(2E)-3-(2-chloro-4-fluorobenzenesulfonyl)prop-2-en-1-yl]-2-oxo-1,2,5,6,7,8-hexahydroquinoline-3-carboxamide

Purity: 97.5%; RT=2.17 min, m/z=425.20 [M+1]; ¹H NMR (400 MHz, DMSO-d6) δ 12.28 (s, 1H), 10.05 (t, J=5.9 Hz, 1H), 8.12 (dd, J=8.9, 6.0 Hz, 1H), 8.04 (s, 1H), 7.78 (dd, J=8.7, 2.5 Hz, 1H), 7.51 (ddd, J=8.9, 8.0, 2.6 Hz, 1H), 7.08 (dt, J=15.1, 4.5 Hz, 1H), 6.73 (dt, J=15.0, 1.9 Hz, 1H), 4.23 (ddd, J=6.3, 4.5, 2.0 Hz, 2H), 2.58 (t, J=5.9 Hz, 2H), 1.78-1.60 (m, 4H).

### Example 34: N-[(2E)-3-(2,4-difluorobenzenesulfonyl)prop-2-en-1-yl]-2-oxo-1H,2H,5H,6H,7H,8H,9H-cyclohepta[b]pyridine-3-carboxamide

Purity: 97.0%; RT=2.24 min, m/z=423.15 [M+1], 421.10 [M−1]; 1H NMR (400 MHz, DMSO-d6) δ 12.43 (s, 1H), 10.03 (t, J=5.9 Hz, 1H), 8.10 (s, 1H), 7.94 (td, J=8.6, 6.2 Hz, 1H), 7.62 (ddd, J=11.3, 9.2, 2.4 Hz, 1H), 7.36 (td, J=8.6, 2.5 Hz, 1H), 7.05 (ddd, J=15.0, 4.9, 3.8 Hz, 1H), 6.71 (dd, J=15.1, 2.2 Hz, 1H), 4.21 (ddd, J=6.3, 4.5, 1.9 Hz, 2H), 2.83-2.73 (m, 2H), 2.66-2.59 (m, 2H), 1.82-1.71 (m, 2H), 1.65-1.44 (m, 4H).

### Example 35: N-[(2E)-3-(4-fluorobenzenesulfonyl)prop-2-en-1-yl]-2-oxo-1H,2H,5H,6H,7H,8H,9H-cyclohepta[b]pyridine-3-carboxamide

Purity: 93.4%; RT=2.16 min, m/z=405.15 [M+1]; ¹H NMR (400 MHz, DMSO-d6) δ 12.41 (s, 1H), 10.00 (t, J=5.8 Hz, 1H), 8.09 (s, 1H), 7.99-7.88 (m, 2H), 7.54-7.43 (m, 2H), 6.95 (dt, J=15.1, 4.4 Hz, 1H), 6.68 (dt, J=15.0, 1.9 Hz, 1H), 4.17 (ddd, J=6.2, 4.5, 1.9 Hz, 2H), 2.83-2.75 (m, 2H), 2.66-2.57 (m, 2H), 1.82-1.70 (m, 2H), 1.62-1.45 (m, 4H).

### Example 36: N-[(2E)-3-(4-chlorobenzenesulfonyl)prop-2-en-1-yl]-2-oxo-1H,2H,5H,6H,7H,8H,9H-cyclohepta[b]pyridine-3-carboxamide

Purity: 92.0%; RT=2.45 min, m/z=421.15 [M+1]419.21 [M−1]; ¹H NMR (400 MHz, DMSO-d6) δ 12.41 (s, 1H), 10.09-9.97 (m, 1H), 8.08 (s, 1H), 7.91-7.83 (m, 2H), 7.76-7.69 (m, 2H), 6.97 (dt, J=15.1, 4.3 Hz, 1H), 6.69 (dt, J=15.1, 1.9 Hz, 1H), 4.18 (q, J=5.2, 3.6 Hz, 2H), 2.77 (d, J=9.9 Hz, 2H), 2.62 (d, J=8.3 Hz, 2H), 1.74 (d, J=10.8 Hz, 2H), 1.54 (d, J=23.5 Hz, 4H).

### Example 37: N-[(2E)-3-(3-fluoro-4-methoxybenzenesulfonyl)prop-2-en-1-yl]-2-oxo-1H,2H,5H,6H,7H,8H,9H-cyclohepta[b]pyridine-3-carboxamide

Purity: 95% (254 nm); RT=2.94 min, m/z=435.14 [M+1]433.12 [M−1]; ¹H NMR (400 MHz, DMSO-d6) δ 12.41 (s, 1H), 10.04 (s, 1H), 8.08 (s, 1H), 7.76-7.64 (m, 2H), 7.40 (t, J=8.3 Hz, 1H), 6.92 (dt, J=15.2, 4.4 Hz, 1H), 6.65 (dt, J=15.0, 1.9 Hz, 1H), 4.22-4.13 (m, 2H), 3.94 (s, 3H), 2.84-2.71 (m, 2H), 2.66-2.58 (m, 2H), 1.83-1.70 (m, 2H), 1.64-1.45 (m, 4H).

### Example 38: N-[(2E)-3-(benzenesulfonyl)prop-2-en-1-yl]-2-oxo-1H,2H,5H,6H,7H,8H,9H-cyclohepta[b]pyridine-3-carboxamide

Purity: 85% (254 nm); RT=2.01 min, m/z=387.18 [M+1]385.34 [M−1]; ¹H NMR (400 MHz, DMSO-d6) δ 12.43 (s, 1H), 10.02 (t, J=5.8 Hz, 1H), 8.09 (s, 1H), 7.89-7.83 (m, 2H), 7.77-7.70 (m, 1H), 7.69-7.61 (m, 2H), 6.95 (dt, J=15.1, 4.4 Hz, 1H), 6.66 (dt, J=15.1, 1.8 Hz, 1H), 4.18 (ddd, J=6.2, 4.5, 1.9 Hz, 2H), 2.82-2.75 (m, 2H), 2.66-2.59 (m, 2H), 1.81-1.70 (m, 2H), 1.61-1.46 (m, 4H).

### Example 39: N-[(2E)-3-(3-fluorobenzenesulfonyl)prop-2-en-1-yl]-2-oxo-1,2,5,6,7,8-hexahydroquinoline-3-carboxamide

Purity: 96.1% (254 nm); RT=1.97 min, m/z=391.16 [M+1], 389.14 [M−1]; ¹H NMR (400 MHz, DMSO-d6) δ 12.27 (s, 1H), 10.02 (t, J=5.9 Hz, 1H), 8.04 (s, 1H), 7.76-7.67 (m, 3H), 7.61 (ddd, J=9.5, 5.5, 2.4 Hz, 1H), 7.01 (dt, J=15.1, 4.3 Hz, 1H), 6.72 (dt, J=15.1, 1.9 Hz, 1H), 4.19 (ddd, J=6.1, 4.4, 1.9 Hz, 2H), 2.58 (t, J=5.9 Hz, 2H), 1.76-1.62 (m, 4H).

### Example 40: N-[(2E)-3-(4-methoxybenzenesulfonyl)prop-2-en-1-yl]-2-oxo-1,2,5,6,7,8-hexahydroquinoline-3-carboxamide

Purity: 98.5% (254 nm); RT=2.37 min, m/z=403.16 [M+1], 400.89 [M−1]; ¹H NMR (400 MHz, DMSO-d6) δ 12.26 (s, 1H), 10.00 (t, J=6.0 Hz, 1H), 8.03 (s, 1H), 7.77 (d, J=8.6 Hz, 2H), 7.15 (d, J=8.6 Hz, 2H), 6.86 (dt, J=15.1, 4.4 Hz, 1H), 6.59 (d, J=15.1 Hz, 1H), 4.16 (d, J=5.8 Hz, 2H), 3.85 (s, 3H), 2.58 (t, J=5.9 Hz, 2H), 1.69 (q, J=8.1 Hz, 4H).

### Example 41: N-[(2E)-3-(2-chlorobenzenesulfonyl)prop-2-en-1-yl]-2-oxo-1,2,5,6,7,8-hexahydroquinoline-3-carboxamide

Purity: 96.3% (254 nm); RT=2.01 min, m/z=407.10 [M+1], 405.38 [M−1]; ¹H NMR (400 MHz, DMSO-d6) δ 12.28 (s, 1H), 10.06 (t, J=5.9 Hz, 1H), 8.12-7.97 (m, 2H), 7.81-7.66 (m, 2H), 7.63 (ddd, J=7.9, 6.8, 1.8 Hz, 1H), 7.08 (dt, J=15.1, 4.5 Hz, 1H), 6.75 (dt, J=15.1, 1.9 Hz, 1H), 4.23 (ddd, J=6.3, 4.6, 1.9 Hz, 2H), 2.58 (t, J=5.9 Hz, 2H), 1.78-1.59 (m, 4H).

### Example 42: N-[(2E)-3-(2,4-difluorobenzenesulfonyl)prop-2-en-1-yl]-2-oxo-1H,2H,5H,6H,7H-cyclopenta[b]pyridine-3-carboxamide

Purity: 82.2% (254 nm); RT=2.26 min, m/z=395.11 [M+1]393.30 [M−1]; ¹H NMR (300 MHz, DMSO-d6) δ 12.76 (s, 1H), 10.09 (t, J=5.9 Hz, 1H), 8.22 (s, 1H), 7.95 (td, J=8.6, 6.2 Hz, 1H), 7.62 (ddd, J=11.2, 9.3, 2.5 Hz, 1H), 7.36 (td, J=8.6, 2.5 Hz, 1H), 7.14-6.97 (m, 1H), 6.78-6.64 (m, 1H), 4.22 (ddd, J=6.3, 4.4, 2.0 Hz, 2H), 2.84 (q, J=7.7 Hz, 2H), 2.76-2.67 (m, 2H), 2.05 (p, J=7.5 Hz, 2H).

### Example 43: N-[(2E)-3-(benzenesulfonyl)prop-2-en-1-yl]-2-oxo-1H,2H,5H,6H,7H-cyclopenta[b]pyridine-3-carboxamide

Purity: 92.0% (254 nm); Rt=2.06 min, m/z=359.14 [M+1], 357.26 [M−1]; ¹H NMR (400 MHz, DMSO-d6) δ 12.64 (s, 1H), 10.07 (t, J=5.9 Hz, 1H), 8.21 (s, 1H), 7.90-7.83 (m, 2H), 7.78-7.70 (m, 1H), 7.68-7.61 (m, 2H), 6.95 (dt, J=15.1, 4.5 Hz, 1H), 6.66 (dt, J=15.1, 1.9 Hz, 1H), 4.18 (ddd, J=6.2, 4.5, 1.9 Hz, 2H), 2.82 (t, J=7.6 Hz, 2H), 2.71 (t, J=7.4 Hz, 2H), 2.05 (p, J=7.8 Hz, 2H).

### Example 44: 2-oxo-N-[(2E)-3-(thiophene-2-sulfonyl)prop-2-en-1-yl]-1,2,5,6,7,8-hexahydroquinoline-3-carboxamide

Purity: 96.3% (254 nm); RT=2.59 m/z=379.09 [M+1]393.30 [M−1]; ¹H NMR (300 MHz, DMSO-d6) δ 12.25 (s, 1H), 10.02 (t, J=6.0 Hz, 1H), 8.10 (dd, J=5.0, 1.4 Hz, 1H), 8.04 (s, 1H), 7.75 (dd, J=3.8, 1.4 Hz, 1H), 7.26 (dd, J=5.0, 3.8 Hz, 1H), 6.93 (dt, J=15.1, 4.5 Hz, 1H), 6.73 (dt, J=15.1, 1.8 Hz, 1H), 4.18 (ddd, J=6.2, 4.5, 1.9 Hz, 2H), 2.58 (t, J=5.9 Hz, 2H), 1.76-1.62 (m, 4H).

### Example 45: N-[(2E)-3-(3,4-dimethoxybenzenesulfonyl)prop-2-en-1-yl]-2-oxo-1,2,5,6,7,8-hexahydroquinoline-3-carboxamide

Purity: 98.5% (254 nm); Rt=2.20 min, m/z=433.14 [M+1]431.36 [M−1]; ¹H NMR (300 MHz, DMSO-d6) δ 12.26 (s, 1H), 10.00 (t, J=5.9 Hz, 1H), 8.03 (s, 1H), 7.43 (dd, J=8.5, 2.2 Hz, 1H), 7.29 (d, J=2.2 Hz, 1H), 7.17 (d, J=8.6 Hz, 1H), 6.87 (dt, J=15.1, 4.5 Hz, 1H), 6.63 (dt, J=15.1, 1.8 Hz, 1H), 4.16 (ddd, J=6.4, 4.5, 1.9 Hz, 2H), 3.85 (s, 3H), 3.83 (s, 3H), 2.63-2.55 (m, 2H), 1.68 (q, J=6.6 Hz, 4H).

### Example 46: N-[(2E)-3-(benzenesulfonyl)prop-2-en-1-yl]-2-oxo-6-(propan-2-yl)-1,2,5,6,7,8-hexahydroquinoline-3-carboxamide

Purity: 95.0% (254 nm); Rt=2.70 min, m/z=415.12 [M+1], 413.12 [M−1]; ¹H NMR (300 MHz, DMSO-d6) δ 12.27 (s, 1H), 10.01 (t, J=5.9 Hz, 1H), 8.05 (s, 1H), 7.90-7.83 (m, 2H), 7.79-7.70 (m, 1H), 7.69-7.60 (m, 2H), 6.95 (dt, J=15.1, 4.3 Hz, 1H), 6.71-6.60 (m, 1H), 4.18 (t, J=4.7 Hz, 2H), 2.61 (d, J=13.5 Hz, 2H), 2.25-2.15 (m, 1H), 1.87 (d, J=11.7 Hz, 1H), 1.53 (p, J=6.3 Hz, 1H), 1.47-1.21 (m, 3H), 0.91 (d, J=6.7 Hz, 6H).

### Example 47: N-[(2E)-3-(benzenesulfonyl)prop-2-en-1-yl]-2-oxo-1,2-dihydro-1,8-naphthyridine-3-carboxamide

Purity: 89.7% (254 nm); Rt=2.01 min, m/z=370.10 [M+1], 368.11 [M−1]; ¹H NMR (400 MHz, DMSO-d6) δ 9.81 (t, J=6.0 Hz, 1H), 8.84 (s, 1H), 8.67 (dd, J=4.7, 1.8 Hz, 1H), 8.41 (dd, J=7.8, 1.9 Hz, 1H), 7.90-7.84 (m, 2H), 7.76-7.70 (m, 1H), 7.68-7.61 (m, 2H), 7.37 (dd, J=7.8, 4.7 Hz, 1H), 6.98 (dt, J=15.1, 4.3 Hz, 1H), 6.80 (dt, J=15.1, 1.9 Hz, 1H), 4.24 (ddd, J=6.1, 4.3, 1.9 Hz, 2H).

### Example 48: N-[(2E)-3-(benzenesulfonyl)prop-2-en-1-yl]-6,6-dimethyl-2-oxo-1,2,5,6,7,8-hexahydroquinoline-3-carboxamide

Purity: 99.9% (254 nm); Rt=2.74 min, m/z=401.16 [M+1], 398.85 [M−1]; ¹H NMR (400 MHz, DMSO-d6) δ 12.30 (s, 1H), 10.01 (t, J=5.9 Hz, 1H), 8.00 (s, 1H), 7.89-7.82 (m, 2H), 7.76-7.70 (m, 1H), 7.67-7.61 (m, 2H), 6.95 (dt, J=15.1, 4.4 Hz, 1H), 6.66 (dt, J=15.1, 1.8 Hz, 1H), 4.17 (ddd, J=6.2, 4.4, 1.9 Hz, 2H), 2.59 (t, J=6.7 Hz, 2H), 2.30 (s, 2H), 1.50 (t, J=6.6 Hz, 2H), 0.93 (s, 6H).

### Example 49: N-[(2E)-3-(benzenesulfonyl)prop-2-en-1-yl]-6,6-difluoro-2-oxo-1,2,5,6,7,8-hexahydroquinoline-3-carboxamide

Purity: 99.9% (254 nm); Rt=2.32 min, m/z=409.13 [M+1], 407.15 [M−1]; ¹H NMR (400 MHz, DMSO-d6) δ 12.50 (s, 1H), 9.93 (t, J=5.9 Hz, 1H), 8.11 (s, 1H), 7.89-7.83 (m, 2H), 7.76-7.70 (m, 1H), 7.68-7.61 (m, 2H), 6.95 (dt, J=15.1, 4.4 Hz, 1H), 6.67 (dt, J=15.1, 1.9 Hz, 1H), 4.18 (ddd, J=6.1, 4.4, 1.9 Hz, 2H), 3.13 (t, J=14.6 Hz, 2H), 2.83 (t, J=6.9 Hz, 2H), 2.25 (tt, J=14.0, 6.8 Hz, 2H).

### Example 50: N-[(2E)-3-(4-tert-butylbenzenesulfonyl)prop-2-en-1-yl]-2-oxo-1,2,5,6,7,8-hexahydroquinoline-3-carboxamide

Purity: 99.8% (254 nm); Rt=2.34 min, m/z=429.18 [M+1]427.41 [M−1]; ¹H NMR (400 MHz, DMSO-d6) δ 11.81 (s, 1H), 10.00 (t, J=5.9 Hz, 1H), 8.03 (s, 1H), 7.81-7.72 (m, 2H), 7.70-7.61 (m, 2H), 6.92 (dt, J=15.1, 4.4 Hz, 1H), 6.62 (dt, J=15.0, 1.9 Hz, 1H), 4.23-4.11 (m, 2H), 2.58 (t, J=6.0 Hz, 2H), 1.78-1.61 (m, 4H), 1.31 (s, 9H).

### Example 51: N-[(2E)-3-(3,4-dimethylbenzenesulfonyl)prop-2-en-1-yl]-2-oxo-1,2,5,6,7,8-hexahydroquinoline-3-carboxamide

Purity: 99.6% (254 nm); RT=2.70 min, m/z=401.15 [M+1]399.31 [M−1]; ¹H NMR (400 MHz, DMSO-d6) δ 12.26 (s, 1H), 10.01 (s, 1H), 8.03 (s, 1H), 7.61 (d, J=2.0 Hz, 1H), 7.56 (dd, J=7.9, 2.1 Hz, 1H), 7.39 (d, J=7.9 Hz, 1H), 6.88 (dt, J=15.2, 4.4 Hz, 1H), 6.58 (dt, J=15.0, 1.9 Hz, 1H), 4.23-4.10 (m, 2H), 2.58 (t, J=5.9 Hz, 2H), 2.30 (s, 6H), 1.77-1.61 (m, 4H).

### Example 52: N-[(2E)-3-[(3,4-dimethoxyphenyl)(imino)oxo-λ⁶-sulfanyl]prop-2-en-1-yl]-2-oxo-1,2,5,6,7,8-hexahydroquinoline-3-carboxamide

Purity: 92.2% (254 nm); RT=6.51 min, m/z=432.15[M+1]430.19 [M−1]; ¹H NMR (400 MHz, DMSO-d6) δ 12.26 (s, 1H), 9.99 (t, J=5.9 Hz, 1H), 8.04 (s, 1H), 7.43 (dd, J=8.5, 2.2 Hz, 1H), 7.32 (d, J=2.2 Hz, 1H), 7.12 (d, J=8.6 Hz, 1H), 6.72 (dt, J=14.9, 4.7 Hz, 1H), 6.48 (dt, J=14.9, 1.8 Hz, 1H), 4.41 (s, 1H), 4.12 (t, J=5.3 Hz, 2H), 3.83 (s, 3H), 3.80 (s, 3H), 2.58 (t, J=5.9 Hz, 2H), 1.76-1.61 (m, 4H).

### Example 53: N-[(2E)-3-(benzenesulfonyl)(1,1-²H2)prop-2-en-1-yl]-2-oxo-1,2,5,6,7,8-hexahydroquinoline-3-carboxamide

Purity: 97.6% (254 nm); Rt=2.27 min, m/z=375.20 [M+1], 372.96 [M−1]; ¹H NMR (400 MHz, DMSO-d6) δ 12.26 (s, 1H), 10.00 (s, 1H), 8.03 (s, 1H), 7.91-7.81 (m, 2H), 7.77-7.69 (m, 1H), 7.69-7.59 (m, 2H), 6.94 (d, J=15.1 Hz, 1H), 6.67 (d, J=15.1 Hz, 1H), 2.58 (t, J=6.0 Hz, 2H), 1.77-1.59 (m, 4H).

### Example 54: N-[(2E)-3-[4-(dimethylamino)benzenesulfonyl]prop-2-en-1-yl]-2-oxo-1,2,5,6,7,8-hexahydroquinoline-3-carboxamide

Purity: 97.3% (254 nm); RT=2.97 min, m/z=416.17 [M+1], 414.16 [M−1]; ¹H NMR (400 MHz, DMSO-d6) δ 12.26 (s, 1H), 9.98 (t, J=6.0 Hz, 1H), 8.03 (s, 1H), 7.60 -7.49 (m, 2H), 6.84-6.68 (m, 3H), 6.48 (dt, J=15.0, 1.8 Hz, 1H), 4.18-4.03 (m, 2H), 3.01 (s, 6H), 2.58 (t, J=6.0 Hz, 2H), 1.78-1.58 (m, 4H).

### Example 55: N-[(2E)-3-(4-cyclopropylbenzenesulfonyl)prop-2-en-1-yl]-2-oxo-1,2,5,6,7,8-hexahydroquinoline-3-carboxamide

Purity: 94.8% (254 nm); RT=3.33 min, m/z=413.3 [M+1]; ¹H NMR (400 MHz, DMSO-d6) δ 12.26 (s, 1H), 9.99 (t, J=5.9 Hz, 1H), 6.89 (dt, J=15.1, 4.4 Hz, 1H), 6.64-6.55 (m, 1H), 4.22-4.10 (m, 2H), 2.58 (t, J=5.9 Hz, 2H), 2.04 (tt, J=8.6, 4.9 Hz, 1H), 1.78-1.59 (m, 4H), 1.12-1.02 (m, 2H), 0.84-0.75 (m, 2H).

### Example 56: N-[(2E)-3-(4-cyanobenzenesulfonyl)prop-2-en-1-yl]-2-oxo-1,2,5,6,7,8-hexahydroquinoline-3-carboxamide

Purity: 95.6% (254 nm); RT=2.91 min, m/z=398.20 [M+1]; ¹H NMR (400 MHz, DMSO-d6) δ 12.28 (s, 1H), 10.04 (t, J=5.6 Hz, 1H), 8.16-8.10 (m, 2H), 8.08-8.01 (m, 3H), 7.06 (dt, J=15.2, 4.3 Hz, 1H), 6.75 (dt, J=15.1, 1.9 Hz, 1H), 4.25-4.17 (m, 2H), 2.58 (t, J=5.8 Hz, 2H), 1.78-1.61 (m, 4H).

### Example 57: N-[(2E)-3-[imino(4-methoxyphenyl)oxo-λ⁶-sulfanyl]prop-2-en-1-yl]-2-oxo-1,2,5,6,7,8-hexahydroquinoline-3-carboxamide

Purity: 90.6% (254 nm); RT=2.58 min, m/z=402.20 [M+1]; ¹H NMR (400 MHz, DMSO-d6) δ 12.26 (s, 1H), 10.04 (s, 1H), 8.03 (s, 1H), 7.82-7.73 (m, 2H), 7.15-7.04 (m, 2H), 6.71 (dt, J=15.1, 4.7 Hz, 1H), 6.44 (d, J=14.8 Hz, 1H), 4.42 (s, 1H), 4.15-4.07 (m, 2H), 3.83 (s, 3H), 2.58 (s, 3H), 1.69 (s, 4H).

### Example 58: N-[(2E)-3-[(3,4-dimethylphenyl)(imino)oxo-λ⁶-sulfanyl]prop-2-en-1-yl]-2-oxo-1,2,5,6,7,8-hexahydroquinoline-3-carboxamide

Purity: 93.8% (254 nm); RT=2.58 min, m/z=400.30 [M+1]; ¹H NMR (400 MHz, DMSO-d6) δ 12.26 (s, 1H), 10.00 (s, 1H), 8.03 (s, 1H), 7.62 (d, J=2.1 Hz, 1H), 7.57 (dd, J=7.9, 2.0 Hz, 1H), 7.33 (d, J=8.0 Hz, 1H), 6.73 (dt, J=14.9, 4.6 Hz, 1H), 6.49-6.39 (m, 1H), 4.43 (s, 1H), 4.12 (t, J=5.3 Hz, 2H), 2.58 (s, 2H), 2.29 (s, 6H), 1.69 (d, J=7.4 Hz, 4H).

### Example 59: 6,6-difluoro-N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide

Purity: 97.3% (254 nm); RT=1.88 min, m/z=426.20 [M+1], 424.15 [M−1]; ¹H NMR (400 MHz, DMSO-d6) δ 12.50 (s, 1H), 10.04-9.93 (m, 1H), 8.10 (s, 1H), 7.96-7.87 (m, 2H), 7.47-7.36 (m, 2H), 6.79 (dt, J=14.9, 4.6 Hz, 1H), 6.56-6.47 (m, 1H), 4.67 (s, 1H), 4.14 (s, 2H), 3.18-3.07 (m, 2H), 2.83 (t, J=6.9 Hz, 2H), 2.25 (tt, J=13.0, 6.5 Hz, 2H).

### Example 60: N-[(2E)-3-[imino(oxo)phenyl-λ⁶-sulfanyl]prop-2-en-1-yl]-2-oxo-1,2,5,6,7,8-hexahydroquinoline-3-carboxamide

Purity: 97.9% (254 nm); RT=2.25 min, m/z=372.22 [M+1], 370.19 [M−1]; ¹H NMR (400 MHz, DMSO-d6) δ 12.24 (s, 1H), 10.00 (t, J=5.9 Hz, 1H), 8.04 (s, 1H), 7.91-7.79 (m, 2H), 7.69-7.52 (m, 3H), 6.79 (dt, J=15.0, 4.7 Hz, 1H), 6.54-6.46 (m, 1H), 4.58 (s, 1H), 4.20-4.05 (m, 2H), 2.58 (t, J=5.9 Hz, 2H), 1.69 (q, J=7.8 Hz, 4H).

### Example 61: N-[(2E)-3-[(4-tert-butylphenyl)(imino)oxo-λ⁶-sulfanyl]prop-2-en-1-yl]-2-oxo-1,2,5,6,7,8-hexahydroquinoline-3-carboxamide

Purity: 84.1% (254 nm); RT=2.25 min, m/z=427.97[M+1]426.28 [M−1]; ¹H NMR (400 MHz, DMSO-d6) δ 12.24 (s, 1H), 10.02 (s, 1H), 8.03 (d, J=5.0 Hz, 1H), 7.87-7.73 (m, 2H), 7.60 (dq, J=9.0, 2.4, 2.0 Hz, 2H), 6.77 (dt, J=15.0, 4.7 Hz, 1H), 6.47 (dt, J=14.9, 1.8 Hz, 1H), 4.48 (s, 1H), 4.13 (t, J=5.4 Hz, 2H), 2.57 (d, J=6.0 Hz, 2H), 1.74-1.63 (m, 4H), 1.30 (d, J=1.8 Hz, 9H).

### Example 62: N-[(2E)-3-[(4-ethoxyphenyl)(imino)oxo-λ⁶-sulfanyl]prop-2-en-1-yl]-2-oxo-1,2,5,6,7,8-hexahydroquinoline-3-carboxamide

Purity: 92.3% (254 nm): RT=1.93 min, m/z=416.18 [M+1]414.23 [M−1]; ¹H NMR (400 MHz, DMSO-d6) δ 12.27 (s, 1H), 9.99 (t, J=5.9 Hz, 1H), 8.04 (s, 1H), 7.79-7.72 (m, 2H), 7.11-7.03 (m, 2H), 6.71 (dt, J=14.9, 4.7 Hz, 1H), 6.44 (dd, J=14.9, 1.9 Hz, 1H), 4.41 (s, 1H), 4.11 (q, J=6.8 Hz, 4H), 2.57 (d, J=6.3 Hz, 2H), 1.78-1.61 (m, 4H), 1.34 (t, J=7.0 Hz, 3H).

### Example 63: N-[(2E)-3-[Imino(4-methoxy-3,5-dimethylphenyl)oxo-λ⁶-sulfanyl]prop-2-en-1-yl]-2-oxo-1,2,5,6,7,8-hexahydroquinoline-3-carboxamide

Purity: 92.7% (254 nm): RT=2.42 min, m/z=430.25 [M+1]428.25 [M−1]; ¹H NMR (400 MHz, DMSO-d6) δ 12.27 (s, 1H), 9.99 (t, J=5.9 Hz, 1H), 8.04 (s, 1H), 7.54 (s, 2H), 6.74 (dt, J=15.0, 4.6 Hz, 1H), 6.45 (d, J=15.0 Hz, 1H), 4.42 (s, 1H), 4.13 (t, J=5.3 Hz, 2H), 3.70 (s, 3H), 2.58 (t, J=5.9 Hz, 2H), 2.28 (s, 6H), 1.74-1.63 (m, 4H).

### Example 64: N-[(2E)-3-[imino(3-methoxyphenyl)oxo-λ⁶-sulfanyl]prop-2-en-1-yl]-2-oxo-1,2,5,6,7,8-hexahydroquinoline-3-carboxamide

Purity: 94.6% (254 nm); RT=2.11 min, m/z=402.23 [M+1]400.20 [M−1]; ¹H NMR (400 MHz, DMSO-d6) δ 12.27 (s, 1H), 10.05-9.95 (m, 1H), 8.04 (s, 1H), 7.50 (t, J=7.9 Hz, 1H), 7.42 (dt, J=7.8, 1.3 Hz, 1H), 7.37-7.33 (m, 1H), 7.19 (ddd, J=8.1, 2.6, 1.0 Hz, 1H), 6.79 (dt, J=14.9, 4.6 Hz, 1H), 6.54-6.46 (m, 1H), 4.58 (s, 1H), 4.21-4.09 (m, 2H), 2.58 (t, J=5.8 Hz, 2H), 1.78-1.60 (m, 4H).

### Example 65: N-[(2E)-3-[imino(oxo)[4-(trifluoromethoxy)phenyl]-%⁶-sulfanyl]prop-2-en-1-yl]-2-oxo-1,2,5,6,7,8-hexahydroquinoline-3-carboxamide

Purity: 92.3% (254 nm); Rt=2.90 min, m/z=456.15 [M+1], 454.17 [M−1]; ¹H NMR (400 MHz, DMSO-d6) δ 12.27 (s, 1H), 10.10-9.99 (m, 1H), 8.03 (s, 1H), 8.02-7.95 (m, 2H), 7.62-7.53 (m, 2H), 6.84 (dt, J=15.0, 4.6 Hz, 1H), 6.54 (dt, J=14.9, 1.8 Hz, 1H), 4.77 (s, 1H), 4.15 (t, J=5.4 Hz, 2H), 2.58 (t, J=5.9 Hz, 2H), 1.77-1.62 (m, 4H).

### Example 66: Preparation of N-[(2E)-3-(benzenesulfonyl)prop-2-en-1-yl]-2-oxo-1,2,5,6,7,8-hexahydro-1,6-naphthyridine-3-carboxamide hydrochloride (Scheme 3)

tert-Butyl 3-{[(2E)-3-(benzenesulfonyl)prop-2-en-1-yl]carbamoyl}-2-oxo-1,2,5,6,7,8-hexahydro-1,6-naphthyridine-6-carboxylate (1.16 g, 2.4 mmol, 1.0 eq) was suspended in dioxane (5 mL, 0.5M) and cooled down to 0° C. HCl 4.0 M in dioxane (6.0 ml, 24 mmol, 10 eq) was added to the reaction mixture and stirred for 2 h. The solution was concentrated to dryness, the residue was triturated with diethyl ether and dried to obtain N-[(2E)-3-(benzenesulfonyl)prop-2-en-1-yl]-2-oxo-1,2,5,6,7,8-hexahydro-1,6-naphthyridine-3-carboxamide hydrochloride (980 mg Y: 98%).

UPLC Rt=1.20 min, m/z=374.2 [M+1]; Purity: 95% (NMR); ¹H NMR (300 MHz, DMSO-d6) 12.67 (s, 1H), 9.87 (t, J=5.9 Hz, 1H), 9.61 (s, 2H), 8.20 (s, 1H), 7.92-7.81 (m, 2H), 7.79-7.69 (m, 1H), 7.64 (dd, J=8.2, 6.6 Hz, 2H), 6.95 (dt, J=15.1, 4.4 Hz, 1H), 6.74-6.61 (m, 1H), 4.19 (ddd, J=6.2, 4.4, 1.9 Hz, 2H), 4.06 (d, J=4.6 Hz, 2H), 3.42-3.27 (m, 2H), 2.89 (t, J=6.2 Hz, 2H).

### Example 67: Preparation of oxetan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate (Scheme 4)

### Scheme 4, Reaction 1: 4-nitrophenyl oxetan-3-yl carbonate

4-Nitrophenyl chloroformate (0.30 g, 1.48 mmol, 1.1 eq) was added portion wise at 0° C. to a solution of oxetan-3-ol (0.1 g, 1.35 mmol, 1.0 eq) and pyridine (0.164 ml, 2.03 mmol, 1.5 eq) in DCM (3.47 ml, 0.4 M). The reaction was allowed to reach room temperature and stirred for 2 h. The reaction was diluted with DCM, washed with 1 M HCl and saturated aqueous NaHCO₃ solution, dried over MgSO₄, filtered, and then concentrated under reduced pressure to give 4-nitrophenyl oxetan-3-yl carbonate (0.281 g, Y: 74%) as slightly yellow oil that was used in the next step without any further purification. ¹H NMR (DMSO-d₆, 400 MHz) confirmed the structure of the product.

¹H NMR (400 MHz, DMSO-d₆) δ 8.37-8.29 (m, 2H), 7.64-7.55 (m, 2H), 5.53 (tt, J=6.2, 4.7 Hz, 1H), 4.85 (ddd, J=7.5, 6.2, 1.1 Hz, 2H), 4.67 (ddd, J=7.8, 4.7, 1.1 Hz, 2H).

### Scheme 4, Reaction 2: oxetan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate

N-[(2E)-3-(benzenesulfonyl)prop-2-en-1-yl]-2-oxo-1,2,5,6,7,8-hexahydro-1,6-naphthyridine-3-carboxamide (0.050 g, 0.12 mmol, 1.0 eq) was dissolved in tetrahydrofuran (1 mL). Next, aqueous NaHCO₃ (0.020 g, 0.23 mmol, 2.0 eq, 1 mL) and a solution of 4-nitrophenyl oxetan-3-yl carbonate (0.036 g, 0.128 mmol, 1.1 eq) in tetrahydrofuran (0.5 mL) were added and the reaction was stirred for 2 h at room temperature. The reaction mixture was concentrated under reduced pressure and dissolved in DCM. The organic layer was washed with saturated aqueous NaHCO₃ solution until the yellow color disappeared. The organic layer was collected, dried over MgSO₄, filtered and concentrated under reduced pressure. The crude was purified by preparative TLC eluting with DCM/MeOH 10% to give (2,2-difluorocyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate as a white solid (21 mg Y: 37%).

Rt: 2.38 min, m/z=474.3 [M+1]; Purity: 97.79% (254 nm); ¹H NMR (400 MHz, DMSO-d₆) δ 12.50 (s, 1H), 9.95 (t, J=5.9 Hz, 1H), 8.15 (s, 1H), 7.90-7.82 (m, 2H), 7.77-7.69 (m, 1H), 7.64 (dd, J=8.3, 6.8 Hz, 2H), 6.95 (dt, J=15.2, 4.4 Hz, 1H), 6.66 (dt, J=15.1, 1.9 Hz, 1H), 5.31 (p, J=5.6 Hz, 1H), 4.77 (dd, J=7.5, 6.3 Hz, 2H), 4.52 (dd, J=7.6, 5.1 Hz, 2H), 4.49-4.41 (m, 1H), 4.40-4.29 (m, 1H), 4.18 (td, J=5.1, 4.2, 2.0 Hz, 2H), 3.78-3.54 (m, 2H), 2.77-2.68 (m, 2H).

### Example 68: Preparation of cyclopropylmethyl 3-{[(2E)-3-[(4-fluorophenyl)(imino)oxo-A*-sulfanyl]prop-2-en-1-yl]carbamoyl}-2-oxo-1,2,5,6,7,8-hexahydro-1,6-naphthyridine-6-carboxylate (Scheme 4)

### Scheme 4, Reaction 1: cyclopropylmethyl 4-nitrophenyl carbonate

Nitrophenyl chloroformate (1.0 g, 5.0 mmol, 1.0 eq) was added portion wise at 0° C. to a solution of cyclopropylmethanol (0.32 g, 4.5 mmol, 0.9 eq) and pyridine (0.6 ml, 7.4 mmol, 1.5 eq) in DCM (12.4 ml, 0.4 M). The reaction was allowed to reach room temperature and stirred for 2 h. The reaction was diluted with DCM, washed with 1 M HCl and saturated aqueous NaHCO₃ solution, dried over MgSO₄ and filtered. The filtrate was concentrated under reduced pressure to give cyclopropyl methyl 4-nitrophenyl carbonate (0.998 g, Y: 75%) as a slightly yellow oil that was used in the next step without any further purification. ¹H NMR (DMSO-d₆, 300 MHz) confirmed the structure and purity of the product.

¹H NMR (300 MHz, DMSO-d₆) δ 8.37-8.27 (m, 2H), 7.64-7.50 (m, 2H), 4.10 (d, J=7.5 Hz, 2H), 1.21 (pt, J=7.7, 4.7 Hz, 1H), 0.68-0.52 (m, 2H), 0.37 (qd, J=4.6, 2.0 Hz, 2H).

### Scheme 4, Reaction 3: 6-cyclopropylmethyl 3-ethyl 2-oxo-1,2,5,6,7,8-hexahydro-1,6-naphthyridine-3,6-dicarboxylate

Ethyl 1,2,5,6,7,8-hexahydro-2-oxo-1,6-naphthyridine-3-carboxylate (0.67 g, 2.6 mmol, 1.0 eq) was dissolved in THF (11 mL). Next, aqueous NaHCO₃ (0.433 g, 5.157 mmol, 2.0 eq) and a solution of cyclopropylmethyl 4-nitrophenyl carbonate (0.70 g, 2.6 mmol, 1.0 eq) in THF (2 mL) were added and the reaction was stirred for 2 h at room temperature. The mixture was concentrated under reduced pressure and dissolved in DCM. The organic layer was washed with saturated aqueous NaHCO₃ solution until the yellow colour disappeared. The organic layer was collected, dried over MgSO₄, filtered and concentrated under reduced pressure to give 6-cyclopropylmethyl 3-ethyl 2-oxo-1,2,5,6,7,8-hexahydro-1,6-naphthyridine-3,6-dicarboxylate (0.625 g, Y: 72%) as a yellow solid. ¹H NMR (DMSO-d₆, 300 MHz) confirmed the structure of the product.

Purity: 90% (¹H-NMR); ¹H NMR (300 MHz, DMSO-d₆) δ 12.01 (s, 1H), 7.93 (s, 1H), 4.32 (s, 2H), 4.19 (q, J=7.1 Hz, 2H), 3.88 (d, J=7.1 Hz, 2H), 3.61 (t, J=5.8 Hz, 2H), 2.63 (t, J=5.9 Hz, 2H), 1.26 (t, J=7.1 Hz, 3H), 1.11 (pt, J=7.2, 4.7 Hz, 1H), 0.58-0.43 (m, 2H), 0.33-0.21 (m, 2H).

### Scheme 4, Reaction 4: 6-[(cyclopropylmethoxy)carbonyl]-2-oxo-1,2,5,6,7,8-hexahydro-1,6-naphthyridine-3-carboxylic acid

6-Cyclopropylmethyl 3-ethyl 2-oxo-1,2,5,6,7,8-hexahydro-1,6-naphthyridine-3,6-dicarboxylate (0.63 g, 1.9 mmol, 1.0 eq) was dissolved in tetrahydrofuran (7.4 ml, 0.25 M) and the solution was cooled to 0° C. Next, 1.0 M aqueous LiOH (4.64 ml, 2.5 eq) was added and the mixture was stirred for 12 h at room temperature. Tetrahydrofuran was evaporated under reduced pressure, 0.5 HCl was added until ~pH 3 and the resulting precipitate was filtered, washed with water and dried under reduced pressure to give 6-[(cyclopropylmethoxy)carbonyl]-2-oxo-1,2,5,6,7,8-hexahydro-1,6-naphthyridine-3-carboxylic acid (0.425 g, Y:78%) as a solid. ¹H NMR (DMSO-d₆, 300 MHz) confirmed the structure of the product.

UPLC: Rt: 1.15 min, m/z: 293.8 [M+1]; Purity (254): 90%; ¹H NMR (300 MHz, DMSO-d₆) δ 14.80 (s, 1H), 13.28 (s, 1H), 8.29 (s, 1H), 4.44 (s, 2H), 3.89 (d, J=7.1 Hz, 2H), 3.66 (t, J=5.9 Hz, 2H), 2.78 (t, J=5.9 Hz, 2H), 1.20-1.03 (m, 1H), 0.58-0.43 (m, 2H), 0.37-0.21 (m, 2H).

### Scheme 4, Reaction 5: cyclopropylmethyl 3-{[(2E)-3-[(4-fluorophenyl)(imino)oxo-λ⁶-sulfanyl]prop-2-en-1-yl]carbamoyl}-2-oxo-1,2,5,6,7,8-hexahydro-1,6-naphthyridine-6-carboxylate

6-[(cyclopropylmethoxy)carbonyl]-2-oxo-1,2,5,6,7,8-hexahydro-1,6-naphthyridine-3-carboxylic acid (0.036 g, 0.122 mmol, 1.0 eq) and HATU (0.046 g, 0.122 mmol, 1.0 eq) were dissolved in anhydrous DMF (0.4 mL). Next, N,N-diisopropylethylamine (0.085 ml, 0.49 mmol, 4.0 eq) was added and the yellow mixture was stirred for 10 min. [(1E)-3-aminoprop-1-en-1-yl](4-fluorophenyl)imino-λ⁶-sulfanone dihydrochloride (0.033 g, 0.11 mmol, 0.9 eq) was added and the mixture was stirred at room temperature for 2 h. The reaction mixture was partitioned between water and DCM, and the organic layer was collected, washed with saturated NaHCO₃ solution, saturated NaCl solution, dried over MgSO₄, filtered, and concentrated under reduced pressure. The residue was purified by preparative TLC eluting with DCM/MeOH 10% to obtain cyclopropylmethyl 3-{[(2E)-3-[(4-fluorophenyl)(imino)oxo-λ⁶-sulfanyl]prop-2-en-1-yl]carbamoyl}-2-oxo-1,2,5,6,7,8-hexahydro-1,6-naphthyridine-6-carboxylate (0.005 g, Y: 7%) as a yellow solid.

Rt: 2.03 min, m/z=489.14 [M+1]; Purity (254): 90.3%; ¹H NMR (400 MHz, DMSO-d₆) δ 12.48 (s, 1H), 10.07-9.95 (m, 1H), 8.14 (s, 1H), 7.96-7.88 (m, 2H), 7.46-7.37 (m, 2H), 6.80 (dt, J=15.0, 4.6 Hz, 1H), 6.56-6.47 (m, 1H), 4.67 (s, 1H), 4.42-4.33 (m, 2H), 4.18-4.12 (m, 2H), 3.89 (d, J=7.1 Hz, 2H), 3.66-3.59 (m, 2H), 2.70-2.64 (m, 2H), 1.15-1.07 (m, 1H), 0.55-0.46 (m, 2H), 0.32-0.24 (m, 2H).

The title compounds of Examples 69-98 and 178-184 were prepared by a method analogous to that described in Example 67 or 68.

### Example 69: cyclopropylmethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 95.6% (254 nm); RT=1.94 min, m/z=471.94 [M+1]470.20 [M−1]; ¹H NMR (400 MHz, DMSO-d6) δ 12.49 (s, 1H), 9.96 (t, J=6.1 Hz, 1H), 8.15 (s, 1H), 7.91-7.82 (m, 2H), 7.78-7.69 (m, 1H), 7.69-7.58 (m, 2H), 6.95 (dt, J=15.1, 4.4 Hz, 1H), 6.67 (dt, J=15.0, 1.8 Hz, 1H), 4.37 (s, 2H), 4.24-4.12 (m, 2H), 3.88 (d, J=7.1 Hz, 2H), 3.72-3.53 (m, 2H), 2.73-2.62 (m, 2H), 1.19-1.03 (m, 1H), 0.55-0.44 (m, 2H), 0.32-0.21 (m, 2H).

### Example 70: cyclobutyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 97.9% (254 nm); RT=1.97 min, m/z=472.20 [M+1]469.91 [M−1]; ¹H NMR (400 MHz, DMSO-d6) δ 12.48 (s, 1H), 9.96 (t, J=5.9 Hz, 1H), 8.13 (s, 1H), 7.90-7.80 (m, 2H), 7.79-7.68 (m, 1H), 7.68-7.56 (m, 2H), 6.95 (dt, J=15.1, 4.4 Hz, 1H), 6.66 (dt, J=15.1, 1.8 Hz, 1H), 4.92-4.79 (m, 1H), 4.48-4.25 (m, 2H), 4.24-4.13 (m, 2H), 3.68-3.52 (m, 2H), 2.73-2.62 (m, 2H), 2.30-2.18 (m, 2H), 2.09-1.94 (m, 2H), 1.72 (q, J=10.4 Hz, 1H), 1.64-1.48 (m, 1H).

### Example 71: (5-methyl-1,3,4-oxadiazol-2-yl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 99.0% (254 nm); RT=1.81 min, m/z=514.18 [M+1]512.21 [M−1]; ¹H NMR (300 MHz, DMSO-d6) δ 12.50 (s, 1H), 9.95 (t, J=5.9 Hz, 1H), 8.15 (s, 1H), 7.92-7.80 (m, 2H), 7.79-7.68 (m, 1H), 7.64 (dd, J=8.2, 6.6 Hz, 2H), 6.95 (dt, J=15.1, 4.4 Hz, 1H), 6.66 (d, J=15.2 Hz, 1H), 5.30 (s, 2H), 4.40 (s, 2H), 4.18 (t, J=4.9 Hz, 2H), 3.64 (s, 2H), 2.70 (s, 1H).

### Example 72: 1,1,1-trifluoropropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 97.5% (254 nm); RT=2.70 min, m/z=512.00 [M−1]; ¹H NMR (300 MHz, DMSO-d6) δ 12.52 (s, 1H), 9.97 (d, J=6.0 Hz, 1H), 8.16 (s, 1H), 7.91-7.80 (m, 2H), 7.79-7.69 (m, 1H), 7.64 (dd, J=8.2, 6.6 Hz, 2H), 6.95 (dt, J=15.2, 4.3 Hz, 1H), 6.66 (d, J=15.1 Hz, 1H), 5.36 (p, J=6.8 Hz, 1H), 4.48-4.32 (m, 2H), 4.25-4.09 (m, 2H), 3.73-3.57 (m, 2H), 2.77-2.64 (m, 2H), 1.38 (d, J=6.6 Hz, 3H). 1H NMR (300 MHz, DMSO-d6) δ 12.52 (s, 1H), 9.97 (d, J=6.0 Hz, 1H), 8.16 (s, 1H), 7.91-7.80 (m, 2H), 7.79-7.69 (m, 1H), 7.64 (dd, J=8.2, 6.6 Hz, 2H), 6.95 (dt, J=15.2, 4.3 Hz, 1H), 6.66 (d, J=15.1 Hz, 1H), 5.36 (p, J=6.8 Hz, 1H), 4.48-4.32 (m, 2H), 4.25-4.09 (m, 2H), 3.73-3.57 (m, 2H), 2.77-2.64 (m, 2H), 1.38 (d, J=6.6 Hz, 3H).

### Example 73: (2,2-dimethylcyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 94.5% (254 nm); RT=2.22 min, m/z=500.2 [M+1]498.40 [M−1]; ¹H NMR (400 MHz, DMSO-d6) δ 12.49 (s, 1H), 9.95 (t, J=5.9 Hz, 1H), 8.15 (s, 1H), 7.90-7.82 (m, 2H), 7.78-7.70 (m, 1H), 7.65 (dd, J=8.4, 6.9 Hz, 2H), 6.95 (dt, J=15.1, 4.4 Hz, 1H), 6.67 (dt, J=15.1, 1.8 Hz, 1H), 4.45-4.29 (m, 2H), 4.29-4.13 (m, 3H), 3.88 (dd, J=11.6, 8.8 Hz, 1H), 3.71-3.56 (m, 2H), 2.74-2.63 (m, 2H), 1.08 (s, 3H), 1.04 (s, 3H), 0.99-0.84 (m, 1H), 0.50 (dd, J=8.6, 4.3 Hz, 1H), 0.25 (t, J=4.8 Hz, 1H).

### Example 74: (2,2-difluorocyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 99.2% (254 nm); RT=2.47 min, m/z=508.41 [M+1]506.11 [M−1]; ¹H NMR (400 MHz, DMSO-d6) δ 12.49 (s, 1H), 9.96 (t, J=5.8 Hz, 1H), 8.15 (s, 1H), 7.91-7.81 (m, 2H), 7.76-7.69 (m, 1H), 7.69-7.59 (m, 2H), 6.95 (dt, J=15.1, 4.4 Hz, 1H), 6.67 (dt, J=15.1, 1.8 Hz, 1H), 4.44-4.31 (m, 2H), 4.25-4.13 (m, 3H), 4.05-3.94 (m, 1H), 3.62 (t, J=6.7 Hz, 2H), 2.74-2.64 (m, 2H), 2.11 (ddt, J=20.2, 14.3, 7.4 Hz, 1H), 1.67 (tdd, J=12.2, 7.8, 4.8 Hz, 1H), 1.53-1.37 (m, 1H).

### Example 75: 1-methoxypropan-2-vi 3-{[(2E)-3-(benzenesulfonyl)prop-2-en-1-yl]carbamoyl}-2-oxo-1,2,5,6,7,8-hexahydro-1,6-naphthyridine-6-carboxylate

Purity 98.0% (254 nm); RT=2.11 min, m/z=490.18 [M+1]487.92 [M−1]; ¹H NMR (300 MHz, DMSO-d6) δ 12.49 (s, 1H), 9.95 (t, J=5.9 Hz, 1H), 8.14 (s, 1H), 7.96-7.81 (m, 2H), 7.77-7.70 (m, 1H), 7.69-7.55 (m, 2H), 6.95 (dt, J=15.1, 4.4 Hz, 1H), 6.66 (dt, J=15.1, 1.8 Hz, 1H), 4.86 (td, J=6.4, 4.1 Hz, 1H), 4.35 (s, 2H), 4.17 (d, J=5.6 Hz, 2H), 3.61 (t, J=5.8 Hz, 2H), 3.48-3.36 (m, 2H), 3.26 (s, 3H), 2.71-2.61 (m, 2H), 1.16 (d, J=6.4 Hz, 3H).

### Example 76: 2-fluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 99.3% (254 nm): RT=2.04 min, m/z=464.15 [M+1]462.12 [M−1]; 1H NMR (400 MHz, DMSO-d6) δ 12.50 (s, 1H), 9.96 (t, J=5.9 Hz, 1H), 8.15 (s, 1H), 7.92-7.82 (m, 2H), 7.78-7.70 (m, 1H), 7.65 (dd, J=8.2, 6.8 Hz, 2H), 6.95 (dt, J=15.2, 4.4 Hz, 1H), 6.67 (dt, J=15.1, 1.9 Hz, 1H), 4.72-4.66 (m, 1H), 4.60-4.54 (m, 1H), 4.39 (s, 2H), 4.35-4.31 (m, 1H), 4.25 (dd, J=4.7, 3.2 Hz, 1H), 4.19 (ddd, J=6.3, 4.6, 2.0 Hz, 2H), 3.64 (s, 2H), 2.73-2.65 (m, 2H).

### Example 77: 2,2-Difluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 99.7% (254 nm); RT=2.25 min, m/z=481.93 [M+1], 480.251 [M−1]; ¹H NMR (400 MHz, DMSO-d6) δ 12.51 (s, 1H), 9.95 (t, J=5.8 Hz, 1H), 8.16 (s, 1H), 7.93-7.81 (m, 2H), 7.78-7.69 (m, 1H), 7.69-7.56 (m, 2H), 6.95 (dt, J=15.1, 4.4 Hz, 1H), 6.67 (dt, J=15.0, 1.8 Hz, 1H), 6.45-6.09 (m, 1H), 4.46-4.28 (m, 2H), 4.23-4.14 (m, 2H), 3.65 (s, 2H), 2.75-2.65 (m, 2H).

### Example 78: cyclopropyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 96.3% (254 nm); RT=2.16 m/z=458.10 [M+1]; ¹H NMR (400 MHz, DMSO-d6) δ 12.48 (s, 1H), 9.95 (t, J=5.9 Hz, 1H), 8.12 (s, 1H), 7.94-7.80 (m, 2H), 7.80-7.68 (m, 1H), 7.68-7.58 (m, 2H), 6.95 (dt, J=15.2, 4.4 Hz, 1H), 6.66 (dt, J=15.1, 1.8 Hz, 1H), 4.40-4.26 (m, 2H), 4.22-4.13 (m, 2H), 4.06-3.98 (m, 1H), 3.66-3.48 (m, 2H), 2.72-2.60 (m, 3H), 0.69-0.60 (m, 4H).

### Example 79: 2-Oxaspiro[3.5]nonan-7-vi 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 96.4% (254 nm); RT=2.19 min, m/z=542.4 [M+1]; ¹H NMR (400 MHz, DMSO-d6) δ 12.48 (s, 1H), 9.96 (t, J=5.8 Hz, 1H), 8.14 (s, 1H), 7.91-7.82 (m, 2H), 7.77-7.69 (m, 1H), 7.69-7.59 (m, 2H), 6.95 (dt, J=15.2, 4.4 Hz, 1H), 6.66 (dt, J=15.1, 1.8 Hz, 1H), 4.64-4.53 (m, 1H), 4.40-4.31 (m, 2H), 4.28 (s, 2H), 4.26 (s, 2H), 4.22-4.14 (m, 2H), 3.66-3.53 (m, 2H), 2.72-2.60 (m, 2H), 1.98-1.84 (m, 2H), 1.76-1.56 (m, 4H), 1.51-1.35 (m, 2H).

### Example 80: 2-Methoxyethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 98.0% (254 nm); RT=1.91 min, m/z=476.10 [M+1]; ¹H NMR (400 MHz, DMSO-d6) δ 12.49 (s, 1H), 9.97 (t, J=5.8 Hz, 1H), 8.14 (s, 1H), 7.93-7.80 (m, 2H), 7.79-7.69 (m, 1H), 7.69-7.56 (m, 2H), 6.95 (dt, J=15.1, 4.4 Hz, 1H), 6.67 (dt, J= 15.1, 1.9 Hz, 1H), 4.37 (s, 2H), 4.27-4.08 (m, 4H), 3.62 (t, J=5.7 Hz, 2H), 3.58-3.50 (m, 2H), 3.27 (s, 3H), 2.68 (t, J=5.6 Hz, 2H).

### Example 81: (3S)-Oxolan-3-vi 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 99.0% (254 nm); RT=1.89 min, m/z=488.30 [M+1]; ¹H NMR (400 MHz, DMSO-d6) δ 12.48 (s, 1H), 9.96 (t, 1H), 8.14 (s, 1H), 7.90-7.81 (m, 2H), 7.77-7.69 (m, 1H), 7.64 (dd, J=8.4, 6.9 Hz, 2H), 6.95 (dt, J=15.1, 4.4 Hz, 1H), 6.66 (dt, J=14.9, 1.8 Hz, 1H), 5.22-5.10 (m, 1H), 4.44-4.29 (m, 2H), 4.23-4.13 (m, 2H), 3.84-3.75 (m, 2H), 3.75-3.67 (m, 2H), 3.60 (t, J=5.9 Hz, 2H), 2.72-2.63 (m, 2H), 2.10 (dt, J=14.7, 7.4 Hz, 1H), 1.93 (dt, J=12.4, 5.2 Hz, 1H).

### Example 82: (3R)-oxolan-3-vi 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 98.9% (254 nm); RT=1.89 min, m/z=488.30 [M+1]; ¹H NMR (400 MHz, DMSO-d6) δ 12.49 (s, 1H), 9.96 (t, J=5.9 Hz, 1H), 8.14 (s, 1H), 7.91-7.82 (m, 2H), 7.78-7.69 (m, 1H), 7.69-7.59 (m, 2H), 6.95 (dt, J=15.1, 4.4 Hz, 1H), 6.67 (dt, J=15.1, 1.8 Hz, 1H), 5.21-5.11 (m, 1H), 4.45-4.28 (m, 2H), 4.25-4.13 (m, 2H), 3.86-3.76 (m, 2H), 3.76-3.68 (m, 2H), 3.61 (t, J=5.9 Hz, 2H), 2.73-2.63 (m, 2H), 2.11 (dt, 1H), 1.95 (dt, 1H).

### Example 83: 2-(2-Oxopiperidin-1-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 97.2% (254 nm); RT=2.17 min, m/z=543.30 [M+1], 541.16 [M−1]; ¹H NMR (400 MHz, DMSO-d6) δ 12.49 (s, 1H), 9.96 (t, J=6.0 Hz, 1H), 8.13 (s, 1H), 7.90-7.82 (m, 2H), 7.78-7.70 (m, 1H), 7.70-7.58 (m, 2H), 6.95 (dt, J=15.1, 4.4 Hz, 1H), 6.66 (d, J=15.1 Hz, 1H), 4.35 (s, 2H), 4.26-4.06 (m, 4H), 3.60 (t, J=5.8 Hz, 2H), 3.56-3.43 (m, 2H), 3.31-3.24 (m, 2H), 2.73-2.61 (m, 2H), 2.23-2.07 (m, 2H), 1.77-1.56 (m, 4H).

### Example 84: cyclopentyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 98.8% (254 nm); RT=2.22 min, m/z=486.03 [M+1]484.30 [M−1]; ¹H NMR (400 MHz, DMSO-d6) δ 12.48 (s, 1H), 9.97 (t, J=5.7 Hz, 1H), 8.14 (s, 1H), 7.91-7.82 (m, 2H), 7.78-7.69 (m, 1H), 7.69-7.58 (m, 2H), 6.95 (dt, J=15.1, 4.4 Hz, 1H), 6.67 (dt, J=15.2, 1.8 Hz, 1H), 5.02 (tt, J=5.6, 2.6 Hz, 1H), 4.34 (s, 2H), 4.23-4.11 (m, 2H), 3.59 (t, J=5.9 Hz, 2H), 2.72-2.61 (m, 3H), 1.86-1.74 (m, 2H), 1.74-1.60 (m, 5H), 1.60-1.46 (m, 2H).

### Example 85: (2,2-dimethylcyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 94.3% (254 nm); RT=2.89 min m/z=534.16 [M+1], 532.15 [M−1]; ¹H NMR (400 MHz, DMSO-d6) δ 12.49 (s, 1H), 9.96 (t, J=5.9 Hz, 1H), 8.15 (s, 1H), 7.93-7.82 (m, 2H), 7.76-7.67 (m, 2H), 6.98 (dt, J=15.1, 4.4 Hz, 1H), 6.75-6.64 (m, 1H), 4.37 (s, 2H), 4.30-4.12 (m, 3H), 3.88 (dd, J=11.6, 8.8 Hz, 1H), 3.71-3.56 (m, 2H), 2.68 (t, J=5.9 Hz, 2H), 1.08 (s, 3H), 1.04 (s, 3H), 0.98-0.86 (m, 1H), 0.49 (dd, J=8.6, 4.3 Hz, 1H), 0.25 (t, J=4.7 Hz, 1H).

### Example 86: (2,2-difluorocyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 97.4% (254 nm); RT=2.41 m/z=542.34 [M+1]540.06 [M−1]; ¹H NMR (400 MHz, DMSO-d6) δ 12.50 (s, 1H), 9.96 (t, 1H), 8.15 (s, 1H), 7.93-7.83 (m, 2H), 7.77-7.67 (m, 2H), 6.98 (dt, J=15.2, 4.4 Hz, 1H), 6.70 (dt, J=15.0, 1.8 Hz, 1H), 4.38 (s, 2H), 4.28-4.13 (m, 3H), 4.01 (dd, J=11.9, 8.3 Hz, 1H), 3.63 (d, 2H), 2.74-2.64 (m, 2H), 2.18-2.02 (m, 1H), 1.74-1.60 (m, 1H), 1.53-1.40 (m, 1H).

### Example 87: oxetan-3-vi 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 97.5% (254 nm); RT=1.66 min, m/z=508.12 [M+1]506.27 [M−1]; ¹H NMR (400 MHz, DMSO-d6) δ 12.51 (s, 1H), 9.95 (t, J=5.9 Hz, 1H), 8.16 (s, 1H), 7.94-7.81 (m, 2H), 7.79-7.65 (m, 2H), 6.98 (dt, J=15.2, 4.4 Hz, 1H), 6.70 (dd, J=15.2, 1.9 Hz, 1H), 5.32 (p, J=5.7 Hz, 1H), 4.77 (dd, J=7.5, 6.3 Hz, 2H), 4.52 (dd, J=7.6, 5.1 Hz, 2H), 4.48 (s, 1H), 4.39-4.29 (m, 1H), 4.23-4.14 (m, 2H), 3.77-3.54 (m, 2H), 2.76-2.69 (m, 2H).

### Example 88: cyclopropylmethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 99.2% (254 nm); RT=2.77 min, m/z=506.10 [M+1]503.80 [M−1]; ¹H NMR (400 MHz, DMSO-d6) δ 12.49 (s, 1H), 9.96 (t, J=5.8 Hz, 1H), 8.15 (s, 1H), 7.92-7.83 (m, 2H), 7.77-7.67 (m, 2H), 6.98 (dt, J=15.2, 4.4 Hz, 1H), 6.70 (dt, J=15.1, 1.9 Hz, 1H), 4.49-4.28 (m, 2H), 4.28-4.12 (m, 2H), 3.89 (d, J=7.2 Hz, 2H), 3.71-3.54 (m, 2H), 2.74-2.63 (m, 2H), 1.19-1.04 (m, 1H), 0.57-0.44 (m, 2H), 0.34-0.22 (m, 2H).

### Example 89: 2-(furan-2-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 95.3% (254 nm); RT=2.55 min, m/z=512.16 [M+1], 509.89 [M−1]; ¹H NMR (400 MHz, DMSO-d6) δ 12.48 (s, 1H), 9.98 (t, 1H), 8.14 (s, 1H), 7.91-7.82 (m, 2H), 7.74 (t, J=7.4 Hz, 1H), 7.65 (t, J=7.6 Hz, 2H), 7.61-7.55 (m, 1H), 7.55-7.48 (m, 1H), 6.96 (dt, J=15.1, 4.5 Hz, 1H), 6.67 (d, J=15.1 Hz, 1H), 6.44 (s, 1H), 4.36 (s, 2H), 4.18 (q, J=6.9, 6.2 Hz, 4H), 3.60 (t, J=5.9 Hz, 2H), 2.72 (t, J=6.6 Hz, 2H), 2.70-2.60 (m, 2H).

### Example 90: cyclohexyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 99.8% (254 nm); RT=2.89 min, m/z=500.20 [M+1], 498.33 [M−1]; ¹H NMR (400 MHz, DMSO-d6) δ 12.48 (s, 1H), 9.96 (t, J=5.9 Hz, 1H), 8.15 (s, 1H), 7.93-7.80 (m, 2H), 7.77-7.68 (m, 1H), 7.68-7.57 (m, 2H), 6.95 (dt, J=15.1, 4.4 Hz, 1H), 6.67 (dt, J=15.1, 1.8 Hz, 1H), 4.60 (dt, J=8.3, 4.6 Hz, 1H), 4.36 (s, 2H), 4.27-4.12 (m, 2H), 3.70-3.55 (m, 2H), 2.67 (t, J=5.7 Hz, 2H), 1.84-1.72 (m, 2H), 1.72-1.60 (m, 2H), 1.53-1.23 (m, 6H).

### Example 91: 1-(2,6-difluorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 98.5% (254 nm); RT=2.976 min, m/z=558.13 [M+1]556.14 [M−1]; ¹H NMR (400 MHz, DMSO-d6) δ 12.48 (s, 1H), 9.94 (t, J=5.9 Hz, 1H), 8.12 (s, 1H), 7.90-7.80 (m, 2H), 7.79-7.69 (m, 1H), 7.69-7.57 (m, 2H), 7.41 (ddd, J=15.0, 8.5, 6.5 Hz, 1H), 7.10 (t, J=8.4 Hz, 2H), 6.95 (dt, J=15.2, 4.4 Hz, 1H), 6.67 (d, J=15.2 Hz, 1H), 6.02 (q, J=6.7 Hz, 1H), 4.49-4.27 (m, 2H), 4.22-4.13 (m, 2H), 3.70-3.51 (m, 2H), 2.74-2.59 (m, 2H), 1.60 (d, J=6.8 Hz, 3H).

### Example 92: 1-acetylpiperidin-4-vi 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 94.6% (254 nm); RT=2.14 min, m/z=577.15 [M+1]575.08 [M−1]; ¹H NMR (400 MHz, DMSO-d6) δ 12.49 (s, 1H), 9.97 (s, 1H), 8.16 (s, 1H), 7.92-7.83 (m, 2H), 7.77-7.68 (m, 2H), 6.98 (dt, J=15.2, 4.4 Hz, 1H), 6.70 (d, J=15.2 Hz, 1H), 4.84 (s, 1H), 4.40 (d, J=22.8 Hz, 2H), 4.19 (t, J=4.9 Hz, 2H), 3.71-3.53 (m, 4H), 3.51-3.37 (m, 2H), 2.73-2.64 (m, 2H), 2.01 (s, 3H), 1.91-1.80 (m, 1H), 1.80-1.70 (m, 1H), 1.65-1.56 (m, 1H), 1.56-1.44 (m, 1H).

### Example 93: 2-fluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 99.0% (254 nm); RT=2.39 min, m/z=498.10 [M+1]496.10 [M−1]; ¹H NMR (400 MHz, DMSO-d6) δ 12.50 (s, 1H), 9.96 (t, J=5.7 Hz, 1H), 8.15 (s, 1H), 7.91-7.83 (m, 2H), 7.75-7.68 (m, 2H), 6.98 (dt, J=15.1, 4.4 Hz, 1H), 6.70 (dt, J=15.1, 1.8 Hz, 1H), 4.72-4.65 (m, 1H), 4.61-4.54 (m, 1H), 4.50-4.35 (m, 2H), 4.35-4.30 (m, 1H), 4.28-4.22 (m, 1H), 4.22-4.13 (m, 2H), 3.71-3.58 (m, 2H), 2.75-2.64 (m, 2H).

### Example 94: 1-methoxypropan-2-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: % (254 nm); RT=1.98 min, m/z=523.80 [M+1]522.20 [M−1]; ¹H NMR (400 MHz, DMSO-d6) δ 12.49 (s, 1H), 9.95 (t, J=5.9 Hz, 1H), 8.14 (s, 1H), 7.92-7.83 (m, 2H), 7.76-7.66 (m, 2H), 6.98 (dt, J=15.1, 4.3 Hz, 1H), 6.70 (dt, J=15.0, 1.8 Hz, 1H), 4.86 (td, J=6.4, 4.0 Hz, 1H), 4.36 (s, 2H), 4.19 (dq, J=4.6, 2.2 Hz, 2H), 3.61 (t, J=5.9 Hz, 2H), 3.46-3.35 (m, 2H), 3.26 (s, 3H), 2.71-2.63 (m, 2H), 1.16 (d, J=6.5 Hz, 3H).

### Example 95: cyclopropylmethyl 3-{[(2E)-3-[(5-chloropyridin-2-yl)sulfonyl]prop-2-en-1-yl]carbamoyl}-2-oxo-1,2,5,6,7,8-hexahydro-1,6-naphthyridine-6-carboxylate

Purity: 90.5% (254 nm); RT=2.42 min, m/z=507.14 [M+1], 505.20 [M−1]; ¹H NMR (300 MHz, DMSO-d6) δ 12.49 (s, 1H), 10.00 (d, J=5.9 Hz, 1H), 8.86 (dd, J=2.5, 0.7 Hz, 1H), 8.28 (dd, J=8.4, 2.4 Hz, 1H), 8.16 (s, 1H), 8.08 (dd, J=8.5, 0.7 Hz, 1H), 7.06 (dt, J=15.3, 4.4 Hz, 1H), 6.80-6.66 (m, 1H), 4.38 (s, 2H), 4.23 (s, 2H), 3.88 (d, J=7.2 Hz, 2H), 3.63 (s, 2H), 2.69 (s, 2H), 1.10 (dd, J=13.4, 5.8 Hz, 1H), 0.58-0.45 (m, 2H), 0.34-0.23 (m, 2H).

### Example 96: 2,2-difluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 89.3% (254 nm); RT=2.89 min, m/z=516.10 [M+1], 514.38 [M−1]; ¹H NMR (400 MHz, DMSO-d6) δ 12.51 (s, 1H), 10.00 (s, 1H), 8.14 (s, 1H), 7.91-7.84 (m, 2H), 7.76-7.68 (m, 2H), 6.98 (dt, J=15.2, 4.4 Hz, 1H), 6.70 (d, J=15.1 Hz, 1H), 6.27 (t, J=54.6 Hz, 1H), 4.46-4.29 (m, 4H), 4.19 (s, 2H), 3.65 (s, 2H), 2.75-2.68 (m, 2H).

### Example 97: 2,2,2-trifluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate

Purity: 92.1% (254 nm); RT=2.43 min, m/z=534.25 [M+1]532.24 [M−1]; ¹H NMR (400 MHz, DMSO-d6) δ 12.51 (s, 1H), 9.98 (s, 1H), 8.16 (s, 1H), 7.91-7.83 (m, 2H), 7.75-7.67 (m, 2H), 6.98 (dt, J=15.1, 4.4 Hz, 1H), 6.69 (d, J=15.1 Hz, 1H), 4.75 (q, J=9.0 Hz, 2H), 4.48-4.37 (m, 2H), 4.22-4.15 (m, 2H), 3.70-3.62 (m, 2H), 2.74-2.69 (m, 2H).

### Example 98: cyclopropylmethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate

Purity: 87.0% (254 nm); RT=2.09 min, m/z=519.20 [M+1], 517.30 [M−1]; ¹H NMR (300 MHz, DMSO-d6) δ 12.47 (s, 1H), 9.99 (s, 1H), 8.14 (s, 1H), 7.70-7.58 (m, 2H), 7.35 (t, J=8.6 Hz, 1H), 6.77 (dt, J=14.9, 4.6 Hz, 1H), 6.49 (d, J=15.0 Hz, 1H), 4.60 (s, 1H), 4.38 (s, 2H), 4.13 (d, J=5.8 Hz, 2H), 3.92 (s, 3H), 3.88 (d, J=7.1 Hz, 2H), 3.63 (s, 2H), 2.69 (d, J=6.4 Hz, 2H), 1.17-1.06 (m, 1H), 0.56-0.46 (m, 2H), 0.33-0.23 (m, 2H).

### Example 99: Preparation of N6-tert-butyl-N3-[(2E)-3-(4-chlorobenzenesulfonyl)prop-2-en-1-yl]-2-oxo-1,2,5,6,7,8-hexahydro-1,6-naphthyridine-3,6-dicarboxamide (Scheme 5)

### Scheme 5, Reaction 1: 4-nitrophenyl N-tert-butylcarbamate

Nitrophenyl chloroformate (0.14 g, 0.68 mmol, 1.0 eq) was added portionwise at 0° C. to a solution of tert-butylamine (0.072 ml, 0.68 mmol, 1.0 eq) and pyridine (0.083 ml, 1.03 mmol, 1.5 eq) in DCM (12.4 ml, 0.4 M). The reaction mixture was allowed to reach room temperature and stirred for 2 h. The reaction was diluted with DCM, washed with 1 M HCl and saturated aqueous NaHCO₃ solution, dried over MgSO₄, filtered, and then concentrated under reduced pressure to give 4-nitrophenyl N-tert-butylcarbamate (0.137 g, Y: 70%) as a pale-yellow solid that was used in the next step without any further purification. ¹H NMR (DMSO-d₆, 400 MHz) confirmed the structure and purity of the product.

¹H NMR (400 MHz, DMSO-d₆) δ 8.30-8.22 (m, 2H), 7.88 (s, 1H), 7.42-7.33 (m, 2H), 1.31 (s, 9H).

### Scheme 5, Reaction 2: N6-tert-butyl-N3-[(2E)-3-(4-chlorobenzenesulfonyl)prop-2-en-1-yl]-2-oxo-1,2,5,6,7,8-hexahydro-1,6-naphthyridine-3,6-dicarboxamide

N-[(2E)-3-(4-chlorobenzenesulfonyl)prop-2-en-1-yl]-2-oxo-1,2,5,6,7,8-hexahydro-1,6-naphthyridine-3-carboxamide hydrochloride (0.04 g, 0.086 mmol, 1.0 eq) was dissolved in THF (1 mL), aqueous NaHCO₃ (0.014 g, 0.17 mmol, 2.0 eq) and a solution of 4-nitrophenyl N-tert-butylcarbamate (0.027 g, 0.094 mmol, 1.1 eq) in anhydrous THF (0.86 ml, 0.1 M) were added and the reaction was stirred for 2 h at room temperature. The mixture was concentrated under reduced pressure and dissolved in DCM. Organic layer was washed with saturated aqueous NaHCO₃ solution until the yellow colour disappeared. The organic layer was collected, dried over MgSO₄, filtered and concentrated under reduced pressure. The crude was purified by preparative TLC eluting with DCM/MeOH 10% to give N6-tert-butyl-N3-[(2E)-3-(4-chlorobenzenesulfonyl)prop-2-en-1-yl]-2-oxo-1,2,5,6,7,8-hexahydro-1,6-naphthyridine-3,6-dicarboxamide (0.005 g, Y: 12%) as a white solid.

Rt=2.55 min, m/z=507.10 [M+1], 505.10 [M−1]; Purity: 99.6% (254 nm); ¹H NMR (400 MHz, DMSO-d₆) δ 12.44 (s, 1H), 9.99 (s, 1H), 8.07 (s, 1H), 7.94-7.82 (m, 2H), 7.78-7.66 (m, 2H), 6.98 (dt, J=15.1, 4.3 Hz, 1H), 6.75-6.61 (m, 1H), 5.89 (s, 1H), 4.26 (s, 2H), 4.19 (d, J=5.0 Hz, 2H), 3.53 (t, J=5.8 Hz, 2H), 2.62 (t, J=5.6 Hz, 2H), 1.26 (s, 9H).

The title compounds of Examples 100 and 101 were prepared by a method analogous to that described in Example 99.

### Example 100: N3-[(2E)-3-(4-chlorobenzenesulfonyl)prop-2-en-1-yl]-N6-(cyclopropylmethyl)-2-oxo-1,2,5,6,7,8-hexahydro-1,6-naphthyridine-3,6-dicarboxamide

Purity: 99.6 (254 nm); RT=3.24 min, m/z=505.1 [M+1]; ¹H NMR (400 MHz, DMSO-d6) δ 12.29 (s, 1H), 9.99 (s, 1H), 8.07 (s, 1H), 7.92-7.82 (m, 2H), 7.77-7.65 (m, 2H), 6.98 (dt, J=15.1, 4.4 Hz, 1H), 6.75-6.65 (m, 2H), 4.30 (s, 2H), 4.22-4.15 (m, 2H), 3.57 (t, J=5.8 Hz, 2H), 2.92 (t, J=6.1 Hz, 2H), 2.63 (t, J=5.8 Hz, 2H), 0.98-0.86 (m, 1H), 0.41-0.33 (m, 2H), 0.18-0.10 (m, 2H).

### Example 101: N3-[(2E)-3-(4-chlorobenzenesulfonyl)prop-2-en-1-yl]-N6-cyclopropyl-2-oxo-1,2,5,6,7,8-hexahydro-1,6-naphthyridine-3,6-dicarboxamide

Purity: 98.4% (254 nm) RT=2.11 min, m/z=491.10 [M+1], 489.23 [M−1]; ¹H NMR (400 MHz, DMSO-d6) δ 12.45 (s, 1H), 9.98 (s, 1H), 8.05 (s, 1H), 7.92-7.82 (m, 2H), 7.76-7.67 (m, 2H), 6.98 (dt, J=15.1, 4.4 Hz, 1H), 6.74-6.64 (m, 2H), 4.25 (s, 2H), 4.18 (dq, J=4.3, 2.1 Hz, 2H), 3.53 (t, J=5.8 Hz, 2H), 2.61 (t, J=5.8 Hz, 2H), 0.55 (td, J=6.9, 4.6 Hz, 2H), 0.42-0.32 (m, 2H).

### Example 102: Preparation of N-[(2E)-3-(benzenesulfonyl)prop-2-en-1-yl]-6-(3,3-difluorocyclobutanecarbonyl)-2-oxo-1,2,5,6,7,8-hexahydro-1,6-naphthyridine-3-carboxamide (Scheme 6, Reaction 1)

4,4-Difluorocyclohexanecarboxylic acid (13 mg, 0.08 mmol, 1.1 eq) was dissolved in DMF (0.8 mL). Then, N,N-diisopropylethylamine (0.051 mL, 0.29 mmol, 4 eq) and T3P, 50% w/w solution in DMF (0.092 mL, 0.15 mmol, 2 eq) were added and the solution was shaken for 10 min after which N-[(2E)-3-(benzenesulfonyl)prop-2-en-1-yl]-2-oxo-1,2,5,6,7,8-hexahydro-1,6-naphthyridine-3-carboxamide (30 mg, 0.07 mmol, 1 eq) was added. The reaction mixture was shaken for additional 2 h and control by UPLC showed consumption of starting material. Next, saturated NaHCO₃ solution and DCM were added, the two phases were separated, the organic phase was collected and concentrated to dryness. The residue was purified by preparative TLC eluting with DCM/MeOH 6%. The desired product was collected and concentrated to dryness to give N-[(2E)-3-(benzenesulfonyl)prop-2-en-1-yl]-6-(3,3-difluorocyclobutanecarbonyl)-2-oxo-1,2,5,6,7,8-hexahydro-1,6-naphthyridine-3-carboxamide (1.68 mg, Y: 4%) as white solid.

Rt=2.24 min, m/z=492.17 [M+1], 490.16 [M−1]; Purity: 95% (254 nm); ¹H NMR (400 MHz, DMSO-d6) δ 12.47 (s, 1H), 9.96 (s, 1H), 8.17 (d, J=13.6 Hz, 1H), 7.86 (dd, J=7.3, 1.7 Hz, 2H), 7.76-7.70 (m, 1H), 7.67-7.61 (m, 2H), 6.95 (dt, J=15.3, 4.4 Hz, 1H), 6.66 (dd, J=15.2, 2.6 Hz, 1H), 4.43 (d, J=6.2 Hz, 2H), 4.19 (s, 2H), 3.72 (t, J=5.9 Hz, 1H), 3.62 (t, J=5.8 Hz, 1H), 2.89-2.75 (m, 4H), 2.75-2.70 (m, 2H), 2.69-2.62 (m, 1H).

### Example 103: N-[(2E)-3-(benzenesulfonyl)prop-2-en-1-yl]-6-(2-methoxyethanesulfonyl)-2-oxo-1,2,5,6,7,8-hexahydro-1,6-naphthyridine-3-carboxamide (Scheme 6, Reaction 2)

N-[(2E)-3-(Benzenesulfonyl)prop-2-en-1-yl]-2-oxo-1,2,5,6,7,8-hexahydro-1,6-naphthyridine-3-carboxamide (30 mg, 0.072 mmol, 1 eq) was dissolved in DMF (0.72 mL) and N,N-diisopropylethylamine (0.038 mL, 0.22 mmol, 3 eq) and 2-methoxyethane-1-sulfonylchloride (0.010 mL, 0.087 mmol, 1.2 eq) were added. The reaction mixture was stirred for 2 h at room temperature. LCMS showed complete consumption of starting material. Next the mixture was poured into water and extracted with DCM. The organic phase was collected, concentrated for half of its volume and passed through silica NH₂ cartridge eluting with MeOH. The filtrate was collected and concentrated under reduced pressure, and purified by preparative TLC eluting with DCM/MeOH 6% to give N-[(2E)-3-(benzenesulfonyl)prop-2-en-1-yl]-6-(2-methoxyethanesulfonyl)-2-oxo-1,2,5,6,7,8-hexahydro-1,6-naphthyridine-3-carboxamide (4.5 mg, Y: 12%) as a white solid.

RT=2.07 min, m/z=496.10 [M+1], 493.93 [M−1]; Purity: 94% (254 nm); ¹H NMR (400 MHz, DMSO-d₆) δ 12.52 (s, 1H), 9.96 (s, 1H), 8.13 (s, 1H), 7.90-7.82 (m, 2H), 7.76-7.70 (m, 1H), 7.68-7.61 (m, 2H), 6.95 (dt, J=15.1, 4.4 Hz, 1H), 6.67 (dt, J=15.0, 1.9 Hz, 1H), 4.22 (s, 2H), 4.21-4.16 (m, 2H), 3.65 (t, J=5.9 Hz, 2H), 3.47 (t, J=5.9 Hz, 2H), 3.41 (t, J=5.9 Hz, 2H), 3.23 (s, 3H), 2.75 (t, J=5.9 Hz, 2H).

### Example 104: N-[(2E)-3-(benzenesulfonyl)prop-2-en-1-yl]-2-oxo-6-[(pyridin-3-yl)methyl]-1,2,5,6,7,8-hexahydro-1,6-naphthyridine-3-carboxamide (Scheme 6, Reaction 3)

N-[(2E)-3-(benzenesulfonyl)prop-2-en-1-yl]-2-oxo-1,2,5,6,7,8-hexahydro-1,6-naphthyridine-3-carboxamide (0.03 g, 0.07 mmol, 1.0 eq) was dissolved in DMF (0.24 ml, 0.3 M). Next, Na₂CO₃ (0.022 g, 0.21 mmol, 3.0 eq) and 3-(bromomethyl)pyridine (0.021 g, 0.08 mmol, 1.0 eq) were added and the reaction was stirred at room temperature for 4 h. Water was added to the reaction mixture and the solid precipitate was filtered, collected and subsequently dissolved in minimal DCM and purified by preparative TLC eluting with DCM/MeOH 5% to give N-[(2E)-3-(benzenesulfonyl)prop-2-en-1-yl]-2-oxo-6-[(pyridin-3-yl)methyl]-1,2,5,6,7,8-hexahydro-1,6-naphthyridine-3-carboxamide (14 mg, Y: 42%) as a white solid.

Rt=1.75 min, m/z=464.96 [M+1]; Purity: 97.54% (210 nm); ¹H NMR (400 MHz, DMSO-d₆) 12.42 (s, 1H), 10.02 (s, 1H), 8.53 (d, J=2.2 Hz, 1H), 8.49 (dd, J=4.8, 1.7 Hz, 1H), 8.01 (s, 1H), 7.86 (t, J=1.3 Hz, 1H), 7.84 (d, J=1.6 Hz, 1H), 7.77-7.69 (m, 2H), 7.67-7.61 (m, 2H), 7.40-7.35 (m, 1H), 6.94 (dt, J=15.1, 4.4 Hz, 1H), 6.65 (dt, J=15.0, 1.8 Hz, 1H), 4.21-4.13 (m, 2H), 3.68 (s, 2H), 3.39 (s, 2H), 2.71-2.63 (m, 4H).

The title compounds of Examples 105-125 and 185 were prepared by a method analogous to that described in Examples 102-104.

### Example 105: N-[(2E)-3-(benzenesulfonyl)prop-2-en-1-yl]-6-(3-methoxypropanoyl)-2-oxo-1,2,5,6,7,8-hexahydro-1,6-naphthyridine-3-carboxamide

Purity: 96.0 (254 nm); RT=2.26 min, m/z=460.48 [M+1], 458.16[M−1]; ¹H NMR (300 MHz, DMSO-d6) 12.48 (s, 1H), 9.95 (t, J=5.9 Hz, 1H), 8.15 (d, J=6.7 Hz, 1H), 7.90-7.81 (m, 2H), 7.78-7.69 (m, 1H), 7.64 (dd, J=8.2, 6.6 Hz, 2H), 6.95 (dt, J=15.1, 4.3 Hz, 1H), 6.66 (d, J=15.6 Hz, 1H), 4.45 (d, J=25.7 Hz, 2H), 4.18 (s, 2H), 3.69 (t, J=5.8 Hz, 2H), 3.56 (d, J=2.7 Hz, 2H), 3.21 (d, J=6.1 Hz, 3H), 2.77-2.58 (m, 4H).

### Example 106: 6-[2-(adamantan-1-yl)acetyl]-N-[(2E)-3-(benzenesulfonyl)prop-2-en-1-yl]-2-oxo-1,2,5,6,7,8-hexahydro-1,6-naphthyridine-3-carboxamide

Purity: 99.3% (254 nm); RT=1.50 min, m/z=550.3 [M+1]; ¹H NMR (300 MHz, DMSO-d6) δ 12.46 (s, 1H), 9.96 (t, J=5.8 Hz, 1H), 8.17 (d, J=22.4 Hz, 1H), 7.92-7.79 (m, 2H), 7.80-7.68 (m, 1H), 7.69-7.57 (m, 2H), 6.95 (dt, J=15.2, 4.3 Hz, 1H), 6.73-6.58 (m, 1H), 4.48 (d, J=37.8 Hz, 2H), 4.26-4.07 (m, 2H), 3.72 (dt, J=11.2, 5.7 Hz, 2H), 2.77-2.57 (m, 2H), 2.17 (d, J=2.8 Hz, 2H), 1.99-1.78 (m, 4H), 1.71-1.47 (m, 11H).

### Example 107: N-[(2E)-3-(benzenesulfonyl)prop-2-en-1-yl]-2-oxo-6-[(1r,4r)-4-methoxycyclohexanecarbonyl]-1,2,5,6,7,8-hexahydro-1,6-naphthyridine-3-carboxamide

Purity: 98.7% (254 nm); RT=1.97 min, m/z=514.4 [M+1]; ¹H NMR (300 MHz, DMSO-d6) δ 12.47 (s, 1H), 9.96 (t, J=5.8 Hz, 1H), 8.17 (d, J=25.3 Hz, 1H), 7.97-7.80 (m, 2H), 7.80-7.52 (m, 3H), 6.96 (dt, J=15.2, 4.4 Hz, 1H), 6.66 (d, J=15.1 Hz, 1H), 4.55 (s, 1H), 4.39 (s, 1H), 4.18 (d, J=6.9 Hz, 2H), 3.70 (dt, J=12.1, 6.0 Hz, 2H), 3.23 (s, 3H), 3.14-2.99 (m, 1H), 2.74 (s, 1H), 2.66-2.57 (m, 2H), 2.09-1.91 (m, 2H), 1.79-1.58 (m, 2H), 1.53-1.27 (m, 2H), 1.28-1.05 (m, 2H).

### Example 108: N-[(2E)-3-(benzenesulfonyl)prop-2-en-1-yl]-6-(4,4-difluorocyclohexanecarbonyl)-2-oxo-1,2,5,6,7,8-hexahydro-1,6-naphthyridine-3-carboxamide

Purity: 94% (254 nm); RT=1.90 min, m/z=520.24 [M+1], 518.36 [M−1]; ¹H NMR (400 MHz, DMSO-d6) δ 10.14-9.77 (m, 1H), 8.24-8.07 (m, 1H), 7.93-7.83 (m, 2H), 7.77-7.57 (m, 3H), 7.14-6.80 (m, 1H), 6.66 (d, J=15.5 Hz, 1H), 4.58 (s, 1H), 4.41 (s, 1H), 4.19 (s, 2H), 3.83-3.62 (m, 2H), 2.95-2.74 (m, 2H), 2.12-1.69 (m, 6H), 1.64-1.51 (m, 2H), 1.24 (s, 1H).

### Example 109: N-[(2E)-3-(benzenesulfonyl)prop-2-en-1-yl]-6-cyclopropanecarbonyl-2-oxo-1,2,5,6,7,8-hexahydro-1,6-naphthyridine-3-carboxamide

Purity: 96.0% (254 nm); RT=1.33 min, m/z=442.23 [M+1], 440.16 [M−1]; ¹H NMR (400 MHz, DMSO-d6) δ 12.49 (s, 1H), 9.96 (t, J=6.0 Hz, 1H), 8.16 (d, J=18.4 Hz, 1H), 7.90-7.83 (m, 2H), 7.78-7.70 (m, 1H), 7.65 (dd, J=8.4, 7.0 Hz, 2H), 6.96 (dt, J=15.2, 4.4 Hz, 1H), 6.67 (dt, J=15.1, 1.8 Hz, 1H), 4.73 (s, 1H), 4.42 (s, 1H), 4.20 (d, J=5.6 Hz, 2H), 3.92 (s, 1H), 3.71 (s, 1H), 2.78 (s, 1H), 2.64 (s, 1H), 2.07 (d, J=8.4 Hz, 1H), 1.37-1.27 (m, 2H), 0.86 (t, J=6.0 Hz, 1H).

### Example 110: N-[(2E)-3-(benzenesulfonyl)prop-2-en-1-yl]-6-{bicyclo[2.2.1]heptane-1-carbonyl}-2-oxo-1,2,5,6,7,8-hexahydro-1,6-naphthyridine-3-carboxamide

Purity: 97.7% (254 nm); RT=1.99 min, m/z=496.20 [M+1], 494.20 [M−1]; ¹H NMR (300 MHz, DMSO-d6) δ 12.48 (s, 1H), 9.96 (t, J=5.9 Hz, 1H), 8.37-8.09 (m, 1H), 7.86 (d, J=7.6 Hz, 2H), 7.80-7.53 (m, 3H), 6.95 (dt, J=15.1, 4.3 Hz, 1H), 6.67 (d, J=15.3 Hz, 1H), 4.48 (s, 2H), 4.18 (d, J=5.5 Hz, 2H), 3.74 (t, J=5.5 Hz, 2H), 2.64 (t, J=5.6 Hz, 2H), 2.11 (s, 1H), 1.93-1.74 (m, 2H), 1.73-1.51 (m, 5H), 1.44-1.27 (m, 2H), 1.14 (t, J=7.2 Hz, 1H).

### Example 111: N-[(2E)-3-(benzenesulfonyl)prop-2-en-1-yl]-6-(1-cyanocyclobutanecarbonyl)-2-oxo-1,2,5,6,7,8-hexahydro-1,6-naphthyridine-3-carboxamide

Purity: 97.0 (254 nm); RT=2.19 min, m/z=481.18 [M+1], 479.19 [M−1]; ¹H NMR (400 MHz, DMSO-d6) δ 12.53 (s, 1H), 9.96 (s, 1H), 8.24 (d, J=43.4 Hz, 1H), 7.91-7.82 (m, 2H), 7.77-7.69 (m, 1H), 7.68-7.59 (m, 2H), 6.95 (dt, J=15.1, 4.4 Hz, 1H), 6.67 (d, J=15.1 Hz, 1H), 4.48 (s, 1H), 4.41 (s, 1H), 4.19 (t, J=5.2 Hz, 2H), 3.77 (d, J=6.0 Hz, 1H), 3.62 (d, J=6.1 Hz, 1H), 2.83-2.62 (m, 6H), 2.20-2.10 (m, 1H), 1.97-1.81 (m, 1H).

### Example 112: N-[(2E)-3-(benzenesulfonyl)prop-2-en-1-yl]-2-oxo-6-(2-phenyl-2H-1,2,3-triazole-4-carbonyl)-1,2,5,6,7,8-hexahydro-1,6-naphthyridine-3-carboxamide

Purity: 94% (254 nm); RT=2.24 min, m/z=545.19 [M+1], 543.18 [M−1]; ¹H NMR (400 MHz, DMSO-d6) δ 12.54 (s, 1H), 10.00 (s, 1H), 8.44 (d, J=18.4 Hz, 1H), 8.20 (d, J=22.4 Hz, 1H), 8.09 (t, J=7.4 Hz, 2H), 7.85 (t, J=8.5 Hz, 2H), 7.72 (d, J=7.6 Hz, 1H), 7.69-7.58 (m, 4H), 7.51 (t, J=7.5 Hz, 1H), 7.00-6.86 (m, 1H), 6.77-6.57 (m, 1H), 4.92 (s, 1H), 4.65 (s, 1H), 4.22-4.15 (m, 2H), 4.14-4.05 (m, 1H), 3.98-3.83 (m, 1H), 2.90-2.81 (m, 1H), 2.82-2.71 (m, 1H).

### Example 113: N-[(2E)-3-(benzenesulfonyl)prop-2-en-1-yl]-2-oxo-6-(oxolane-3-carbonyl)-1,2,5,6,7,8-hexahydro-1,6-naphthyridine-3-carboxamide

Purity: 99% (254 nm); RT=1.68 min, m/z=472.20 [M+1], 470.31 [M−1]; ¹H NMR (400 MHz, DMSO-d6) δ 12.50 (s, 1H), 9.97 (s, 1H), 8.17 (d, J=19.5 Hz, 1H), 7.91-7.83 (m, 2H), 7.78-7.69 (m, 1H), 7.68-7.61 (m, 2H), 6.96 (dt, J=15.1, 4.4 Hz, 1H), 6.74-6.58 (m, 1H), 4.55 (d, J=3.8 Hz, 1H), 4.43 (s, 1H), 4.22-4.14 (m, 2H), 3.89 (q, J=7.9 Hz, 1H), 3.71 (ddt, J=13.6, 9.9, 6.7 Hz, 5H), 3.42 (dt, J=14.1, 7.3 Hz, 1H), 2.77-2.71 (m, 1H), 2.66-2.60 (m, 1H), 2.12-1.90 (m, 2H).

### Example 114: N-[(2E)-3-(benzenesulfonyl)prop-2-en-1-yl]-6-{3-methylbicyclo[1.1.1]pentane-1-carbonyl}-2-oxo-1,2,5,6,7,8-hexahydro-1,6-naphthyridine-3-carboxamide

Purity: 99.4% (254 nm); RT=2.31 min, m/z=482.21 [M+1]; ¹H NMR (300 MHz, DMSO-d6) δ 12.49 (s, 1H), 9.96 (q, J=6.4 Hz, 1H), 8.37-8.04 (m, 1H), 7.92-7.80 (m, 2H), 7.78-7.55 (m, 3H), 7.04-6.85 (m, 1H), 6.67 (dd, J=15.1, 1.9 Hz, 1H), 4.55 (s, 1H), 4.37 (s, 1H), 4.18 (d, J=5.0 Hz, 2H), 3.79 (t, J=5.7 Hz, 1H), 3.65 (t, J=5.9 Hz, 1H), 2.76-2.56 (m, 2H), 1.97 (s, 6H), 1.16 (d, J=2.4 Hz, 3H).

### Example 115: N-[(2E)-3-(benzenesulfonyl)prop-2-en-1-yl]-6-(1-methylcyclobutanecarbonyl)-2-oxo-1,2,5,6,7,8-hexahydro-1,6-naphthyridine-3-carboxamide

Purity: 98.9% (254 nm); RT=2.16 min, m/z=470.22 [M+1]; ¹H NMR (400 MHz, DMSO-d6) δ 12.50 (s, 1H), 9.97 (t, J=5.9 Hz, 1H), 8.18 (d, J=27.0 Hz, 1H), 7.89-7.83 (m, 2H), 7.78-7.69 (m, 1H), 7.69-7.59 (m, 2H), 6.95 (dt, J=15.1, 4.4 Hz, 1H), 6.67 (dt, J=15.0, 1.8 Hz, 1H), 4.46-4.23 (m, 2H), 4.19 (ddd, J=6.3, 4.4, 2.0 Hz, 2H), 3.76-3.42 (m, 2H), 2.80-2.56 (m, 2H), 2.46-2.26 (m, 2H), 2.03-1.78 (m, 3H), 1.69-1.53 (m, 1H), 1.45-1.23 (m, 3H).

### Example 116: N-[(2E)-3-(benzenesulfonyl)prop-2-en-1-yl]-6-(4-cyclopropyl-1,3-thiazole-2-carbonyl)-2-oxo-1,2,5,6,7,8-hexahydro-1,6-naphthyridine-3-carboxamide

Purity: 92.2% (254 nm); RT=2.61 min, m/z=525.15 [M+1], 523.22 [M−1]; ¹H NMR (400 MHz, DMSO-d6) δ 12.54 (s, 1H), 9.98 (s, 1H), 8.14 (d, J=45.1 Hz, 1H), 8.01-7.81 (m, 2H), 7.77-7.69 (m, 1H), 7.67-7.58 (m, 3H), 6.96 (dt, J=15.1, 4.4 Hz, 1H), 6.67 (d, J=15.2 Hz, 1H), 5.28-5.11 (m, 1H), 4.65-4.55 (m, 1H), 4.44-4.29 (m, 1H), 4.26-4.13 (m, 2H), 3.95-3.81 (m, 1H), 2.92-2.74 (m, 2H), 2.22-2.11 (m, 1H), 1.02-0.93 (m, 2H), 0.91-0.80 (m, 2H).

### Example 117: N-[(2E)-3-(benzenesulfonyl)prop-2-en-1-yl]-6-(2-cyclopropylacetyl)-2-oxo-1,2,5,6,7,8-hexahydro-1,6-naphthyridine-3-carboxamide

Purity: 97.9% (254 nm); RT=1.99 min, m/z=456.22 [M+1]; ¹H NMR (400 MHz, DMSO-d6) δ 12.49 (s, 1H), 9.96 (t, J=5.9 Hz, 1H), 8.15 (d, J=5.1 Hz, 1H), 7.92-7.81 (m, 2H), 7.79-7.69 (m, 1H), 7.69-7.54 (m, 2H), 6.95 (dt, J=15.2, 4.4 Hz, 1H), 6.72-6.60 (m, 1H), 4.44 (d, J=19.8 Hz, 2H), 4.18 (t, J=4.7 Hz, 2H), 3.68 (dt, J=15.1, 5.8 Hz, 2H), 2.81-2.55 (m, 2H), 2.33 (dd, J=9.7, 6.7 Hz, 2H), 1.05-0.87 (m, 1H), 0.51-0.35 (m, 2H), 0.19-0.03 (m, 2H).

### Example 118: N-[(2E)-3-(benzenesulfonyl)prop-2-en-1-yl]-2-oxo-6-(1-phenylpyrrolidine-3-carbonyl)-1,2,5,6,7,8-hexahydro-1,6-naphthyridine-3-carboxamide

Purity: 95.9% (254 nm); RT=2.73 min, m/z=547.20 [M+1]; ¹H NMR (300 MHz, DMSO-d6) δ 12.51 (s, 1H), 9.98 (t, 1H), 8.18 (d, J=17.5 Hz, 1H), 7.93-7.81 (m, 2H), 7.78-7.69 (m, 1H), 7.69-7.58 (m, 2H), 7.15 (t, J=7.7 Hz, 2H), 6.96 (dt, J=15.1, 4.4 Hz, 1H), 6.72-6.49 (m, 4H), 4.64 (s, 1H), 4.45 (s, 1H), 4.28-4.09 (m, 2H), 3.87-3.78 (m, 1H), 3.78-3.68 (m, 1H), 3.67-3.54 (m, 1H), 3.49 (t, J=8.5 Hz, 1H), 3.30-3.18 (m, 3H), 2.85-2.76 (m, 1H), 2.70-2.61 (m, 1H), 2.25-1.99 (m, 2H).

### Example 119: 2-methylpropyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 96.9% (254 nm); RT=2.58 min, m/z=474.2 [M+1], 472.19 [M−1]; ¹H NMR (400 MHz, DMSO-d6) δ 12.48 (s, 1H), 9.96 (t, J=6.0 Hz, 1H), 8.16 (s, 1H), 7.92-7.80 (m, 2H), 7.78-7.69 (m, 1H), 7.69-7.59 (m, 2H), 6.95 (dt, J=15.1, 4.4 Hz, 1H), 6.67 (dt, J=15.1, 1.8 Hz, 1H), 4.50-4.27 (m, 2H), 4.24-4.13 (m, 2H), 3.82 (d, J=6.6 Hz, 2H), 3.71-3.53 (m, 2H), 2.69-2.65 (m, 2H), 1.88 (dq, J=13.3, 6.6 Hz, 1H), 0.91 (d, J=6.7 Hz, 6H).

### Example 120: N-[(2E)-3-(benzenesulfonyl)prop-2-en-1-yl]-6-(3,3-dimethylbutanoyl)-2-oxo-1,2,5,6,7,8-hexahydro-1,6-naphthyridine-3-carboxamide

Purity: 91.9% (254 nm); RT=2.34 min, m/z=472.24 [M+1], 470.47 [M−1]; ¹H NMR (300 MHz, DMSO-d6) 12.49 (s, 1H), 9.95 (t, J=5.9 Hz, 1H), 8.16 (d, J=19.0 Hz, 1H), 7.92-7.82 (m, 2H), 7.77-7.69 (m, 1H), 7.64 (dd, J=8.2, 6.6 Hz, 2H), 6.95 (dt, J=15.2, 4.4 Hz, 1H), 6.66 (d, J=15.1 Hz, 1H), 4.54 (s, 1H), 4.42 (s, 1H), 4.18 (s, 2H), 3.72 (d, J=6.1 Hz, 2H), 2.66 (d, J=30.6 Hz, 2H), 2.30 (d, J=4.0 Hz, 2H), 0.98 (d, J=11.6 Hz, 9H).

### Example 121: N-[(2E)-3-(benzenesulfonyl)prop-2-en-1-yl]-6-[(1-methyl-1H-indazol-4-yl)methyl]-2-oxo-1,2,5,6,7,8-hexahydro-1,6-naphthyridine-3-carboxamide

Purity: 82.1% (254 nm); RT=3.42 min, m/z=518.18 [M+1], 516.17 [M−1]; ¹H NMR (400 MHz, DMSO-d6) 12.45 (s, 1H), 10.02 (s, 1H), 8.18 (d, J=1.0 Hz, 1H), 7.98 (s, 1H), 7.93-7.82 (m, 2H), 7.77-7.69 (m, 1H), 7.69-7.52 (m, 3H), 7.35 (ddd, J=8.5, 6.9, 2.2 Hz, 1H), 7.10 (t, J=5.4 Hz, 1H), 6.94 (dt, J=15.1, 4.4 Hz, 1H), 6.65 (dt, J=15.1, 1.9 Hz, 1H), 4.21-4.13 (m, 2H), 4.04 (d, J=1.2 Hz, 3H), 3.96 (s, 2H), 3.44 (s, 2H), 2.70 (dd, J=8.5, 4.3 Hz, 4H).

### Example 122: 2-(4-chlorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate

Purity: 90.0% (254 nm); RT=2.07 min, m/z=556.12 [M+1]; ¹H NMR (400 MHz, DMSO-d6) δ 12.46 (s, 1H), 9.97 (s, 1H), 8.12 (s, 1H), 7.90-7.83 (m, 2H), 7.77-7.69 (m, 1H), 7.64 (tt, J=6.6, 1.5 Hz, 2H), 7.29 (s, 4H), 6.96 (dt, J=15.2, 4.4 Hz, 1H), 6.66 (dt, J=15.1, 1.9 Hz, 1H), 4.31 (s, 2H), 4.25-4.16 (m, 4H), 3.56 (t, J=5.7 Hz, 2H), 2.90 (t, J=6.5 Hz, 2H), 2.65-2.59 (m, 2H).

### Example 123: N-[(2E)-3-(benzenesulfonyl)prop-2-en-1-yl]-2-oxo-6-[(pyridazin-3-yl)methyl]-1,2,5,6,7,8-hexahydro-1,6-naphthyridine-3-carboxamide

Purity: 96.1% (254 nm); RT=1.80 min, m/z 465.81 [M+1], 464.04 [M−1]; ¹H NMR (400 MHz, DMSO-d6) 12.44 (s, 1H), 10.02 (t, J=5.7 Hz, 1H), 9.16 (dd, J=4.8, 1.8 Hz, 1H), 8.01 (s, 1H), 7.89-7.83 (m, 2H), 7.77-7.60 (m, 5H), 6.95 (dt, J=15.1, 4.4 Hz, 1H), 6.66 (dt, J=15.1, 1.8 Hz, 1H), 4.17 (ddd, J=6.3, 4.5, 1.9 Hz, 2H), 3.98 (s, 2H), 3.47 (s, 2H), 2.72 (dd, J=12.9, 4.7 Hz, 4H).

### Example 124: N-[(2E)-3-(benzenesulfonyl)prop-2-en-1-yl]-6-(3-methoxy-3-methylbutanoyl)-2-oxo-1,2,5,6,7,8-hexahydro-1,6-naphthyridine-3-carboxamide

Purity: 79.2% (254 nm); RT=1.93 min, m/z=488.11 [M+1]; ¹H NMR (400 MHz, DMSO-d6) 12.51 (s, 1H), 9.98 (s, 1H), 8.15 (d, J=15.8 Hz, 1H), 7.89-7.84 (m, 2H), 7.77-7.71 (m, 1H), 7.68-7.57 (m, 2H), 6.95 (dt, J=15.1, 4.4 Hz, 1H), 6.66 (dd, J=15.2, 1.9 Hz, 1H), 4.55 (s, 1H), 4.42 (s, 1H), 4.19 (s, 2H), 3.72 (dt, J=14.2, 5.9 Hz, 2H), 3.08 (d, J=27.1 Hz, 3H), 2.72 (t, J=6.9 Hz, 1H), 2.62 (d, J=5.7 Hz, 1H), 2.57 (d, J=3.9 Hz, 2H), 1.20 (d, J=13.5 Hz, 6H).

### Example 125: N-[(2E)-3-(benzenesulfonyl)prop-2-en-1-yl]-2-oxo-6-(2-phenylcyclopropanecarbonyl)-1,2,5,6,7,8-hexahydro-1,6-naphthyridine-3-carboxamide

Purity: 91% (254 nm); RT=2.21 min, m/z=518.09 [M+1], 516.04 [M−1]; ¹H NMR (400 MHz, DMSO-d6) δ 12.49 (s, 1H), 10.06 (s, 1H), 8.12 (s, 1H), 7.88-7.83 (m, 2H), 7.76-7.70 (m, 1H), 7.67-7.61 (m, 2H), 7.31-7.25 (m, 2H), 7.23-7.16 (m, 3H), 6.95 (dt, J=15.1, 4.4 Hz, 1H), 6.70-6.61 (m, 1H), 4.84-4.60 (m, 1H), 4.45 (s, 1H), 4.25-4.06 (m, 2H), 3.89-3.61 (m, 2H), 2.78-2.61 (m, 2H), 2.44 (dt, J=8.8, 4.9 Hz, 1H), 2.36-2.30 (m, 1H), 1.48-1.36 (m, 1H), 1.29-1.12 (m, 1H).

### Example 126: N-[(2E)-3-(2,4-dichlorobenzenesulfonyl)prop-2-en-1-yl]-2-oxo-5-phenyl-1,2-dihydropyridine-3-carboxamide

Purity: 94% (254 nm); Rt=2.27 min, m/z=462.83[M+1], 461.02 [M−1]; 1H NMR (400 MHz, DMSO-d6) δ 12.86 (s, 1H), 10.04 (t, J=5.9 Hz, 1H), 8.60 (d, J=2.9 Hz, 1H), 8.09-8.03 (m, 2H), 7.95 (d, J=2.0 Hz, 1H), 7.72 (dd, J=8.5, 2.1 Hz, 1H), 7.65-7.60 (m, 2H), 7.50-7.40 (m, 2H), 7.39-7.31 (m, 1H), 7.12 (dt, J=15.1, 4.4 Hz, 1H), 6.81 (dt, J=15.1, 1.8 Hz, 1H), 4.33-4.24 (m, 2H).

### Example 127: N-[(2E)-3-(2,4-difluorobenzenesulfonyl)prop-2-en-1-yl]-2-oxo-5-phenyl-1,2-dihydropyridine-3-carboxamide

Purity: 93% (254 nm); Rt=2.36 min, m/z=429.20[M−1]; ¹H NMR (400 MHz, DMSO-d6) δ 12.86 (s, 1H), 10.02 (t, J=5.9 Hz, 1H), 8.60 (d, J=2.9 Hz, 1H), 8.07 (d, J=2.9 Hz, 1H), 7.95 (td, J=8.6, 6.2 Hz, 1H), 7.70-7.58 (m, 3H), 7.53-7.40 (m, 2H), 7.41-7.30 (m, 2H), 7.15-7.01 (m, 1H), 6.85-6.72 (m, 1H), 4.27 (ddd, J=6.2, 4.4, 1.9 Hz, 2H).

### Example 128: N-[(2E)-3-(2-chloro-4-fluorobenzenesulfonyl)prop-2-en-1-yl]-2-oxo-5-phenyl-1,2-dihydropyridine-3-carboxamide

Purity: 95% (254 nm); Rt=1.94 min, m/z=447.1 [M+1], 445.08 [M−1]; ¹H NMR (400 MHz, DMSO-d6) δ 12.86 (s, 1H), 10.03 (t, J=5.9 Hz, 1H), 8.60 (d, J=2.9 Hz, 1H), 8.13 (dd, J=8.9, 5.9 Hz, 1H), 8.07 (d, J=2.9 Hz, 1H), 7.78 (dd, J=8.7, 2.5 Hz, 1H), 7.65-7.60 (m, 2H), 7.51 (td, J=8.5, 2.6 Hz, 1H), 7.48-7.41 (m, 2H), 7.39- 7.30 (m, 1H), 7.11 (dt, J=15.1, 4.4 Hz, 1H), 6.80 (dt, J=15.1, 1.9 Hz, 1H), 4.28 (ddd, J=6.2, 4.4, 1.9 Hz, 2H).

### Example 129: N-[(2E)-3-(2-chlorobenzenesulfonyl)prop-2-en-1-yl]-2-oxo-5-phenyl-1,2-dihydropyridine-3-carboxamide

Purity: 93% (254 nm); Rt=2.36 min, m/z=429.1 [M+1]; ¹H NMR (400 MHz, DMSO-d6) δ 12.87 (s, 1H), 10.03 (s, 1H), 8.61 (d, J=2.8 Hz, 1H), 8.10-8.05 (m, 2H), 7.78-7.69 (m, 2H), 7.66-7.59 (m, 3H), 7.50-7.41 (m, 2H), 7.40-7.31 (m, 1H), 7.11 (dt, J=15.1, 4.4 Hz, 1H), 6.82 (dt, J=15.0, 1.8 Hz, 1H), 4.34-4.21 (m, 2H).

### Example 130: N-[(2E)-3-(4-chlorobenzenesulfonyl)prop-2-en-1-yl]-2-oxo-5-phenyl-1,2-dihydropyridine-3-carboxamide

Purity: 97.2% (254 nm); Rt=2.55 min, m/z=427.01 [M+1]; ¹H NMR (400 MHz, DMSO-d6) δ 12.85 (s, 1H), 10.03 (t, J=6.0 Hz, 1H), 8.59 (d, J=2.9 Hz, 1H), 8.07 (d, J=2.9 Hz, 1H), 7.93-7.85 (m, 2H), 7.76-7.69 (m, 2H), 7.65-7.59 (m, 2H), 7.45 (t, J=7.7 Hz, 2H), 7.39-7.31 (m, 1H), 7.01 (dt, J=15.1, 4.3 Hz, 1H), 6.78 (dt, J=15.1, 1.9 Hz, 1H), 4.23 (ddd, J=6.2, 4.4, 1.9 Hz, 2H).

### Example 131: N-[(2E)-3-[(4-chlorophenyl)(imino)oxo-λ⁶-sulfanyl]prop-2-en-1-yl]-2-oxo-1,2,5,6,7,8-hexahydroquinoline-3-carboxamide

Purity: 93.2% (254 nm); RT=4.96 min, m/z=406.17 [M+1]; ¹H NMR (400 MHz, DMSO-d6) δ 12.26 (s, 1H), 10.00 (t, J=6.0 Hz, 1H), 8.04 (s, 1H), 7.91-7.82 (m, 2H), 7.71-7.58 (m, 2H), 6.81 (dt, J=15.0, 4.6 Hz, 1H), 6.50 (dt, J=14.8, 1.8 Hz, 1H), 4.73 (s, 1H), 4.18-4.06 (m, 2H), 2.58 (t, J=5.9 Hz, 2H), 1.74-1.64 (m, 4H).

### Example 132: N-[(2E)-3-[(2-fluorophenyl)(imino)oxo-λ⁶-sulfanyl]prop-2-en-1-yl]-2-oxo-1,2,5,6,7,8-hexahydroquinoline-3-carboxamide

Purity: 92.2% (254 nm); RT=3.54 min, m/z=390.21 [M+1]; ¹H NMR (400 MHz, DMSO-d6) δ 12.25 (s, 1H), 10.04 (t, J=6.0 Hz, 1H), 8.05 (s, 1H), 7.88 (td, J=7.8, 1.9 Hz, 1H), 7.74-7.64 (m, 1H), 7.44-7.34 (m, 2H), 6.96-6.85 (m, 1H), 6.58 (d, J=14.9 Hz, 1H), 5.03 (s, 1H), 4.23-4.15 (m, 2H), 2.59 (t, J=6.0 Hz, 2H), 1.74-1.64 (m, 4H).

### Example 133: N-[(2E)-3-[(3-chloro-4-methoxyphenyl)(imino)oxo-λ⁶-sulfanyl]prop-2-en-1-yl]-2-oxo-1,2,5,6,7,8-hexahydroquinoline-3-carboxamide

Purity: 93.4% (254 nm); RT=2.06 min, m/z=436.0 [M+1]; ¹H NMR (400 MHz, DMSO-d6) δ 12.27 (s, 1H), 9.99 (t, J=5.9 Hz, 1H), 8.04 (s, 1H), 7.83 (d, J=2.2 Hz, 1H), 7.79 (dd, J=8.7, 2.3 Hz, 1H), 7.33 (d, J=8.8 Hz, 1H), 6.77 (dt, J=14.9, 4.6 Hz, 1H), 6.50 (dt, J=14.9, 1.8 Hz, 1H), 4.63 (s, 1H), 4.18-4.08 (m, 2H), 3.94 (s, 3H), 2.58 (t, J=5.9 Hz, 2H), 1.77-1.61 (m, 4H).

### Example 134: N-[(2E)-3-[(3-chlorophenyl)(imino)oxo-λ⁶-sulfanyl]prop-2-en-1-yl]-2-oxo-1,2,5,6,7,8-hexahydroquinoline-3-carboxamide

Purity: 91.7% (254 nm); RT=2.09 min, m/z=406.06 [M+1]; ¹H NMR (400 MHz, DMSO-d6) δ 12.27 (s, 1H), 10.00 (t, J=6.0 Hz, 1H), 8.04 (s, 1H), 7.86 (t, J=1.9 Hz, 1H), 7.81 (dt, J=7.8, 1.4 Hz, 1H), 7.72 (ddd, J=8.0, 2.1, 1.1 Hz, 1H), 7.62 (t, J=7.9 Hz, 1H), 6.85 (dt, J=15.1, 4.6 Hz, 1H), 6.54 (d, J=15.0 Hz, 1H), 4.81 (s, 1H), 4.15 (t, J=5.6 Hz, 2H), 2.59 (d, J=5.6 Hz, 2H), 1.77-1.62 (m, 4H).

### Example 135: N-[(2E)-3-[(2,4-difluorophenyl)(imino)oxo-λ⁶-sulfanyl]prop-2-en-1-yl]-2-oxo-1,2,5,6,7,8-hexahydroquinoline-3-carboxamide

Purity: 93.1% (254 nm); RT=2.43 min, m/z=408.09 [M+1]; ¹H NMR (400 MHz, DMSO-d6) δ 12.29 (s, 1H), 10.04 (t, J=6.0 Hz, 1H), 8.05 (s, 1H), 7.93 (td, J=8.7, 6.4 Hz, 1H), 7.49 (ddd, J=10.4, 9.2, 2.5 Hz, 1H), 7.30 (td, J=8.7, 2.3 Hz, 1H), 6.95-6.86 (m, 1H), 6.56 (d, J=15.0 Hz, 1H), 5.11 (s, 1H), 4.24-4.13 (m, 2H), 2.59 (t, J=6.0 Hz, 2H), 1.69 (q, J=8.2 Hz, 4H).

### Example 136: N-[(2E)-3-[(4-cyanophenyl)(imino)oxo-λ⁶-sulfanyl]prop-2-en-1-yl]-2-oxo-1,2,5,6,7,8-hexahydroquinoline-3-carboxamide

Purity: 97.0% (254 nm); RT=2.27 min, m/z=397.08 [M+1]; ¹H NMR (400 MHz, DMSO-d6) δ 12.27 (s, 1H), 10.01 (t, J=5.9 Hz, 1H), 8.09-7.99 (m, 4H), 6.88 (dt, J=14.9, 4.6 Hz, 1H), 6.59-6.50 (m, 1H), 4.93 (s, 1H), 4.16 (t, J=5.2 Hz, 2H), 2.58 (t, J=6.0 Hz, 2H), 1.76-1.62 (m, 4H).

### Example 137: N-[(2E)-3-[imino(4-methoxy-3-methylphenyl)oxo-λ⁶-sulfanyl]prop-2-en-1-yl]-2-oxo-1,2,5,6,7,8-hexahydroquinoline-3-carboxamide

Purity: 95.7% (254 nm); RT=1.89 min, m/z=416.22 [M+1]; ¹H NMR (400 MHz, DMSO-d6) δ 12.26 (s, 1H), 9.98 (t, J=5.9 Hz, 1H), 8.04 (s, 1H), 7.68 (dd, J=8.6, 2.5 Hz, 1H), 7.61 (dd, J=2.4, 1.0 Hz, 1H), 7.10 (d, J=8.7 Hz, 1H), 6.70 (dt, J=15.0, 4.7 Hz, 1H), 6.43 (dt, J=14.8, 1.8 Hz, 1H), 4.35 (s, 1H), 4.11 (t, J=5.1 Hz, 2H), 3.86 (s, 3H), 2.58 (t, J=6.0 Hz, 2H), 2.19 (s, 3H), 1.69 (q, J=8.2 Hz, 4H).

### Example 138: N-[(2E)-3-[imino(oxo)[4-(propan-2-yloxy)phenyl]-λ⁶-sulfanyl]prop-2-en-1-yl]-2-oxo-1,2,5,6,7,8-hexahydroquinoline-3-carboxamide

Purity: 91.9% (254 nm); RT=2.12 min, m/z=430.23 [M+1]; ¹H NMR (400 MHz, DMSO-d6) δ 12.26 (s, 1H), 10.01 (s, 1H), 8.04 (s, 1H), 7.79-7.69 (m, 2H), 7.10-7.02 (m, 2H), 6.71 (dt, J=15.0, 4.6 Hz, 1H), 6.49-6.40 (m, 1H), 4.72 (p, J=6.0 Hz, 1H), 4.39 (s, 1H), 4.12 (t, J=5.2 Hz, 2H), 2.57 (d, J=6.3 Hz, 2H), 1.67 (dd, J=15.1, 6.5 Hz, 4H), 1.29 (d, J=6.0 Hz, 6H).

### Example 139: N-[(2E)-3-[imino(4-methoxy-2-methylphenyl)oxo-λ⁶-sulfanyl]prop-2-en-1-yl]-2-oxo-1,2,5,6,7,8-hexahydroquinoline-3-carboxamide

Purity: 92.5% (254 nm); RT=1.84 min, m/z=416.2 [M+1]; ¹H NMR (400 MHz, DMSO-d6) δ 12.25 (s, 1H), 10.02 (t, J=5.9 Hz, 1H), 8.04 (s, 1H), 7.91 (d, J=8.5 Hz, 1H), 6.93 (d, J=8.8 Hz, 2H), 6.74 (dt, J=15.0, 4.8 Hz, 1H), 6.44-6.37 (m, 1H), 4.46 (s, 1H), 4.15 (t, J=5.2 Hz, 2H), 3.81 (s, 3H), 2.58 (s, 2H), 1.69 (q, J=7.6 Hz, 4H).

### Example 140: N-[(2E)-3-[imino(oxo)(4-phenoxyphenyl)-λ⁶-sulfanyl]prop-2-en-1-yl]-2-oxo-1,2,5,6,7,8-hexahydroquinoline-3-carboxamide

Purity: 92.0% (254 nm); RT=2.53 min, m/z=464.08 [M+1]; ¹H NMR (400 MHz, DMSO-d6) δ 12.27 (s, 1H), 10.01 (t, J=5.9 Hz, 1H), 8.05 (s, 1H), 7.89-7.79 (m, 2H), 7.54-7.43 (m, 2H), 7.26 (t, J=7.4 Hz, 1H), 7.19-7.06 (m, 4H), 6.76 (dt, J=14.9, 4.6 Hz, 1H), 6.54-6.42 (m, 1H), 4.53 (s, 1H), 4.14 (t, J=5.1 Hz, 2H), 2.64-2.56 (m, 2H), 1.78-1.56 (m, 4H).

### Example 141: N-[(2E)-3-{[4-(dimethylamino)phenyl](imino)oxo-λ⁶-sulfanyl}prop-2-en-1-yl]-2-oxo-1,2,5,6,7,8-hexahydroquinoline-3-carboxamide

Purity: 92.9% (254 nm); RT=1.56 min, m/z=415.24 [M+1]; ¹H NMR (400 MHz, DMSO-d6) δ 12.27 (s, 1H), 9.98 (t, J=6.0 Hz, 1H), 8.04 (s, 1H), 7.60 (d, J=8.8 Hz, 2H), 6.77 (d, J=8.9 Hz, 2H), 6.63 (dt, J=14.9, 4.8 Hz, 1H), 6.39 (d, J=14.9 Hz, 1H), 4.29 (s, 1H), 4.10 (t, J=5.4 Hz, 2H), 2.99 (s, 6H), 2.63-2.53 (m, 4H), 1.77-1.61 (m, 4H).

### Example 142: N-[(2E)-3-[(3-fluoro-4-methoxyphenyl)(imino)oxo-λ⁶-sulfanyl]prop-2-en-1-yl]-6-methyl-2-oxo-1,2,5,6,7,8-hexahydroquinoline-3-carboxamide

Purity: 97.3% (254 nm); RT=2.91 min, m/z=434.01 [M+1]; ¹H NMR (400 MHz, DMSO-d6) δ 9.99 (t, J=5.9 Hz, 1H), 8.03 (s, 1H), 7.69-7.60 (m, 2H), 7.35 (t, J=8.4 Hz, 1H), 6.76 (dt, J=14.9, 4.6 Hz, 1H), 6.52-6.45 (m, 1H), 4.60 (s, 1H), 4.16-4.09 (m, 2H), 3.92 (s, 3H), 2.65-2.60 (m, 2H), 2.16-2.07 (m, 2H), 1.84-1.74 (m, 2H), 1.36-1.29 (m, 1H), 1.00 (d, J=6.5 Hz, 3H).

### Example 143: N-[(2E)-3-[(3-fluoro-4-methoxyphenyl)(imino)oxo-λ⁶-sulfanyl]prop-2-en-1-yl]-2-oxo-1,2-dihydroquinoline-3-carboxamide

Purity: 92.3% (254 nm); RT=3.12 min, m/z=416.04 [M+1]; ¹H NMR (400 MHz, DMSO-d6) δ 12.49 (s, 1H), 9.95 (t, J=6.0 Hz, 1H), 8.83 (s, 1H), 7.95 (dd, J=7.9, 1.4 Hz, 1H), 7.73-7.57 (m, 3H), 7.43 (d, J=8.3 Hz, 1H), 7.39-7.24 (m, 2H), 6.79 (dt, 1H), 6.60 (d, 1H), 4.61 (s, 1H), 4.19 (t, J=4.9 Hz, 2H), 3.92 (s, 3H).

### Example 144: N-[(2E)-3-[(3-fluoro-4-methoxyphenyl)(imino)oxo-λ⁶-sulfanyl]prop-2-en-1-yl]-2-oxo-6-(propan-2-yl)-1,2,5,6,7,8-hexahydroquinoline-3-carboxamide

Purity: 97.5% (254 nm); RT=2.66 min, m/z=462.12 [M+1]; ¹H NMR (400 MHz, DMSO-d6) δ 12.28 (s, 1H), 9.99 (t, J=5.9 Hz, 1H), 8.06 (s, 1H), 7.69-7.59 (m, 2H), 7.35 (t, J=8.4 Hz, 1H), 6.76 (dt, J=14.9, 4.6 Hz, 1H), 6.48 (d, J=15.0 Hz, 1H), 4.60 (s, 1H), 4.18-4.08 (m, 2H), 3.92 (s, 3H), 2.62-2.53 (m, 2H), 2.30-2.17 (m, 1H), 1.91-1.83 (m, 1H), 1.61-1.49 (m, 1H), 1.46-1.21 (m, 3H), 0.97-0.89 (m, 6H).

### Example 145: N-[(2E)-3-[(3-fluoro-4-methoxyphenyl)(imino)oxo-λ⁶-sulfanyl]prop-2-en-1-yl]-2-oxo-1H,2H,5H,7H,8H-pyrano[4,3-b]pyridine-3-carboxamide

Purity: 79.1% (254 nm); RT=2.03 min, m/z=422.1 [M+1]; ¹H NMR (400 MHz, DMSO-d6) δ 12.50 (s, 1H), 9.95 (t, J=5.9 Hz, 1H), 8.05 (s, 1H), 7.64 (ddd, J=10.5, 8.0, 2.4 Hz, 2H), 7.35 (t, J=8.6 Hz, 1H), 6.76 (dt, J=15.0, 4.6 Hz, 1H), 6.49 (d, J=14.9 Hz, 1H), 4.59 (s, 1H), 4.52-4.47 (m, 2H), 4.14 (t, J=5.6 Hz, 2H), 3.92 (s, 3H), 3.85 (t, J=5.7 Hz, 2H), 2.67 (t, J=6.1 Hz, 2H).

### Example 146: N-[(2E)-3-[imino(oxo)(3-phenoxyphenyl)-λ⁶-sulfanyl]prop-2-en-1-yl]-2-oxo-1,2,5,6,7,8-hexahydroquinoline-3-carboxamide

Purity: 86.5% (254 nm); RT=2.61 min, m/z=464.18 [M+1]; ¹H NMR (400 MHz, DMSO-d6) δ 12.28 (s, 1H), 10.02 (s, 1H), 8.04 (s, 1H), 7.64-7.55 (m, 2H), 7.48-7.36 (m, 3H), 7.29-7.18 (m, 2H), 7.13-7.06 (m, 2H), 6.77 (dt, J=14.9, 4.7 Hz, 1H), 6.54-6.45 (m, 1H), 4.65 (s, 1H), 4.14 (t, J=5.3 Hz, 2H), 2.58 (t, J=5.8 Hz, 2H), 1.77-1.62 (m, 4H).

### Example 147: N-[(2E)-3-[(3-fluoro-4-methoxyphenyl)(imino)oxo-λ⁶-sulfanyl]prop-2-en-1-yl]-2-oxo-6-(trifluoromethyl)-1,2,5,6,7,8-hexahydroquinoline-3-carboxamide

Purity: 93.1% (254 nm); RT=2.31 min, m/z=488.39 [M+1]; ¹H NMR (400 MHz, DMSO-d6) δ 12.41 (s, 1H), 9.98 (s, 1H), 8.13 (s, 1H), 7.70-7.60 (m, 2H), 7.35 (t, J=8.5 Hz, 1H), 6.77 (dt, J=14.9, 4.6 Hz, 1H), 6.48 (d, J=15.0 Hz, 1H), 4.60 (s, 1H), 4.13 (t, J=5.5 Hz, 2H), 3.92 (s, 3H), 2.87-2.64 (m, 5H), 2.08 (d, J=13.0 Hz, 1H), 1.67-1.56 (m, 1H).

### Example 148: 6-tert-butyl-N-[(2E)-3-[(3-fluoro-4-methoxyphenyl)(imino)oxo-A*-sulfanyl]prop-2-en-1-yl]-2-oxo-1,2,5,6,7,8-hexahydroquinoline-3-carboxamide

Purity: 95.8% (254 nm); RT=1.86 min, m/z=476.27 [M+1]; ¹H NMR (400 MHz, DMSO-d6) δ 12.28 (s, 1H), 10.00 (t, J=5.9 Hz, 1H), 8.07 (s, 1H), 7.70-7.59 (m, 2H), 7.35 (t, J=8.4 Hz, 1H), 6.77 (dt, J=15.0, 4.6 Hz, 1H), 6.52-6.45 (m, 1H), 4.60 (s, 1H), 4.12 (d, J=5.2 Hz, 2H), 3.92 (s, 3H), 2.63-2.55 (m, 2H), 2.30-2.20 (m, 1H), 1.99-1.89 (m, 1H), 1.38-1.20 (m, 3H), 0.91 (s, 9H).

### Example 149: N-[(2E)-3-[(3-fluoro-4-methoxyphenyl)(imino)oxo-λ⁶-sulfanyl]prop-2-en-1-yl]-2-oxo-5-phenyl-1,2-dihydropyridine-3-carboxamide

Purity: 87.9% (254 nm); RT=2.76 min, m/z=442.3 [M+1]; ¹H NMR (400 MHz, DMSO-d6) δ 10.15 (s, 1H), 8.57 (d, J=3.0 Hz, 1H), 8.46 (s, 1H), 8.09 (d, J=2.8 Hz, 1H), 7.72-7.56 (m, 5H), 7.51-7.40 (m, 2H), 7.40-7.29 (m, 1H), 6.80 (dt, J=15.0, 4.6 Hz, 1H), 6.62-6.49 (m, 1H), 4.61 (s, 1H), 4.21-4.14 (m, 2H), 3.92 (s, 3H).

### Example 150: N-[(2E)-3-[(3-fluoro-4-methoxyphenyl)(imino)oxo-λ⁶-sulfanyl]prop-2-en-1-yl]-2-oxo-1H,2H,5H,6H,7H,8H,9H-cyclohepta[b]pyridine-3-carboxamide

Purity: 93.0% (254 nm); RT=2.1 min, m/z=434.29 [M+1]; ¹H NMR (400 MHz, DMSO-d6) δ 12.41 (s, 1H), 10.05-9.94 (m, 1H), 8.10 (s, 1H), 7.69-7.60 (m, 2H), 7.35 (t, J=8.5 Hz, 1H), 6.77 (dt, J=15.0, 4.6 Hz, 1H), 6.53-6.45 (m, 1H), 4.59 (s, 1H), 4.20-4.09 (m, 2H), 3.92 (s, 3H), 2.84-2.74 (m, 2H), 2.66-2.59 (m, 2H), 1.82-1.72 (m, 2H), 1.64-1.46 (m, 4H).

### Example 151: N-[(2E)-3-[(3-fluoro-4-methoxyphenyl)(imino)oxo-λ⁶-sulfanyl]prop-2-en-1-yl]-2-oxo-1H,2H,5H,6H,7H-cyclopenta[b]pyridine-3-carboxamide

Purity: 92.7% (254 nm); RT=1.9 min, m/z=406.19 [M+1]; ¹H NMR (400 MHz, DMSO-d6) δ 12.74 (s, 1H), 10.09 (s, 1H), 8.21 (s, 1H), 7.69-7.59 (m, 2H), 7.35 (t, J=8.4 Hz, 1H), 6.77 (dt, J=14.9, 4.6 Hz, 1H), 6.49 (dt, J=15.0, 1.9 Hz, 1H), 4.60 (s, 1H), 4.13 (t, J=5.4 Hz, 2H), 3.92 (s, 3H), 2.82 (t, J=7.6 Hz, 2H), 2.71 (t, J=7.3 Hz, 2H), 2.05 (p, J=7.6 Hz, 2H).

### Example 152: tert-butyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate

Purity: 94.9% (254 nm); RT=2.24 min, m/z=521.22 [M+1]; ¹H NMR (400 MHz, DMSO-d6) δ 12.49 (s, 1H), 9.94 (t, J=5.9 Hz, 1H), 8.13 (s, 1H), 7.71-7.58 (m, 2H), 7.35 (t, J=8.4 Hz, 1H), 6.77 (dt, J=14.9, 4.6 Hz, 1H), 6.54-6.43 (m, 1H), 4.60 (s, 1H), 4.32 (s, 2H), 4.14 (d, J=4.6 Hz, 2H), 3.92 (s, 3H), 3.57 (t, J=5.9 Hz, 2H), 2.65 (d, J=6.0 Hz, 2H), 1.42 (s, 9H).

### Example 153: 6-benzyl-N-[(2E)-3-[(3-fluoro-4-methoxyphenyl)(imino)oxo-A*-sulfanyl]prop-2-en-1-yl]-2-oxo-1,2,5,6,7,8-hexahydro-1,6-naphthyridine-3-carboxamide

Purity: 86.9% (254 nm); RT=2.83 min, m/z=511.23 [M+1]; ¹H NMR (400 MHz, DMSO-d6) δ 12.43 (s, 1H), 9.97 (t, J=6.0 Hz, 1H), 8.01 (s, 1H), 7.69-7.60 (m, 2H), 7.34 (d, J=4.2 Hz, 5H), 7.27 (dt, J=8.9, 4.2 Hz, 1H), 6.76 (dt, J=15.1, 4.6 Hz, 1H), 6.53-6.45 (m, 1H), 4.60 (s, 1H), 4.12 (t, J=5.2 Hz, 2H), 3.92 (s, 3H), 3.66 (s, 2H), 3.39 (s, 2H), 2.71-2.64 (m, 4H).

### Example 154: N-[(2E)-3-[imino(oxo)[4-(trifluoromethyl)phenyl]-λ⁶-sulfanyl]prop-2-en-1-yl]-2-oxo-1,2,5,6,7,8-hexahydroquinoline-3-carboxamide

Purity: 88.0% (254 nm); RT=2.34 min, m/z=440.23 [M+1]; ¹H NMR (400 MHz, DMSO-d6) δ 12.25 (s, 1H), 10.07-9.97 (m, 1H), 8.07 (d, J=8.2 Hz, 2H), 8.04 (s, 1H), 7.97 (d, J=8.3 Hz, 2H), 6.88 (dt, J=15.0, 4.6 Hz, 1H), 6.55 (d, J=15.0 Hz, 1H), 4.89 (s, 1H), 4.21-4.11 (m, 2H), 2.62-2.55 (m, 2H), 1.77-1.63 (m, 4H).

### Example 155: N-[(2E)-3-[(3-fluoro-4-methoxyphenyl)(imino)oxo-λ⁶-sulfanyl]prop-2-en-1-yl]-8,8-dimethyl-2-oxo-1,2,5,6,7,8-hexahydroquinoline-3-carboxamide

Purity: 97.0% (254 nm); RT=2.36 min, m/z=448.0 [M+1]; ¹H NMR (400 MHz, DMSO-d6) δ 11.95 (s, 1H), 10.06-9.88 (m, 1H), 8.03 (s, 1H), 7.69-7.60 (m, 2H), 7.35 (t, J=8.5 Hz, 1H), 6.77 (dt, J=15.0, 4.5 Hz, 1H), 6.50 (dd, J=14.9, 1.8 Hz, 1H), 4.59 (s, 1H), 4.23-4.11 (m, 2H), 3.92 (s, 3H), 1.74-1.55 (m, 4H), 1.30 (s, 6H).

### Example 156: N-[(2E)-3-({[1,1′-biphenyl]-4-yl}(imino)oxo-λ⁶-sulfanyl)prop-2-en-1-yl]-2-oxo-1,2,5,6,7,8-hexahydroquinoline-3-carboxamide

Purity: 97.3% (254 nm); RT=2.49 min, m/z=448.44 [M+1]; ¹H NMR (400 MHz, DMSO-d6) δ 12.26 (s, 1H), 10.03 (t, J=5.3 Hz, 1H), 8.04 (s, 1H), 7.96-7.90 (m, 2H), 7.89-7.83 (m, 2H), 7.77-7.70 (m, 2H), 7.52 (dd, J=8.3, 6.7 Hz, 2H), 7.48-7.41 (m, 1H), 6.83 (dt, J=15.0, 4.6 Hz, 1H), 6.57-6.49 (m, 1H), 4.63 (s, 1H), 4.21-4.11 (m, 2H), 2.58 (t, J=5.8 Hz, 2H), 1.77-1.62 (m, 4H).

### Example 157: N-[(2E)-3-[benzyl(imino)oxo-λ⁶-sulfanyl]prop-2-en-1-yl]-2-oxo-1,2,5,6,7,8-hexahydroquinoline-3-carboxamide

Purity: 94.9% (254 nm); RT=1.82 min, m/z=386.2 [M+1]; ¹H NMR (400 MHz, DMSO-d6) δ 12.30 (s, 1H), 10.02 (t, J=5.9 Hz, 1H), 8.10 (s, 1H), 7.35-7.26 (m, 5H), 6.53 (dt, J=15.1, 4.6 Hz, 1H), 6.38-6.28 (m, 1H), 4.30 (s, 2H), 4.15-4.08 (m, 2H), 3.86 (s, 1H), 2.64-2.54 (m, 4H), 1.77-1.64 (m, 4H).

### Example 158: N-[(2E)-3-[(3-fluoro-4-methoxyphenyl)(imino)oxo-λ⁶-sulfanyl]prop-2-en-1-yl]-2-oxo-1,2-dihydro-1,8-naphthyridine-3-carboxamide

Purity: 88.6% (254 nm); RT=3.05 min, m/z=417.3 [M+1]; ¹H NMR (400 MHz, DMSO-d6) δ 12.86 (s, 1H), 9.95-9.82 (m, 1H), 8.83 (s, 1H), 8.67 (dd, J=4.7, 1.8 Hz, 1H), 8.40 (d, J=7.7 Hz, 1H), 7.70-7.60 (m, 2H), 7.35 (t, J=8.2 Hz, 2H), 6.80 (dt, J=15.0, 4.5 Hz, 1H), 6.60 (d, J=14.9 Hz, 1H), 4.60 (s, 1H), 4.23-4.16 (m, 2H), 3.92 (s, 3H).

### Example 159: N-[(2E)-3-[(3-fluoro-4-methoxyphenyl)(imino)oxo-λ⁶-sulfanyl]prop-2-en-1-yl]-2-oxo-1,2-dihydro-1,6-naphthyridine-3-carboxamide

Purity: 94.9% (254 nm); RT=1.64 min, m/z=417.14 [M+1]; ¹H NMR (400 MHz, DMSO-d6) δ 12.68 (s, 1H), 9.74 (s, 1H), 9.11 (s, 1H), 8.89 (s, 1H), 8.57 (d, J=5.7 Hz, 1H), 7.70-7.58 (m, 2H), 7.35 (t, J=8.5 Hz, 1H), 7.30 (d, J=5.7 Hz, 1H), 6.79 (dt, J=15.0, 4.5 Hz, 1H), 6.60 (d, J=14.7 Hz, 1H), 4.59 (s, 1H), 4.19 (d, J=4.5 Hz, 2H), 3.92 (s, 3H).

### Example 160: N-[(2E)-3-{imino[4-(morpholin-4-yl)phenyl]oxo-λ⁶-sulfanyl}prop-2-en-1-yl]-2-oxo-1,2,5,6,7,8-hexahydroquinoline-3-carboxamide

Purity: 91.4% (254 nm); RT=2.15 min, m/z=457.25 [M+1]; ¹H NMR (400 MHz, DMSO-d6) δ 12.23 (s, 1H), 9.97 (t, J=6.0 Hz, 1H), 8.03 (s, 1H), 7.63 (d, J=9.0 Hz, 2H), 7.03 (d, J=9.0 Hz, 2H), 6.65 (dt, J=14.9, 4.7 Hz, 1H), 6.39 (d, J=15.0 Hz, 1H), 4.24 (s, 1H), 4.10 (t, J=5.2 Hz, 2H), 3.72 (t, J=4.9 Hz, 4H), 3.25 (t, J=5.0 Hz, 4H), 2.56 (d, J=6.2 Hz, 2H), 1.68 (s, 4H).

### Example 161: N-[(2E)-3-[imino(oxo)[4-(piperidin-1-yl)phenyl]-λ⁶-sulfanyl]prop-2-en-1-yl]-2-oxo-1,2,5,6,7,8-hexahydroquinoline-3-carboxamide

Purity: 91.3% (254 nm); RT=5.58 min, m/z=455.24 [M+1]; ¹H NMR (400 MHz, DMSO-d6) δ 10.56 (s, 1H), 7.90 (s, 1H), 7.64-7.51 (m, 2H), 7.03-6.93 (m, 2H), 6.64 (dt, J=14.9, 4.6 Hz, 1H), 6.43-6.32 (m, 1H), 4.15 (s, 1H), 4.08 (d, J=5.9 Hz, 2H), 1.68 (d, J=12.1 Hz, 4H), 1.57 (d, J=2.5 Hz, 6H).

### Example 162: N-[(2E)-3-[imino(oxo)[4-(Pyrrolidin-1-yl)phenyl]-λ⁶-sulfanyl]prop-2-en-1-yl]-2-oxo-1,2,5,6,7,8-hexahydroquinoline-3-carboxamide

Purity: 96.4% (254 nm); RT=2.01 min, m/z=441.21 [M+1]; ¹H NMR (400 MHz, DMSO-d6) δ 12.25 (s, 1H), 9.99 (d, J=6.2 Hz, 1H), 8.02 (s, 1H), 7.63-7.51 (m, 2H), 6.66-6.48 (m, 3H), 6.47-6.30 (m, 1H), 4.08 (q, J=3.7, 3.0 Hz, 3H), 3.27 (d, J=6.3 Hz, 4H), 2.56 (d, J=6.1 Hz, 2H), 2.02-1.89 (m, 4H), 1.77-1.52 (m, 4H).

### Example 163: N-[(2E)-3-[(4-acetamidophenyl)(imino)oxo-λ⁶-sulfanyl]prop-2-en-1-yl]-2-oxo-1,2,5,6,7,8-hexahydroquinoline-3-carboxamide

Purity: 87.7% (254 nm); RT=1.98 min, m/z=429.0 [M+1]; ¹H NMR (400 MHz, DMSO-d6) δ 12.25 (s, 1H), 10.33 (s, 1H), 10.03 (s, 1H), 8.02 (s, 1H), 7.80-7.70 (m, 4H), 6.71 (dt, J=14.9, 4.6 Hz, 1H), 6.44 (d, J=15.0 Hz, 1H), 4.43 (s, 1H), 4.10 (d, J=5.7 Hz, 2H), 2.56 (d, J=6.1 Hz, 2H), 2.07 (s, 3H), 1.75-1.59 (m, 4H).

### Example 164: N-[(2E)-3-[(4-cyclopropoxyphenyl)(imino)oxo-λ⁶-sulfanyl]prop-2-en-1-yl]-2-oxo-1,2,5,6,7,8-hexahydroquinoline-3-carboxamide

Purity: 99.5% (254 nm); RT=4.28 min, m/z=428.19 [M+1]; ¹H NMR (400 MHz, DMSO-d6) δ 12.26 (s, 1H), 10.00 (t, J=5.9 Hz, 1H), 8.04 (s, 1H), 7.82-7.74 (m, 2H), 7.24-7.17 (m, 2H), 6.73 (dt, J=14.9, 4.6 Hz, 1H), 6.45 (dt, J=14.9, 1.8 Hz, 1H), 4.42 (s, 1H), 4.12 (t, J=5.0 Hz, 2H), 3.94 (tt, J=6.1, 3.0 Hz, 1H), 2.58 (t, J=5.9 Hz, 2H), 1.69 (q, J=8.4, 7.9 Hz, 4H), 0.85-0.77 (m, 2H), 0.69 (tt, J=5.0, 3.2 Hz, 2H).

### Example 165: N-[(2E)-3-{[3-(dimethylamino)phenyl](imino)oxo-λ⁶-sulfanyl}prop-2-en-1-yl]-2-oxo-1,2,5,6,7,8-hexahydroquinoline-3-carboxamide

Purity: 58.2% (254 nm); RT=4.13 min, m/z=415.22 [M+1]; ¹H NMR (400 MHz, DMSO-d6) δ 12.27 (s, 1H), 10.03 (t, J=5.9 Hz, 1H), 8.03 (s, 1H), 7.44 (t, J=8.0 Hz, 1H), 7.19-7.15 (m, 1H), 7.13 (t, J=2.2 Hz, 1H), 7.08-6.96 (m, 2H), 6.81 (d, J=14.9 Hz, 1H), 4.21 (d, J=5.4 Hz, 2H), 2.97 (s, 6H), 2.58 (t, J=4.5 Hz, 2H), 1.67 (d, J=12.0 Hz, 4H).

### Example 166: N-[(2E)-3-[(4-cyclohexylphenyl)(imino)oxo-λ⁶-sulfanyl]prop-2-en-1-yl]-2-oxo-1,2,5,6,7,8-hexahydroquinoline-3-carboxamide

Purity: 99.2% (254 nm); RT=5.65 min, m/z=454.22 [M+1]; ¹H NMR (400 MHz, DMSO-d6) δ 12.23 (s, 1H), 10.01 (t, J=5.9 Hz, 1H), 8.04 (s, 1H), 7.78-7.73 (m, 2H), 7.45-7.40 (m, 2H), 6.77 (dt, J=14.9, 4.7 Hz, 1H), 6.47 (dd, J=14.9, 1.9 Hz, 1H), 4.46 (s, 1H), 4.13 (t, J=5.0 Hz, 2H), 2.58 (t, J=6.0 Hz, 3H), 1.84-1.76 (m, 4H), 1.69 (q, J=10.1, 7.3 Hz, 5H), 1.40 (q, J=11.5 Hz, 4H), 1.26 (t, J=12.1 Hz, 1H).

### Example 167: N-[(2E)-3-[(2,3-dihydro-1-benzofuran-5-yl)(imino)oxo-λ⁶-sulfanyl]Prop-2-en-1-yl]-2-oxo-1,2,5,6,7,8-hexahydroquinoline-3-carboxamide

Purity: 95.6% (254 nm); RT=2.23 min, m/z=414.17 [M+1]; ¹H NMR (400 MHz, DMSO-d6) δ 12.25 (s, 1H), 9.98 (t, J=5.9 Hz, 1H), 8.03 (s, 1H), 7.68 (d, J=1.9 Hz, 1H), 7.60 (dd, J=8.4, 2.1 Hz, 1H), 6.89 (d, J=8.4 Hz, 1H), 6.69 (dt, J=14.9, 4.7 Hz, 1H), 6.42 (dt, J=14.8, 1.8 Hz, 1H), 4.63 (t, J=8.8 Hz, 2H), 4.34 (s, 1H), 4.11 (t, J=5.2 Hz, 2H), 3.23 (t, J=8.8 Hz, 2H), 2.57 (t, J=5.9 Hz, 2H), 1.76-1.60 (m, 4H).

### Example 168: N-[(2E)-3-[imino(oxo)[3-(trifluoromethoxy)phenyl]-λ⁶-sulfanyl]prop-2-en-1-yl]-2-oxo-1,2,5,6,7,8-hexahydroquinoline-3-carboxamide

Purity: 99.1% (254 nm); RT=4.22 min, m/z=456.1 [M+1]; ¹H NMR (400 MHz, Acetonitrile-d3) δ 10.10 (s, 1H), 9.96 (s, 1H), 8.10 (s, 1H), 7.89 (ddd, J=7.9, 1.7, 1.0 Hz, 1H), 7.81 (d, J=2.5 Hz, 1H), 7.64 (t, J=8.1 Hz, 1H), 7.53 (ddt, J=8.3, 2.3, 1.1 Hz, 1H), 6.94 (dt, J=14.9, 4.3 Hz, 1H), 6.48 (dt, J=14.9, 1.9 Hz, 1H), 4.16 (ddt, J=6.0, 4.4, 1.7 Hz, 2H), 3.48 (d, J=24.0 Hz, 1H), 2.60 (t, J=6.0 Hz, 2H), 2.52 (t, J=6.0 Hz, 2H), 1.80-1.68 (m, 4H).

### Example 169: N-[(2E)-3-[(3-fluoro-4-methoxyphenyl)(imino)oxo-λ⁶-sulfanyl]prop-2-en-1-yl]-6-methoxy-2-oxo-1,2,5,6,7,8-hexahydroquinoline-3-carboxamide

Purity: 94.3% (254 nm); RT=2.23 min, m/z=450.2 [M+1]; ¹H NMR (400 MHz, Methanol-d4) δ 8.18 (s, 1H), 7.74 (d, 1H), 7.66 (dd, J=10.6, 2.3 Hz, 1H), 7.29 (t, J=8.4 Hz, 1H), 6.96 (dt, J=15.0, 4.5 Hz, 1H), 6.61 (d, J=15.2 Hz, 1H), 4.25 (d, 2H), 3.97 (s, 3H), 3.75 (t, J=4.8 Hz, 1H), 3.40 (s, 3H), 2.83 (td, J=14.0, 12.1, 5.9 Hz, 2H), 2.66 (dd, J=17.1, 6.2 Hz, 2H), 2.00 (q, J=6.2 Hz, 2H), 1.39 (d, J=6.5 Hz, 1H).

### Example 170: N-[(2E)-3-[(3-fluoro-4-methoxyphenyl)(imino)oxo-λ⁶-sulfanyl]prop-2-en-1-yl]-2-oxo-6-(2,2,2-trifluoroethyl)-1,2,5,6,7,8-hexahydro-1,6-naphthyridine-3-carboxamide

Purity: 93.7% (254 nm); RT=3.2 min, m/z=503.08 [M+1]; ¹H NMR (400 MHz, DMSO-d6) δ 12.44 (s, 1H), 9.96 (t, J=5.9 Hz, 1H), 8.04 (s, 1H), 7.72-7.57 (m, 2H), 7.35 (t, J=8.4 Hz, 1H), 6.77 (dt, J=14.9, 4.6 Hz, 1H), 6.49 (dt, J=14.9, 1.9 Hz, 1H), 4.60 (s, 1H), 4.17-4.10 (m, 2H), 3.92 (s, 3H), 3.65 (s, 2H), 3.41-3.34 (m, 2H), 2.93 (t, J=5.8 Hz, 2H), 2.73-2.65 (m, 2H).

### Example 171: N-[(2E)-3-[(3-fluoro-4-methoxyphenyl)(imino)oxo-λ⁶-sulfanyl]prop-2-en-1-yl]-6-[(4-methoxyphenyl)methyl]-2-oxo-1,2,5,6,7,8-hexahydro-1,6-naphthyridine-3-carboxamide

Purity: 84.1% (254 nm); RT=2.94 min, m/z=541.24 [M+1]; ¹H NMR (400 MHz, DMSO-d6) δ 12.42 (s, 1H), 9.96 (s, 1H), 8.00 (s, 1H), 7.69-7.59 (m, 2H), 7.35 (t, J=8.4 Hz, 1H), 7.29-7.19 (m, 2H), 6.94-6.87 (m, 2H), 6.76 (dt, J=15.0, 4.6 Hz, 1H), 6.53-6.45 (m, 1H), 4.60 (s, 1H), 4.17-4.08 (m, 2H), 3.92 (s, 3H), 3.75 (s, 3H), 3.58 (s, 2H), 3.35 (s, 2H), 2.68-2.60 (m, 4H).

### Example 172: 6-fluoro-N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide

Purity: 97.2% (254 nm); RT=2.25 min, m/z=438.15 [M+1]; ¹H NMR (400 MHz, DMSO-d6) δ 12.40 (s, 1H), 9.95 (t, J=5.9 Hz, 1H), 8.08 (s, 1H), 7.70-7.59 (m, 2H), 7.35 (t, J=8.4 Hz, 1H), 6.77 (dt, J=14.9, 4.6 Hz, 1H), 6.49 (d, J=15.0 Hz, 1H), 5.10 (dd, J=48.5, 4.9 Hz, 1H), 4.59 (s, 1H), 4.13 (t, J=5.3 Hz, 2H), 3.92 (s, 3H), 2.98-2.76 (m, 2H), 2.68 (d, J=6.4 Hz, 1H), 2.14-1.85 (m, 3H).

### Example 173: N-[(2E)-3-{[3-cyano-4-(piperidin-1-yl)phenyl](imino)oxo-λ⁶-sulfanyl}prop-2-en-1-yl]-2-oxo-1,2,5,6,7,8-hexahydroquinoline-3-carboxamide

Purity: 96.2% (254 nm); RT=3.07 min, m/z=480.28 [M+1]; ¹H NMR (400 MHz, DMSO-d6) δ 9.98 (t, J=5.8 Hz, 1H), 8.04 (s, 1H), 7.99 (d, J=2.4 Hz, 1H), 7.87 (dd, J=9.0, 2.3 Hz, 1H), 7.22 (d, J=9.0 Hz, 1H), 6.77 (dt, J=14.9, 4.5 Hz, 1H), 6.48 (d, J=15.0 Hz, 1H), 4.61 (s, 1H), 4.12 (s, 2H), 2.57 (d, J=6.1 Hz, 2H), 1.67 (s, 5H), 1.60 (s, 2H).

### Example 174: N-[(2E)-3-{imino[4-(methylamino)phenyl]oxo-λ⁶-sulfanyl}prop-2-en-1-yl]-2-oxo-1,2,5,6,7,8-hexahydroquinoline-3-carboxamide

Purity: 88.9% (254 nm); RT=1.98 min, m/z=401.21 [M+1]; ¹H NMR (400 MHz, DMSO-d6) δ 12.16 (s, 1H), 9.97 (t, J=5.9 Hz, 1H), 8.03 (s, 1H), 7.55-7.46 (m, 2H), 6.64-6.50 (m, 4H), 6.36 (dt, J=14.8, 1.8 Hz, 1H), 4.12-4.06 (m, 2H), 4.03 (s, 1H), 2.71 (d, J=4.9 Hz, 3H), 2.57 (t, J=6.0 Hz, 2H), 1.68 (t, J=4.3 Hz, 4H).

### Example 175: N-[(2E)-3-{[3-cyano-4-(morpholin-4-yl)phenyl](imino)oxo-λ⁶-sulfanyl}prop-2-en-1-yl]-2-oxo-1,2,5,6,7,8-hexahydroquinoline-3-carboxamide

Purity: 97.9% (254 nm); RT=1.88 min, m/z=482.18 [M+1]; ¹H NMR (400 MHz, DMSO-d6) δ 12.24 (s, 1H), 10.05 (s, 1H), 8.05 (d, J=2.3 Hz, 1H), 8.02 (s, 1H), 7.93 (dd, J=8.9, 2.4 Hz, 1H), 7.26 (d, J=8.9 Hz, 1H), 6.79 (dt, J=14.9, 4.6 Hz, 1H), 6.49 (dt, J=14.8, 1.8 Hz, 1H), 4.67 (s, 1H), 4.19-4.07 (m, 2H), 3.82-3.69 (m, 4H), 2.62-2.54 (m, 2H), 1.68 (q, J=7.7 Hz, 4H).

### Example 176: N-[(2E)-3-[imino(oxo)[4-(2-oxopiperidin-1-yl)phenyl]-λ⁶-sulfanyl]prop-2-en-1-yl]-2-oxo-1,2,5,6,7,8-hexahydroquinoline-3-carboxamide

Purity: 93.2% (254 nm); RT=2.67 min, m/z=469.37 [M+1]; ¹H NMR (400 MHz, DMSO-d6) δ 10.03 (t, 1H), 8.04 (s, 1H), 7.86-7.81 (m, 2H), 7.52-7.47 (m, 2H), 6.79 (dt, J=14.9, 4.7 Hz, 1H), 6.53-6.47 (m, 1H), 4.58 (s, 1H), 4.13 (d, J=6.0 Hz, 2H), 3.66 (t, J=5.6 Hz, 2H), 2.59 (d, J=5.9 Hz, 2H), 2.43 (t, J=6.3 Hz, 2H), 1.90-1.82 (m, 4H), 1.78-1.74 (m, 2H), 1.71-1.64 (m, 4H).

### Example 177: N-[(2E)-3-[imino(1-methyl-1,2,3,4-tetrahydroquinolin-6-yl)oxo-A*-sulfanyl]prop-2-en-1-yl]-2-oxo-1,2,5,6,7,8-hexahydroquinoline-3-carboxamide

Purity: 95.4% (254 nm); RT=2.22 min, m/z=441.24 [M+1]; ¹H NMR (400 MHz, DMSO-d6) δ 12.12 (s, 1H), 9.97 (t, J=5.8 Hz, 1H), 8.03 (s, 1H), 7.42 (dd, J=8.7, 2.4 Hz, 1H), 7.29 (d, J=2.3 Hz, 1H), 6.65-6.53 (m, 2H), 6.35 (dd, J=15.0, 1.7 Hz, 1H), 4.13-4.04 (m, 2H), 4.02 (s, 1H), 2.90 (s, 3H), 2.70 (t, J=6.4 Hz, 2H), 2.57 (d, J=6.2 Hz, 2H), 1.86 (p, J=6.2 Hz, 2H), 1.68 (s, 4H).

### Example 178: 2,2-difluoroethyl 3-{[(2E)-3-[(3-fluoro-4-methoxyphenyl)(imino) oxo-λ⁶-sulfanyl]prop-2-en-1-yl]carbamoyl}-2-oxo-1,2,5,6,7,8-hexahydro-1,6-naphthyridine-6-carboxylate

Purity: 95.1% (254 nm); RT=1.46 min, m/z=529.3 [M+1]; ¹H NMR (400 MHz, DMSO-d6) δ 12.52 (s, 1H), 9.92 (t, J=5.9 Hz, 1H), 8.15 (d, J=8.9 Hz, 1H), 7.69-7.60 (m, 2H), 7.35 (t, J=8.4 Hz, 1H), 6.77 (dt, J=14.9, 4.6 Hz, 1H), 6.54-6.46 (m, 1H), 6.43-6.10 (m, 2H), 4.60 (s, 1H), 4.34 (td, J=15.3, 3.3 Hz, 5H), 4.14 (t, J=5.4 Hz, 2H), 3.92 (s, 3H), 3.65 (s, 2H), 2.71 (t, J=5.7 Hz, 2H).

### Example 179: cyclopropylmethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate

Purity: 93.8% (254 nm); RT=1.9 min, m/z=471.09 [M+1]; ¹H NMR (400 MHz, DMSO-d6) δ 12.50 (s, 1H), 9.94 (t, J=5.8 Hz, 1H), 8.15 (s, 1H), 7.90-7.83 (m, 2H), 7.68-7.54 (m, 3H), 6.79 (dt, J=15.0, 4.6 Hz, 1H), 6.50 (dt, J=14.8, 1.8 Hz, 1H), 4.58 (s, 1H), 4.38 (s, 2H), 4.14 (q, J=5.0, 4.4 Hz, 2H), 3.89 (d, J=7.2 Hz, 2H), 3.69-3.56 (m, 2H), 2.69 (d, J=5.2 Hz, 2H), 1.11 (qq, J=7.6, 4.8, 3.8 Hz, 1H), 0.56-0.46 (m, 2H), 0.32-0.24 (m, 2H).

### Example 180: 2-fluoroethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate

Purity: 95.5% (254 nm); RT=2.12 min, m/z=511.13 [M+1]; ¹H NMR (400 MHz, DMSO-d6) δ 12.50 (s, 1H), 9.92 (t, J=6.0 Hz, 1H), 8.15 (s, 1H), 7.68-7.59 (m, 2H), 7.34 (t, J=8.5 Hz, 1H), 6.76 (dt, J=14.9, 4.6 Hz, 1H), 6.49 (dt, J=14.9, 1.8 Hz, 1H), 4.71-4.54 (m, 3H), 4.44-4.35 (m, OH), 4.35-4.21 (m, 2H), 4.17-4.10 (m, 2H), 3.95-3.89 (m, 3H), 3.68-3.58 (m, 2H), 2.74-2.63 (m, 2H).

### Example 181: 2-fluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate

Purity: 91.2% (254 nm); RT=3.26 min, m/z=463.05 [M+1]; ¹H NMR (400 MHz, DMSO-d6) δ 12.50 (s, 1H), 9.94 (t, J=6.0 Hz, 1H), 8.16 (s, 1H), 7.89-7.83 (m, 2H), 7.69-7.55 (m, 3H), 6.79 (dt, J=14.9, 4.6 Hz, 1H), 6.50 (dt, J=14.9, 1.8 Hz, 1H), 4.73-4.54 (m, 3H), 4.38 (s, 2H), 4.29 (ddd, J=30.3, 4.6, 3.2 Hz, 2H), 4.14 (d, J=5.6 Hz, 2H), 3.64 (s, 2H), 2.70 (t, J=5.7 Hz, 2H).

### Example 182: 2,2-difluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate

Purity: 93.4% (254 nm); RT=1.72 min, m/z=480.84 [M+1]; ¹H NMR (400 MHz, DMSO-d6) δ 12.50 (s, 1H), 9.93 (t, J=6.0 Hz, 1H), 8.16 (s, 1H), 7.91-7.82 (m, 2H), 7.68-7.54 (m, 3H), 6.79 (dt, J=15.0, 4.6 Hz, 1H), 6.50 (dt, J=14.8, 1.9 Hz, 1H), 6.43-6.11 (m, 2H), 4.58 (s, 1H), 4.47-4.28 (m, 4H), 4.14 (t, J=5.3 Hz, 2H), 3.65 (s, 2H), 2.70 (t, J=6.0 Hz, 2H).

### Example 183: 4,4-difluorocyclohexyl 3-{[(2E)-3-[(3-fluoro-4-methoxyphenyl) (imino)oxo-λ⁶-sulfanyl]prop-2-en-1-yl]carbamoyl}-2-oxo-1,2,5,6,7,8-hexahydro-1,6-naphthyridine-6-carboxylate

Purity: 94.7% (254 nm); RT=2.35 min, m/z=583.32 [M+1]; ¹H NMR (400 MHz, DMSO-d6) δ 12.43 (s, 1H), 9.95 (t, J=6.3 Hz, 1H), 8.19 (s, 1H), 7.72-7.56 (m, 2H), 7.35 (t, J=8.4 Hz, 1H), 6.77 (dt, J=15.0, 4.6 Hz, 1H), 6.49 (d, J=15.0 Hz, 1H), 4.85-4.80 (m, 1H), 4.60 (s, 1H), 4.49-4.29 (m, 2H), 4.16-4.12 (m, 2H), 3.92 (s, 3H), 3.65-3.61 (m, 2H), 2.73-2.67 (m, 2H), 2.00-1.96 (m, 4H), 1.83-1.77 (m, 4H).

### Example 184: cyclohexyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate

Purity: 95.5% (254 nm); RT=2.47 min, m/z=547.2 [M+1]; ¹H NMR (400 MHz, DMSO-d6) δ 12.49 (s, 1H), 9.95 (t, J=5.9 Hz, 1H), 8.15 (s, 1H), 7.69-7.59 (m, 2H), 7.35 (t, J=8.4 Hz, 1H), 6.77 (dt, J=14.9, 4.6 Hz, 1H), 6.49 (dt, J=14.9, 1.9 Hz, 1H), 4.62-4.58 (m, 2H), 4.37 (s, 2H), 4.14 (t, J=5.4 Hz, 2H), 3.92 (s, 3H), 3.64-3.59 (m, 2H), 2.70-2.64 (m, 2H), 1.79-1.75 (m, 2H), 1.72-1.63 (m, 2H), 1.48-1.29 (m, 6H).

### Example 185: 6-(cyclopropylmethyl)-N-[(2E)-3-[(3-fluoro-4-methoxyphenyl) (imino)oxo-λ⁶-sulfanyl]prop-2-en-1-yl]-2-oxo-1,2,5,6,7,8-hexahydro-1,6-naphthyridine-3-carboxamide

Purity: 88.9% (254 nm); RT=1.81 min, m/z=475.22 [M+1]; ¹H NMR (400 MHz, DMSO-d6) δ 12.40 (s, 1H), 10.00 (s, 1H), 8.05 (s, 1H), 7.70-7.59 (m, 2H), 7.40-7.31 (m, 1H), 6.77 (dt, J=14.9, 4.5 Hz, 1H), 6.50 (d, J=15.1 Hz, 1H), 4.60 (s, 1H), 4.18-4.08 (m, 2H), 3.92 (s, 3H), 3.48-3.40 (m, 2H), 2.75-2.69 (m, 2H), 2.53 (s, 2H), 2.36 (s, 2H), 0.92-0.86 (m, 1H), 0.54-0.45 (m, 2H), 0.16-0.08 (m, 2H).

### Example 186: N-[(2E)-3-[(3-fluoro-4-methoxyphenyl)(imino)oxo-λ⁶-sulfanyl]prop-2-en-1-yl]-2-oxo-1,2-dihydropyridine-3-carboxamide

Purity: 84.3% (254 nm); RT=1.93 min, m/z=366.1 [M+1]; ¹H NMR (400 MHz, DMSO-d6) δ 12.54 (d, J=6.1 Hz, 1H), 9.94 (t, J=5.9 Hz, 1H), 8.35-8.28 (m, 1H), 7.73 (tt, J=7.1, 3.5 Hz, 1H), 7.70-7.61 (m, 2H), 7.35 (t, J=8.6 Hz, 1H), 6.78 (dt, J=14.9, 4.6 Hz, 1H), 6.57-6.44 (m, 2H), 4.15 (dt, J=6.0, 3.0 Hz, 2H), 3.92 (s, 3H).

### Biological Assays

### Cell-Free HTRF-Based HOIP Inhibition Assay

The cell-free homogenous time-resolved fluorescence (HTRF) HOIP inhibition assay was performed to evaluate compound inhibition of HOIP activity similar to assays described in Katsuya et al (SLAS Discov 2018). This assay was performed in the following steps:
a) synthesis of recombinant fragment of human E3 ubiquitin-protein ligase RNF31 (HOIP), expressed with purity >90% in E. coli system (the protein was prepared as 1 mg/mL stock in a standard formulation);
b) incubation of the compounds in a typical concentration range: 100-0.005 μM, at a final DMSO conc. 1% with a mixture of HOIP and ubiquitin activating enzyme E1 at 25° C., for 1 h at 25° C.;
c) followed by addition of a ubiquitination solution including E2 and substrates (the reaction proceeds for 2.5 h at 25° C.); and
d) HTRF detection was performed using a PHERAstar FSX (BMG Labtech) plate reader (excitation at 337 nm, dual emissions at 665 and 620 nm).

### Cell-Based Dual Luciferase NF-κB Reporter Assay

Dual-luciferase reporter assay was designed to find inhibitors of HOIP affecting NF-κB signaling pathway. The assay was performed in HEK293 cells which were transfected with plasmids coding for Firefly and NanoLuc luciferases. The NanoLuc luciferase plasmid contains NF-κB response element which determines its expression.

The assay was run following standard dual-luciferase protocols. The assay consists of a few steps:
compounds were dispensed at a concentration range: 100-0.005 μM with a final DMSO concentration of 1% of final volume (25 μL);
cells were stimulated after 30 min with 2.5 ng/mL TNF; and
after 6 h Firefly and NanoLuc luciferase activities are determined according to the procedure described by the manufacturer of Nano-Glo Dual-Luciferase Reporter Assay System.

### TNF-α-Induced Cytotoxicity Assay

A viability assay, using bladder carcinoma RT4 cell line, was used for identifying the potential inhibitory effect of tested compounds on HOIP activity. The viability was determined by CellTiter-Glo Luminescent Cell Viability Assay (G7571, Promega) which measures the ability of generating a luminescent signal proportional to the amount of ATP present in living cells. The assay consists of a few steps:
a) the cells were routinely sub cultivated twice a week with standard complete growth medium composition;
b) cells were preparated for the assay by creating a cell suspension at the density of 0.05 mln cells/mL and dispensed into plates (ViewPlate-384, PerkinElmer) resulting in 1000 cells/well;
c) cells were stimulated with 1.25 ng/mL TNFα and with compounds and incubated for 72 h at 37° C. in the atmosphere with 5% CO₂; and
d) for detection cytotoxicity was measured with CellTiter-Glo® Luminescent Cell Viability Assay based on the method described by the manufacturer.

### Results Biological Assays

The compounds of the present invention (examples 5-186) were tested in one or more of the biological assays listed above. The below table 1 includes all IC50 values for biochemical (HTRF) and cellular (TNF, NFkB) activities of the compounds tested.

TABLE 1
IC50 values for biochemical and cellular activities

| Ex. | HTRF | | NF-κB | | TNF-α | |
|---|---|---|---|---|---|---|
| 5 | IC50 ≤ | 100 nM | 100 nM < | IC50 ≤ 1.0 μM | 100 nM < | IC50 ≤ 1.0 μM |
| 6 | 100 nM < | IC50 ≤ 1.0 μM | 1.0 μM < | IC50 ≤ 10 μM | 100 nM < | IC50 ≤ 1.0 μM |
| 7 | 100 nM < | IC50 ≤ 1.0 μM | 100 nM < | IC50 ≤ 1.0 μM | 100 nM < | IC50 ≤ 1.0 μM |
| 8 | 100 nM < | IC50 ≤ 1.0 μM | 100 nM < | IC50 ≤ 1.0 μM | 100 nM < | IC50 ≤ 1.0 μM |
| 9 | 100 nM < | IC50 ≤ 1.0 μM | 1.0 μM < | IC50 ≤ 10 μM | 1.0 μM < | IC50 ≤ 10 μM |
| 10 | IC50 ≤ | 100 nM | 100 nM < | IC50 ≤ 1.0 μM | 1.0 μM < | IC50 ≤ 10 μM |
| 11 | IC50 ≤ | 100 nM | 100 nM < | IC50 ≤ 1.0 μM | 100 nM < | IC50 ≤ 1.0 μM |
| 12 | 100 nM < | IC50 ≤ 1.0 μM | 1.0 μM < | IC50 ≤ 10 μM | 1.0 μM < | IC50 ≤ 10 μM |
| 13 | 1.0 μM < | IC50 ≤ 10 μM | — | | — | |
| 14 | 10 μM < | IC50 ≤ 100 μM | — | | — | |
| 15 | 1.0 μM < | IC50 ≤ 10 μM | — | | — | |
| 16 | IC50 ≤ | 100 nM | 100 nM < | IC50 ≤ 1.0 μM | 100 nM < | IC50 ≤ 1.0 μM |
| 17 | 1.0 μM < | IC50 ≤ 10 μM | — | | — | |
| 18 | 1.0 μM < | IC50 ≤ 10 μM | — | | — | |
| 19 | IC50 ≤ | 100 nM | 100 nM < | IC50 ≤ 1.0 μM | 100 nM < | IC50 ≤ 1.0 μM |
| 20 | 10 μM < | IC50 ≤ 100 μM | — | | — | |
| 21 | IC50 ≤ | 100 nM | IC50 ≤ | 100 nM | 100 nM < | IC50 ≤ 1.0 μM |
| 22 | IC50 ≤ | 100 nM | 100 nM < | IC50 ≤ 1.0 μM | 100 nM < | IC50 ≤ 1.0 μM |
| 23 | 10 μM < | IC50 ≤ 100 μM | — | | — | |
| 24 | IC50 ≤ | 100 nM | 100 nM < | IC50 ≤ 1.0 μM | 100 nM < | IC50 ≤ 1.0 μM |
| 25 | IC50 ≤ | 100 nM | 100 nM < | IC50 ≤ 1.0 μM | 1.0 μM < | IC50 ≤ 10 μM |
| 26 | IC50 ≤ | 100 nM | 100 nM < | IC50 ≤ 1.0 μM | 1.0 μM < | IC50 ≤ 10 μM |
| 27 | IC50 ≤ | 100 nM | 100 nM < | IC50 ≤ 1.0 μM | 100 nM < | IC50 ≤ 1.0 μM |
| 28 | IC50 ≤ | 100 nM | 100 nM < | IC50 ≤ 1.0 μM | 100 nM < | IC50 ≤ 1.0 μM |
| 29 | IC50 ≤ | 100 nM | 100 nM < | IC50 ≤ 1.0 μM | 100 nM < | IC50 ≤ 1.0 μM |
| 30 | IC50 ≤ | 100 nM | 100 nM < | IC50 ≤ 1.0 μM | 100 nM < | IC50 ≤ 1.0 μM |
| 31 | IC50 ≤ | 100 nM | 100 nM < | IC50 ≤ 1.0 μM | 100 nM < | IC50 ≤ 1.0 μM |
| 32 | IC50 ≤ | 100 nM | 100 nM < | IC50 ≤ 1.0 μM | 100 nM < | IC50 ≤ 1.0 μM |
| 33 | IC50 ≤ | 100 nM | 100 nM < | IC50 ≤ 1.0 μM | 100 nM < | IC50 ≤ 1.0 μM |
| 34 | 100 nM < | IC50 ≤ 1.0 μM | 100 nM < | IC50 ≤ 1.0 μM | 100 nM < | IC50 ≤ 1.0 μM |
| 35 | 100 nM < | IC50 ≤ 1.0 μM | 100 nM < | IC50 ≤ 1.0 μM | 1.0 μM < | IC50 ≤ 10 μM |
| 36 | 100 nM < | IC50 ≤ 1.0 μM | 10 μM < | IC50 ≤ 100 μM | 1.0 μM < | IC50 ≤ 10 μM |
| 37 | 1.0 μM < | IC50 ≤ 10 μM | — | | — | |
| 38 | 100 nM < | IC50 ≤ 1.0 μM | 100 nM < | IC50 ≤ 1.0 μM | 100 nM < | IC50 ≤ 1.0 μM |
| 39 | IC50 ≤ | 100 nM | 100 nM < | IC50 ≤ 1.0 μM | 100 nM < | IC50 ≤ 1.0 μM |
| 40 | 100 nM < | IC50 ≤ 1.0 μM | 100 nM < | IC50 ≤ 1.0 μM | 100 nM < | IC50 ≤ 1.0 μM |
| 41 | IC50 ≤ | 100 nM | 100 nM < | IC50 ≤ 1.0 μM | 100 nM < | IC50 ≤ 1.0 μM |
| 42 | 100 nM < | IC50 ≤ 1.0 μM | 1.0 μM < | IC50 ≤ 10 μM | 100 nM < | IC50 ≤ 1.0 μM |
| 43 | IC50 ≤ | 100 nM | 1.0 μM < | IC50 ≤ 10 μM | 1.0 μM < | IC50 ≤ 10 μM |
| 44 | 100 nM < | IC50 ≤ 1.0 μM | 100 nM < | IC50 ≤ 1.0 μM | 100 nM < | IC50 ≤ 1.0 μM |
| 45 | 100 nM < | IC50 ≤ 1.0 μM | 100 nM < | IC50 ≤ 1.0 μM | 1.0 μM < | IC50 ≤ 10 μM |
| 46 | 100 nM < | IC50 ≤ 1.0 μM | 100 nM < | IC50 ≤ 1.0 μM | 100 nM < | IC50 ≤ 1.0 μM |
| 47 | 100 nM < | IC50 ≤ 1.0 μM | 1.0 μM < | IC50 ≤ 10 μM | 1.0 μM < | IC50 ≤ 10 μM |
| 48 | 100 nM < | IC50 ≤ 1.0 μM | 100 nM < | IC50 ≤ 1.0 μM | 100 nM < | IC50 ≤ 1.0 μM |
| 49 | 100 nM < | IC50 ≤ 1.0 μM | 1.0 μM < | IC50 ≤ 10 μM | 1.0 μM < | IC50 ≤ 10 μM |
| 50 | 1.0 μM < | IC50 ≤ 10 μM | 1.0 μM < | IC50 ≤ 10 μM | — | |
| 51 | 100 nM < | IC50 ≤ 1.0 μM | 100 nM < | IC50 ≤ 1.0 μM | 100 nM < | IC50 ≤ 1.0 μM |
| 52 | 100 nM < | IC50 ≤ 1.0 μM | 1.0 μM < | IC50 ≤ 10 μM | 100 nM < | IC50 ≤ 1.0 μM |
| 53 | 100 nM < | IC50 ≤ 1.0 μM | 100 nM < | IC50 ≤ 1.0 μM | 100 nM < | IC50 ≤ 1.0 μM |
| 54 | 100 nM < | IC50 ≤ 1.0 μM | 100 nM < | IC50 ≤ 1.0 μM | 100 nM < | IC50 ≤ 1.0 μM |
| 55 | IC50 ≤ | 100 nM | 100 nM < | IC50 ≤ 1.0 μM | 100 nM < | IC50 ≤ 1.0 μM |
| 56 | IC50 ≤ | 100 nM | IC50 ≤ | 100 nM | 100 nM < | IC50 ≤ 1.0 μM |
| 57 | 100 nM < | IC50 ≤ 1.0 μM | 100 nM < | IC50 ≤ 1.0 μM | 100 nM < | IC50 ≤ 1.0 μM |
| 58 | IC50 ≤ | 100 nM | 100 nM < | IC50 ≤ 1.0 μM | 100 nM < | IC50 ≤ 1.0 μM |
| 59 | 100 nM < | IC50 ≤ 1.0 μM | 1.0 μM < | IC50 ≤ 10 μM | 100 nM < | IC50 ≤ 1.0 μM |
| 60 | 100 nM < | IC50 ≤ 1.0 μM | 100 nM < | IC50 ≤ 1.0 μM | 1.0 μM < | IC50 ≤ 10 μM |
| 61 | IC50 ≤ | 100 nM | 100 nM < | IC50 ≤ 1.0 μM | 100 nM < | IC50 ≤ 1.0 μM |
| 62 | 100 nM < | IC50 ≤ 1.0 μM | 100 nM < | IC50 ≤ 1.0 μM | 100 nM < | IC50 ≤ 1.0 μM |
| 63 | 100 nM < | IC50 ≤ 1.0 μM | 100 nM < | IC50 ≤ 1.0 μM | 100 nM < | IC50 ≤ 1.0 μM |
| 64 | 100 nM < | IC50 ≤ 1.0 μM | 100 nM < | IC50 ≤ 1.0 μM | 100 nM < | IC50 ≤ 1.0 μM |
| 65 | 100 nM < | IC50 ≤ 1.0 μM | 100 nM < | IC50 ≤ 1.0 μM | 100 nM < | IC50 ≤ 1.0 μM |
| 66 | 1.0 μM < | IC50 ≤ 10 μM | 1.0 μM < | IC50 ≤ 10 μM | 1.0 μM < | IC50 ≤ 10 μM |
| 67 | 100 nM < | IC50 ≤ 1.0 μM | 1.0 μM < | IC50 ≤ 10 μM | 10 μM < | IC50 ≤ 100 μM |
| 68 | IC50 ≤ | 100 nM | 1.0 μM < | IC50 ≤ 10 μM | 1.0 μM < | IC50 ≤ 10 μM |
| 69 | IC50 ≤ | 100 nM | 100 nM < | IC50 ≤ 1.0 μM | 100 nM < | IC50 ≤ 1.0 μM |
| 70 | IC50 ≤ | 100 nM | 100 nM < | IC50 ≤ 1.0 μM | 100 nM < | IC50 ≤ 1.0 μM |
| 71 | 100 nM < | IC50 ≤ 1.0 μM | 10 μM < | IC50 ≤ 100 μM | 10 μM < | IC50 ≤ 100 μM |
| 72 | 100 nM < | IC50 ≤ 1.0 μM | 100 nM < | IC50 ≤ 1.0 μM | 100 nM < | IC50 ≤ 1.0 μM |
| 73 | IC50 ≤ | 100 nM | 100 nM < | IC50 ≤ 1.0 μM | 100 nM < | IC50 ≤ 1.0 μM |
| 74 | 100 nM < | IC50 ≤ 1.0 μM | 100 nM < | IC50 ≤ 1.0 μM | 1.0 μM < | IC50 ≤ 10 μM |
| 75 | 100 nM < | IC50 ≤ 1.0 μM | 1.0 μM < | IC50 ≤ 10 μM | 1.0 μM < | IC50 ≤ 10 μM |
| 76 | 100 nM < | IC50 ≤ 1.0 μM | 1.0 μM < | IC50 ≤ 10 μM | 1.0 μM < | IC50 ≤ 10 μM |
| 77 | IC50 ≤ | 100 nM | 100 nM < | IC50 ≤ 1.0 μM | 1.0 μM < | IC50 ≤ 10 μM |
| 78 | 100 nM < | IC50 ≤ 1.0 μM | 100 nM < | IC50 ≤ 1.0 μM | 1.0 μM < | IC50 ≤ 10 μM |
| 79 | IC50 ≤ | 100 nM | 1.0 μM < | IC50 ≤ 10 μM | 1.0 μM < | IC50 ≤ 10 μM |
| 80 | 100 nM < | IC50 ≤ 1.0 μM | 10 μM < | IC50 ≤ 100 μM | 1.0 μM < | IC50 ≤ 10 μM |
| 81 | 100 nM < | IC50 ≤ 1.0 μM | 1.0 μM < | IC50 ≤ 10 μM | 1.0 μM < | IC50 ≤ 10 μM |
| 82 | 100 nM < | IC50 ≤ 1.0 μM | 1.0 μM < | IC50 ≤ 10 μM | 1.0 μM < | IC50 ≤ 10 μM |
| 83 | 100 nM < | IC50 ≤ 1.0 μM | 10 μM < | IC50 ≤ 100 μM | 10 μM < | IC50 ≤ 100 μM |
| 84 | IC50 ≤ | 100 nM | 100 nM < | IC50 ≤ 1.0 μM | 100 nM < | IC50 ≤ 1.0 μM |
| 85 | IC50 ≤ | 100 nM | 100 nM < | IC50 ≤ 1.0 μM | 100 nM < | IC50 ≤ 1.0 μM |
| 86 | IC50 ≤ | 100 nM | 100 nM < | IC50 ≤ 1.0 μM | 1.0 μM < | IC50 ≤ 10 μM |
| 87 | IC50 ≤ | 100 nM | 1.0 μM < | IC50 ≤ 10 μM | 10 μM < | IC50 ≤ 100 μM |
| 88 | IC50 ≤ | 100 nM | 100 nM < | IC50 ≤ 1.0 μM | 100 nM < | IC50 ≤ 1.0 μM |
| 89 | 100 nM < | IC50 ≤ 1.0 μM | 1.0 μM < | IC50 ≤ 10 μM | 100 nM < | IC50 ≤ 1.0 μM |
| 90 | IC50 ≤ | 100 nM | 100 nM < | IC50 ≤ 1.0 μM | 100 nM < | IC50 ≤ 1.0 μM |
| 91 | 100 nM < | IC50 ≤ 1.0 μM | 100 nM < | IC50 ≤ 1.0 μM | 100 nM < | IC50 ≤ 1.0 μM |
| 92 | IC50 ≤ | 100 nM | 1.0 μM < | IC50 ≤ 10 μM | 10 μM < | IC50 ≤ 100 μM |
| 93 | IC50 ≤ | 100 nM | 100 nM < | IC50 ≤ 1.0 μM | 100 nM < | IC50 ≤ 1.0 μM |
| 94 | IC50 ≤ | 100 nM | 100 nM < | IC50 ≤ 1.0 μM | 1.0 μM < | IC50 ≤ 10 μM |
| 95 | IC50 ≤ | 100 nM | 100 nM < | IC50 ≤ 1.0 μM | 1.0 μM < | IC50 ≤ 10 μM |
| 96 | 100 nM < | IC50 ≤ 1.0 μM | 100 nM < | IC50 ≤ 1.0 μM | 1.0 μM < | IC50 ≤ 10 μM |
| 97 | IC50 ≤ | 100 nM | 100 nM < | IC50 ≤ 1.0 μM | 1.0 μM < | IC50 ≤ 10 μM |
| 98 | IC50 ≤ | 100 nM | 100 nM < | IC50 ≤ 1.0 μM | 1.0 μM < | IC50 ≤ 10 μM |
| 99 | 100 nM < | IC50 ≤ 1.0 μM | 1.0 μM < | IC50 ≤ 10 μM | 1.0 μM < | IC50 ≤ 10 μM |
| 100 | 100 nM < | IC50 ≤ 1.0 μM | 10 μM < | IC50 ≤ 100 μM | 10 μM < | IC50 ≤ 100 μM |
| 101 | 100 nM < | IC50 ≤ 1.0 μM | 10 μM < | IC50 ≤ 100 μM | 10 μM < | IC50 ≤ 100 μM |
| 102 | 1.0 μM < | IC50 ≤ 10 μM | — | | — | |
| 103 | 100 nM < | IC50 ≤ 1.0 μM | 10 μM < | IC50 ≤ 100 μM | 10 μM < | IC50 ≤ 100 μM |
| 104 | 100 nM < | IC50 ≤ 1.0 μM | 1.0 μM < | IC50 ≤ 10 μM | 1.0 μM < | IC50 ≤ 10 μM |
| 105 | 100 nM < | IC50 ≤ 1.0 μM | — | | — | |
| 106 | 100 nM < | IC50 ≤ 1.0 μM | — | | 1.0 μM < | IC50 ≤ 10 μM |
| 107 | 100 nM < | IC50 ≤ 1.0 μM | 10 μM < | IC50 ≤ 100 μM | 10 μM < | IC50 ≤ 100 μM |
| 108 | 1.0 μM < | IC50 ≤ 10 μM | — | | — | |
| 109 | 100 nM < | IC50 ≤ 1.0 μM | 10 μM < | IC50 ≤ 100 μM | 10 μM < | IC50 ≤ 100 μM |
| 110 | 100 nM < | IC50 ≤ 1.0 μM | 1.0 μM < | IC50 ≤ 10 μM | 1.0 μM < | IC50 ≤ 10 μM |
| 111 | 1.0 μM < | IC50 ≤ 10 μM | — | | — | |
| 112 | 100 nM < | IC50 ≤ 1.0 μM | 1.0 μM < | IC50 ≤ 10 μM | 1.0 μM < | IC50 ≤ 10 μM |
| 113 | 100 nM < | IC50 ≤ 1.0 μM | 10 μM < | IC50 ≤ 100 μM | 10 μM < | IC50 ≤ 100 μM |
| 114 | 100 nM < | IC50 ≤ 1.0 μM | 1.0 μM < | IC50 ≤ 10 μM | 10 μM < | IC50 ≤ 100 μM |
| 115 | 100 nM < | IC50 ≤ 1.0 μM | 10 μM < | IC50 ≤ 100 μM | 10 μM < | IC50 ≤ 100 μM |
| 116 | IC50 ≤ | 100 nM | 1.0 μM < | IC50 ≤ 10 μM | 1.0 μM < | IC50 ≤ 10 μM |
| 117 | 100 nM < | IC50 ≤ 1.0 μM | 10 μM < | IC50 ≤ 100 μM | 10 μM < | IC50 ≤ 100 μM |
| 118 | 100 nM < | IC50 ≤ 1.0 μM | — | | — | |
| 119 | IC50 ≤ | 100 nM | 100 nM < | IC50 ≤ 1.0 μM | 100 nM < | IC50 ≤ 1.0 μM |
| 120 | 100 nM < | IC50 ≤ 1.0 μM | 1.0 μM < | IC50 ≤ 10 μM | 1.0 μM < | IC50 ≤ 10 μM |
| 121 | IC50 ≤ | 100 nM | 100 nM < | IC50 ≤ 1.0 μM | 1.0 μM < | IC50 ≤ 10 μM |
| 122 | 100 nM < | IC50 ≤ 1.0 μM | 100 nM < | IC50 ≤ 1.0 μM | 100 nM < | IC50 ≤ 1.0 μM |
| 123 | 100 nM < | IC50 ≤ 1.0 μM | 10 μM < | IC50 ≤ 100 μM | 10 μM < | IC50 ≤ 100 μM |
| 124 | 1.0 μM < | IC50 ≤ 10 μM | — | | — | |
| 125 | 100 nM < | IC50 ≤ 1.0 μM | 1.0 μM < | IC50 ≤ 10 μM | 1.0 μM < | IC50 ≤ 10 μM |
| 126 | IC50 ≤ | 100 nM | 100 nM < | IC50 ≤ 1.0 μM | 1.0 μM < | IC50 ≤ 10 μM |
| 127 | 100 nM < | IC50 ≤ 1.0 μM | 100 nM < | IC50 ≤ 1.0 μM | 1.0 μM < | IC50 ≤ 10 μM |
| 128 | 100 nM < | IC50 ≤ 1.0 μM | 1.0 μM < | IC50 ≤ 10 μM | 1.0 μM < | IC50 ≤ 10 μM |
| 129 | 100 nM < | IC50 ≤ 1.0 μM | — | | 1.0 μM < | IC50 ≤ 10 μM |
| 130 | 1.0 μM < | IC50 ≤ 10 μM | — | | — | |
| 131 | IC50 ≤ | 100 NM | 100 NM < | IC50 ≤ 1.0 μM | 100 NM < | IC50 ≤ 1.0 μM |
| 132 | 100 NM < | IC50 ≤ 1.0 μM | 100 NM < | IC50 ≤ 1.0 μM | 1.0 μM < | IC50 ≤ 10 μM |
| 133 | IC50 ≤ | 100 NM | 100 NM < | IC50 ≤ 1.0 μM | 100 NM < | IC50 ≤ 1.0 μM |
| 134 | 100 NM < | IC50 ≤ 1.0 μM | 100 NM < | IC50 ≤ 1.0 μM | 100 NM < | IC50 ≤ 1.0 μM |
| 135 | 100 NM < | IC50 ≤ 1.0 μM | 100 NM < | IC50 ≤ 1.0 μM | 1.0 μM < | IC50 ≤ 10 μM |
| 136 | IC50 ≤ | 100 NM | 100 NM < | IC50 ≤ 1.0 μM | 1.0 μM < | IC50 ≤ 10 μM |
| 137 | 100 NM < | IC50 ≤ 1.0 μM | 100 NM < | IC50 ≤ 1.0 μM | 100 NM < | IC50 ≤ 1.0 μM |
| 138 | 100 NM < | IC50 ≤ 1.0 μM | 100 NM < | IC50 ≤ 1.0 μM | 100 NM < | IC50 ≤ 1.0 μM |
| 139 | 100 NM < | IC50 ≤ 1.0 μM | 100 NM < | IC50 ≤ 1.0 μM | 100 NM < | IC50 ≤ 1.0 μM |
| 140 | 100 NM < | IC50 ≤ 1.0 μM | 100 NM < | IC50 ≤ 1.0 μM | 100 NM < | IC50 ≤ 1.0 μM |
| 141 | 100 NM < | IC50 ≤ 1.0 μM | 100 NM < | IC50 ≤ 1.0 μM | 100 NM < | IC50 ≤ 1.0 μM |
| 142 | 100 NM < | IC50 ≤ 1.0 μM | 100 NM < | IC50 ≤ 1.0 μM | 100 NM < | IC50 ≤ 1.0 μM |
| 143 | IC50 ≤ | 100 NM | 100 NM < | IC50 ≤ 1.0 μM | 100 NM < | IC50 ≤ 1.0 μM |
| 144 | IC50 ≤ | 100 NM | 100 NM < | IC50 ≤ 1.0 μM | 100 NM < | IC50 ≤ 1.0 μM |
| 145 | 100 NM < | IC50 ≤ 1.0 μM | 10 μM < | IC50 ≤ 100 μM | 1.0 μM < | IC50 ≤ 10 μM |
| 146 | IC50 ≤ | 100 NM | 100 NM < | IC50 ≤ 1.0 μM | 100 NM < | IC50 ≤ 1.0 μM |
| 147 | 100 NM < | IC50 ≤ 1.0 μM | 1.0 μM < | IC50 ≤ 10 μM | 1.0 μM < | IC50 ≤ 10 μM |
| 148 | 100 NM < | IC50 ≤ 1.0 μM | 100 NM < | IC50 ≤ 1.0 μM | 100 NM < | IC50 ≤ 1.0 μM |
| 149 | 100 NM < | IC50 ≤ 1.0 μM | 1.0 μM < | IC50 ≤ 10 μM | 1.0 μM < | IC50 ≤ 10 μM |
| 150 | 100 NM < | IC50 ≤ 1.0 μM | 1.0 μM < | IC50 ≤ 10 μM | 100 NM < | IC50 ≤ 1.0 μM |
| 151 | 100 NM < | IC50 ≤ 1.0 μM | 1.0 μM < | IC50 ≤ 10 μM | 100 NM < | IC50 ≤ 1.0 μM |
| 152 | IC50 ≤ | 100 NM | 1.0 μM < | IC50 ≤ 10 μM | 1.0 μM < | IC50 ≤ 10 μM |
| 153 | 100 NM < | IC50 ≤ 1.0 μM | 100 NM < | IC50 ≤ 1.0 μM | 100 NM < | IC50 ≤ 1.0 μM |
| 154 | IC50 ≤ | 100 NM | — | | 100 NM < | IC50 ≤ 1.0 μM |
| 155 | 1.0 μM < | IC50 ≤ 10 μM | — | | 1.0 μM < | IC50 ≤ 10 μM |
| 156 | IC50 ≤ | 100 NM | — | | 100 NM < | IC50 ≤ 1.0 μM |
| 157 | 1.0 μM < | IC50 ≤ 10 μM | — | | — | |
| 158 | 100 NM < | IC50 ≤ 1.0 μM | — | | 1.0 μM < | IC50 ≤ 10 μM |
| 159 | 100 NM < | IC50 ≤ 1.0 μM | — | | 10 μM < | IC50 ≤ 100 μM |
| 160 | 100 NM < | IC50 ≤ 1.0 μM | — | | 100 NM < | IC50 ≤ 1.0 μM |
| 161 | 100 NM < | IC50 ≤ 1.0 μM | — | | 100 NM < | IC50 ≤ 1.0 μM |
| 162 | IC50 ≤ | 100 NM | — | | 100 NM < | IC50 ≤ 1.0 μM |
| 163 | 100 NM < | IC50 ≤ 1.0 μM | — | | 10 μM < | IC50 ≤ 100 μM |
| 164 | IC50 ≤ | 100 NM | — | | 100 NM < | IC50 ≤ 1.0 μM |
| 165 | 100 NM < | IC50 ≤ 1.0 μM | — | | 100 NM < | IC50 ≤ 1.0 μM |
| 166 | IC50 ≤ | 100 NM | — | | 100 NM < | IC50 ≤ 1.0 μM |
| 167 | 100 NM < | IC50 ≤ 1.0 μM | — | | 100 NM < | IC50 ≤ 1.0 μM |
| 168 | IC50 ≤ | 100 NM | — | | 100 NM < | IC50 ≤ 1.0 μM |
| 169 | 100 NM < | IC50 ≤ 1.0 μM | 1.0 μM < | IC50 ≤ 10 μM | 1.0 μM < | IC50 ≤ 10 μM |
| 170 | 100 NM < | IC50 ≤ 1.0 μM | 1.0 μM < | IC50 ≤ 10 μM | 1.0 μM < | IC50 ≤ 10 μM |
| 171 | 100 NM < | IC50 ≤ 1.0 μM | — | | 100 NM < | IC50 ≤ 1.0 μM |
| 172 | 100 NM < | IC50 ≤ 1.0 μM | — | | 1.0 μM < | IC50 ≤ 10 μM |
| 173 | IC50 ≤ | 100 NM | — | | 100 NM < | IC50 ≤ 1.0 μM |
| 174 | 100 NM < | IC50 ≤ 1.0 μM | — | | 100 NM < | IC50 ≤ 1.0 μM |
| 175 | IC50 ≤ | 100 NM | — | | 1.0 μM < | IC50 ≤ 10 μM |
| 176 | 100 NM < | IC50 ≤ 1.0 μM | — | | — | |
| 177 | IC50 ≤ | 100 NM | — | | 100 NM < | IC50 ≤ 1.0 μM |
| 178 | IC50 ≤ | 100 NM | 1.0 μM < | IC50 ≤ 10 μM | 1.0 μM < | IC50 ≤ 10 μM |
| 179 | 100 NM < | IC50 ≤ 1.0 μM | 1.0 μM < | IC50 ≤ 10 μM | 1.0 μM < | IC50 ≤ 10 μM |
| 180 | IC50 ≤ | 100 NM | 1.0 μM < | IC50 ≤ 10 μM | 1.0 μM < | IC50 ≤ 10 μM |
| 181 | 100 NM < | IC50 ≤ 1.0 μM | 10 μM < | IC50 ≤ 100 μM | 1.0 μM < | IC50 ≤ 10 μM |
| 182 | 100 NM < | IC50 ≤ 1.0 μM | 10 μM < | IC50 ≤ 100 μM | 1.0 μM < | IC50 ≤ 10 μM |

| | | | | | |
|---|---|---|---|---|---|
| 183 | IC50 ≤ | 100 NM | — | 1.0 μM < | IC50 ≤ 10 μM |
| 184 | IC50 ≤ | 100 NM | — | 100 NM < | IC50 ≤ 1.0 μM |
| 185 | 1.0 μM < | IC50 ≤ 10 μM | — | 10 μM < | IC50 ≤ 100 μM |
| 186 | 100 NM < | IC50 ≤ 1.0 μM | — | 1.0 μM < | IC50 ≤ 10 μM |

### BRIEF DESCRIPTION OF THE DRAWINGS

FIG. 1 shows the percentage of inhibition of RNF31 activity in the HTRF assay, wherein the activity of compounds A and B (selected from the compounds of the present invention) is shown. It was found that the compounds show an improved activity compared to compounds known in the art having the same mechanism of action.

FIG. 2 shows the percentage of inhibition of NF-κB activity in the luciferase assay, wherein the activity of compounds A and B (selected from the compounds of the present invention) is shown. It was found that the compounds show an improved activity compared to compounds known in the art having the same mechanism of action.

FIG. 3A shows the cell viability of RT4 (Bladder carcinoma) after titration with TNF cytokine, whereas FIG. 3B shows the cell viability of RT4 after titration of compound A with or without a fixed concentration of TNF (1.25 ng/ml).

FIG. 4A-J shows the cell viability of various cell lines treated with a fixed concentration of compound A with titration of TNF in the TNF cytotoxicity assay. The cell lines used include bladder carcinoma (FIG. 4A), cutaneous melanoma (FIG. 4B), colorectal adenocarcinoma (FIG. 4C), pancreatic ductal adenocarcinoma (FIG. 4D), ovarian serous adenocarcinoma (FIG. 4E), triple negative breast cancer (FIG. 4F), lung adenocarcinoma (FIG. 4G), osteosarcoma (FIG. 4H), acute monocytic leukemia (FIG. 4I), and glioblastoma (FIG. 4J).

FIG. 5A shows G150 cell lines exposed to titrated levels of TNF for 72 hours in the TNF cytotoxicity assay, whereas FIG. 5B shows G150 cell lines treated with a fixed concentration of compound A with titration of TNF for 72 hours in the TNF cytotoxicity assay.

FIG. 6A-D shows the monotherapy effect of compound A. The cell viability of mantle cell lymphoma (FIG. 6A), lung adenocarcinoma FIG. 6B), ovarian serous adenocarcinoma (FIG. 6C), and diffuse large B-cell lymphoma (FIG. 6D) is shown 72 hours after titration of compound A.

FIG. 7A shows the cell viability of PBMC (healthy peripheral blood mononuclear cell) after titration with compound A after 72 hours, whereas FIG. 7B shows the cell viability of PBMC treated with a titration of TNF for 72 hours with or without a fixed concentration of compound A. The results provided in FIG. 7 suggest an on tumor effect, even with the addition of TNF.

FIG. 8A shows tumor growth over time of EMT6 (triple negative breast cancer) tumor cells transplanted into syngenic mice. The mice were treated daily for 10 days with compound A. FIG. 8B shows the differences of tumor volumes at day 15 between mice treated with compound A and mice treated with a vehicle only.

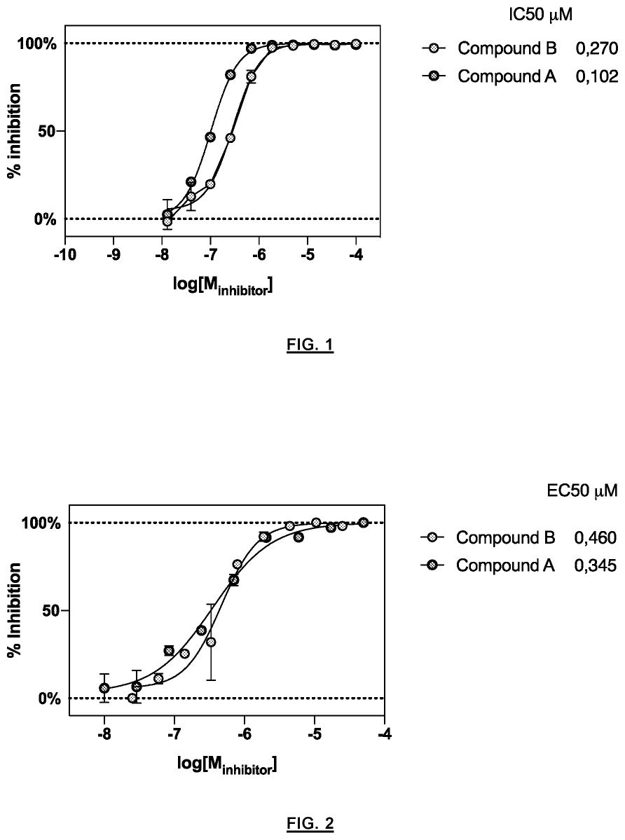

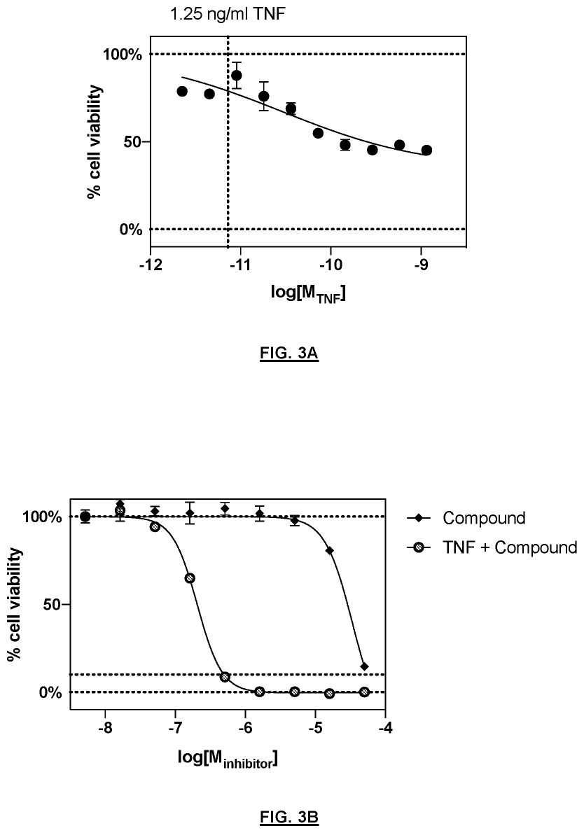

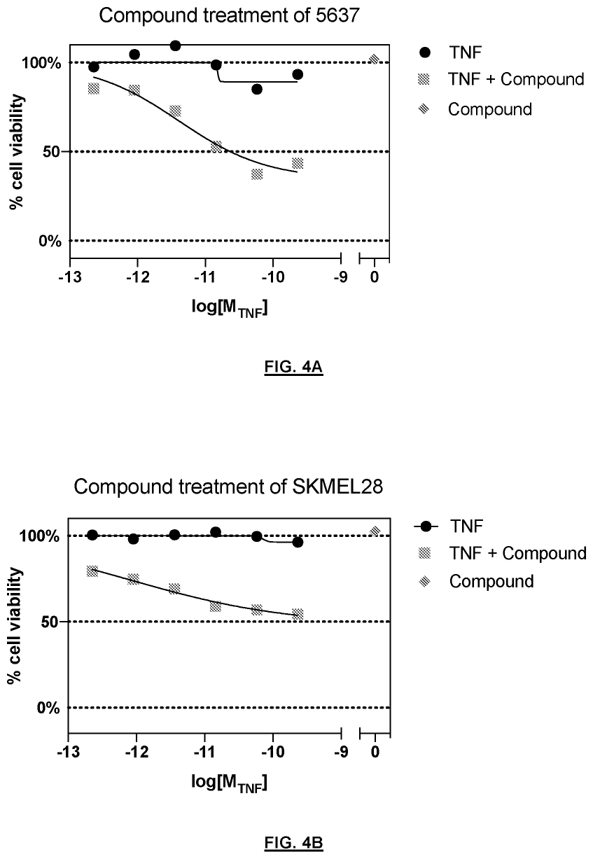

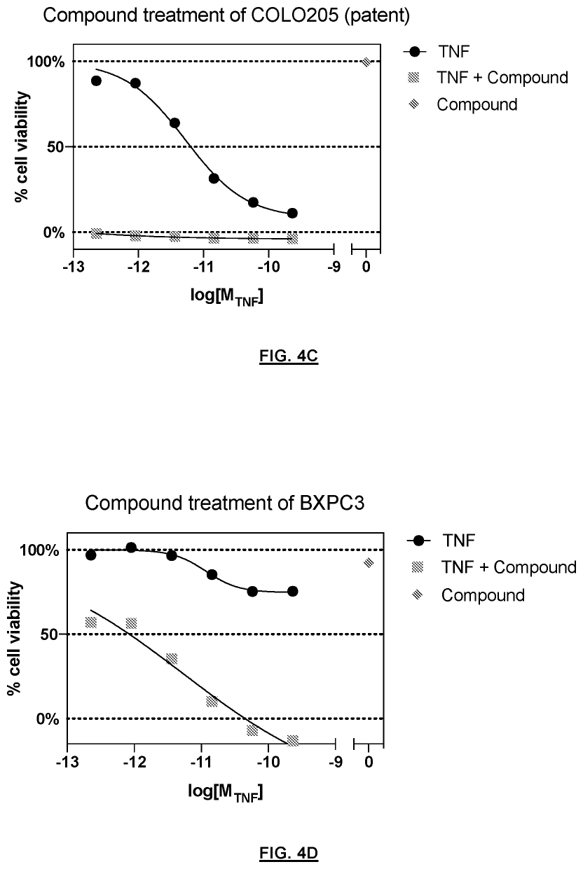

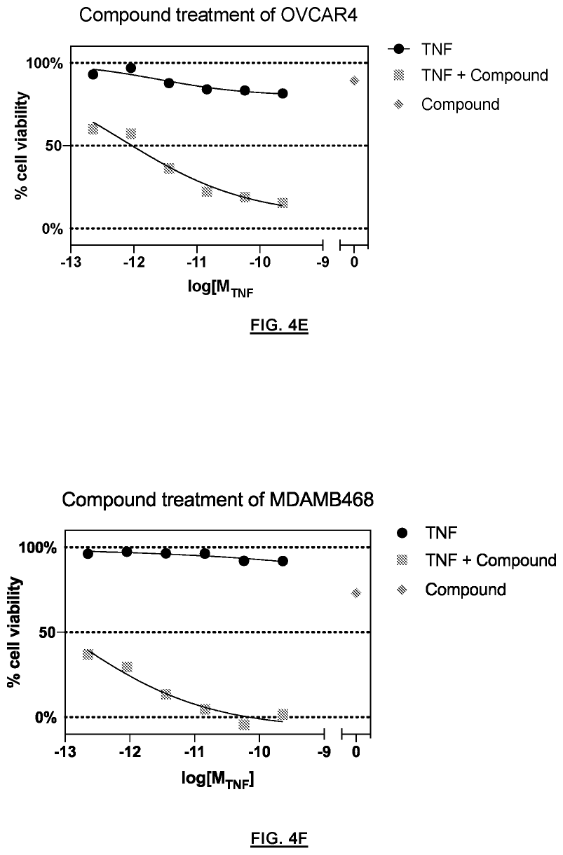

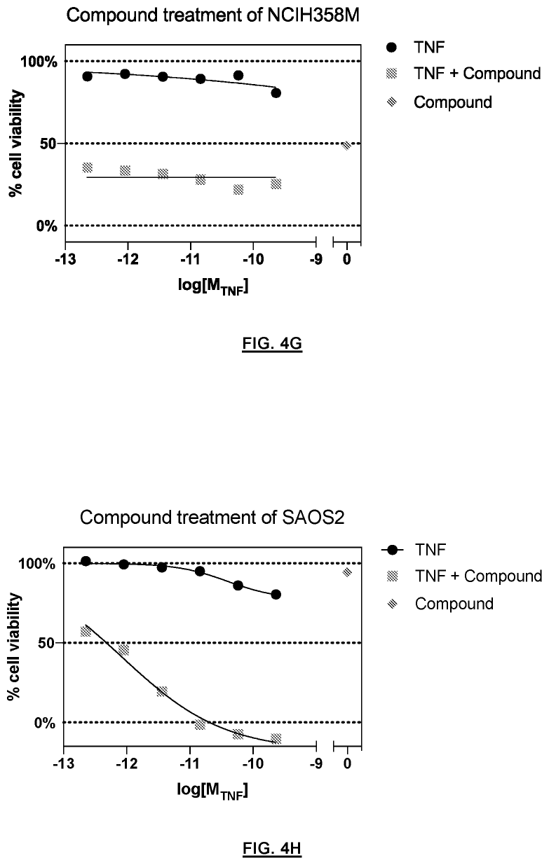

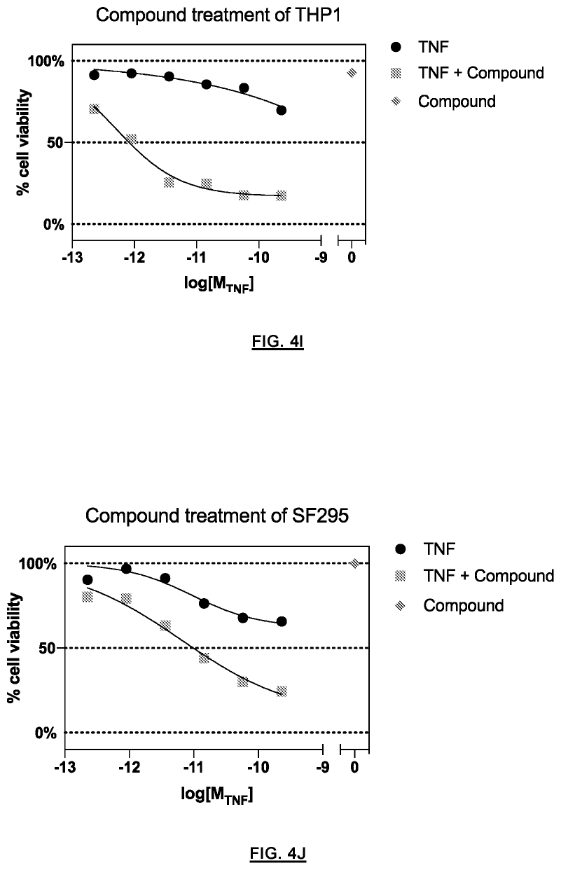

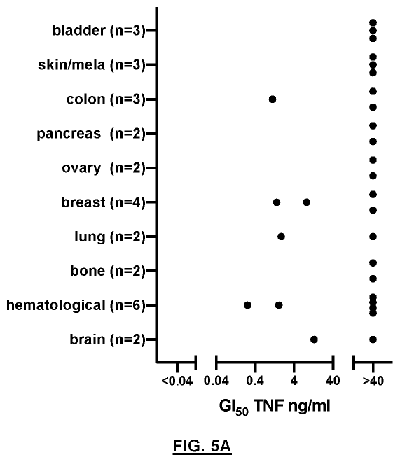

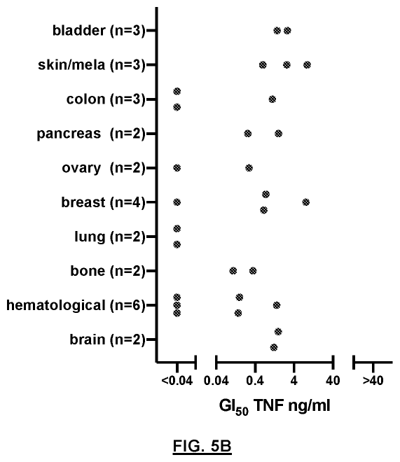

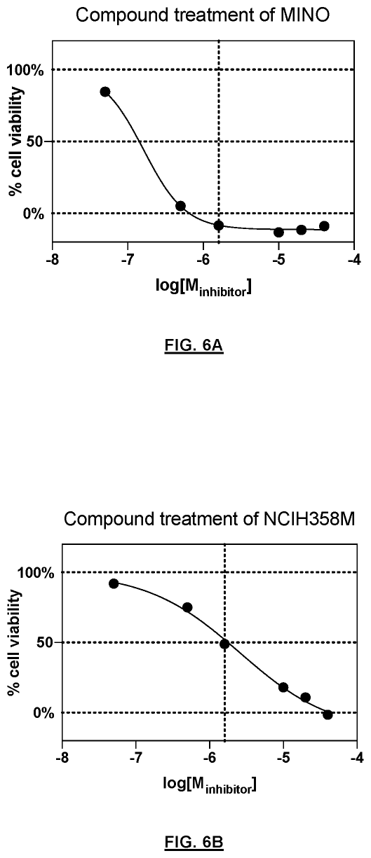

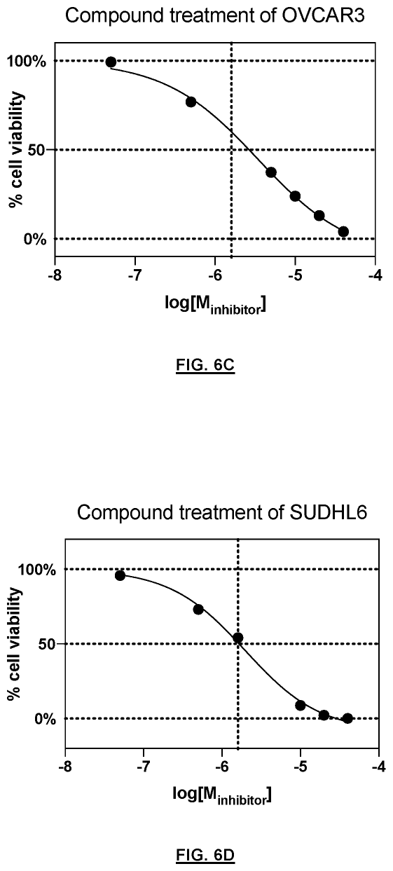

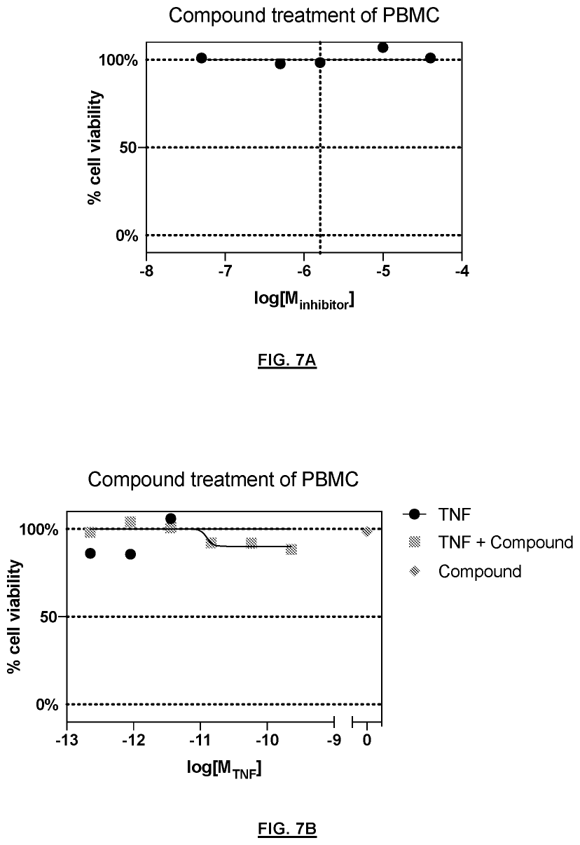

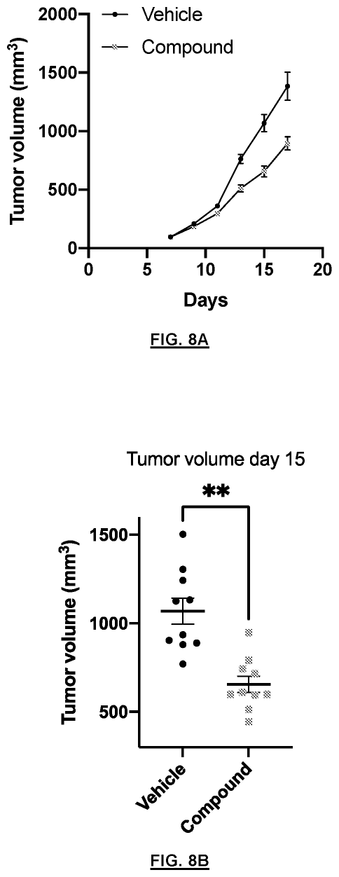

1. Compound having the formula (I): 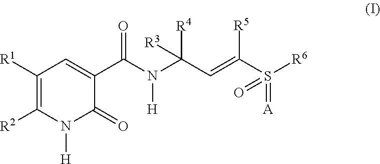
or the pharmaceutical acceptable salt thereof, wherein R¹ and R² are both hydrogen or wherein: 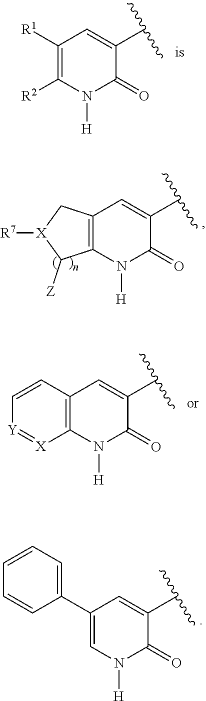
wherein:
n is 1, 2, 3 or 4;
A is oxygen or nitrogen;
X is carbon or nitrogen or wherein R⁷—X is oxygen;
Y is carbon or nitrogen;
Z is hydrogen, (C1-C6)alkyl or di-(C1-C6)alkyl;
R³ and R⁴ are each independently selected from the group consisting of hydrogen, (C1-C4)alkyl or wherein R³ and R⁴ together with the carbon to which they are attached form (C3-C6)cycloalkyl;
R⁵ is selected from the group consisting of hydrogen, halo or (C1-C6)alkyl;
R⁶ is selected from the group consisting of (C1-C6)alkyl, (C2-C6)alkenyl, (C2-C6)alkynyl, (C3-C10)cycloalkyl, (C2-C9)heterocycloalkyl, (C6-C10)aryl, (C6-C10)aryl(C1-C6)alkyl or (C2-C9)heteroaryl, wherein the cycloalkyl, heterocycloalkyl, aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino;
when X is carbon, R⁷ is R^{7′} and R^{7″}, wherein R^{7′} is selected from the group consisting of hydrogen, halo or (C1-C6)alkyl, and wherein R^{7″} is selected from the group consisting of hydrogen, halo, (C1-C6)alkyl, (C1-C6)alkoxy, (C2-C6)alkenyl, (C2-C6)alkynyl, (C6-C10)aryl(C1-C6)alkyl, (C2-C9)heteroaryl(C1-C6)alkyl, wherein the aryl and heteroaryl groups are optionally substituted with one to three (C1-C6)alkyl;
when X is nitrogen, R⁷ is selected from the group consisting of hydrogen, (C1-C6)alkyl, (C3-C10)cycloalkyl(C1-C6)alkyl, (C6-C10)aryl(C1-C6)alkyl, (C2-C9)heteroaryl(C1-C6)alkyl, wherein the aryl and heteroaryl groups are optionally substituted with one to three (C1-C6)alkyl or wherein R⁷ is R⁸M−, wherein:
R⁸M- is selected from the group consisting of R⁸—C(O)—, R⁸—O—C(O)—, R⁸—NH—C(O)— or R⁸—S(O)₂—; and
R⁸ is selected from the group consisting of (C1-C6)alkyl, (C3-C10)cycloalkyl, (C3-C10)cycloalkyl(C1-C6)alkyl, (C2-C9)heterocycloalkyl, (C4-C9)spiroheterocycloalkyl, (C1-C6)alkoxy(C1-C6)alkyl, (C6-C10)aryl(C1-C6)alkyl, (C2-C9)heteroaryl, (C2-C9)heteroaryl(C1-C6)alkyl, (C4-C9)bicycloalkyl, wherein the alkyl, cycloalkyl, heterocycloalkyl, aryl, heteroaryl and bicycloalkyl groups are optionally substituted with one to four halo, (C1-C6)alkyl, (C3-C6)cycloalkyl, (C1-C6)acyl, (C6-C10)aryl, (C1-C6)alkoxy, cyano, oxo or hydroxy.

2. Compound according to claim 1, wherein:
R³, R⁴ and R⁵ are hydrogen; and
R⁶ is selected from the group consisting of (C6-C10)aryl or (C2-C9)heteroaryl, wherein the aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino.

3. Compound according to claim 1, wherein:
R³, R⁴ and R⁵ are hydrogen; and
when X is carbon, R⁷ is R^{7′} and R^{7″}, wherein R^{7′} is selected from the group consisting of hydrogen, halo or (C1-C6)alkyl, and wherein R^{7″} is selected from the group consisting of hydrogen, (C1-C6)alkyl, (C6-C10)aryl(C1-C6)alkyl, (C2-C9)heteroaryl(C1-C6)alkyl, wherein the aryl and heteroaryl groups are optionally substituted with one to three (C1-C6)alkyl;
when X is nitrogen, R⁷ is selected from the group consisting of hydrogen, (C1-C6)alkyl, (C6-C10)aryl(C1-C6)alkyl, (C2-C9)heteroaryl(C1-C6)alkyl, wherein the aryl and heteroaryl groups are optionally substituted with one to three (C1-C6)alkyl or wherein R⁷ is R⁸M−, wherein:
R⁸M- is selected from the group consisting of R⁸—O—C(O)—, R⁸—NH—C(O)— or R⁸—S(O)₂—; and
R⁸ is selected from the group consisting of (C1-C6)alkyl, (C3-C10)cycloalkyl, (C3-C10)cycloalkyl(C1-C6)alkyl, (C2-C9)heterocycloalkyl, (C4-C9)spiroheterocycloalkyl, (C1-C6)alkoxy(C1-C6)alkyl, (C6-C10)aryl(C1-C6)alkyl, (C2-C9)heteroaryl, (C2-C9)heteroaryl(C1-C6)alkyl, (C4-C9)bicycloalkyl, wherein the alkyl, cycloalkyl, heterocycloalkyl, aryl, heteroaryl and bicycloalkyl groups are optionally substituted with one to four halo, (C1-C6)alkyl, (C3-C6)cycloalkyl, (C1-C6)acyl, (C6-C10)aryl, (C1-C6)alkoxy, cyano, oxo or hydroxy.

4. Compound according to claim 1, wherein:
n is 1, 2 or 3;

5. Compound according to claim 1, wherein when X is nitrogen, R⁷ is not hydrogen.

6. Compound having the formula (II): 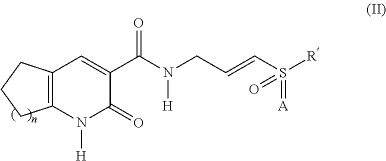
or the pharmaceutical acceptable salt thereof, wherein:
n is 1, 2 or 3;
A is oxygen or nitrogen; and
R′ is selected from the group consisting of (C6-C10)aryl or (C2-C9)heteroaryl, wherein the aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino.

7. Compound according to claim 6, wherein (C6-C10)aryl is phenyl or (C2-C9)heteroaryl is pyridinyl, wherein the phenyl and pyridinyl groups are optionally substituted with one to three fluorine, chlorine, bromine or methoxy.

8. Compound having the formula (III): 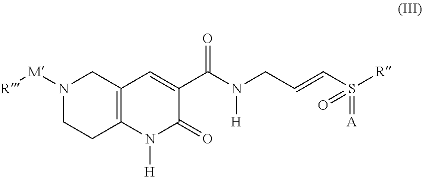
or the pharmaceutical acceptable salt thereof, wherein:
A is oxygen or nitrogen;
R″ is selected from the group consisting of (C6-C10)aryl or (C2-C9)heteroaryl, wherein the aryl and heteroaryl groups are optionally substituted with one to four halo, (C1-C6)alkyl, (C1-C6)alkoxy, (C3-C6)cycloalkyl, (C2-C9)heterocycloalkyl, (C3-C6)cycloalkoxy, (C6-C10)aryl, (C6-C10)aryloxy, cyano, primary, secondary or tertiary amino;
M′ is carbon or —O—C(O)—; and
R′″ is selected from the group consisting of (C1-C6)alkyl, (C3-C10)cycloalkyl, (C3-C10)cycloalkyl(C1-C6)alkyl, (C2-C9)heterocycloalkyl, (C4-C9)spiroheterocycloalkyl, (C1-C6)alkoxy(C1-C6)alkyl, (C6-C10)aryl(C1-C6)alkyl, (C2-C9)heteroaryl, (C2-C9)heteroaryl(C1-C6)alkyl, (C4-C9)bicycloalkyl, wherein the alkyl, cycloalkyl, heterocycloalkyl, aryl, heteroaryl and bicycloalkyl groups are optionally substituted with one to four halo, (C1-C6)alkyl, (C3-C6)cycloalkyl, (C1-C6)acyl, (C6-C10)aryl, (C1-C6)alkoxy, cyano, oxo or hydroxy.

9. Compound according to claim 1, wherein:
A is nitrogen.

10. Compound according to claim 1, wherein the compound is selected from the group consisting of:
N-[(2E)-3-(benzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-6-benzyl-2-oxo-1,2,5,6,7,8-hexahydro-1,6-naphthyridine-3-carboxamide;
N-[(2Z)-3-(benzenesulfonyl)-3-fluoroprop-2-en-1-yl]-2-oxo-1,2,5,6,7,8-hexahydroquinoline-3-carboxamide;
N-[(2E)-3-(benzenesulfonyl)-3-fluoroprop-2-en-1-yl]-2-oxo-1,2,5,6,7,8-hexahydroquinoline-3-carboxamide;
N-[(2Z)-3-(benzenesulfonyl)prop-2-en-1-yl]-2-oxo-1,2,5,6,7,8-hexahydroquinoline-3-carboxamide;
tert-butyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-1,2,5,6,7,8-hexahydro-1,6-naphthyridine-3-carboxamide;
N-[(2E)-3-(benzenesulfonyl)prop-2-en-1-yl]-2-oxo-5-phenyl-1,2-dihydropyridine-3-carboxamide;
N-[(3E)-4-(benzenesulfonyl)-2-methylbut-3-en-2-yl]-2-oxo-1,2,5,6,7,8-hexahydroquinoline-3-carboxamide;
N-{1-[(E)-2-(benzenesulfonyl)ethenyl]cyclopropyl}-2-oxo-1,2,5,6,7,8-hexahydroquinoline-3-carboxamide;
6-acetyl-N-[(2E)-3-(benzenesulfonyl)prop-2-en-1-yl]-2-oxo-1,2,5,6,7,8-hexahydro-1,6-naphthyridine-3-carboxamide;
N-[(2E)-3-(benzenesulfonyl)prop-2-en-1-yl]-6-(3-methoxypropanoyl)-2-oxo-1,2,5,6,7,8-hexahydro-1,6-naphthyridine-3-carboxamide;
N-[(3E)-4-(benzenesulfonyl)but-3-en-2-yl]-2-oxo-1,2,5,6,7,8-hexahydroquinoline-3-carboxamide;
N-[(2E)-3-(benzenesulfonyl)prop-2-en-1-yl]-6-(2-methoxyethanesulfonyl)-2-oxo-1,2,5,6,7,8-hexahydro-1,6-naphthyridine-3-carboxamide;
N-[(2E)-3-methanesulfonylprop-2-en-1-yl]-2-oxo-1,2,5,6,7,8-hexahydroquinoline-3-carboxamide;
6-[2-(adamantan-1-yl)acetyl]-N-[(2E)-3-(benzenesulfonyl)prop-2-en-1-yl]-2-oxo-1,2,5,6,7,8-hexahydro-1,6-naphthyridine-3-carboxamide;
N-[(2E)-3-(benzenesulfonyl)prop-2-en-1-yl]-2-oxo-6-[(1 r,4r)-4-methoxycyclohexanecarbonyl]-1,2,5,6,7,8-hexahydro-1,6-naphthyridine-3-carboxamide;
N-[(2E)-3-(benzenesulfonyl)prop-2-en-1-yl]-6-(4,4-difluorocyclohexanecarbonyl)-2-oxo-1,2,5,6,7,8-hexahydro-1,6-naphthyridine-3-carboxamide;
N-[(2E)-3-(benzenesulfonyl)prop-2-en-1-yl]-6-cyclopropanecarbonyl-2-oxo-1,2,5,6,7,8-hexahydro-1,6-naphthyridine-3-carboxamide;
N-[(2E)-3-(benzenesulfonyl)prop-2-en-1-yl]-6-{bicyclo[2.2.1]heptane-1-carbonyl}-2-oxo-1,2,5,6,7,8-hexahydro-1,6-naphthyridine-3-carboxamide;
N-[(2E)-3-(benzenesulfonyl)prop-2-en-1-yl]-6-(3,3-difluorocyclobutanecarbonyl)-2-oxo-1,2,5,6,7,8-hexahydro-1,6-naphthyridine-3-carboxamide;
N-[(2E)-3-(benzenesulfonyl)prop-2-en-1-yl]-6-(1-cyanocyclobutanecarbonyl)-2-oxo-1,2,5,6,7,8-hexahydro-1,6-naphthyridine-3-carboxamide;
N-[(2E)-3-(benzenesulfonyl)prop-2-en-1-yl]-2-oxo-6-(2-phenyl-2H-1,2,3-triazole-4-carbonyl)-1,2,5,6,7,8-hexahydro-1,6-naphthyridine-3-carboxamide;
N-[(2E)-3-(benzenesulfonyl)prop-2-en-1-yl]-2-oxo-6-(oxolane-3-carbonyl)-1,2,5,6,7,8-hexahydro-1,6-naphthyridine-3-carboxamide;
N-[(2E)-3-(benzenesulfonyl)prop-2-en-1-yl]-6-{3-methylbicyclo[1.1.1]pentane-1-carbonyl}-2-oxo-1,2,5,6,7,8-hexahydro-1,6-naphthyridine-3-carboxamide;
N-[(2E)-3-(4-fluorobenzenesulfonyl)prop-2-en-1-yl]-2-oxo-1,2,5,6,7,8-hexahydroquinoline-3-carboxamide;
N-[(2E)-3-(cyclopentanesulfonyl)prop-2-en-1-yl]-2-oxo-1,2,5,6,7,8-hexahydroquinoline-3-carboxamide;
2-oxo-N-[(2E)-3-(propane-2-sulfonyl)prop-2-en-1-yl]-1,2,5,6,7,8-hexahydroquinoline-3-carboxamide;
N-[(2E)-3-(3-fluoro-4-methoxybenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-6-(1-methylcyclobutanecarbonyl)-2-oxo-1,2,5,6,7,8-hexahydro-1,6-naphthyridine-3-carboxamide;
N-[(2E)-3-(benzenesulfonyl)prop-2-en-1-yl]-6-(4-cyclopropyl-1,3-thiazole-2-carbonyl)-2-oxo-1,2,5,6,7,8-hexahydro-1,6-naphthyridine-3-carboxamide;
N-[(2E)-3-(benzenesulfonyl)prop-2-en-1-yl]-6-(2-cyclopropylacetyl)-2-oxo-1,2,5,6,7,8-hexahydro-1,6-naphthyridine-3-carboxamide;
2-oxo-N-[(2Z)-3-(propane-2-sulfonyl)prop-2-en-1-yl]-1,2,5,6,7,8-hexahydroquinoline-3-carboxamide;
N-[(2E)-3-(4-chlorobenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-2-oxo-6-(1-phenylpyrrolidine-3-carbonyl)-1,2,5,6,7,8-hexahydro-1,6-naphthyridine-3-carboxamide;
2-methylpropyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(2-fluorobenzenesulfonyl)prop-2-en-1-yl]carbamoyl}-2-oxo-1,2,5,6,7,8-hexahydro-1,6-naphthyridine-6-carboxylate;
N-[(2E)-3-(2-methylpropane-2-sulfonyl)prop-2-en-1-yl]-2-oxo-1,2,5,6,7,8-hexahydroquinoline-3-carboxamide;
N-[(2E)-3-(2-fluorobenzenesulfonyl)prop-2-en-1-yl]-2-oxo-1,2,5,6,7,8-hexahydroquinoline-3-carboxamide;
tert-butyl 3-{[(2E)-3-(4-fluorobenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-6-(3,3-dimethylbutanoyl)-2-oxo-1,2,5,6,7,8-hexahydro-1,6-naphthyridine-3-carboxamide;
tert-butyl 3-{[(2E)-3-(3-fluoro-4-methoxybenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-6-[(1-methyl-1H-indazol-4-yl)methyl]-2-oxo-1,2,5,6,7,8-hexahydro-1,6-naphthyridine-3-carboxamide;
2-(4-chlorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
tert-butyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-6-[(pyridin-3-yl)methyl]-1,2,5,6,7,8-hexahydro-1,6-naphthyridine-3-carboxamide;
N-[(2E)-3-(benzenesulfonyl)prop-2-en-1-yl]-2-oxo-6-[(pyridazin-3-yl)methyl]-1,2,5,6,7,8-hexahydro-1,6-naphthyridine-3-carboxamide;
N-[(2E)-3-(benzenesulfonyl)prop-2-en-1-yl]-6-(3-methoxy-3-methylbutanoyl)-2-oxo-1,2,5,6,7,8-hexahydro-1,6-naphthyridine-3-carboxamide;
N-[(2E)-3-(4-chlorobenzenesulfonyl)prop-2-en-1-yl]-2-oxo-1,2-dihydroquinoline-3-carboxamide;
6-benzyl-N-[(2E)-3-(4-chlorobenzenesulfonyl)prop-2-en-1-yl]-2-oxo-1,2,5,6,7,8-hexahydro-1,6-naphthyridine-3-carboxamide;
N-[(2E)-3-(4-chlorobenzenesulfonyl)prop-2-en-1-yl]-2-oxo-5-phenyl-1,2-dihydropyridine-3-carboxamide;
N-[(2Z)-3-(2-fluorobenzenesulfonyl)but-2-en-1-yl]-2-oxo-1,2,5,6,7,8-hexahydroquinoline-3-carboxamide;
N-[(2E)-3-(2-fluorobenzenesulfonyl)but-2-en-1-yl]-2-oxo-1,2,5,6,7,8-hexahydroquinoline-3-carboxamide;
cyclopropylmethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclobutyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(5-methyl-1,3,4-oxadiazol-2-yl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1,1,1-trifluoropropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-difluorocyclopropyl)methyl 3-{[(2E)-3-(benzenesulfonyl)prop-2-en-1-yl]carbamoyl}-2-oxo-1,2,5,6,7,8-hexahydro-1,6-naphthyridine-6-carboxylate;
oxetan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
1-methoxypropan-2-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
2-fluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
2,2-difluoroethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclopropyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-6-(2-phenylcyclopropanecarbonyl)-1,2,5,6,7,8-hexahydro-1,6-naphthyridine-3-carboxamide;
2-oxaspiro[3.5]nonan-7-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
2-methoxyethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2,4-difluorobenzenesulfonyl)prop-2-en-1-yl]-2-oxo-1,2,5,6,7,8-hexahydroquinoline-3-carboxamide;
N-[(2E)-3-(2,4-dichlorobenzenesulfonyl)prop-2-en-1-yl]-2-oxo-1,2,5,6,7,8-hexahydroquinoline-3-carboxamide;
(3S)-oxolan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
(3R)-oxolan-3-yl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2-chloro-4-fluorobenzenesulfonyl)prop-2-en-1-yl]-2-oxo-1,2,5,6,7,8-hexahydroquinoline-3-carboxamide;
N-[(2E)-3-(2,4-difluorobenzenesulfonyl)prop-2-en-1-yl]-2-oxo-5-phenyl-1,2-dihydropyridine-3-carboxamide;
N-[(2E)-3-(2,4-dichlorobenzenesulfonyl)prop-2-en-1-yl]-2-oxo-5-phenyl-1,2-dihydropyridine-3-carboxamide;
N-[(2E)-3-(2-chloro-4-fluorobenzenesulfonyl)prop-2-en-1-yl]-2-oxo-5-phenyl-1,2-dihydropyridine-3-carboxamide;
N-[(2E)-3-(2,4-difluorobenzenesulfonyl)prop-2-en-1-yl]-2-oxo-1H,2H,5H,6H,7H,8H,9H-cyclohepta[b]pyridine-3-carboxamide;
N-[(2E)-3-(4-fluorobenzenesulfonyl)prop-2-en-1-yl]-2-oxo-1H,2H,5H,6H,7H,8H,9H-cyclohepta[b]pyridine-3-carboxamide;
N-[(2E)-3-(4-chlorobenzenesulfonyl)prop-2-en-1-yl]-2-oxo-1H,2H,5H,6H,7H,8H,9H-cyclohepta[b]pyridine-3-carboxamide;
N-[(2Z)-3-(4-chlorobenzenesulfonyl)but-2-en-1-yl]-2-oxo-1,2,5,6,7,8-hexahydroquinoline-3-carboxamide;
N-[(2E)-3-(4-chlorobenzenesulfonyl)but-2-en-1-yl]-2-oxo-1,2,5,6,7,8-hexahydroquinoline-3-carboxamide;
N-[(2E)-3-(3-fluoro-4-methoxybenzenesulfonyl)prop-2-en-1-yl]-2-oxo-1H,2H,5H,6H,7H,8H,9H-cyclohepta[b]pyridine-3-carboxamide;
N-[(2E)-3-(benzenesulfonyl)prop-2-en-1-yl]-2-oxo-1H,2H,5H,6H,7H,8H,9H-cyclohepta[b]pyridine-3-carboxamide;
N-[(2Z)-3-(4-chlorobenzenesulfonyl)but-2-en-1-yl]-2-oxo-5-phenyl-1,2-dihydropyridine-3-carboxamide;
N-[(2E)-3-(4-chlorobenzenesulfonyl)but-2-en-1-yl]-2-oxo-5-phenyl-1,2-dihydropyridine-3-carboxamide;
N-[(2E)-3-(2-chlorobenzenesulfonyl)prop-2-en-1-yl]-2-oxo-5-phenyl-1,2-dihydropyridine-3-carboxamide;
N-[(2E)-3-(3-fluorobenzenesulfonyl)prop-2-en-1-yl]-2-oxo-1,2,5,6,7,8-hexahydroquinoline-3-carboxamide;
N-[(2E)-3-(4-methoxybenzenesulfonyl)prop-2-en-1-yl]-2-oxo-1,2,5,6,7,8-hexahydroquinoline-3-carboxamide;
2-(2-oxopiperidin-1-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
N-[(2E)-3-(2-chlorobenzenesulfonyl)prop-2-en-1-yl]-2-oxo-1,2,5,6,7,8-hexahydroquinoline-3-carboxamide;
cyclopentyl 3-{[(2E)-3-(benzenesulfonyl)prop-2-en-1-yl]carbamoyl}-2-oxo-1,2,5,6,7,8-hexahydro-1,6-naphthyridine-6-carboxylate;
(2,2-dimethylcyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
(2,2-difluorocyclopropyl)methyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
oxetan-3-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(4-chlorobenzenesulfonyl)-3-fluoroprop-2-en-1-yl]-2-oxo-1,2,5,6,7,8-hexahydroquinoline-3-carboxamide;
N-[(2Z)-3-(4-chlorobenzenesulfonyl)-3-fluoroprop-2-en-1-yl]-2-oxo-1,2,5,6,7,8-hexahydroquinoline-3-carboxamide;
cyclopropylmethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(2,4-difluorobenzenesulfonyl)prop-2-en-1-yl]-2-oxo-1H,2H,5H,6H,7H-cyclopenta[b]pyridine-3-carboxamide;
N-[(2E)-3-(benzenesulfonyl)prop-2-en-1-yl]-2-oxo-1H,2H,5H,6H,7H-cyclopenta[b]pyridine-3-carboxamide;
2-(furan-2-yl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
cyclohexyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1-(2,6-difluorophenyl)ethyl 3-{[(2E)-3-(benzenesulfonyl)prop-2-en-1-yl]carbamoyl}-2-oxo-1,2,5,6,7,8-hexahydro-1,6-naphthyridine-6-carboxylate;
1-acetylpiperidin-4-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
2-fluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
1-methoxypropan-2-yl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-[(5-chloropyridin-2-yl)sulfonyl]prop-2-en-1-yl]-2-oxo-1,2,5,6,7,8-hexahydroquinoline-3-carboxamide;
2-oxo-N-[(2E)-3-(thiophene-2-sulfonyl)prop-2-en-1-yl]-1,2,5,6,7,8-hexahydroquinoline-3-carboxamide;
cyclopropylmethyl 3-{[(2E)-3-[(5-chloropyridin-2-yl)sulfonyl]prop-2-en-1-yl]carbamoyl}-2-oxo-1,2,5,6,7,8-hexahydro-1,6-naphthyridine-6-carboxylate;
2,2-difluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
2,2,2-trifluoroethyl 3-{[(2E)-3-(4-chlorobenzenesulfonyl)prop-2-en-1-yl]carbamoyl}-2-oxo-1,2,5,6,7,8-hexahydro-1,6-naphthyridine-6-carboxylate;
N-[(2E)-3-(4-bromobenzenesulfonyl)prop-2-en-1-yl]-2-oxo-1,2,5,6,7,8-hexahydroquinoline-3-carboxamide;
N-[(2E)-3-(3,4-dimethoxybenzenesulfonyl)prop-2-en-1-yl]-2-oxo-1,2,5,6,7,8-hexahydroquinoline-3-carboxamide;
N-[(2E)-3-(benzenesulfonyl)prop-2-en-1-yl]-2-oxo-6-(propan-2-yl)-1,2,5,6,7,8-hexahydroquinoline-3-carboxamide;
cyclopropylmethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-(benzenesulfonyl)prop-2-en-1-yl]-2-oxo-1,2-dihydro-1,8-naphthyridine-3-carboxamide;
N-[(2E)-3-(benzenesulfonyl)prop-2-en-1-yl]-6,6-dimethyl-2-oxo-1,2,5,6,7,8-hexahydroquinoline-3-carboxamide;
N-[(2E)-3-(benzenesulfonyl)prop-2-en-1-yl]-6,6-difluoro-2-oxo-1,2,5,6,7,8-hexahydroquinoline-3-carboxamide;
N3-[(2E)-3-(4-chlorobenzenesulfonyl)prop-2-en-1-yl]-N6-(cyclopropylmethyl)-2-oxo-1,2,5,6,7,8-hexahydro-1,6-naphthyridine-3,6-dicarboxamide;
N3-[(2E)-3-(4-chlorobenzenesulfonyl)prop-2-en-1-yl]-N6-cyclopropyl-2-oxo-1,2,5,6,7,8-hexahydro-1,6-naphthyridine-3,6-dicarboxamide;
N6-tert-butyl-N3-[(2E)-3-(4-chlorobenzenesulfonyl)prop-2-en-1-yl]-2-oxo-1,2,5,6,7,8-hexahydro-1,6-naphthyridine-3,6-dicarboxamide;
N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide;
N-[(2E)-3-(4-tert-butylbenzenesulfonyl)prop-2-en-1-yl]-2-oxo-1,2,5,6,7,8-hexahydroquinoline-3-carboxamide;
N-[(2E)-3-(3,4-dimethylbenzenesulfonyl)prop-2-en-1-yl]-2-oxo-1,2,5,6,7,8-hexahydroquinoline-3-carboxamide;
N-[(2E)-3-[(3,4-dimethoxyphenyl)(imino)oxo-λ⁶-sulfanyl]prop-2-en-1-yl]-2-oxo-1,2,5,6,7,8-hexahydroquinoline-3-carboxamide;
N-[(2E)-3-(benzenesulfonyl)(1,1-²H2)prop-2-en-1-yl]-2-oxo-1,2,5,6,7,8-hexahydroquinoline-3-carboxamide;
N-[(2E)-3-[4-(dimethylamino)benzenesulfonyl]prop-2-en-1-yl]-2-oxo-1,2,5,6,7,8-hexahydroquinoline-3-carboxamide;
N-[(2E)-3-(4-cyclopropylbenzenesulfonyl)prop-2-en-1-yl]-2-oxo-1,2,5,6,7,8-hexahydroquinoline-3-carboxamide;
N-[(2E)-3-(4-cyanobenzenesulfonyl)prop-2-en-1-yl]-2-oxo-1,2,5,6,7,8-hexahydroquinoline-3-carboxamide;
N-[(2E)-3-[imino(4-methoxyphenyl)oxo-λ⁶-sulfanyl]prop-2-en-1-yl]-2-oxo-1,2,5,6,7,8-hexahydroquinoline-3-carboxamide;
N-[(2E)-3-[(3,4-dimethylphenyl)(imino)oxo-λ⁶-sulfanyl]prop-2-en-1-yl]-2-oxo-1,2,5,6,7,8-hexahydroquinoline-3-carboxamide;
6,6-difluoro-N-[(2E)-3-[(4-fluorophenyl)(imino)oxo-λ⁶-sulfanyl]prop-2-en-1-yl]-2-oxo-1,2,5,6,7,8-hexahydroquinoline-3-carboxamide;
cyclopropylmethyl 3-{[(2E)-3-[(4-fluorophenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)phenyl-λ⁶-sulfanyl]prop-2-en-1-yl]-2-oxo-1,2,5,6,7,8-hexahydroquinoline-3-carboxamide;
N-[(2E)-3-[(4-tert-butylphenyl)(imino)oxo-λ⁶-sulfanyl]prop-2-en-1-yl]-2-oxo-1,2,5,6,7,8-hexahydroquinoline-3-carboxamide;
N-[(2E)-3-[(4-ethoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3,5-dimethylphenyl)oxo-λ⁶-sulfanyl]prop-2-en-1-yl]-2-oxo-1,2,5,6,7,8-hexahydroquinoline-3-carboxamide;
N-[(2E)-3-[imino(3-methoxyphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(trifluoromethoxy)phenyl]-λ⁶-sulfanyl]prop-2-en-1-yl]-2-oxo-1,2,5,6,7,8-hexahydroquinoline-3-carboxamide;
N-[(2E)-3-[(4-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[(2-fluorophenyl)(imino)oxo-λ⁶-sulfanyl]prop-2-λ⁶-1-yl]-2-oxo-1,2,5,6,7,8-hexahydroquinoline-3-carboxamide;
N-[(2E)-3-[(3-chloro-4-methoxyphenyl)(imino)oxo-λ⁶-sulfanyl]prop-2-en-1-yl]-2-oxo-1,2,5,6,7,8-hexahydroquinoline-3-carboxamide;
2,2-difluoroethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[(3-chlorophenyl)(imino)oxo-λ⁶-sulfanyl]prop-2-en-1-yl]-2-oxo-1,2,5,6,7,8-hexahydroquinoline-3-carboxamide;
N-[(2E)-3-[(2,4-difluorophenyl)(imino)oxo-λ⁶-sulfanyl]prop-2-en-1-yl]-2-oxo-1,2,5,6,7,8-hexahydroquinoline-3-carboxamide;
N-[(2E)-3-[(4-cyanophenyl)(imino)oxo-λ⁶-sulfanyl]prop-2-en-1-yl]-2-oxo-1,2,5,6,7,8-hexahydroquinoline-3-carboxamide;
N-[(2E)-3-[imino(4-methoxy-3-methylphenyl)oxo-λ⁶-sulfanyl]prop-2-en-1-yl]-2-oxo-1,2,5,6,7,8-hexahydroquinoline-3-carboxamide;
N-[(2E)-3-[imino(oxo)[4-(propan-2-yloxy)phenyl]-λ⁶-sulfanyl]prop-2-en-1-yl]-2-oxo-1,2,5,6,7,8-hexahydroquinoline-3-carboxamide;
N-[(2E)-3-[imino(4-methoxy-2-methylphenyl)oxo-λ⁶-sulfanyl]prop-2-en-1-yl]-2-oxo-1,2,5,6,7,8-hexahydroquinoline-3-carboxamide;
cyclopropylmethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
2-fluoroethyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
2-fluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
2,2-difluoroethyl 3-{[(2E)-3-[imino(oxo)phenyl-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)(4-phenoxyphenyl)-λ⁶-sulfanyl]prop-2-en-1-yl]-2-oxo-1,2,5,6,7,8-hexahydroquinoline-3-carboxamide;
N-[(2E)-3-{[4-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-6-methyl-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1,2-dihydroquinoline-3-carboxamide;
N-[(2E)-3-[(3-fluoro-4-methoxyphenyl)(imino)oxo-λ⁶-sulfanyl]prop-2-en-1-yl]-2-oxo-6-(propan-2-yl)-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-6-methoxy-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-6-(2,2,2-trifluoroethyl)-1,2,5,6,7,8-hexahydro-1,6-naphthyridine-3-carboxamide
N-[(2E)-3-[(3-fluoro-4-methoxyphenyl)(imino)oxo-λ⁶-sulfanyl]prop-2-en-1-yl]-2-oxo-1H,2H,5H,7H,8H-pyrano[4,3-b]pyridine-3-carboxamide;
N-[(2E)-3-[imino(oxo)(3-phenoxyphenyl)-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-6-(trifluoromethyl)-1,2,5,6,7,8-hexahydroquinoline-3-carboxamide;
6-tert-butyl-N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-5-phenyl-1,2-dihydropyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H,8H,9H-cyclohepta[b]pyridine-3-carboxamide;
N-[(2E)-3-[(3-fluoro-4-methoxyphenyl)(imino)oxo-λ⁶-sulfanyl]prop-2-en-1-yl]-2-oxo-1H,2H,5H,6H,7H-cyclopenta[b]pyridine-3-carboxamide;
tert-butyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
6-benzyl-N-[(2E)-3-[(3-fluoro-4-methoxyphenyl)(imino)oxo-λ⁶-sulfanyl]prop-2-en-1-yl]-2-oxo-1,2,5,6,7,8-hexahydro-1,6-naphthyridine-3-carboxamide;
N-[(2E)-3-[(3-fluoro-4-methoxyphenyl)(imino)oxo-λ⁶-sulfanyl]prop-2-en-1-yl]-6-[(4-methoxyphenyl)methyl]-2-oxo-1,2,5,6,7,8-hexahydro-1,6-naphthyridine-3-carboxamide;
4,4-difluorocyclohexyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
cyclohexyl 3-{[(2E)-3-[(3-fluoro-4-methoxyphenyl)(imino)oxo-λ⁶-sulfanyl]prop-2-en-1-yl]carbamoyl}-2-oxo-1,2,5,6,7,8-hexahydro-1,6-naphthyridine-6-carboxylate;
N-[(2E)-3-[imino(oxo)[4-(trifluoromethyl)phenyl]-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-8,8-dimethyl-2-oxo-1,2,5,6,7,8-hexahydroquinoline-3-carboxamide;
6-(cyclopropylmethyl)-N-[(2E)-3-[(3-fluoro-4-methoxyphenyl)(imino)oxo-λ⁶-sulfanyl]prop-2-en-1-yl]-2-oxo-1,2,5,6,7,8-hexahydro-1,6-naphthyridine-3-carboxamide;
N-[(2E)-3-({[1,1′-biphenyl]-4-yl}(imino)oxo-λ⁶-sulfanyl)prop-2-en-1-yl]-2-oxo-1,2,5,6,7,8-hexahydroquinoline-3-carboxamide;
N-[(2E)-3-[benzyl(imino)oxo-λ⁶-sulfanyl]prop-2-en-1-yl]-2-oxo-1,2,5,6,7,8-hexahydroquinoline-3-carboxamide;
N-[(2E)-3-[(3-fluoro-4-methoxyphenyl)(imino)oxo-λ⁶-sulfanyl]prop-2-en-1-yl]-2-oxo-1,2-dihydro-1,8-naphthyridine-3-carboxamide;
N-[(2E)-3-[(3-fluoro-4-methoxyphenyl)(imino)oxo-λ⁶-sulfanyl]prop-2-en-1-yl]-2-oxo-1,2-dihydro-1,6-naphthyridine-3-carboxamide;
6-fluoro-N-[(2E)-3-[(3-fluoro-4-methoxyphenyl)(imino)oxo-λ⁶-sulfanyl]prop-2-en-1-yl]-2-oxo-1,2,5,6,7,8-hexahydroquinoline-3-carboxamide;
N-[(2E)-3-{imino[4-(morpholin-4-yl)phenyl]oxo-λ⁶-sulfanyl}prop-2-en-1-yl]-2-oxo-1,2,5,6,7,8-hexahydroquinoline-3-carboxamide;
N-[(2E)-3-{[3-cyano-4-(piperidin-1-yl)phenyl](imino)oxo-λ⁶-sulfanyl}prop-2-en-1-yl]-2-oxo-1,2,5,6,7,8-hexahydroquinoline-3-carboxamide;
N-[(2E)-3-[imino(oxo)[4-(piperidin-1-yl)phenyl]-λ⁶-sulfanyl]prop-2-en-1-yl]-2-oxo-1,2,5,6,7,8-hexahydroquinoline-3-carboxamide;
N-[(2E)-3-[imino(oxo)[4-(pyrrolidin-1-yl)phenyl]-λ⁶-sulfanyl]prop-2-en-1-yl]-2-oxo-1,2,5,6,7,8-hexahydroquinoline-3-carboxamide;
N-[(2E)-3-{imino[4-(methylamino)phenyl]oxo-λ⁶-sulfanyl}prop-2-en-1-yl]-2-oxo-1,2,5,6,7,8-hexahydroquinoline-3-carboxamide;
N-[(2E)-3-{[3-cyano-4-(morpholin-4-yl)phenyl](imino)oxo-λ⁶-sulfanyl}prop-2-en-1-yl]-2-oxo-1,2,5,6,7,8-hexahydroquinoline-3-carboxamide;
N-[(2E)-3-[imino(oxo)[4-(2-oxopiperidin-1-yl)phenyl]-λ⁶-sulfanyl]prop-2-en-1-yl]-2-oxo-1,2,5,6,7,8-hexahydroquinoline-3-carboxamide;
N-[(2E)-3-[imino(1-methyl-1,2,3,4-tetrahydroquinolin-6-yl)oxo-λ⁶-sulfanyl]prop-2-en-1-yl]-2-oxo-1,2,5,6,7,8-hexahydroquinoline-3-carboxamide;
N-[(2E)-3-[(4-acetamidophenyl)(imino)oxo-λ⁶-sulfanyl]prop-2-en-1-yl]-2-oxo-1,2,5,6,7,8-hexahydroquinoline-3-carboxamide;
N-[(2E)-3-[(4-cyclopropoxyphenyl)(imino)oxo-λ⁶-sulfanyl]prop-2-en-1-yl]-2-oxo-1,2,5,6,7,8-hexahydroquinoline-3-carboxamide;
N-[(2E)-3-{[3-(dimethylamino)phenyl](imino)oxo-λ⁶-sulfanyl}prop-2-en-1-yl]-2-oxo-1,2,5,6,7,8-hexahydroquinoline-3-carboxamide;
N-[(2E)-3-[(4-cyclohexylphenyl)(imino)oxo-λ⁶-sulfanyl]prop-2-en-1-yl]-2-oxo-1,2,5,6,7,8-hexahydroquinoline-3-carboxamide;
N-[(2E)-3-[(2,3-dihydro-1-benzofuran-5-yl)(imino)oxo-λ⁶-sulfanyl]prop-2-en-1-yl]-2-oxo-1,2,5,6,7,8-hexahydroquinoline-3-carboxamide;
N-[(2E)-3-[imino(oxo)[3-(trifluoromethoxy)phenyl]-λ⁶-sulfanyl]prop-2-en-1-yl]-2-oxo-1,2,5,6,7,8-hexahydroquinoline-3-carboxamide; and
N-[(2E)-3-[(3-fluoro-4-methoxyphenyl)(imino)oxo-λ⁶-sulfanyl]prop-2-en-1-yl]-2-oxo-1,2-dihydropyridine-3-carboxamide.

11. A method of treatment of cancer, said method comprising administering to a subject in need thereof a compound according to claim 1.

12. A method of treatment according to claim 11, wherein said method is combined with radiotherapy, agents suitable for use in chemotherapy, and/or agents suitable for use in immunotherapy, in particular wherein use of the compound is combined with chemotherapeutic agents, immunotherapeutic agents, targeted therapy and/or radiotherapy that cause increased levels of tumor necrosis factor (TNF).

13. (canceled)

14. A method of treatment according to claim 11, wherein the cancer is of melanoma, bladder cancer, colon cancer, pancreatic cancer, ovarian cancer, breast cancer, bone cancer, lung cancer or haematological malignancies.

15. A method of treatment of ovarian cancer, lung cancer or haematological malignancies, said method comprising administering to a subject in need thereof a compound according to claim 1.

16. Method for inhibiting Met1-linked ubiquitination, wherein the method comprises the administration of the compound according to claim 1.

17. Method according to claim 16, wherein the inhibition of the Met1-linked ubiquitination comprises the inhibition of linear ubiquitin (Ub) chain assembly complex (LUBAC), preferably the inhibition of RNF31 (HOIP).

18. Method according to claim 16, wherein the inhibition of the Met1-linked ubiquitination comprises the irreversible or reversible inhibition of linear ubiquitin (Ub) chain assembly complex (LUBAC) via a covalent binding of the compound, preferably the irreversible inhibition of RNF31 (HOIP).

19. (canceled)

20. Pharmaceutical composition comprising the compound according to claim 1.

21. Pharmaceutical composition according to claim 20, wherein the composition further comprises at least one pharmaceutically acceptable carrier.

22. Pharmaceutical composition according to claim 20, wherein the composition further comprises tumor necrosis factor (TNF).
